(19) United States
(12) Patent Application Publication
(10) Pub. No.: US 2026/0021193 A1
(43) Pub. Date: Jan. 22, 2026

(54) IMMUNOCONJUGATES AND METHODS

(71) Applicant: Immunome, Inc., Bothell, WA (US)

(72) Inventors: Xiaojun Han, San Diego, CA (US); Suvi Tuula Marjukka Orr, San Diego, CA (US); Peter Qinhua Huang, San Diego, CA (US); Kevin Duane Bunker, Escondido, CA (US); Kimberlee Fischer, San Diego, CA (US)

(21) Appl. No.: 18/997,978

(22) PCT Filed: Jul. 25, 2023
(86) PCT No.: PCT/US2023/070971

§ 371 Date: Jan. 23, 2025

Related U.S. Application Data
(60) Provisional application No. 63/369,464, filed on Jul. 26, 2022.

(51) Int. Cl. A61K 47/68; A61P 35/00

(52) U.S. Cl. CPC A61K 47/68037; A61K 47/6849; A61K 47/6855; A61K 47/6889; A61P 35/00

(57) ABSTRACT

Immunoconjugates of the Formula (I) include a linking group for linking an antibody targeting ligand (Ab) to a drug (D). Embodiments of such immunoconjugates are useful for delivering the drug to selected cells or tissues, e.g., for the treatment of cancer. Formula (I)

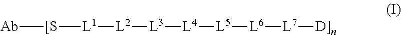

### INCORPORATION BY REFERENCE TO ANY PRIORITY APPLICATIONS

Any and all applications for which a foreign or domestic priority claim is identified in the Application Data Sheet as filed with the present application are hereby expressly incorporated by reference under 37 CFR 1.57, and Rules 4.18 and 20.6, including U.S. Provisional Application No. 63/369,464, filed Jul. 26, 2022.

### SEQUENCE STATEMENT

This application contains a Sequence Listing, which has been submitted electronically and is hereby incorporated by reference in its entirety. The sequence listing, was created on Jul. 21, 2023, is named ZENO144.xml and is approximately 56 kb in size.

### BACKGROUND

### Field

The application relates to conjugates that include a linking group for linking an antibody targeting ligand to a cell-killing moiety (such as a drug), methods of making such conjugates, and methods of using such conjugates to deliver the cell-killing moiety to selected cells or tissues, e.g., for the treatment or inhibition of a cancer.

### Description

A number of antibody-drug conjugates (ADC) have been developed for medical uses. See, e.g., Nejadmoghaddam, M. et al., “Antibody-Drug Conjugates: Possibilities and Challenges”, Avicenna J Med Biotech 11(1), 3-23 (2019). The antibody in the ADC functions as a targeting agent to deliver the drug to a selected cell or tissue such as a cancer cell or tumor. In the United States, the U.S. Food and Drug Administration (FDA) has approved several ADC formulations, including inotuzumab ozogamicin (tradename BESPONSA), gemtuzumab ozogamicin (tradename MYLOTARG), brentuximab vedotin (tradename ADCETRIS), and ado-trastuzumab emtansine (tradename KADCYLA).

U.S. Pat. No. 10,155,821 discloses ADCs in which an antitumor compound is conjugated to an anti-HER2 antibody via a linker. See also U.S. Patent Publication Nos. 2020/0385486 and 2019/0077880. Trastuzumab deruxtecan is an example of an ADC in which an anti-HER2 antibody (trastuzumab) is attached via a cleavable malcimide tetrapeptide linker to an antitumor compound (deruxtecan). The FDA has approved a formulation known as fam-trastuzumab deruxtecan-nxki (tradename ENHERTU) for the treatment of adult patients with unresectable or metastatic HER2-positive breast cancer who have received two or more prior anti-HER2-based regimens in the metastatic setting. FIG. 1 illustrates the manner in which it is believed the linker connects the antibody (mAb) to the drug moiety.

The FDA approvals represent milestones in the ongoing development of therapeutic ADCs. However, there remains a need for improved ADCs to help address the long-felt need for additional options to treat cancer and/or deliver therapeutic payloads to selected cells or tissues.

### SUMMARY

Some embodiments provide an immunoconjugate of Formula (I) that comprises an antibody or an antigen-binding fragment thereof (Ab), a drug moiety (D) and a linker connecting Ab to D. In an embodiment, the immunoconjugate of Formula (I) comprises a drug moiety of the Formula (II).

An embodiment provides an immunoconjugate having Formula (I),

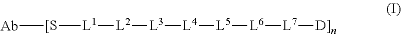

wherein:
Ab can be an antibody or an antigen-binding fragment thereof;
L¹ can be

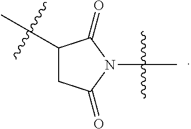 [SMILES: *C1CC(=O)N(*)C1=O]

L² can be absent,

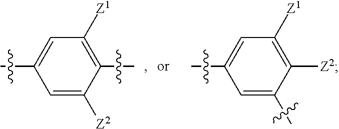

Z¹ and Z² each individually can be hydrogen, halogen, NO₂, —O—(C₁-C₆ alkyl), or C₁-C₆ alkyl;
L³ can be —(CH₂)n¹-C(═O)— or —(CH₂CH₂O)n¹-(CH₂)n¹C(═O)—;
each n¹ independently can be integers of 0 to 12;
L⁴ can be a tetrapeptide residue;
L⁵ can be absent or —[NH(CH₂)n²]n³-;
n² can be an integer of 0 to 6;
n³ can be an integer of 0 to 2;
L⁶ can be absent or

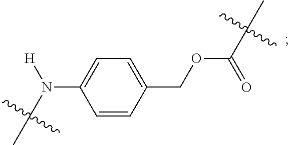 [SMILES: *Nc1ccc(COC(*)=O)cc1]

L⁷ can be absent,

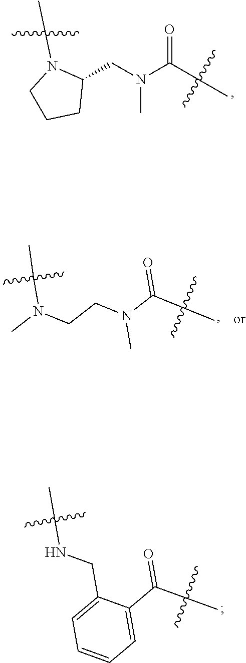

D can be a drug moiety; and
n can be an integer from 1 to 10.

In an embodiment, D in Formula (I) can be a drug moiety of Formula (II) having the structure:

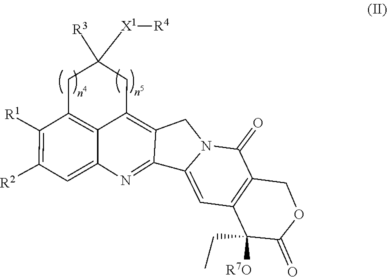

wherein:
R¹ and R² each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² can be taken together to form a ring;
R³ and R⁴ each individually can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and [(CY₂)ₚO(CY₂)_{q}]ₜCY₃, with the proviso that R³ and R⁴ both cannot be hydrogen; or R³ and R⁴, can be taken together with the carbon atom to which they are attached to form a substituted or unsubstituted 4-, 5- or 6-membered heterocyclyl or a 4-, 5- or 6-membered heterocyclyl that can be substituted with an optionally substituted C₁-C₃ alkyl; or one of R³ and R⁴ can be a substituted or an unsubstituted —(C₁-C₆ alkyl)-X², a substituted or an unsubstituted —(C₁-C₆ haloalkyl)-X², a substituted or an unsubstituted —(C₁-C₆ alkenyl)-X², a substituted or an unsubstituted —(C₁-C₆ haloalkenyl)-X², a substituted or an unsubstituted —(C₁-C₆alkynyl)-X², or a substituted or an unsubstituted —(C₁-C₆ haloalkynyl)-X²;
X² can be —OR⁹, —SR⁹ or —NHR⁹;
R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
n⁴ and n⁵ each individually can be 0, 1 or 2, with the proviso that n⁴ and n⁵ cannot both be 0;
each Y individually can be H or halogen;
each m individually can be 1 or 2;
each p individually can be 1, 2, 3, 4, 5, or 6;
each q individually can be 0, 1, 2, 3, 4, 5, or 6;
each t individually can be 1, 2, 3, 4, 5, or 6;
R⁷ can be H, —COR⁸, —CO₂R⁸, —(CO)—NHR⁸, L⁴, L⁵, L⁶, or L⁷;
R⁸ can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ can be H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
each X³ individually can be —H, —OH, —SH, or —NH₂.

An embodiment provides a compound of Formula (IV), or a pharmaceutically acceptable salt thereof, having the structure:

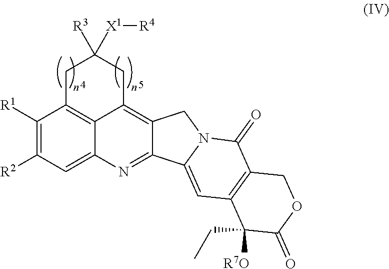

wherein:
R¹ and R² each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), and a substituted or an unsubstituted —O—(C₁-C₆ haloalkyl), —[(CY₂)ₚO(CY₂)_{q}]ₜCY₃, or a substituted or an unsubstituted —O—(CR⁵R⁶)ₘ—O— such that R¹ and R² can be taken together to form a ring;
R³ and R⁴ are each individually can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and [(CY₂)ₚO(CY₂)_{q}]ₜCY₃, with the proviso that R³ and R⁴ both cannot be hydrogen; or R³ and R⁴ can be taken together with the carbon atom to which they are attached to form an unsubstituted 4-, 5- or 6-membered heterocyclyl or a 4-, 5- or 6-membered heterocyclyl that can be substituted with an optionally substituted C₁-C₃ alkyl;
R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
n⁴ and n⁵ each individually can be 0, 1 or 2, with the proviso that n⁴ and n⁵ cannot both be 0;
each Y individually can be H or halogen;
each m individually can be 1 or 2;
each p individually can be 1, 2, 3, 4, 5, or 6;
each q individually can be 0, 1, 2, 3, 4, 5, or 6; and
each t individually can be 1, 2, 3, 4, 5, or 6;
R⁷ can be H, —COR⁸, —CO₂R⁸, or —(CO)—NHR⁸; and
R⁸ can be a substituted or an unsubstituted C₁-C₆ alkyl, a substituted or an unsubstituted C₁-C₆ haloalkyl, or —[(CY₂)ₚO(CY₂)_{q}]ₜCY₃.

An embodiment provides a pharmaceutical composition comprising an immunoconjugate as described herein, a drug compound as described herein, or a pharmaceutically active salt thereof, and a pharmaceutically acceptable carrier, diluent, excipient or combination thereof.

An embodiment provides a method for treating a cancer or a tumor comprising administering an effective amount of an immunoconjugate as described herein, a drug compound as described herein, or a pharmaceutically active salt thereof, or a pharmaceutical composition as described herein, to a subject having the cancer or the tumor.

An embodiment provides a use of an effective amount of an immunoconjugate as described herein, a drug compound as described herein, or a pharmaceutically active salt thereof, or a pharmaceutical composition as described herein, in the manufacture of a medicament for treating a cancer or a tumor.

Some embodiments provide a conjugate of Formula (III) that comprises a functional group M1, a drug moiety (D) and a linker connecting Mi to D. In an embodiment, the conjugate of Formula (III) comprises a drug moiety of the Formula (II).

An embodiment provides a conjugate having Formula (III),

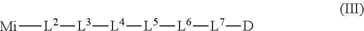

wherein:
Mi can be

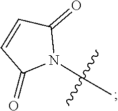 [SMILES: *N1C(=O)C=CC1=O]

L² can be absent,

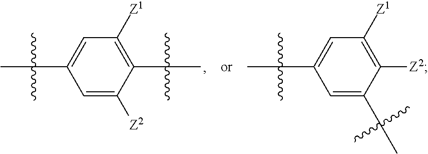

Z¹ and Z² each individually can be hydrogen, halogen, NO₂, —O—(C₁-C₆ alkyl), or C₁-C₆alkyl;
L³ can be —(CH₂)n¹-C(═O)— or —(CH₂CH₂O)n¹-(CH₂)n¹C(═O)—;
each n¹ independently can be integers of 0 to 12;
L⁴ can be a tetrapeptide residue;
L⁵ can be absent or —[NH(CH₂)n²]n³-;
n² can be an integer of 0 to 6;
n³ can be an integer of 0 to 2;
L⁶ can be absent or

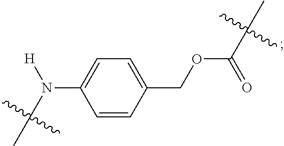 [SMILES: *Nc1ccc(COC(*)=O)cc1]

L⁷ can be absent,

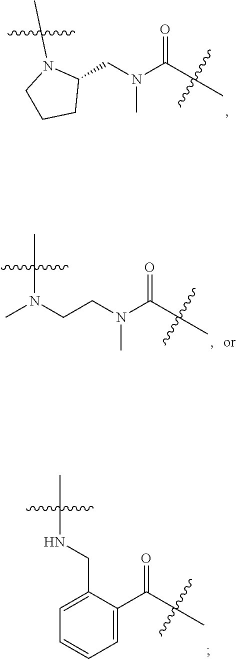

and
D can be a drug moiety.

An embodiment provides a process of producing an immunoconjugate, comprising: reacting an effective amount of a thiol-functionalized antibody or an antigen-binding fragment thereof with a conjugate as described herein under reaction conditions effective to form an immunoconjugate as described herein.

An embodiment provides an immunoconjugate, pharmaceutical composition, method of treatment, use, or process of making as described herein, wherein Ab is an antibody or an antigen-binding fragment thereof comprising:
a) a heavy chain comprising:
VHCDR 1 comprising an amino acid sequence having at least 95% sequence identity to the amino acid sequence of SEQ ID NO:1;
VHCDR 2 comprising an amino acid sequence having at least 95% sequence identity to the amino acid sequence of SEQ ID NO:2; and
VHCDR 3 comprising an amino acid sequence having at least 95% sequence identity to the amino acid sequence of SEQ ID NO:3; and
b) a light chain comprising:
VLCDR 1 comprising an amino acid sequence having at least 95% sequence identity to the amino acid sequence of SEQ ID NO:8;
VLCDR 2 comprising an amino acid sequence having at least 95% sequence identity to the amino acid sequence of AAS; and
VLCDR 3 comprising an amino acid sequence having at least 95% sequence identity to the amino acid sequence of SEQ ID NO:10;
wherein the antibody or an antigen-binding fragment thereof specifically binds to the extracellular domain of human receptor tyrosine kinase like orphan receptor 1 (ROR1).

An embodiment provides an immunoconjugate, pharmaceutical composition, method of treatment, use, or process of making as described herein, wherein Ab is an antibody or an antigen-binding fragment thereof comprising:
a) a heavy chain comprising:
VHCDR 1 comprising an amino acid sequence having at least 95% sequence identity to the amino acid sequence of SEQ ID NO:15;
VHCDR 2 comprising an amino acid sequence having at least 95% sequence identity to the amino acid sequence of SEQ ID NO:16; and
VHCDR 3 comprising an amino acid sequence having at least 95% sequence identity to the amino acid sequence of SEQ ID NO:17; and
b) a light chain comprising:
VLCDR 1 comprising an amino acid sequence having at least 95% sequence identity to the amino acid sequence of SEQ ID NO:22;
VLCDR 2 comprising an amino acid sequence having at least 95% sequence identity to the amino acid sequence of DAY; and
VLCDR 3 comprising an amino acid sequence having at least 95% sequence identity to the amino acid sequence of SEQ ID NO:24;
wherein the antibody or an antigen-binding fragment thereof specifically binds to the extracellular domain of human receptor tyrosine kinase like orphan receptor 1 (ROR1).

An embodiment provides an immunoconjugate, pharmaceutical composition, method of treatment, use, or process of making as described herein, wherein Ab is an antibody or an antigen-binding fragment thereof comprising:
a) a heavy chain comprising:
VHCDR 1 comprising an amino acid sequence having at least 95% sequence identity to the amino acid sequence of SEQ ID NO:29;
VHCDR 2 comprising an amino acid sequence having at least 95% sequence identity to the amino acid sequence of SEQ ID NO:30; and
VHCDR 3 comprising an amino acid sequence having at least 95% sequence identity to the amino acid sequence of SEQ ID NO:31; and
b) a light chain comprising:
VLCDR 1 comprising an amino acid sequence having at least 95% sequence identity to the amino acid sequence of SEQ ID NO:36;
VLCDR 2 comprising an amino acid sequence having at least 95% sequence identity to the amino acid sequence of DAS; and
VLCDR 3 comprising an amino acid sequence having at least 95% sequence identity to the amino acid sequence of SEQ ID NO:38;
wherein the antibody or an antigen-binding fragment thereof specifically binds to the extracellular domain of human receptor tyrosine kinase like orphan receptor 1 (ROR1).

These and other embodiments are described in greater detail below.

### BRIEF DESCRIPTION OF THE DRAWINGS

FIG. 1 illustrates a trastuzumab deruxtecan antibody-drug conjugate.

FIG. 2 illustrates a reaction scheme for making a compound of the Formula (II), Formula (II*), Formula (IV) or Formula (IV*) with n⁴=2 and n⁵=0.

FIG. 3 illustrates a reaction scheme for making a compound of the Formula (IV-12).

FIG. 4 illustrates a reaction scheme for making a compound of the Formula (IV-17).

FIG. 5 illustrates a reaction scheme for making a compound of the Formula (V-6).

FIG. 6 illustrates a reaction scheme for making a conjugate of the Formula (IIIC-6).

FIG. 7A illustrates a reaction scheme for making an immunoconjugate of the Formula (I). FIG. 7B illustrates a reaction scheme for making an exemplary conjugate of Formula (III) where D (the drug moiety) in Formula (III) is a compound of Formula (II).

FIG. 8 illustrates a reaction scheme for making compounds 1-16 and 1-17 (see Example 1).

FIG. 9 illustrates a reaction scheme for compound 2-20 (see Example 2).

FIG. 10 illustrates a reaction scheme for making compounds 3-27 and 3-28 (see Example 3).

FIG. 11 illustrates a reaction scheme for making compounds 4-38 and 4-39 (see Example 4).

FIG. 12 illustrates a reaction scheme for making compounds 5-40 and 5-41 (see Example 5).

FIG. 13 illustrates a reaction scheme for making compound 6-44 (see Example 6).

FIG. 14 illustrates the structure of compound 7-45 (see Example 7).

FIG. 15 illustrates a reaction scheme for making compound 8-47 (see Example 8)

FIG. 16 illustrates the structure of compound 9-48 (see Example 9).

FIG. 17 illustrates a reaction scheme for making compound 10-49 (see Example 10).

FIG. 18 illustrates a reaction scheme for making compound 11-52 (see Example 11).

FIG. 19 illustrates a reaction scheme for making compounds 12-58 and 12-59 (see Example 12).

FIG. 20 illustrates a reaction scheme for making compounds 13-63 and 13-64 (see Example 13).

FIG. 21 illustrates a reaction scheme for making compounds 14-70 and 14-71 (see Example 14).

FIG. 22 illustrates a reaction scheme for making compounds 15-75 and 15-76 (see Example 15).

FIG. 23 illustrates a reaction scheme for making compounds 16-82 and 16-83 (see Example 16).

FIG. 24 illustrates a reaction scheme for making compounds 17-86 and 17-87 (see Example 17).

FIG. 25 illustrates a reaction scheme for making compound 18-94 (see Example 18).

FIG. 26 illustrates a reaction scheme for making compound 19-97 (see Example 19).

FIGS. 27A-27B illustrate a measurement of cell binding saturation data for the anti-ROR-1 antibodies generated by the methods described herein. A ROR-1 positive cell line JeKo-1 was incubated in a titration series with the anti-ROR-1 antibodies ATX-P-875 (FIG. 27A), ATX-P-885 (FIG. 27B) and ATX-P-890 (FIG. 27A) in comparison to the positive control antibody UC-961. Cells were washed, stained with secondary antibody and cell binding saturation was detected by flow cytometry and reported as mean fluorescent intensity (MFI).

FIGS. 28A-28B illustrate ROR-1 receptor internalization data for the anti-ROR-1 antibodies ATX-875, ATX-P-885, ATX-P-890. ROR-1 positive cell lines JeKo-1 (FIG. 28A) and MDA-MB-468 (FIG. 28B) were incubated with the anti-ROR-1 antibodies ATX-P-875, ATX-P-885, and ATX-P-890 and positive control antibody UC961 at super saturating conditions so as to bind all available ROR-1 receptors. Cells were washed and incubated at 4 different timepoints (30 min, 1 hour, 2 hours and 4 hours) at 37° C. before internalization was halted by placing the cells in ice. Receptor internalization was determined by flow cytometry and reported as percent receptor internalization relative to zero hours.

FIG. 29A-29H illustrate cellular binning data for the anti-ROR-1 antibodies ATX-P-875, ATX-P-885, and ATX-P-890. A cellular binning assay was performed to assess if ATX-P-875, ATX-P-885, and ATX-P-890 bound the same epitopes on the ROR-1 receptor as control antibodies UC-961 and 4A5. FIG. 29A depicts a staining profile for antibodies that bind the same epitope. FIG. 29B depicts the staining profile for antibodies that bind different epitopes. ATX-P-875, ATX-P-885 and ATX-P-890 were separately incubated with ROR-1+MDA-MB-468 at various amounts. Next, the anti-ROR-1 antibodies were fluorescently labeled with a secondary antibody. Finally, MDA-MB-468 cells coated with the anti-ROR-1 antibodies were incubated with a saturating dose of a fluorescently labeled UC-961 (FIGS. 29C-29E) or 4A5 (FIGS. 29F-29H) and analyzed by flow cytometry and the ATX-P-875, ATX-P-885 and ATX-P-890 antibody signal were compared with the UC-961 or 4A5 signal.

FIG. 30 illustrates AC-SINS data for the anti-ROR-1 antibodies ATX-P-875, ATX-P-885, and ATX-P-890. Antibody developability was assessed by performing an AC-SINS assay and evaluating the potential for self-interaction. Rituximab and Infliximab were used as controls to demonstrate a low and high shift, respectively. Assay results for ATX-P-875, ATX-P-885, and ATX-P-890 fell within the range determined by the control antibodies.

FIG. 31 illustrates biochemical binning data by SPR for the anti-ROR1 antibodies ATX-P-875, ATX-P-885, ATX-P-890 as compared against control anti-ROR-1 antibodies UC961 (ATX-P-453) and 4a5.

FIG. 32 illustrates nucleotide and amino acid sequences for anti-ROR-1 antibodies ATX-P-875, ATX-P-885, and ATX-P-890.

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, a “conjugate” is a compound that comprises two or more substances (such as an antibody, a linker moiety and/or a drug moiety) joined together by chemical bonds. Examples of conjugates include antibody-drug conjugates (which may optionally include a linker moiety), drug-linker conjugates, and antibody-linker conjugates. An “immunoconjugate” is a conjugate that comprise an immunological substance such as an antibody.

As used herein, an “antibody” (Ab) is a protein made by the immune system, or a synthetic variant thereof, that binds to specific sites on cells or tissues. An “antigen-binding fragment” (Fab) is a portion of an antibody that binds to a specific antigen. Monoclonal antibodies are a type of synthetic antibody. In cancer treatment, monoclonal antibodies may kill cancer cells directly, they may block development of tumor blood vessels, or they may help the immune system kill cancer cells.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), cycloalkyl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, nitro, sulfenyl, sulfinyl, sulfonyl, haloalkyl, haloalkoxy, an amino, a mono-substituted amine group, a di-substituted amine group, a mono-substituted amine(alkyl) and a di-substituted amine(alkyl).

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if ortho R¹ and R² substituents on a phenyl ring are indicated to be —O—(CR⁵R⁶)ₘ—O— such that R¹ and R² “taken together” form a ring, it means that the —O—(CR⁵R⁶)ₘ—O— is covalently bonded to the phenyl ring at the R¹ and R² positions to form a heterocyclic ring:

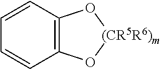

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkyl” where no numerical range is designated). The alkyl group may also be a medium size alkyl having 1 to 12 carbon atoms. The alkyl group could also be a lower alkyl having 1 to 6 carbon atoms. An alkyl group may be substituted or unsubstituted. An alkyl group is typically monovalent unless the context indicates otherwise. For example, those skilled in the art recognize that C₁-C₆ alkyl is bivalent in the following formula: —(C₁-C₆ alkyl)-X².

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

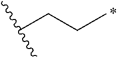 [SMILES: *CCC]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 4 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group (e.g.,

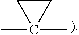

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

The term “halogen atom” or “halogen” as used herein, means any one of the radio-stable atoms of column 7 of the Periodic Table of the Elements, such as, fluorine, chlorine, bromine and iodine.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkenyl” refers to an alkenyl group in which one or more of the hydrogen atoms are replaced by a halogen (e.g., mono-haloalkenyl, di-haloalkenyl, tri-haloalkenyl and polyhaloalkenyl).

As used herein, “haloalkynyl” refers to an alkynyl group in which one or more of the hydrogen atoms are replaced by a halogen (e.g., mono-haloalkynyl, di-haloalkynyl, tri-haloalkynyl and polyhaloalkynyl).

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused, bridged or spiro fashion. As used herein, the term “fused” refers to two rings which have two atoms and one bond in common. As used herein, the term “bridged heterocyclyl” or “bridged heteroalicyclyl” refers to compounds wherein the heterocyclyl or heteroalicyclyl contains a linkage of one or more atoms connecting non-adjacent atoms. As used herein, the term “spiro” refers to two rings which have one atom in common and the two rings are not linked by a bridge. Heterocyclyl and heteroalicyclyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). For example, five carbon atoms and one heteroatom; four carbon atoms and two heteroatoms; three carbon atoms and three heteroatoms; four carbon atoms and one heteroatom; three carbon atoms and two heteroatoms; two carbon atoms and three heteroatoms; one carbon atom and four heteroatoms; three carbon atoms and one heteroatom; or two carbon atoms and one heteroatom. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isooxazoline, isoxazolidine, oxazoline, oxazolidine, oxazolidinone, thiazoline, thiazolidine, morpholine, oxirane, piperidine N-Oxide, piperidine, piperazine, pyrrolidine, azepane, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl). Examples of spiro heterocyclyl groups include 2-azaspiro[3.3]heptane, 2-oxaspiro[3.3]heptane, 2-oxa-6-azaspiro[3.3]heptane, 2,6-diazaspiro[3.3]heptane, 2-oxaspiro[3.4]octane and 2-azaspiro[3.4]octane.

Where the number of substituents is not specified (e.g. haloalkyl, haloalkenyl, haloalkynyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), a sulfuric acid, a nitric acid and a phosphoric acid (such as 2,3-dihydroxypropyl dihydrogen phosphate). Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, trifluoroacetic, benzoic, salicylic, 2-oxopentanedioic or naphthalenesulfonic acid. Pharmaceutical salts can also be obtained by reacting a compound with a base to form a salt such as an ammonium salt, an alkali metal salt, such as a sodium, a potassium or a lithium salt, an alkaline earth metal salt, such as a calcium or a magnesium salt, a salt of a carbonate, a salt of a bicarbonate, a salt of organic bases such as dicyclohexylamine, N-methyl-D-glucamine, tris(hydroxymethyl)methylamine, C₁-C₇ alkylamine, cyclohexylamine, triethanolamine, ethylenediamine and salts with amino acids such as arginine and lysine. For compounds of Formula (I), those skilled in the art understand that when a salt is formed by protonation of a nitrogen-based group (for example, NH₂), the nitrogen-based group can be associated with a positive charge (for example, NH₂ can become NH₃⁺) and the positive charge can be balanced by a negatively charged counterion (such as Cl⁻).

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched or a stereoisomeric mixture. As used herein, whenever a stereochemistry is “arbitrarily assigned” in a compound, it refers to a stereocenter that can be (R) or (S) in configuration and the compound may be the opposite enantiomer of that depicted. In addition, it is understood that, in any compound described herein having one or more double bond(s) generating geometrical isomers that can be defined as E or Z, each double bond may independently be E or Z a mixture thereof. Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium). Compounds described herein can also include all isotopes of atoms occurring in the intermediates or final compounds. Isotopes include those atoms having the same atomic number but different mass numbers. For example, isotopes of hydrogen include tritium and deuterium.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium), hydrogen-2 (deuterium), and hydrogen-3 (tritium). Thus, reference herein to a compound encompasses all potential isotopic forms unless the context clearly dictates otherwise.

It is understood that the methods and combinations described herein include crystalline forms (also known as polymorphs, which include the different crystal packing arrangements of the same elemental composition of a compound), amorphous phases, salts, solvates and hydrates. In some embodiments, the compounds described herein exist in solvated forms with pharmaceutically acceptable solvents such as water, ethanol or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol or the like. Hydrates are formed when the solvent is water or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### Compounds

Various embodiments disclosed herein relate to a compound of Formula (IV), or a pharmaceutically acceptable salt thereof, having the structure:

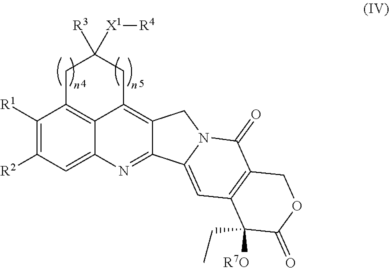

wherein:
R¹ and R² each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² can be taken together to form a ring;
R³ and R⁴ each individually can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and [(CY₂)ₚO(CY₂)_{q}]ₜCY₃, with the proviso that R³ and R⁴ cannot both be hydrogen; or R³ and R⁴ can be taken together with the carbon atom to which they are attached to form an unsubstituted 4-, 5- or 6-membered heterocyclyl or a 4-, 5- or 6-membered heterocyclyl that can be substituted with an optionally substituted C₁-C₃ alkyl;
R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
n⁴ and n⁵ each individually can be 0, 1 or 2, with the proviso that n⁴ and n⁵ cannot both be 0;
each Y individually can be H or halogen;
each m individually can be 1 or 2;
each p individually can be 1, 2, 3, 4, 5, or 6;
each q individually can be 0, 1, 2, 3, 4, 5, or 6; and
each t individually can be 1, 2, 3, 4, 5, or 6;
R⁷ can be H, —COR⁸, —CO₂R⁸, or —(CO)—NHR⁸; and
R⁸ can be a substituted or an unsubstituted C₁-C₆ alkyl, a substituted or an unsubstituted C₁-C₆ haloalkyl, or —[(CY₂)ₚO(CY₂)_{q}]ₜCY₃.

Various embodiments disclosed herein relate to a compound of Formula (IV*), or a pharmaceutically acceptable salt thereof, having the structure:

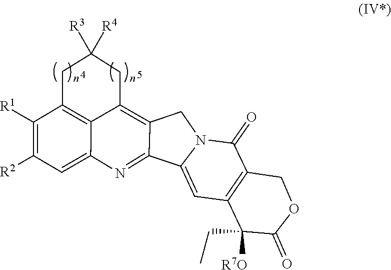

wherein:
R¹ and R² each individually can be selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, and a substituted or an unsubstituted —O—(CR⁵R⁶)ₘ—O— such that R¹ and R² can be taken together to form a ring;
R³ and R⁴ each individually can be selected from hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ cannot both be hydrogen; or R³ and R⁴ can be taken together with the carbon atom to which they are attached to form an unsubstituted 4-, 5- or 6-membered heterocyclyl or a 4-, 5- or 6-membered heterocyclyl that can be substituted with an optionally substituted C₁-C₃ alkyl;
R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
n⁴ and n⁵ each individually can be 0, 1 or 2, with the proviso that n⁴ and n⁵ cannot both be 0;
each Y individually can be H or halogen;
each m individually can be 1 or 2;
each p individually can be 1, 2, 3, 4, 5, or 6;
each q individually can be 0, 1, 2, 3, 4, 5, or 6; and
each t individually can be 1, 2, 3, 4, 5, or 6;
R⁷ can be H, —COR⁸, —CO₂R⁸, or —(CO)—NHR⁸; and
R⁸ can be a substituted or an unsubstituted C₁-C₆ alkyl, a substituted or an unsubstituted C₁-C₆ haloalkyl, or —[(CY₂)ₚO(CY₂)_{q}]ₜCY₃.

In various embodiments, R¹ and R² in Formula (IV) or Formula (IV*) each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² can be taken together to form a ring. In an embodiment, at least one of R¹ and R² can be hydrogen. In an embodiment, at least one of R¹ and R² can be halogen. For example, in an embodiment, at least one of R¹ and R² can be fluoro. In an embodiment, R¹ and R² can be each fluoro. In another embodiment, R¹ can be each fluoro and R² can be hydrogen. In still another embodiment, R¹ can be each hydrogen and R² can be fluoro. In an embodiment, at least one of R¹ and R² can be chloro. In one embodiment, one of R¹ and R² can be fluoro and the other of R¹ and R² can be chloro. In one embodiment, R¹ can be chloro and R² can be fluoro. In an embodiment, at least one of R¹ and R² can be —CN. In an embodiment, at least one of R¹ and R² can be —OR⁵, wherein R can be a substituted or an unsubstituted C₁-C₆ alkyl. For example, in an embodiment, at least one of R¹ and R² can be methoxy. In one embodiment, R¹ and R² can be a substituted or an unsubstituted —O—(CR⁵R⁶)ₘ—O— such that R¹ and R² can be taken together to form a ring. In an embodiment, one of R¹ and R² can be a substituted or an unsubstituted —O—(CH₂)—O— such that R¹ and R² can be taken together to form 1,3-dioxolane.

In an embodiment, at least one of R¹ and R² in Formula (IV) or Formula (IV*) can be —NR⁵R⁶, wherein R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl.

In an embodiment, at least one of R¹ and R² in Formula (IV) or Formula (IV*) can be a substituted or an unsubstituted C₁-C₆ alkyl. In an embodiment, at least one of R¹ and R² can be a C₁-C₃ alkyl. For example, in an embodiment, at least one of R¹ and R² can be methyl. In an embodiment, at least one of R¹ and R² can be C₁-C₃ alkyl and the other can be a halogen. For example, in an embodiment, at least one of R¹ and R² can be methyl and the other can be fluoro. In an embodiment, R¹ can be methyl and R² other can be fluoro.

In an embodiment, at least one of R¹ and R² in Formula (IV) or Formula (IV*) can be a substituted or an unsubstituted C₁-C₆ haloalkyl. For example, in an embodiment, at least one of R¹ and R² can be difluoromethyl. In an embodiment, at least one of R¹ and R² can be a substituted or an unsubstituted —O—(C₁-C₆ alkyl). For example, in an embodiment, at least one of R¹ and R² can be methoxy. In an embodiment, at least one of R¹ and R² can be —[(CY₂)ₚO(CY₂)_{q}]ₜCY₃. In an embodiment, R¹ and R² can be a substituted or an unsubstituted —O—(CR⁵R⁶)ₘ—O— such that R¹ and R² can be taken together to form a ring in which the ends of the —O—(CR⁵R⁶)ₘ—O— are covalently bonded to the phenyl ring at the R¹ and R² positions of Formula (IV) or Formula (IV*) to form a heterocyclic ring.

In an embodiment, one of R¹ and R² in Formula (IV) or Formula (IV*) can be hydrogen and the other of R¹ and R² can be halogen. In an embodiment, one of R¹ and R² in Formula (IV) or Formula (IV*) can be hydrogen and the other of R¹ and R² can be fluoro. In an embodiment, R¹ in Formula (IV) or Formula (IV*) can be fluoro and R² can be hydrogen. In an embodiment, R¹ in Formula (IV) or Formula (IV*) can be hydrogen and R² can be fluoro. In an embodiment, one of R¹ and R² can be hydrogen and the other of R¹ and R² can be a substituted or an unsubstituted C₁-C₆ alkyl. In an embodiment, one of R¹ and R² can be hydrogen and the other of R¹ and R² can be a substituted or an unsubstituted C₁-C₆ haloalkyl. In an embodiment, one of R¹ and R² can be hydrogen and the other of R¹ and R² can be a substituted or an unsubstituted —O—(C₁-C₆ alkyl). In an embodiment, both R¹ and R² can be hydrogen. In an embodiment, neither R¹ nor R² can be hydrogen.

In an embodiment, one of R¹ and R² in Formula (IV) or Formula (IV*) can be halogen and the other of R¹ and R² can be a substituted or an unsubstituted C₁-C₆ alkyl. In an embodiment, one of R¹ and R² can be halogen and the other of R¹ and R² can be a substituted or an unsubstituted C₁-C₆ haloalkyl. In an embodiment, one of R¹ and R² can be halogen and the other of R¹ and R² can be a substituted or an unsubstituted —O—(C₁-C₆ alkyl). In an embodiment, both R¹ and R² independently can be halogen. In an embodiment, neither R¹ nor R² can be halogen.

In an embodiment, one of R¹ and R² in Formula (IV) or Formula (IV*) can be a substituted or an unsubstituted C₁-C₆ alkyl and the other of R¹ and R² can be a substituted or an unsubstituted C₁-C₆ haloalkyl. In an embodiment, one of R¹ and R² can be a substituted or an unsubstituted C₁-C₆ alkyl and the other of R¹ and R² can be a substituted or an unsubstituted —O—(C₁-C₆ alkyl). In an embodiment, both R¹ and R² independently can be a substituted or an unsubstituted C₁-C₆ alkyl. In an embodiment, neither R¹ nor R² can be a substituted or an unsubstituted C₁-C₆ alkyl.

In an embodiment, one of R¹ and R² in Formula (IV) or Formula (IV*) can be a substituted or an unsubstituted C₁-C₆ haloalkyl and the other of R¹ and R² can be a substituted or an unsubstituted —O—(C₁-C₆ alkyl). In an embodiment, both R¹ and R² independently can be a substituted or an unsubstituted C₁-C₆ haloalkyl. In an embodiment, neither R¹ nor R² can be a substituted or an unsubstituted C₁-C₆ haloalkyl.

In an embodiment, one of R¹ and R² in Formula (IV) or Formula (IV*) can be a substituted or an unsubstituted —O—(C₁-C₆ alkyl). In an embodiment, both R¹ and R² independently can be a substituted or an unsubstituted —O—(C₁-C₆ alkyl). In an embodiment, neither R¹ nor R² can be a substituted or an unsubstituted —O—(C₁-C₆ alkyl). In an embodiment, R¹ and R² can be a substituted or an unsubstituted —O—(CR⁵R⁶)ₘ—O— such that R¹ and R² can be taken together to form a ring. In various embodiments, R¹ and R² each individually can be selected from hydrogen, fluoro, methoxy, methyl, difluoromethyl, and —O—(CH₂)—O— such that R¹ and R² can be taken together to form a ring.

In various embodiments, R³ in Formula (IV) can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R³ can be hydrogen. In an embodiment, R³ can be —OR⁵, such as —OCH₃. In an embodiment, R³ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R³ can be a substituted or an unsubstituted C₁-C₆ alkyl. For example, in an embodiment, R³ can be methyl, —CH₂OH or —CH₂CH₂OH. As another example, in an embodiment, R³ can be —(CH₂)₃OH or —CH₂O(CH₂)₂OH. In an embodiment, R³ can be a substituted or an unsubstituted C₂-C₆ alkenyl. For example, in an embodiment, R³ can be —CH═CH₂. In another embodiment, R³ can be —CH₂CH═CH₂. In an embodiment, R³ can be a substituted or an unsubstituted C₂-C₆ alkynyl. For example, in an embodiment, R³ can be —C≡CH. In an embodiment, R³ can be —[(CY₂)ₚO(CY₂)_{q}]ₜCY₃, where each Y individually can be H or halogen.

In various embodiments, R⁴ in Formula (IV) can be selected from hydrogen, —OR⁵, —NR⁵R⁶, a substituted or an unsubstituted C₁-C₆ alkyl, a substituted or an unsubstituted C₂-C₆ alkenyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R⁴ can be hydrogen. In an embodiment, R⁴ can be —OR⁵, such as —OCH₃. In an embodiment, R⁴ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R⁴ can be a substituted or an unsubstituted C₁-C₆ alkyl. For example, in an embodiment, R⁴ can be methyl, —CH₂OH or —CH₂CH₂OH. As another example, in an embodiment, R⁴ can be —(CH₂)₃OH or —CH₂O(CH₂)₂OH. In an embodiment, R⁴ can be a substituted or an unsubstituted C₂-C₆ alkenyl. For example, in an embodiment, R⁴ can be —CH═CH₂. In another embodiment, R⁴ can be —CH₂CH═CH₂. In an embodiment, R⁴ can be a substituted or an unsubstituted C₂-C₆ alkynyl. For example, in an embodiment, R⁴ can be —C≡CH. In an embodiment, R⁴ can be —[(CY₂)ₚO(CY₂)_{q}]ₜCY₃, where each Y individually can be H or halogen.

In various embodiments, R³ in Formula (IV*) can be selected from hydrogen, —OR⁵, —NR⁵R⁶, a substituted or an unsubstituted C₁-C₆ alkyl, a substituted or unsubstituted 4- or 5-membered heterocyclyl, and —[(CY₂)ₚO(CY₂)_{q}]ₜCY₃, with the proviso that R³ and R⁴ cannot both hydrogen. In an embodiment, R³ can be hydrogen. In an embodiment, R³ can be —OR⁵, such as —OCH₃. In an embodiment, R³ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R³ can be a substituted or an unsubstituted C₁-C₆ alkyl. For example, in an embodiment, R³ can be methyl, —CH₂OH, or —CH₂CH₂OH. As another example, in an embodiment, R³ can be —(CH₂)₃OH or —CH₂O(CH₂)₂OH. In an embodiment, R³ can be —[(CY₂)ₚO(CY₂)_{q}]ₜCY₃, where each Y individually can be H or halogen.

In various embodiments, R⁴ in Formula (IV*) can be selected from hydrogen, —OR⁵, —NR⁵R⁶, a substituted or an unsubstituted C₁-C₆ alkyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R⁴ can be hydrogen. In an embodiment, R⁴ can be —OR⁵, such as —OCH₃. In an embodiment, R⁴ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R⁴ can be a substituted or an unsubstituted C₁-C₆ alkyl. For example, in an embodiment, R⁴ can be methyl, —CH₂OH. or —CH₂CH₂OH. As another example, in an embodiment, R⁴ can be —(CH₂)₃OH or —CH₂O(CH₂)₂OH. In an embodiment, R⁴ can be —[(CY₂)ₚO(CY₂)_{q}]ₜCY₃, where each Y individually can be H or halogen.

In some embodiments, one of R³ and R⁴ in Formula (IV) or Formula (IV*) can be hydrogen and the other of R³ and R⁴ can be a substituted or an unsubstituted C₁-C₆ alkyl. In other embodiments, one of R³ and R⁴ can be hydrogen and the other of R³ and R⁴ can be —NR⁵R⁶. In some embodiments, one of R³ and R⁴ can be CH₃, with the proviso that R³ and R⁴ cannot both be —CH₃. For example, in some embodiments, one of R³ and R⁴ can be —CH₃ and the other of R³ and R⁴ can be a substituted or an unsubstituted C₁-C₆ alkyl that cannot be —CH₃, such as —CH₂OH or —CH₂CH₂OH. In some embodiments, one of R³ and R⁴ can be —CH₃, and the other of R³ and R⁴ can be —NR⁵R⁶.

In some embodiments, R³ and/or R⁴ in Formula (IV) or Formula (IV*) can be —NR⁵R⁶ as described herein. For example, in an embodiment, R⁵ and R⁶ each individually can be a substituted or an unsubstituted C₁-C₆ alkyl (such as —CH₃, —CH₂CH₂OH, or

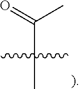 [SMILES: *C(C)=O]

In other embodiments where R³ and/or R⁴ in Formula (IV) or Formula (IV*) can be —NR⁵R⁶ described herein, R⁵ and R⁶ can be taken together with the nitrogen atom to which they are attached to form a substituted or unsubstituted 4- or 5-membered heterocyclyl. For example, in some embodiments, the —NR⁵R⁶ can be

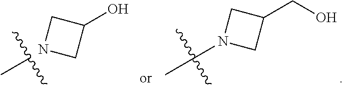

In other embodiments, the —NR⁵R⁶ can be

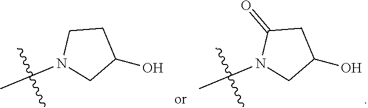 [SMILES: *N1CCC(O)C1 ; *N1CC(O)CC1=O]

In some embodiments, R³ and/or R⁴ in Formula (IV) or Formula (IV*) can be —[(CY₂)ₚO(CY₂)_{q}]ₜCY₃, such as —CH₂OCH₃. For example, in some embodiments, one of R³ and R⁴ can be —CH₂OH or —CH₂CH₂OH, and the other of R³ and R⁴ can be —CH₂OCH₃.

In some embodiments, R³ and R⁴ in Formula (IV) or Formula (IV*) can be taken together with the carbon atom to which they are attached to form an unsubstituted 4-, 5- or 6-membered heterocyclyl or a 4-, 5- or 6-membered heterocyclyl that can be substituted with an optionally substituted C₁-C₃ alkyl. For example, in some embodiments, R³ and R⁴, taken together with the carbon atom to which they are attached, can be selected from

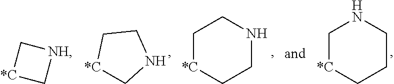

where * indicates the carbon atom to which R³ and R⁴ can be attached. In other embodiments, R³ and R⁴, taken together with the carbon atom to which they are attached, can be selected from

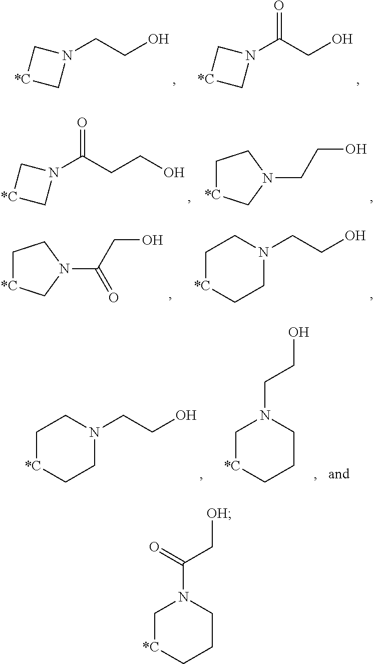

where * indicates the carbon atom to which R³ and R⁴ are attached.

In one embodiment, one of R³ and R⁴ in Formula (IV) or Formula (IV*) can be hydrogen and the other of R³ and R⁴ can be —CH₂OH. In one embodiment, one of R³ and R⁴ in Formula (IV) or Formula (IV*) can be —CH₃ and the other of R³ and R⁴ can be —CH₂OH. In one embodiment, one of R³ and R⁴ in Formula (IV) or Formula (IV*) can be —OCH₃ and the other of R³ and R⁴ can be —CH₂OH. In one embodiment, one of R³ and R⁴ in Formula (IV) or Formula (IV*) can be —C₂H₅ and the other of R³ and R⁴ can be CH₂OH. In one embodiment, one of R³ and R⁴ in Formula (IV) can be —CH═CH₂ and the other of R³ and R⁴ can be —CH₂OH. In one embodiment, one of R³ and R⁴ in Formula (IV) can be —C≡CH and the other of R³ and R⁴ can be —CH₂OH. In one embodiment, one of R³ and R⁴ in Formula (IV) or Formula (IV*) can be hydrogen and the other of R³ and R⁴ can be —(CH₂)₂OH. In one embodiment, one of R³ and R⁴ in Formula (IV) or Formula (IV*) can be —CH₃ and the other of R³ and R⁴ can be —(CH₂)₂OH. In one embodiment, one of R³ and R⁴ in Formula (IV) or Formula (IV*) can be —OCH₃ and the other of R³ and R⁴ can be —(CH₂)₂OH. In one embodiment, one of R³ and R⁴ in Formula (IV) or Formula (IV*) can be —C₂H₅ and the other of R³ and R⁴ can be —(CH₂)₂OH. In one embodiment, one of R³ and R⁴ in Formula (IV) can be —CH═CH₂ and the other of R³ and R⁴ can be —(CH₂)₂OH. In one embodiment, one of R³ and R⁴ in Formula (IV) can be —C≡CH and the other of R³ and R⁴ can be —(CH₂)₂OH. In one embodiment, one of R³ and R⁴ in Formula (IV) or Formula (IV*) can be hydrogen and the other of R³ and R⁴ can be —(CH₂)₃OH. In one embodiment, one of R³ and R⁴ in Formula (IV) or Formula (IV*) can be —CH₃ and the other of R³ and R⁴ can be —(CH₂)₃OH. In one embodiment, one of R³ and R⁴ in Formula (IV) or Formula (IV*) can be —OCH₃ and the other of R³ and R⁴ can be —(CH₂)₃OH. In one embodiment, one of R³ and R⁴ in Formula (IV) or Formula (IV*) can be —C₂H₅ and the other of R³ and R⁴ can be —(CH₂)₃OH. In one embodiment, one of R³ and R⁴ in Formula (IV) can be —CH═CH₂ and the other of R³ and R⁴ can be —(CH₂)₃OH. In one embodiment, one of R³ and R⁴ in Formula (IV) can be —C≡CH and the other of R³ and R⁴ can be —(CH₂)₃OH. In one embodiment, one of R³ and R⁴ in Formula (IV) can be hydrogen and the other of R³ and R⁴ can be —CH₂—CH═CH₂. In one embodiment, one of R³ and R⁴ in Formula (IV) can be hydrogen and the other of R³ and R⁴ can be —C≡CH. In one embodiment, R³ and R⁴ both can be —CH₂OH. In one embodiment, one of R³ and R⁴ in Formula (IV) or Formula (IV*) can be hydrogen and the other of R³ and R⁴ can be —CH₂O(CH₂)₂OH. In one embodiment, one of R³ and R⁴ in Formula (IV) or Formula (IV*) can be —CH₃ and the other of R³ and R⁴ can be —CH₂O(CH₂)₂OH. In one embodiment, one of R³ and R⁴ in Formula (IV) or Formula (IV*) can be —OCH₃ and the other of R³ and R⁴ can be —CH₂O(CH₂)₂OH. In one embodiment, one of R³ and R⁴ in Formula (IV) or Formula (IV*) can be —C₂H₅ and the other of R³ and R⁴ can be —CH₂O(CH₂)₂OH. In one embodiment, one of R³ and R⁴ in Formula (IV) can be —CH═CH₂ and the other of R³ and R⁴ can be —(CH₂)₂OH. In one embodiment, one of R³ and R⁴ in Formula (IV) can be —CH═CH₂ and the other of R³ and R⁴ can be —CH₂O(CH₂)₂OH. In one embodiment, one of R³ and R⁴ in Formula (IV) can be —C≡CH and the other of R³ and R⁴ can be —CH₂O(CH₂)₂OH. In one embodiment, one of R³ and R⁴ in Formula (IV) or Formula (IV*) can be hydrogen or —CH₃ and the other of R³ and R⁴ can be

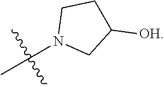 [SMILES: *N1CCC(O)C1]

In one embodiment, R³ and R⁴ both can be —CH₂OH. In one embodiment, one of R³ and R⁴ in Formula (IV) or Formula (IV*) can be —CH₂OH or —(CH₂)₂OH and the other of R³ and R⁴ can be —CH₂OCH₃. In one embodiment, one of R³ and R⁴ in Formula (IV) or Formula (IV*) can be hydrogen or —CH₃ and the other of R³ and R⁴ can be —N(CH₃)CH₂CH₂OH. In one embodiment, one of R³ and R⁴ in Formula (IV) or Formula (IV*) can be hydrogen or —CH₃ and the other of R³ and R⁴ can be —N(C═OCH₃)CH₂CH₂OH. In one embodiment, one of R³ and R⁴ in Formula (IV) or Formula (IV*) can be hydrogen or —CH₃ and the other of R³ and R⁴ can be

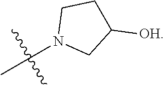 [SMILES: *N1CCC(O)C1]

In one embodiment, one of R³ and R⁴ in Formula (IV) or Formula (IV*) can be hydrogen or —CH₃ and the other of R³ and R⁴ can be

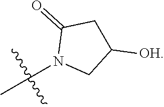 [SMILES: *N1CC(O)CC1=O]

In one embodiment, one of R³ and R⁴ in Formula (IV) or Formula (IV*) can be hydrogen or —CH₃ and the other of R³ and R⁴ can be

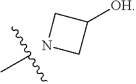

In one embodiment, one of R³ and R⁴ in Formula (IV) or Formula (IV*) can be hydrogen or —CH₃ and the other of R³ and R⁴ can be

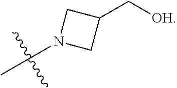 [SMILES: *N1CC(CO)C1]

In various embodiments, R⁷ in Formula (IV) or Formula (IV*) can be H, —COR⁸, —CO₂R⁸, or —(CO)—NHR⁸, wherein R⁸ is described elsewhere herein. In an embodiment, R⁷ can be H. In an embodiment, R⁷ can be —COR⁸. In an embodiment, R⁷ can be —CO₂R⁸. In an embodiment, R⁷ can be —(CO)—NHR⁸.

In various embodiments, R⁸ in Formula (IV) or Formula (IV*) can be a substituted or an unsubstituted C₁-C₆ alkyl, a substituted or an unsubstituted C₁-C₆ haloalkyl, or —[(CY₂)ₚO(CY₂)_{q}]ₜCY₃, where the variables p, q, t and Y are described elsewhere herein. In an embodiment, R⁸ can be a substituted or an unsubstituted C₁-C₆ alkyl. In an embodiment, R⁸ can be a substituted or an unsubstituted C₁-C₆ haloalkyl. In an embodiment, R⁸ can be a —[(CY₂)ₚO(CY₂)_{q}]ₜCY₃.

In various embodiments, m in Formula (IV) or Formula (IV*) can be 1 or 2. In an embodiment, m can 1. In another embodiment, m can be 2.

In various embodiments, n⁴ and n⁵ in Formula (IV) or Formula (IV*) each individually can be 0, 1 or 2, with the proviso that n⁴ and n⁵ cannot both be 0. In an embodiment, n⁴ and n⁵ both can be 1. In an embodiment, n⁴ can be 0 and n⁵ can be 1. In an embodiment, n⁴ can be 0 and n⁵ can be 2. In an embodiment, n⁴ can be 1 and n⁵ can be 0. In an embodiment, n⁴ can be 2 and n⁵ can be 0.

In various embodiments, each Y in Formula (IV) or Formula (IV*) can be individually H or halogen. In an embodiment, each Y can be hydrogen. In an embodiment, —CY₂ can be —CH₂. In an embodiment, —CY₃ can be —CH₃. In an embodiment, —CY₃ can be —CHF₂. In an embodiment, —CY₃ can be —CH₂F. In an embodiment, —CY₃ can be —CF₃.

In various embodiments, each p in Formula (IV) or Formula (IV*) individually can be 1, 2, 3, 4, 5, or 6. In an embodiment, p can be 1. In an embodiment, p is 2.

In various embodiments, each q in Formula (IV) or Formula (IV*) individually can be 0, 1, 2, 3, 4, 5, or 6. In an embodiment, q can be 1. In an embodiment, q can be 2.

In various embodiments, each t in Formula (IV) or Formula (IV*) individually can be 1, 2, 3, 4, 5, or 6. In an embodiment, t can be 1. In an embodiment, p can be t.

In various embodiments, R¹ and R² can be each individually selected from hydrogen, halogen, and an unsubstituted C₁-C₆ alkyl, or a substituted or an unsubstituted —O—(CR⁵R⁶)ₘ—O— such that R¹ and R² can be taken together form a ring; R³ and R⁴ can be each individually selected from hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ are not both hydrogen; or R³ and R⁴, can be taken together with the carbon atom to which they are attached, form an unsubstituted 4-, 5- or 6-membered heterocyclyl or a 4-, 5- or 6-membered heterocyclyl that is substituted with an unsubstituted C₁-C₃ alkyl; R⁵ and R⁶ can be each individually an unsubstituted C₁-C₆ alkyl, or R⁵ and R⁶, can be taken together with the nitrogen atom to which they are attached, form a substituted or unsubstituted 4- or 5-membered heterocyclyl; n⁴ can be 2; n⁵ can be 0; each Y can be H; each m can be 1; p can be 1; q can be 2; t can be 1; and R⁷ can be H. In various embodiments, R¹ and R² can be each individually selected from hydrogen, halogen and an unsubstituted C₁-C₆ alkyl; R³ and R⁴ can be each individually selected from hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ are not both hydrogen; or R³ and R⁴, can be taken together with the carbon atom to which they are attached, form an unsubstituted 4-, 5- or 6-membered heterocyclyl or a 4-, 5- or 6-membered heterocyclyl that is substituted with an unsubstituted C₁-C₃ alkyl; R⁵ and R⁶ can be each individually an unsubstituted C₁-C₆ alkyl, or R⁵ and R⁶, can be taken together with the nitrogen atom to which they are attached, form a substituted or unsubstituted 4- or 5-membered heterocyclyl; n⁴ can be 2; n⁵ can be 0; each Y can be H; each m can be 1; p can be 1; q can be 2; t can be 1; and R⁷ can be H.

In various embodiments, the compound of Formula (IV) and/or Formula (IV*), or alternatively, the present disclosure provides a compound that can be represented by a structure selected from the following, or a pharmaceutically acceptable salt thereof:

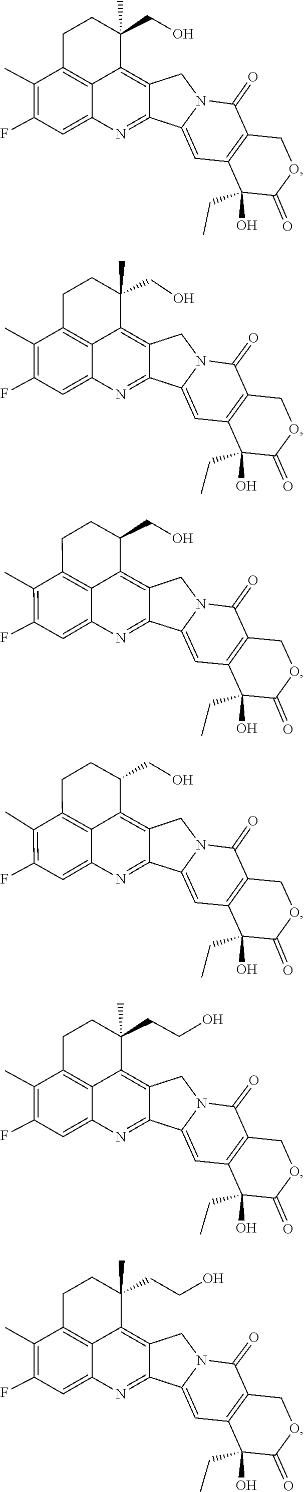

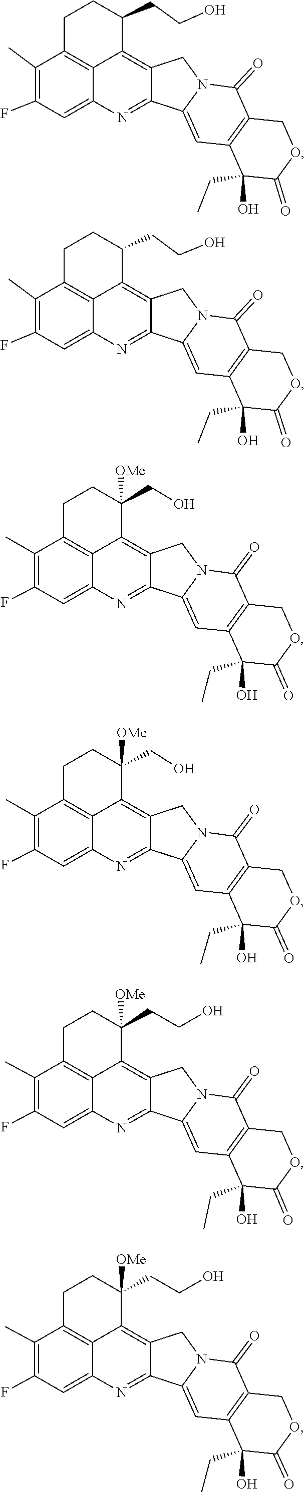

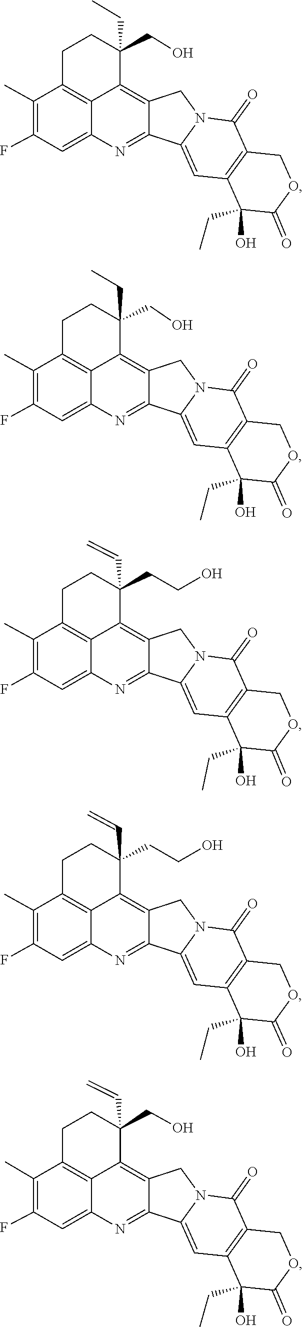 [SMILES: CC[C@@]1(CO)CCc2c(C)c(F)cc3nc4c(c1c23)Cn1c-4cc2c(c1=O)COC(=O)[C@]2(O)CC ; CC[C@]1(CO)CCc2c(C)c(F)cc3nc4c(c1c23)Cn1c-4cc2c(c1=O)COC(=O)[C@]2(O)CC ; C=C[C@@]1(CCO)CCc2c(C)c(F)cc3nc4c(c1c23)Cn1c-4cc2c(c1=O)COC(=O)[C@]2(O)CC ; C=C[C@]1(CCO)CCc2c(C)c(F)cc3nc4c(c1c23)Cn1c-4cc2c(c1=O)COC(=O)[C@]2(O)CC ; C=C[C@@]1(CO)CCc2c(C)c(F)cc3nc4c(c1c23)Cn1c-4cc2c(c1=O)COC(=O)[C@]2(O)CC]

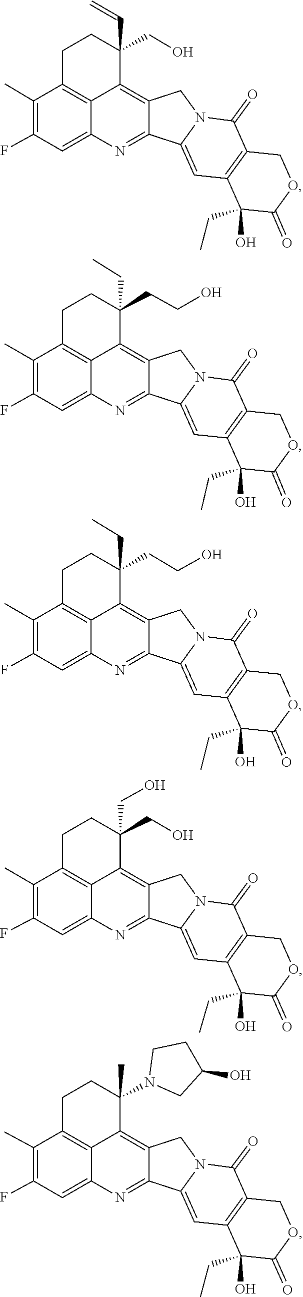

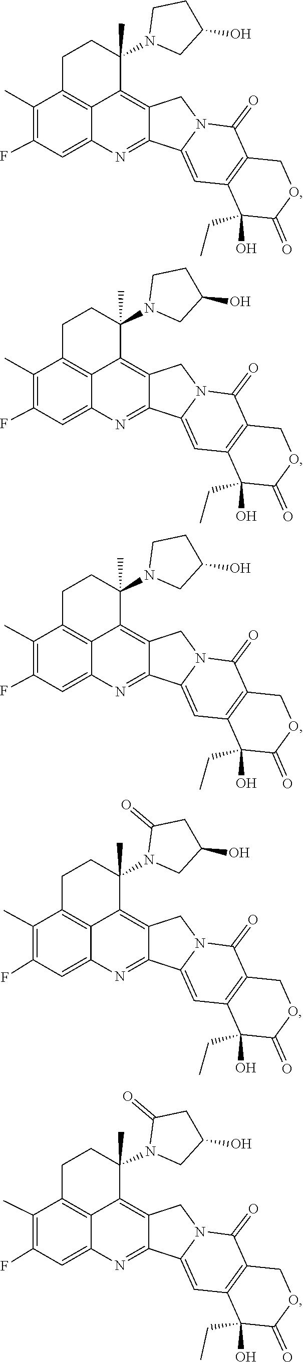

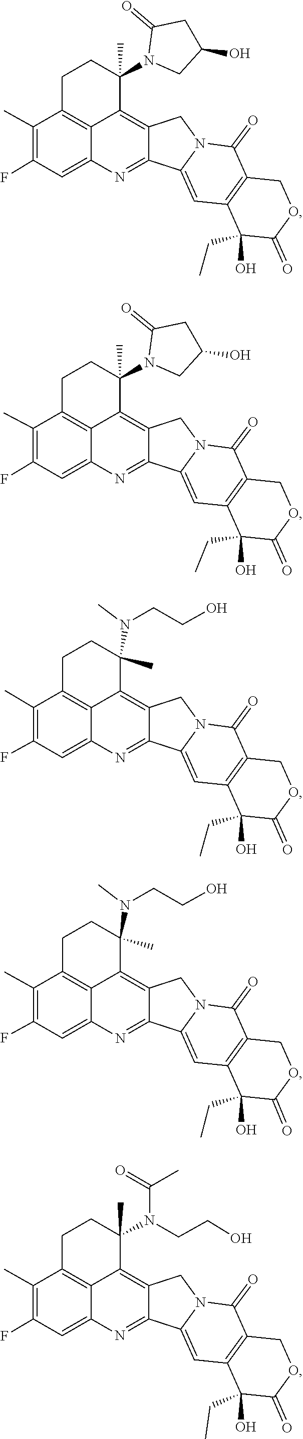

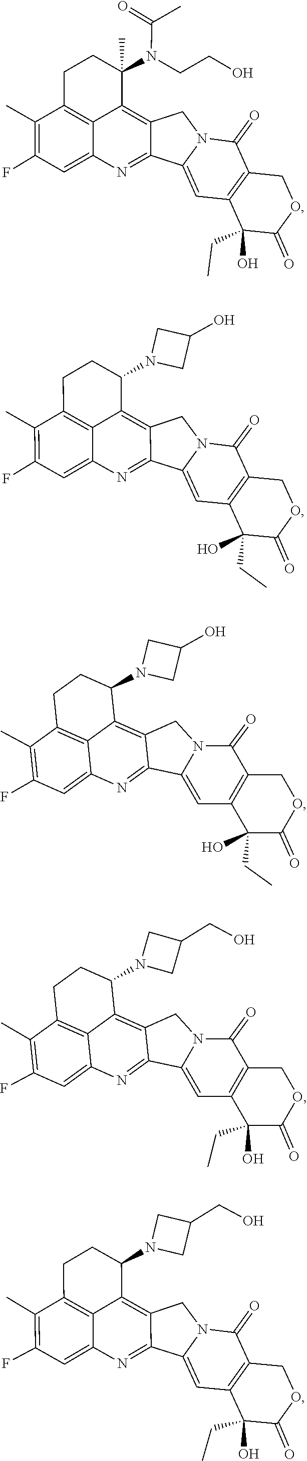

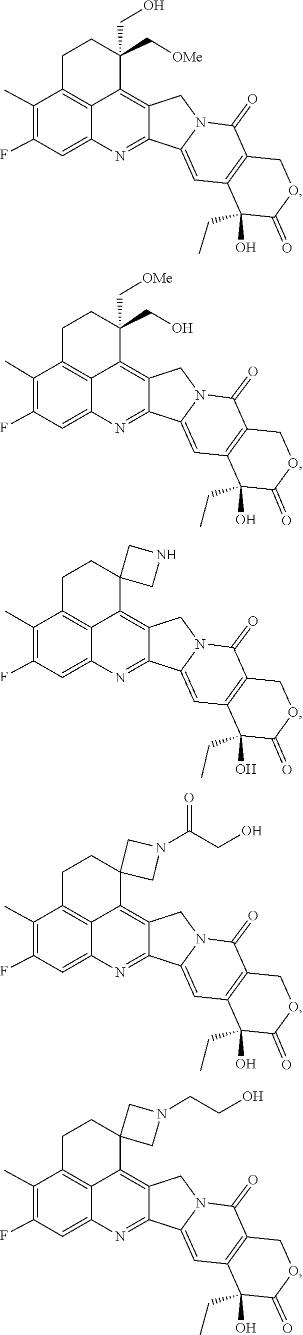

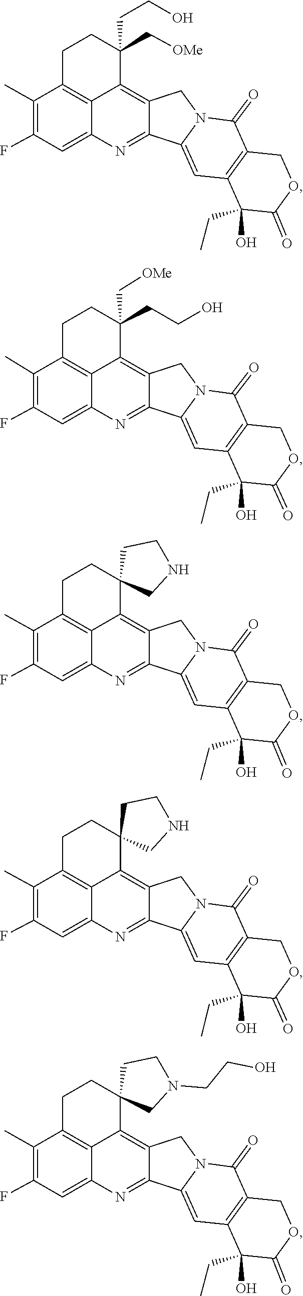

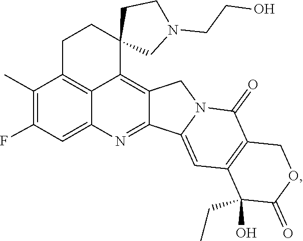 [SMILES: CC[C@@]1(O)C(=O)OCc2c1cc1n(c2=O)Cc2c-1nc1cc(F)c(C)c3c1c2[C@@]1(CC3)CCN(CCO)C1]

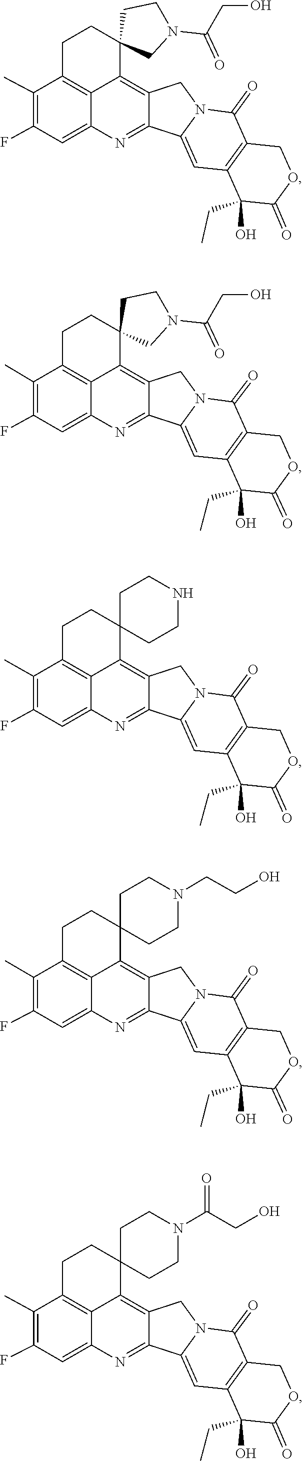 [SMILES: CC[C@@]1(O)C(=O)OCc2c1cc1n(c2=O)Cc2c-1nc1cc(F)c(C)c3c1c2[C@]1(CC3)CCN(C(=O)CO)C1 ; CC[C@@]1(O)C(=O)OCc2c1cc1n(c2=O)Cc2c-1nc1cc(F)c(C)c3c1c2[C@@]1(CC3)CCN(C(=O)CO)C1 ; CC[C@@]1(O)C(=O)OCc2c1cc1n(c2=O)Cc2c-1nc1cc(F)c(C)c3c1c2C1(CCNCC1)CC3 ; CC[C@@]1(O)C(=O)OCc2c1cc1n(c2=O)Cc2c-1nc1cc(F)c(C)c3c1c2C1(CC3)CCN(CCO)CC1 ; CC[C@@]1(O)C(=O)OCc2c1cc1n(c2=O)Cc2c-1nc1cc(F)c(C)c3c1c2C1(CC3)CCN(C(=O)CO)CC1]

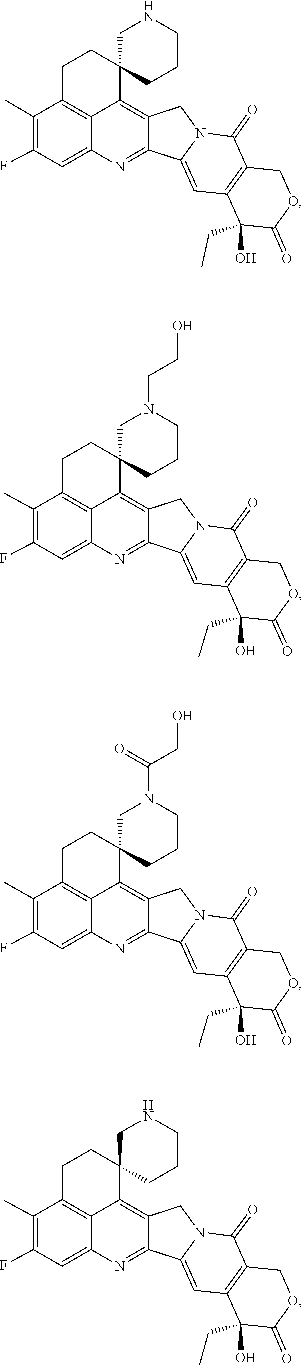 [SMILES: CC[C@@]1(O)C(=O)OCc2c1cc1n(c2=O)Cc2c-1nc1cc(F)c(C)c3c1c2[C@]1(CCCNC1)CC3 ; CC[C@@]1(O)C(=O)OCc2c1cc1n(c2=O)Cc2c-1nc1cc(F)c(C)c3c1c2[C@]1(CCCN(CCO)C1)CC3 ; CC[C@@]1(O)C(=O)OCc2c1cc1n(c2=O)Cc2c-1nc1cc(F)c(C)c3c1c2[C@]1(CCCN(C(=O)CO)C1)CC3 ; CC[C@@]1(O)C(=O)OCc2c1cc1n(c2=O)Cc2c-1nc1cc(F)c(C)c3c1c2[C@@]1(CCCNC1)CC3]

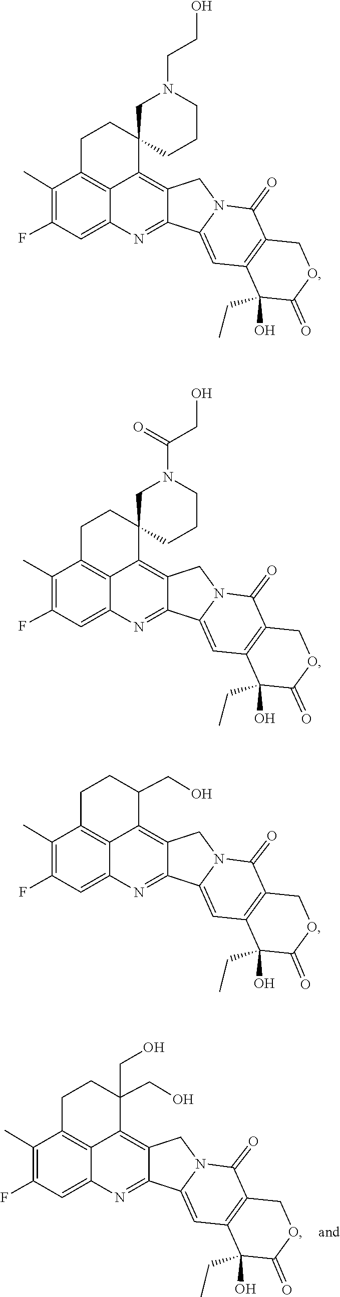 [SMILES: CC[C@@]1(O)C(=O)OCc2c1cc1n(c2=O)Cc2c-1nc1cc(F)c(C)c3c1c2[C@@]1(CCCN(CCO)C1)CC3 ; CC[C@@]1(O)C(=O)OCc2c1cc1n(c2=O)Cc2c-1nc1cc(F)c(C)c3c1c2[C@@]1(CCCN(C(=O)CO)C1)CC3 ; CC[C@@]1(O)C(=O)OCc2c1cc1n(c2=O)Cc2c-1nc1cc(F)c(C)c3c1c2C(CO)CC3 ; CC[C@@]1(O)C(=O)OCc2c1cc1n(c2=O)Cc2c-1nc1cc(F)c(C)c3c1c2C(CO)(CO)CC3]

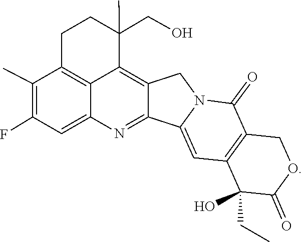 [SMILES: CC[C@@]1(O)C(=O)OCc2c1cc1n(c2=O)Cc2c-1nc1cc(F)c(C)c3c1c2C(C)(CO)CC3]

In various embodiments, the compound of Formula (IV) and/or Formula (IV*) can be represented by a structure selected from the following, or a pharmaceutically acceptable salt thereof:

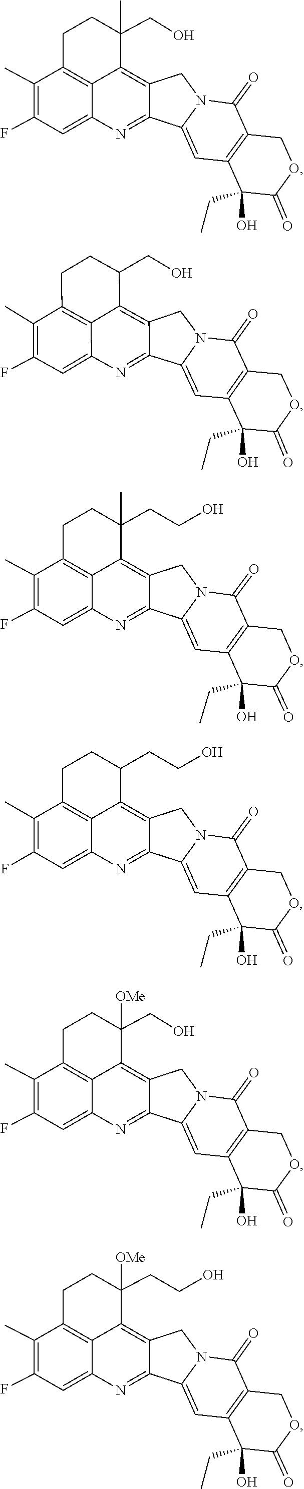

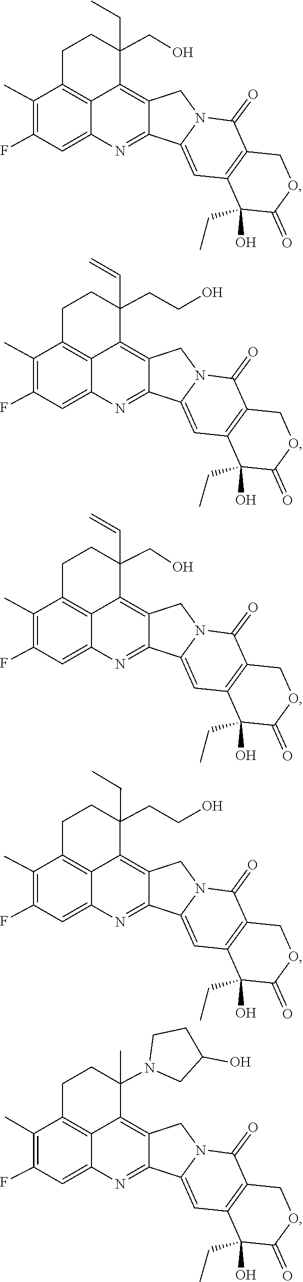

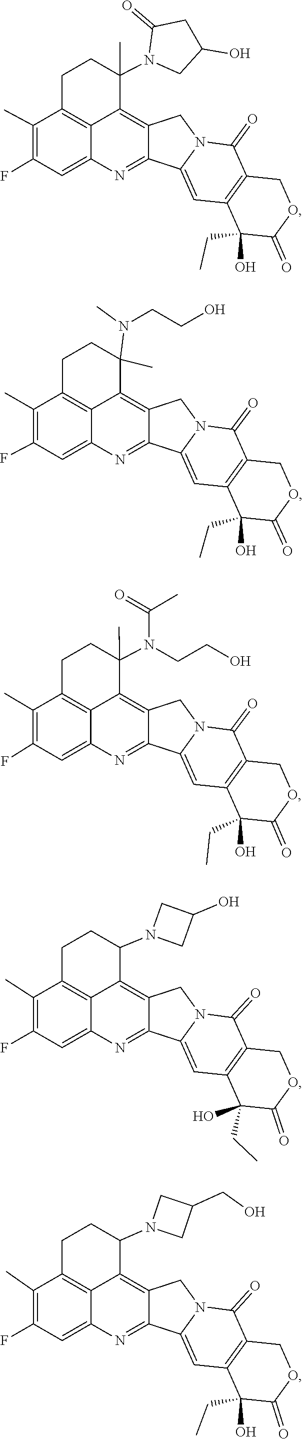

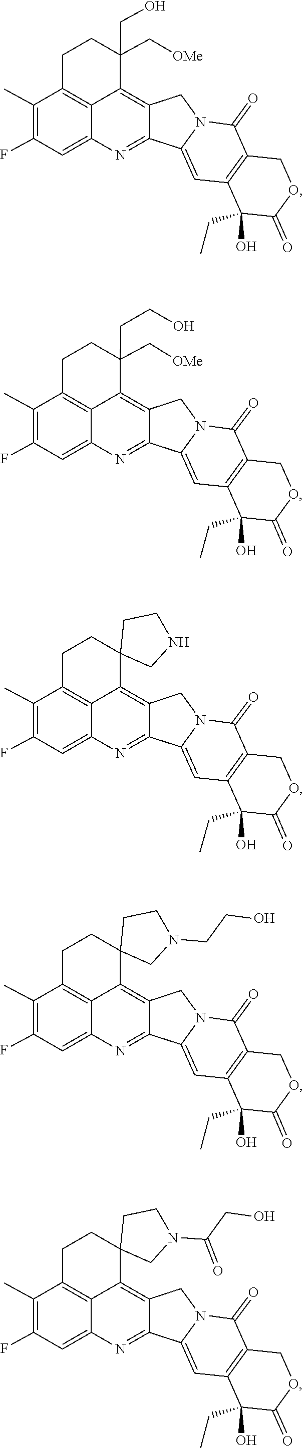 [SMILES: CC[C@@]1(O)C(=O)OCc2c1cc1n(c2=O)Cc2c-1nc1cc(F)c(C)c3c1c2C(CO)(COC)CC3 ; CC[C@@]1(O)C(=O)OCc2c1cc1n(c2=O)Cc2c-1nc1cc(F)c(C)c3c1c2C(CCO)(COC)CC3 ; CC[C@@]1(O)C(=O)OCc2c1cc1n(c2=O)Cc2c-1nc1cc(F)c(C)c3c1c2C1(CCNC1)CC3 ; CC[C@@]1(O)C(=O)OCc2c1cc1n(c2=O)Cc2c-1nc1cc(F)c(C)c3c1c2C1(CC3)CCN(CCO)C1 ; CC[C@@]1(O)C(=O)OCc2c1cc1n(c2=O)Cc2c-1nc1cc(F)c(C)c3c1c2C1(CC3)CCN(C(=O)CO)C1]

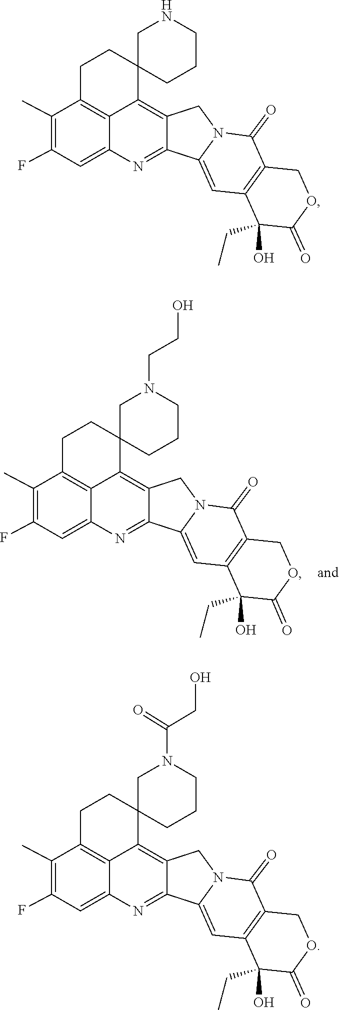

In various embodiments, the compound of Formula (IV) and/or Formula (IV*) can be represented by a structure selected from the following, or a pharmaceutically acceptable salt thereof:

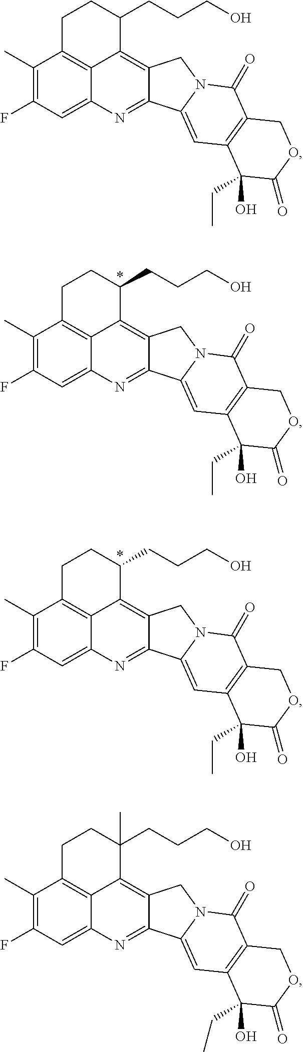

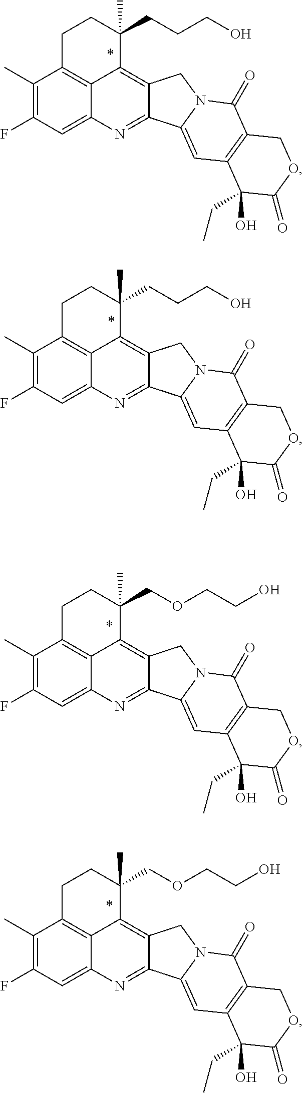

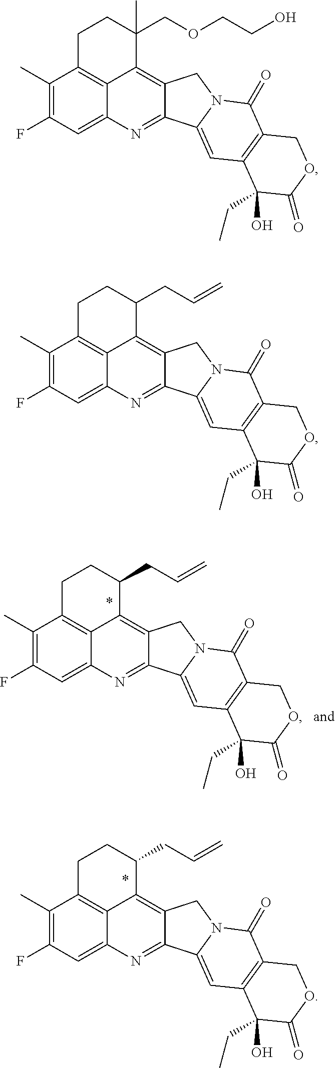

In various embodiments, the compound of Formula (IV) and/or Formula (IV*) can be represented by a structure selected from the following, or a pharmaceutically acceptable salt thereof:

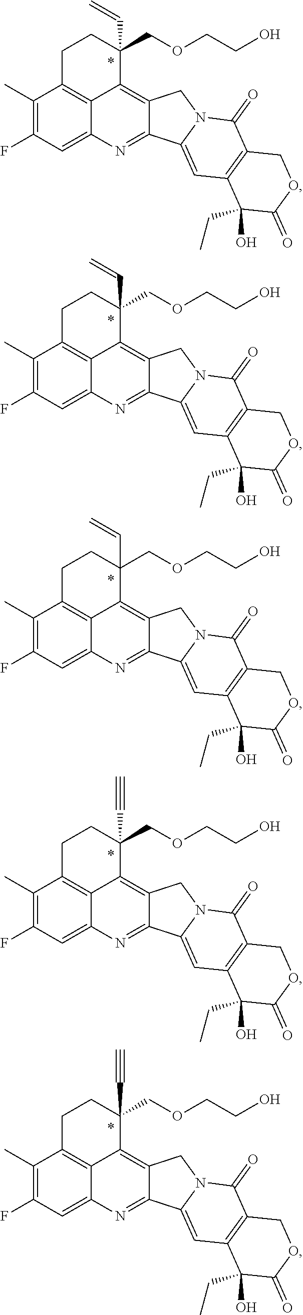

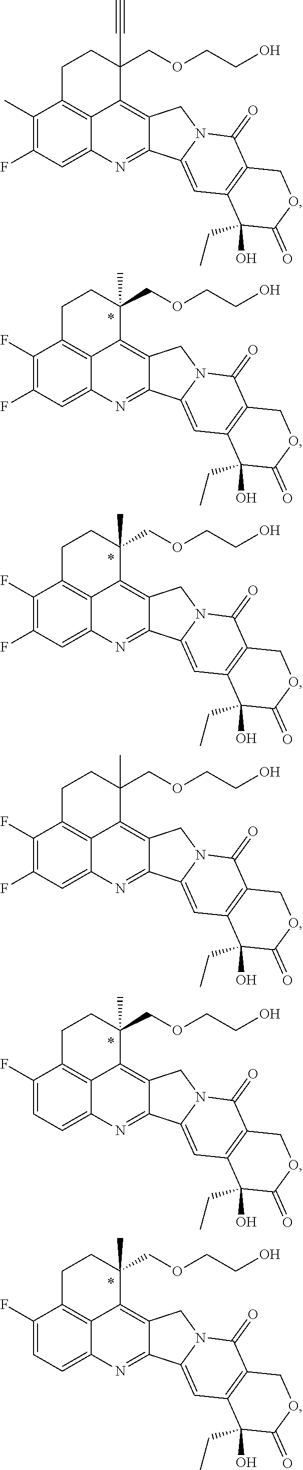

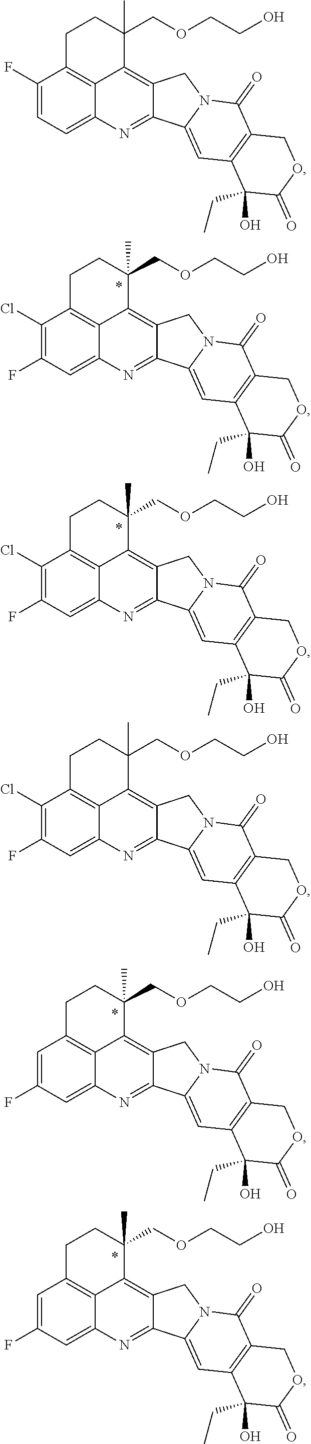

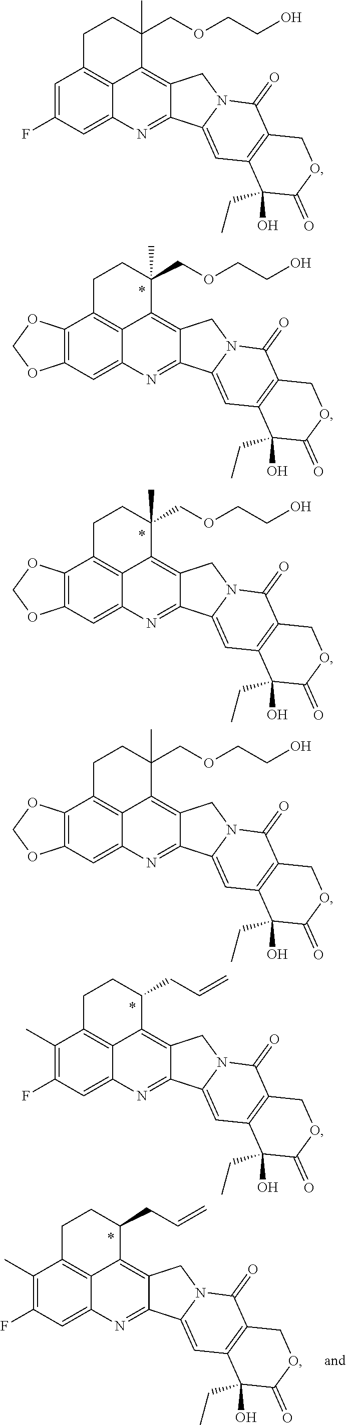

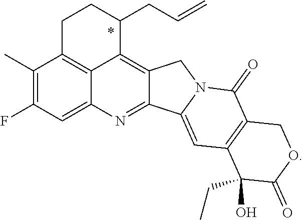 [SMILES: C=CCC1CCc2c(C)c(F)cc3nc4c(c1c23)Cn1c-4cc2c(c1=O)COC(=O)[C@]2(O)CC]

### Conjugates (Toxin-Linkers or Linker-Payloads)

Various embodiments disclosed herein relate to a conjugate of Formula (III), having the structure:

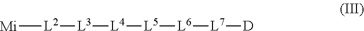

In various embodiments, Mi in Formula (III) can be

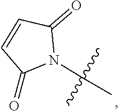 [SMILES: *N1C(=O)C=CC1=O]

D can be a drug moiety and -L²-L³-L⁴-L⁵-L⁶-L⁷- can be a linker that connects Mi to D.

In various embodiments, L² in Formula (III) can be absent,

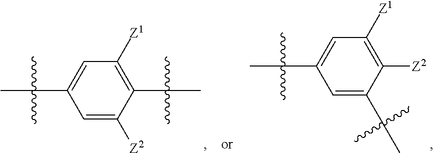

where Z¹ and Z² each individually can be hydrogen, halogen, NO₂, —O—(C₁-C₆ alkyl), or C₁-C₆ alkyl. In an embodiment, L² in Formula (III) can be absent. In an embodiment, L² in Formula (III) can be

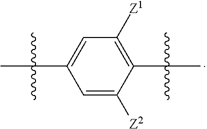

In an embodiment, L² in Formula (III) can be

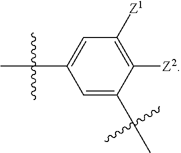

In various embodiments, Z¹ and Z² in Formula (III) each individually can be hydrogen, halogen, NO₂, —O—(C₁-C₆ alkyl), or C₁-C₆ alkyl. In an embodiment, at least one of Z¹ and Z² can be hydrogen. In an embodiment, at least one of Z¹ and Z² can be halogen. In an embodiment, at least one of Z¹ and Z² can be NO₂. In an embodiment, at least one of Z¹ and Z² can be —O—(C₁-C₆ alkyl). For example, in an embodiment, at least one of Z¹ and Z² can be methoxy. In an embodiment, at least one of Z¹ and Z² can be C₁-C₆ alkyl. For example, in an embodiment, at least one of Z¹ and Z² can be methyl.

In various embodiments, L³ in Formula (III) can be —(CH₂)n¹-C(═O)— or —(CH₂CH₂O)n¹-(CH₂)n¹C(═O)—, where each n¹ independently can be an integer of 0 to 12. In an embodiment, L³ can be —(CH₂)n¹-C(═O)—. For example, in an embodiment, L³ can be —C(═O)—. In an embodiment, L³ can be —(CH₂CH₂O)n¹-(CH₂)n¹C(═O)—. For example, in an embodiment, L³ can be —CH₂C(═O)—. In embodiment, n¹ can be an integer of 1 to 12, such as 1 to 6 or 1 to 3.

In various embodiments, L⁴ in Formula (III) can be a tetrapeptide residue. For example, in an embodiment, L⁴ can be a tetrapeptide residue selected from SEQ ID NO: 43 GGFG (gly-gly-phe-gly), SEQ ID NO: 44 EGGF (glu-gly-gly-phe), SEQ ID NO: 45 SGGF (ser-gly-gly-phe) and SEQ ID NO: 46 KGGF (lys-gly-gly-phe).

In various embodiments, L⁵ in Formula (III) can be absent or —[NH(CH₂)n²]n³-, where n² can be an integer of 0 to 6 and n³ can be an integer of 0 to 2. In an embodiment, L⁵ can be absent. In an embodiment, L⁵ can be —[NH(CH₂)n²]n³-. For example, in an embodiment, L⁵ can be —NH—. In another embodiment, L⁵ can be —NHCH₂—.

In various embodiments, L⁶ in Formula (III) can be absent or

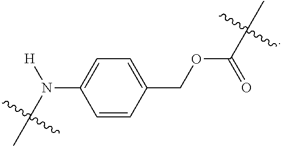 [SMILES: *Nc1ccc(COC(*)=O)cc1]

In an embodiment, L⁶ can be absent. In another embodiment, L⁶ can be

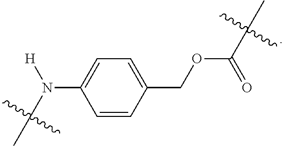 [SMILES: *Nc1ccc(COC(*)=O)cc1]

In various embodiments, L⁷ in Formula (III) can be absent,

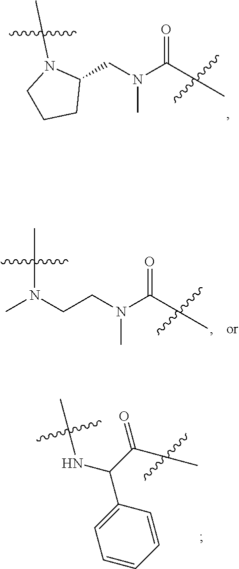

In an embodiment, L⁷ can be absent. In an embodiment, L⁷ can be

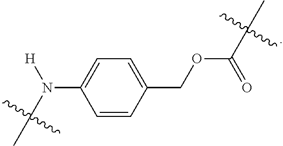 [SMILES: *Nc1ccc(COC(*)=O)cc1]

In an embodiment, L⁷ can be

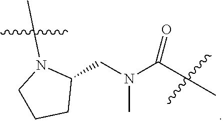 [SMILES: *C(=O)N(C)C[C@@H]1CCCN1*]

In an embodiment, L⁷ can be

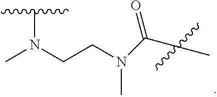 [SMILES: *C(=O)N(C)CCN(*)C]

In an embodiment, L⁷ can be

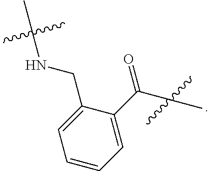 [SMILES: *NCc1ccccc1C(*)=O]

In various embodiments, D in a conjugate of Formula (III) can be a drug moiety as described herein (e.g., under the heading “Drug Moieties” below or under the heading “Compounds” above). In various embodiments, D in a conjugate of Formula (III) can be a compound of Formula (II). In various embodiments, D in a conjugate of Formula (III) can be a compound of Formula (II*). In various embodiments, D in a conjugate of Formula (III) can be a compound of Formula (IV). In various embodiments, D in a conjugate is Formula (III) can be a compound of Formula (IV*). In an embodiment, D can be a cytotoxic anti-cancer drug moiety.

In various embodiments, the conjugate of Formula (III) can be represented by a structure selected from the following:

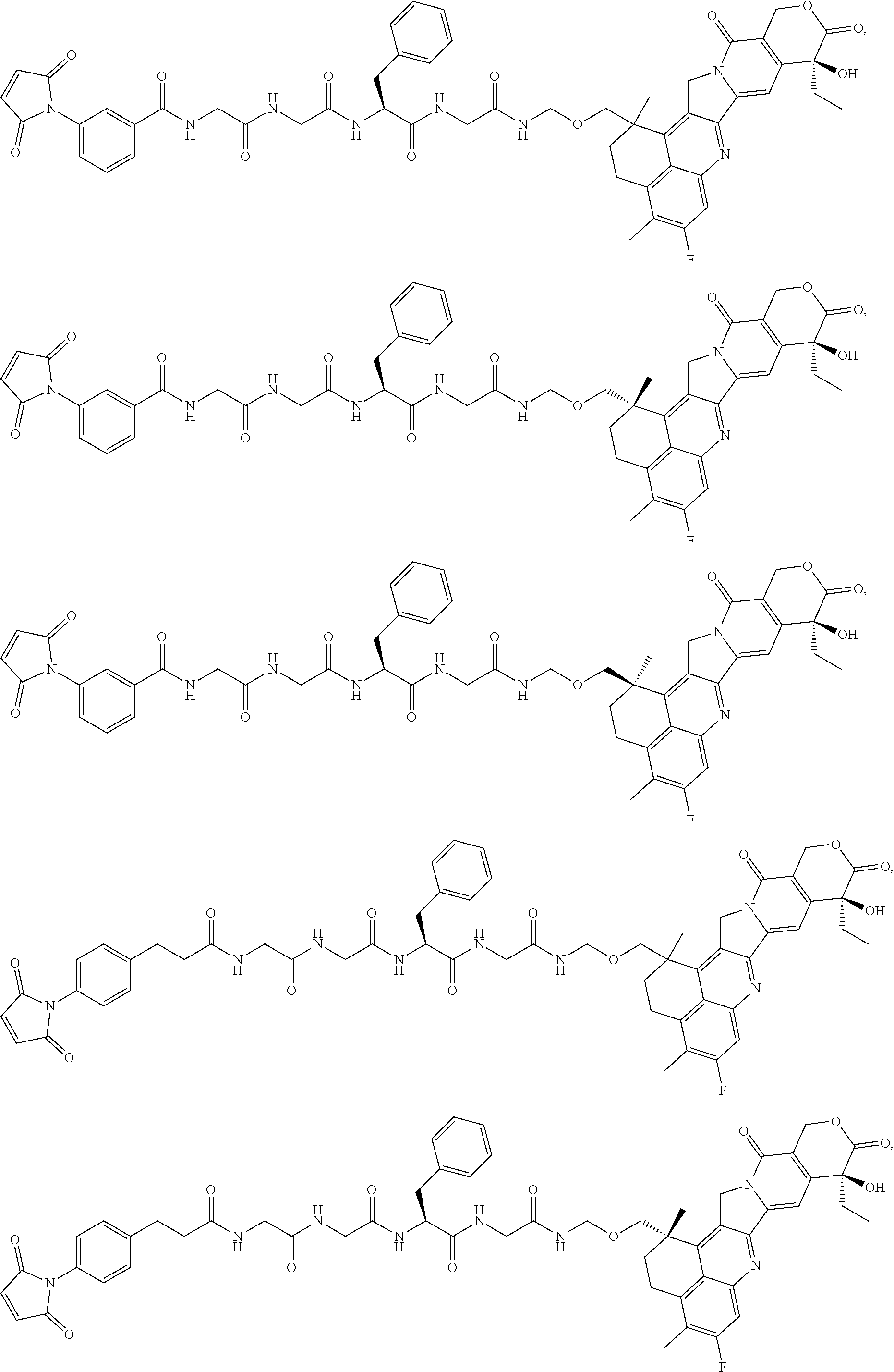

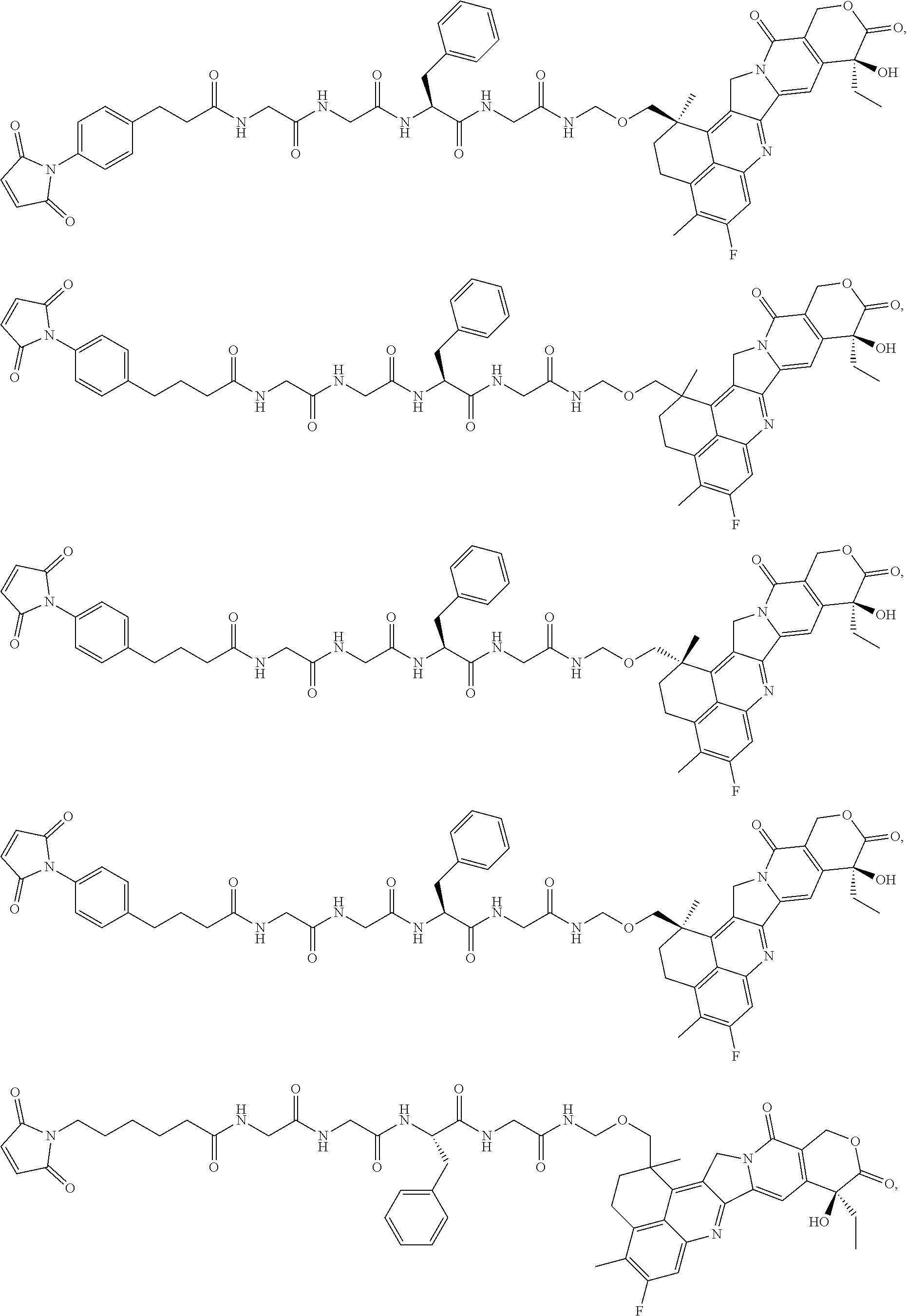

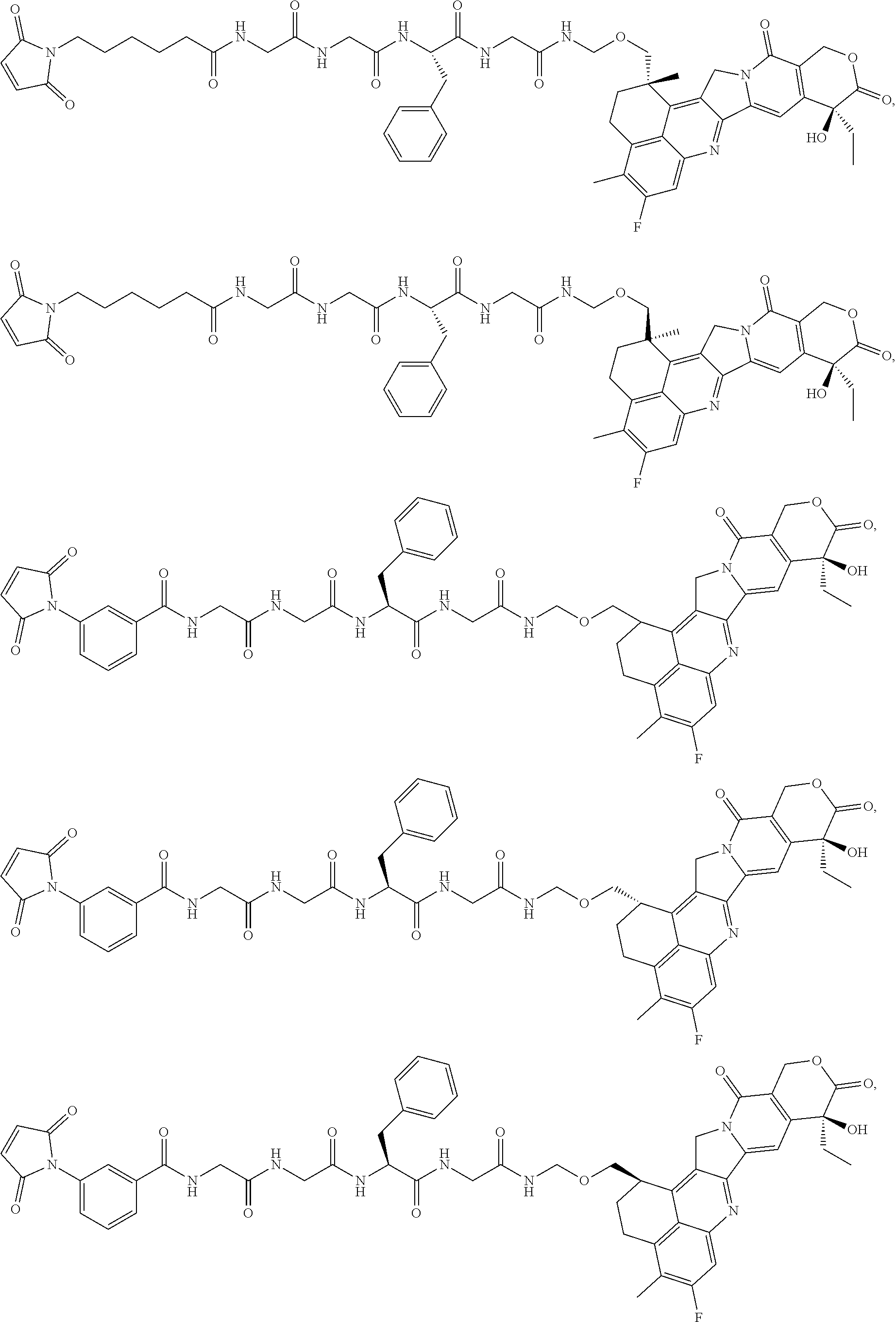

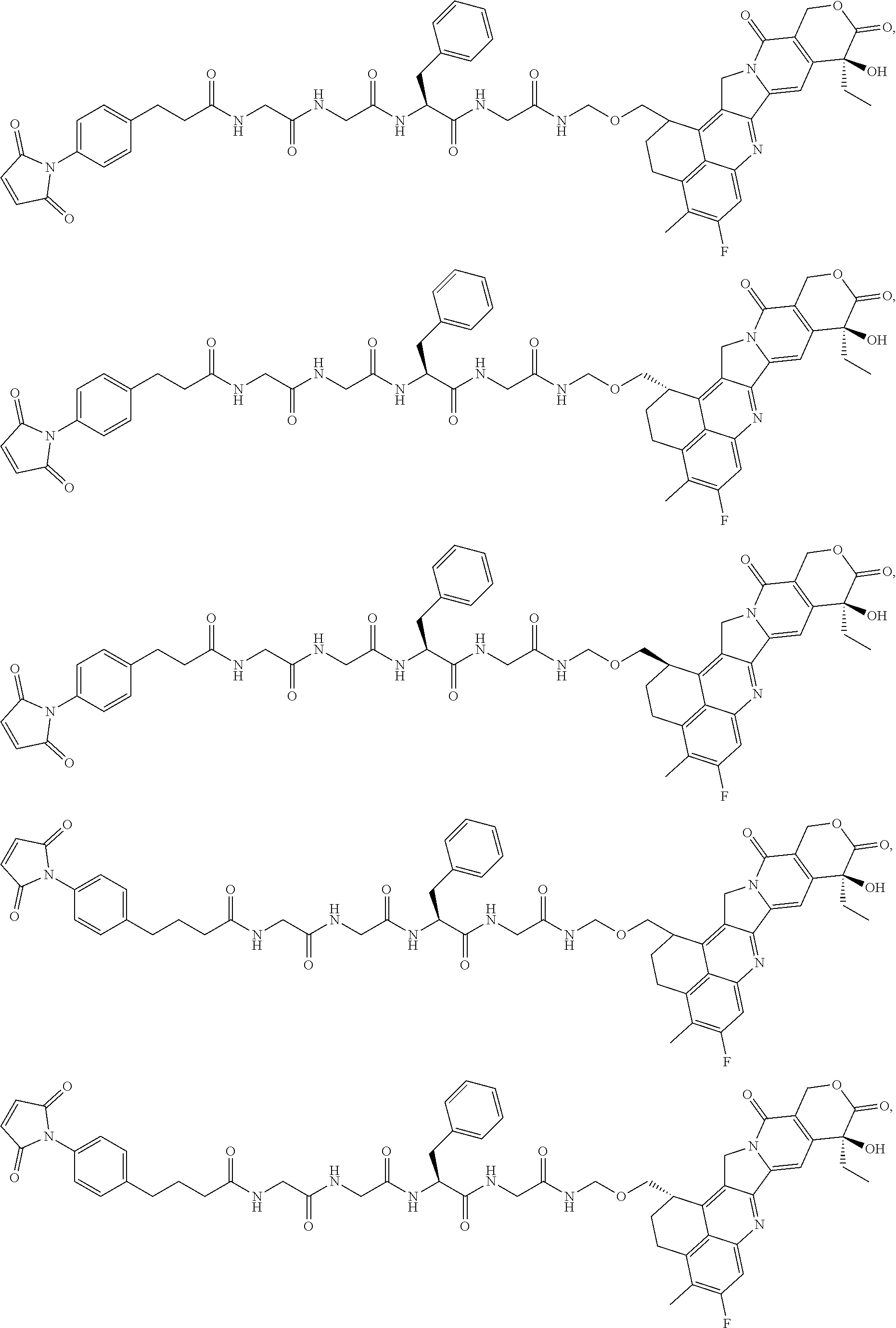

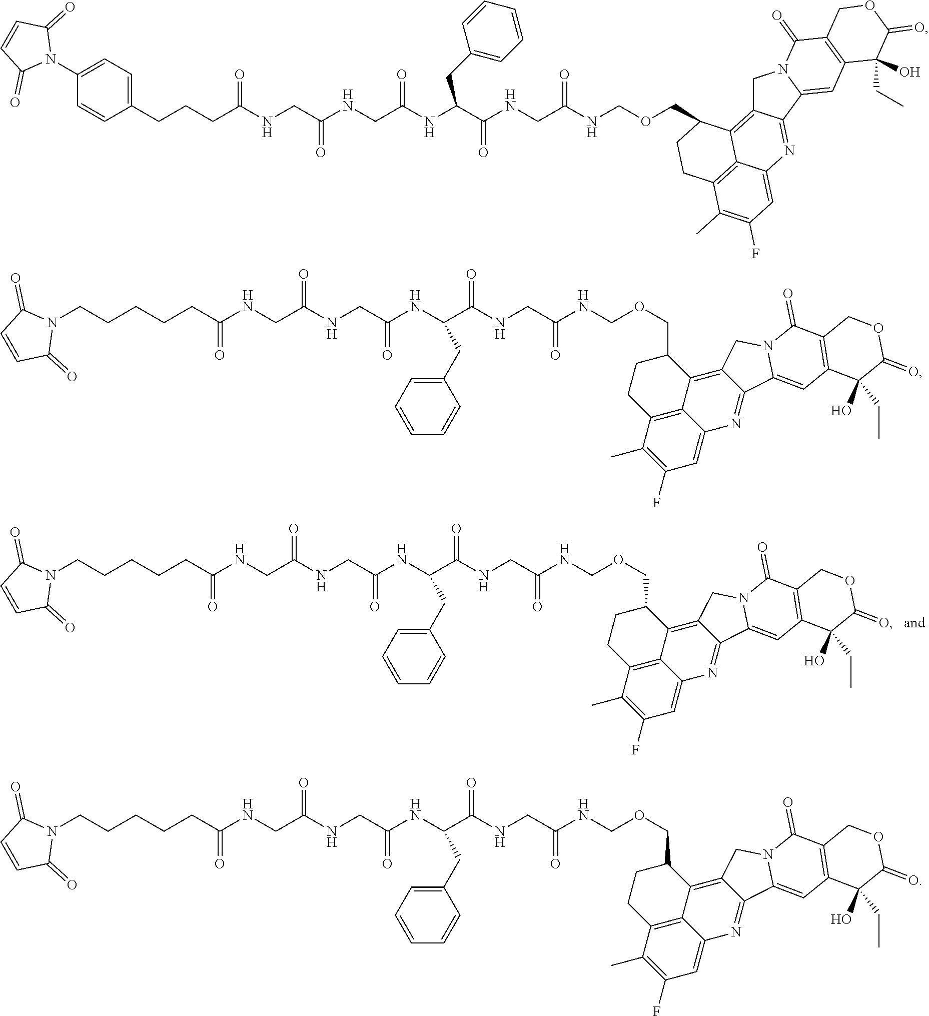

In various embodiments, the conjugate of Formula (III) can be represented by a structure selected from the following:

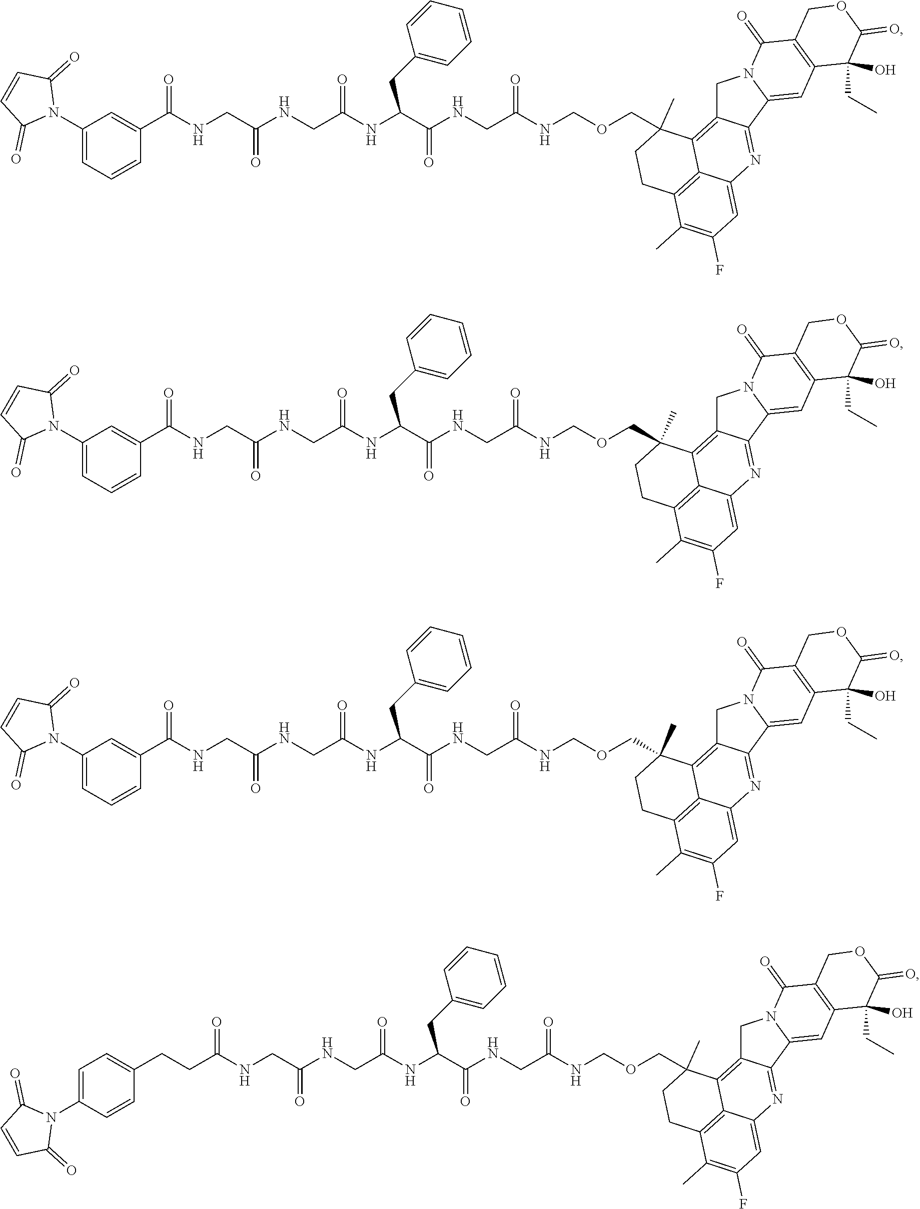

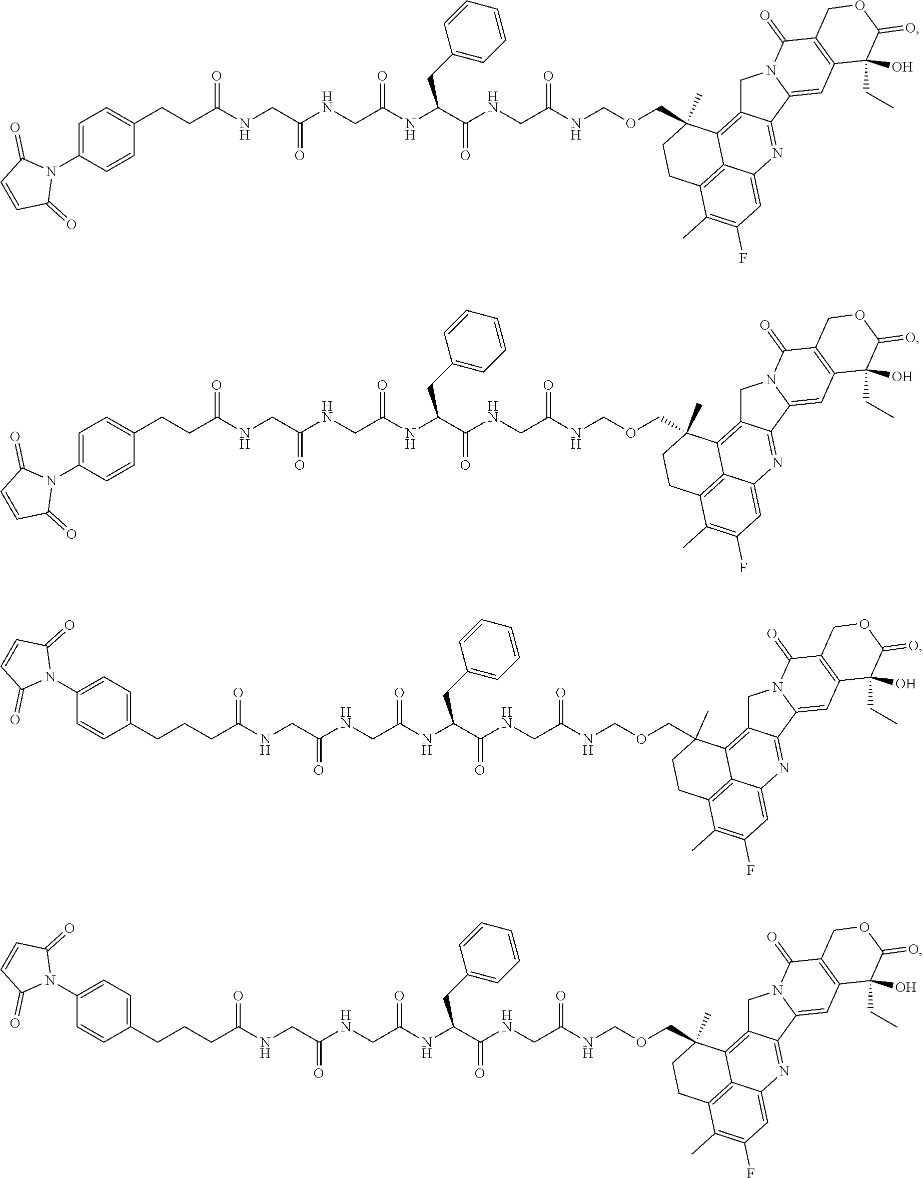

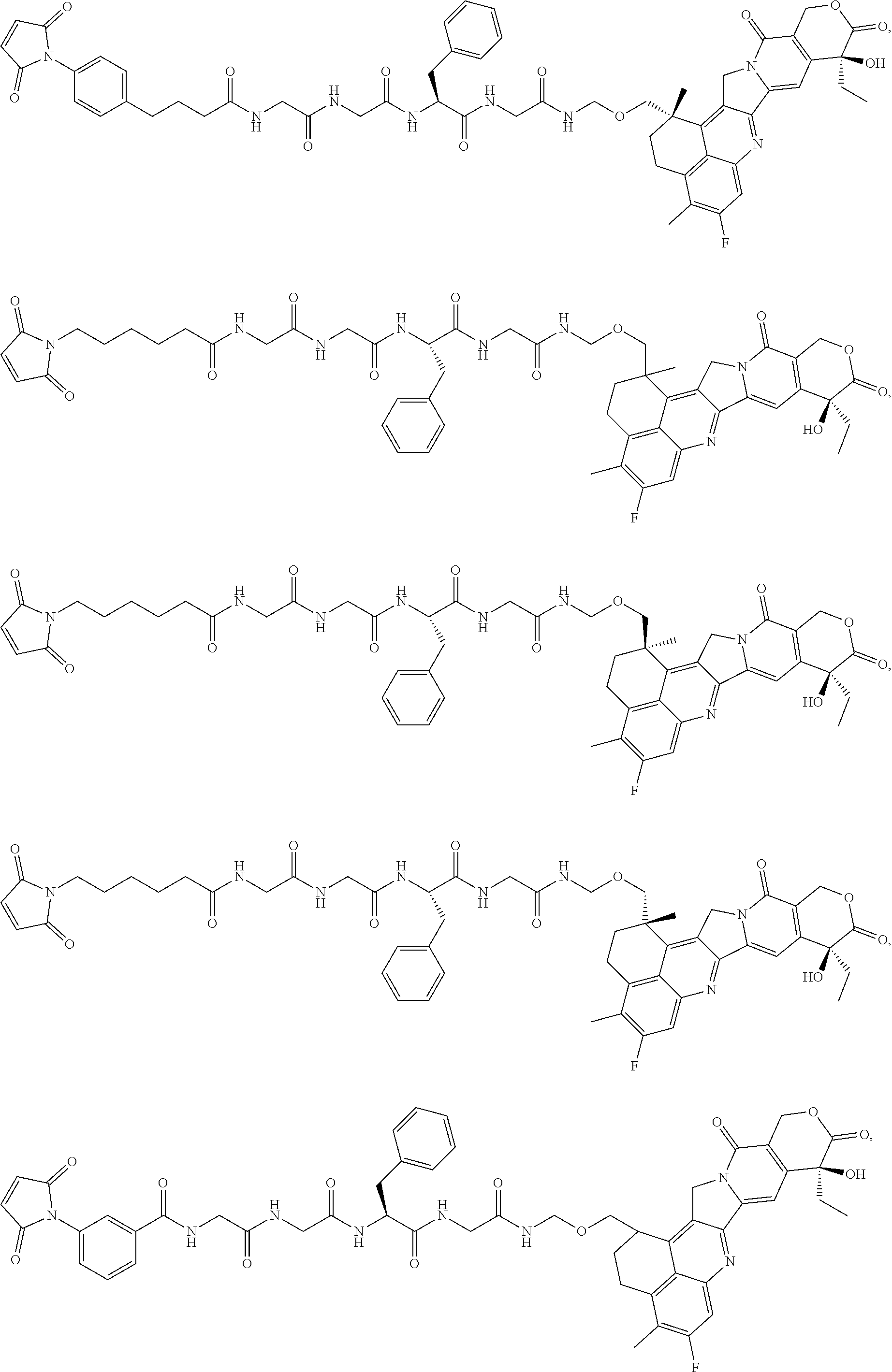

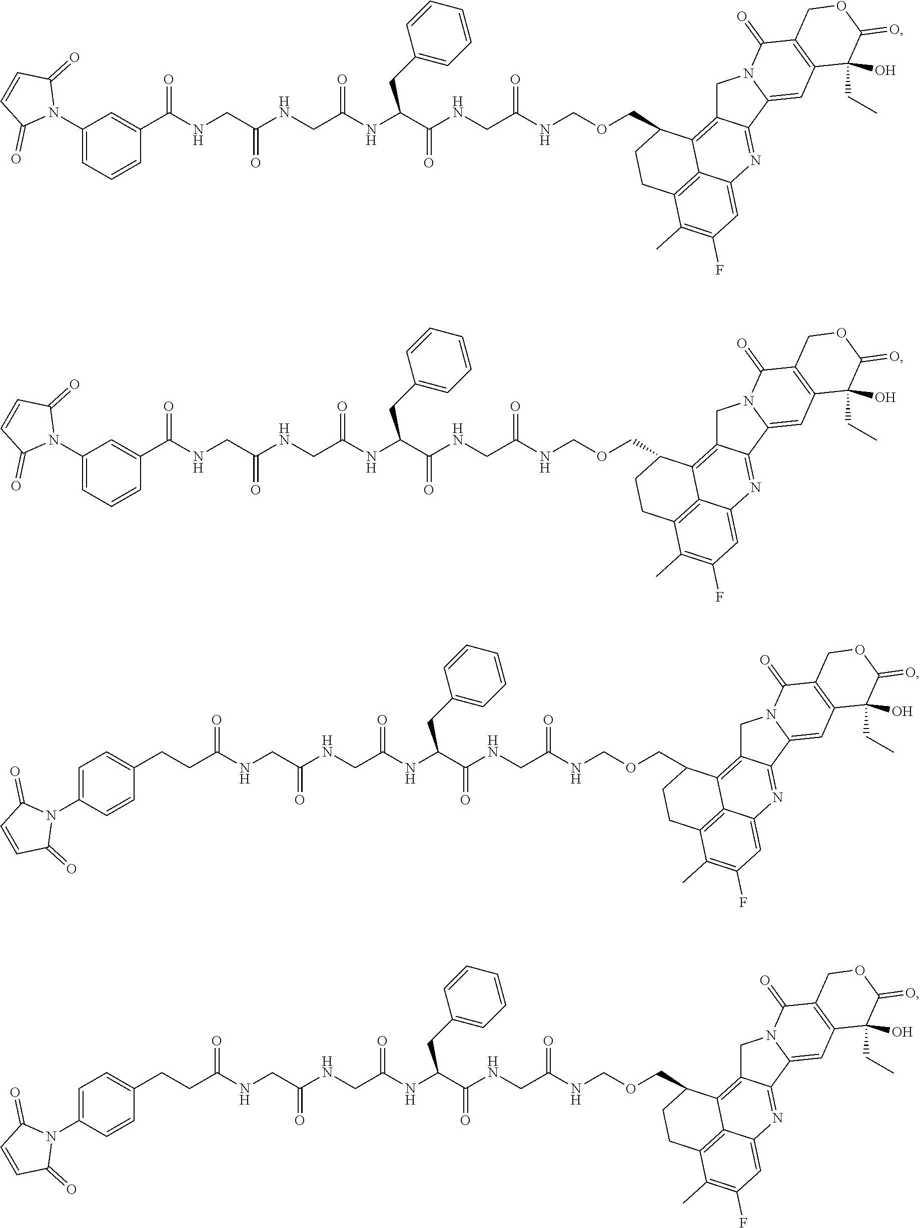

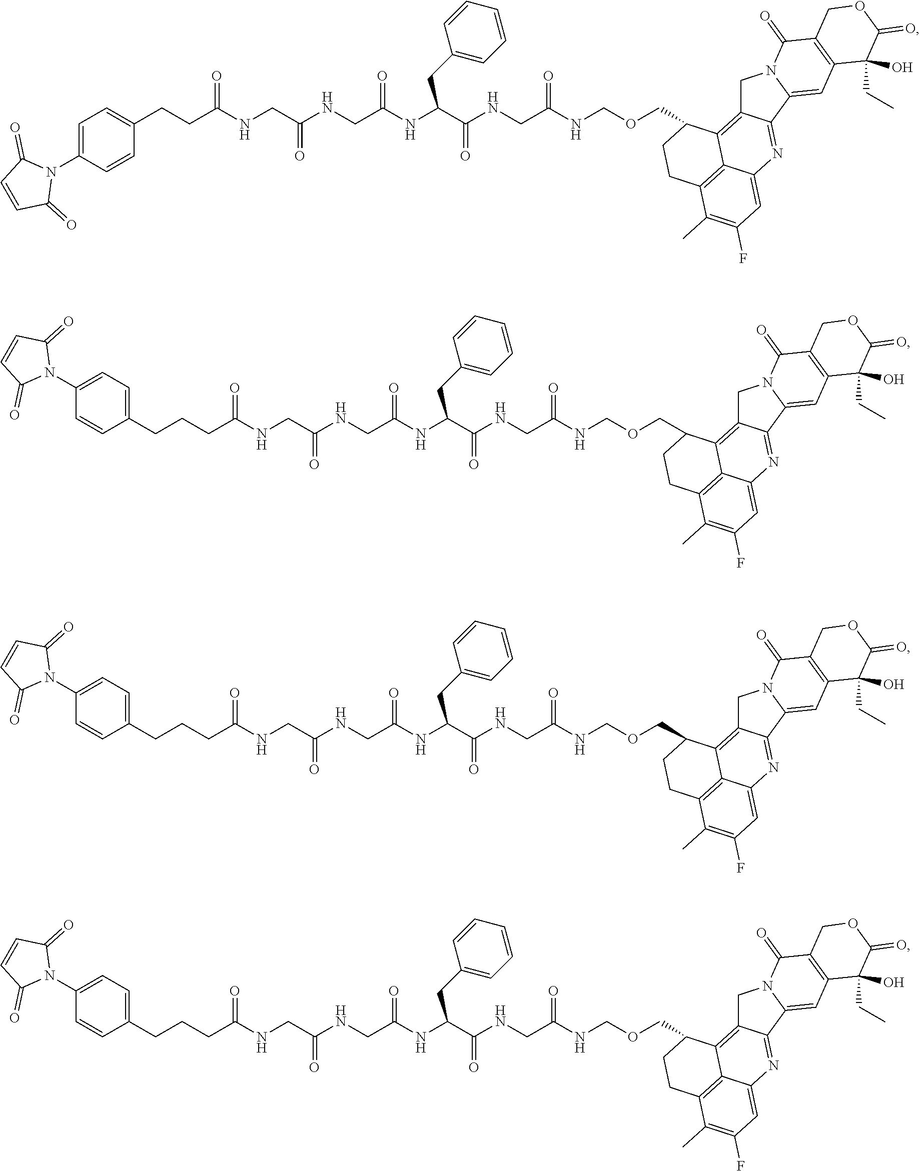

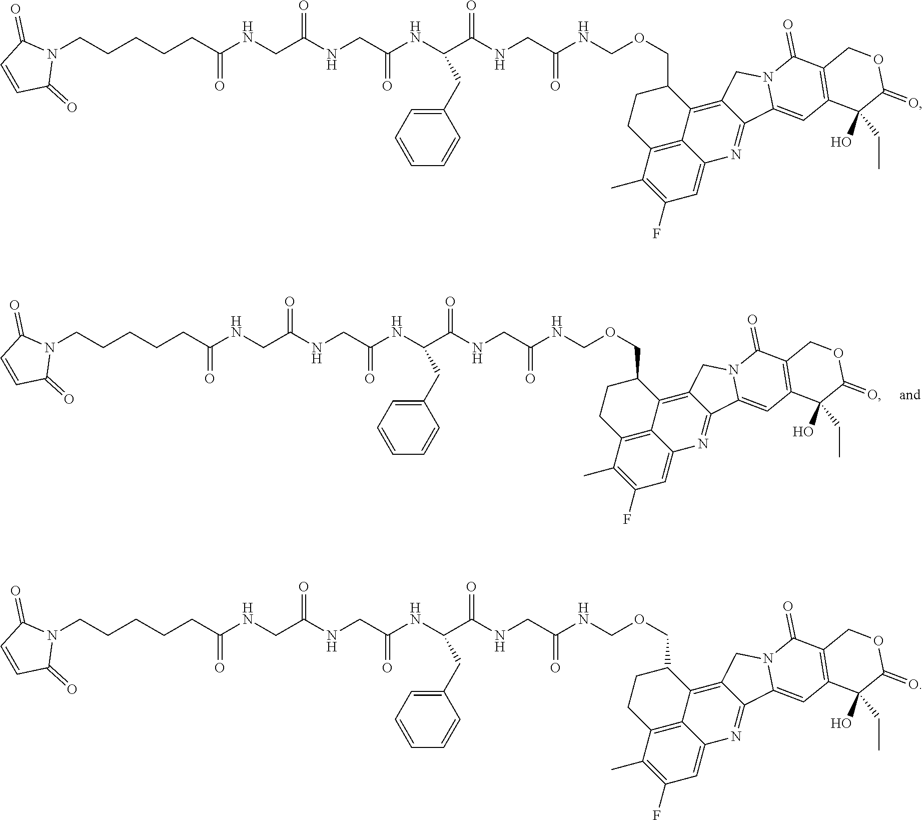

In various embodiments, the conjugate of Formula (III) can be represented by a structure selected from the following:

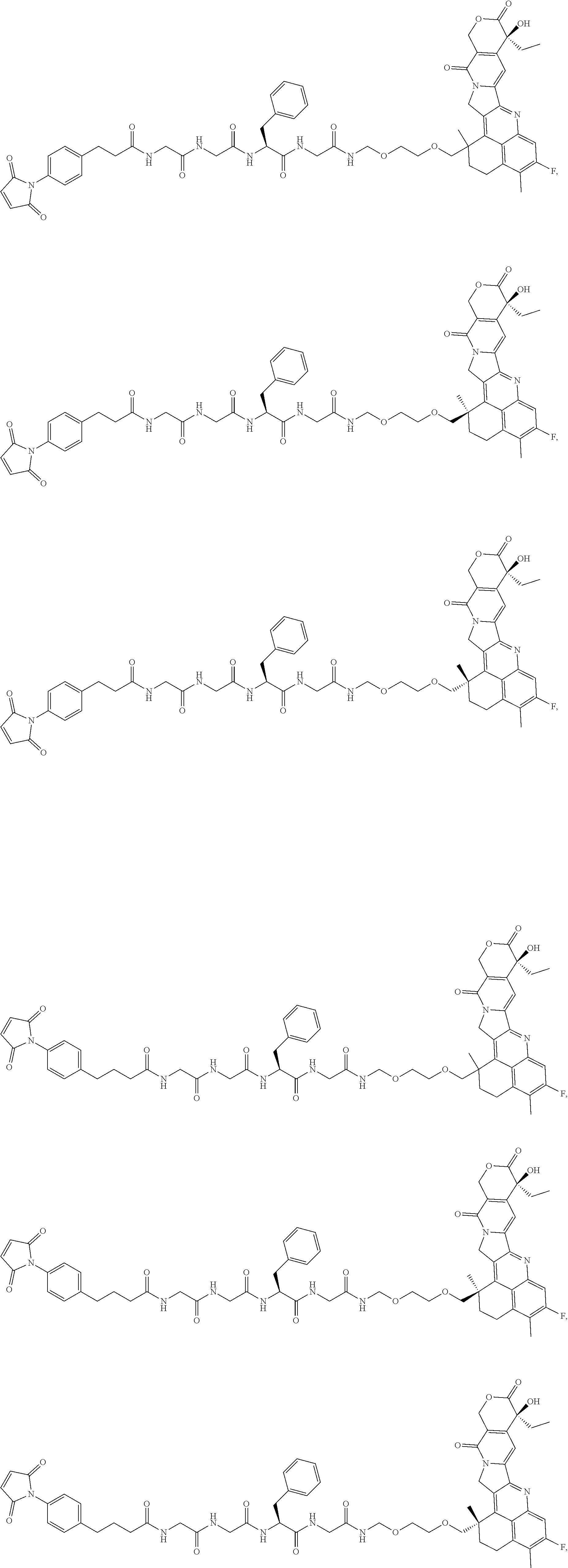

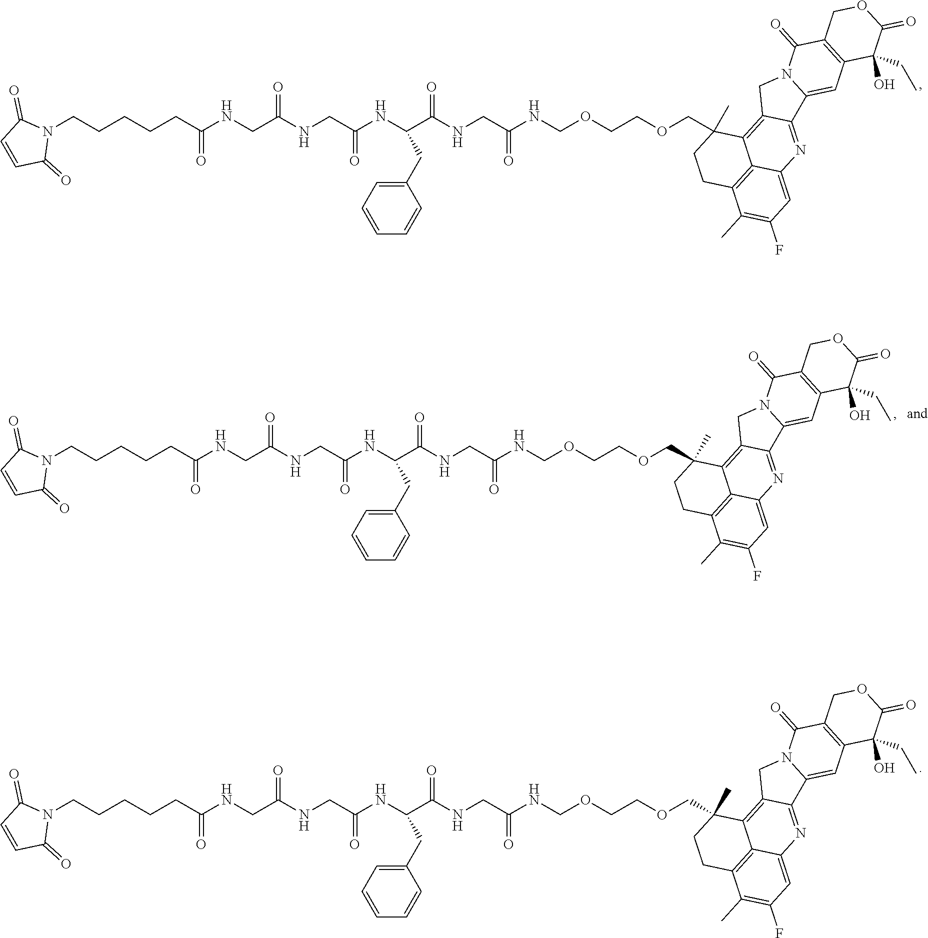 [SMILES: CC[C@@]1(O)C(=O)OCc2c1cc1n(c2=O)Cc2c-1nc1cc(F)c(C)c3c1c2C(C)(COCCOCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@](C)(COCCOCNC(=O)CNC(=O)[C@H](Cc1ccccc1)NC(=O)CNC(=O)CNC(=O)CCCCCN1C(=O)C=CC1=O)CC3 ; CC[C@@]1(O)C(=O)OCc2c1cc1n(c2=O)Cc2c-1nc1cc(F)c(C)c3c1c2[C@@](C)(COCCOCNC(=O)CNC(=O)[C@H](Cc1ccccc1)NC(=O)CNC(=O)CNC(=O)CCCCCN1C(=O)C=CC1=O)CC3]

### Drug Moieties

In various embodiments, D in the immunoconjugate of Formula (I) or in the conjugate of Formula (III) can be a drug moiety. The drug moiety may be any compound of the Formula (IV) or Formula (IV*) as described herein (e.g., as described above under the heading “Compounds”), with appropriate modification so that the linker -L²-L³-L⁴-L⁵-L⁶-L⁷ connects to D. For example, in various embodiments the drug moiety D can be a compound of Formula (II) having the structure:

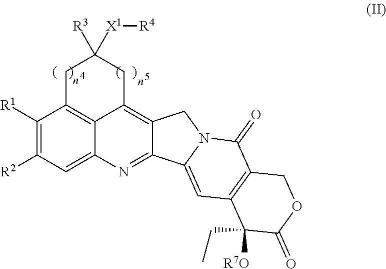

wherein:
   R¹ and R² each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² can be taken together to form a ring;
   R³ and R⁴ each individually can be selected from hydrogen, —OR⁵, —NR⁵R⁶, a substituted or an unsubstituted C₁-C₆ alkyl, a substituted or an unsubstituted C₂-C₆ alkenyl, a substituted or unsubstituted 4- or 5-membered heterocyclyl, and [(CY₂)ₚO(CY₂)_{q}]ₜCY₃, with the proviso that R³ and R⁴ cannot both be hydrogen; or R³ and R⁴ can be taken together with the carbon atom to which they are attached to form a substituted or unsubstituted 4-, 5- or 6-membered heterocyclyl or a 4-, 5- or 6-membered heterocyclyl that can be substituted with an optionally substituted C₁-C₃ alkyl; or one of R³ and R⁴ can be a substituted or an unsubstituted —(C₁-C₆ alkyl)-X², a substituted or an unsubstituted —(C₁-C₆ haloalkyl)-X², a substituted or an unsubstituted —(C₁-C₆ alkenyl)-X², a substituted or an unsubstituted —(C₁-C₆ haloalkenyl)-X², a substituted or an unsubstituted —(C₁-C₆ alkynyl)-X², or a substituted or an unsubstituted —(C₁-C₆ haloalkynyl)-X²;
   X² is —OR⁹, —SR⁹, or —NHR⁹;
   R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
   n⁴ and n⁵ each individually can be 0, 1 or 2, with the proviso that n⁴ and n⁵ cannot both be 0;
   each Y individually can be H or halogen;
   each m individually can be 1 or 2;
   each p individually can be 1, 2, 3, 4, 5, or 6;
   each q individually can be 0, 1, 2, 3, 4, 5, or 6;
   each t individually can be 1, 2, 3, 4, 5, or 6;
   R⁷ can be H, —COR⁸, —CO₂R⁸, —(CO)—NHR⁸, L⁴, L⁵, L⁶, or L⁷;
   R⁸ can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
   R⁹ can be H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
   each X³ individually can be —H, —OH, —SH, or —NH₂.

In alternative embodiments, the drug moiety D in the immunoconjugate of Formula (I) or the conjugate of Formula (III) can be a compound of Formula (II*) having the structure:

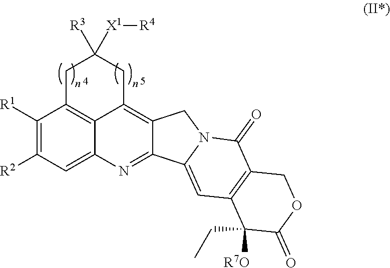

wherein:
   R¹ and R² each individually can be selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), —[(CY₂)ₚO(CY₂)_{q}]ₜCY₃, and a substituted or an unsubstituted —O—(CR⁵R⁶)ₘ—O— such that R¹ and R² taken together form a ring;
   R³ and R⁴ each individually can be selected from the group consisting of hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ cannot both be hydrogen; or R³ and R⁴ can be taken together with the carbon atom to which they are attached to form a substituted or unsubstituted 4-, 5- or 6-membered heterocyclyl or a 4-, 5- or 6-membered heterocyclyl that can be substituted with an optionally substituted C₁-C₃ alkyl; or one of R³ and R⁴ can be a substituted or an unsubstituted —(C₁-C₆ alkyl)-X², a substituted or an unsubstituted —(C₁-C₆ haloalkyl)-X², a substituted or an unsubstituted —(C₁-C₆ alkenyl)-X², a substituted or an unsubstituted —(C₁-C₆ haloalkenyl)-X², a substituted or an unsubstituted —(C₁-C₆ alkynyl)-X², or a substituted or an unsubstituted —(C₁-C₆ haloalkynyl)-X²;
   X² can be —OR⁹, —SR⁹, or —NHR⁹;
   R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl;
   n⁴ and n⁵ each individually can be 0, 1 or 2, with the proviso that n⁴ and n⁵ cannot both be 0;
   each Y individually can be H or halogen;
   each m individually can be 1 or 2;
   each p individually can be 1, 2, 3, 4, 5, or 6;
   each q individually can be 0, 1, 2, 3, 4, 5, or 6;
   each t individually can be 1, 2, 3, 4, 5, or 6;
   R⁷ can be H, —COR⁸, —CO₂R⁸, —(CO)—NHR⁸, L⁴, L⁵, L⁶, or L⁷;
   R⁸ can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
   R⁹ can be H, —COR⁸, —CO₂R⁸, —(CO)—NHR⁸, L⁴, L⁵, L⁶, or L⁷, with the proviso that exactly one of R⁷ and R⁹ can be L⁴, L⁵, L⁶, or L⁷; and
   each X³ individually can be —H, —OH, —SH, or —NH₂.

Those skilled in the art will appreciate that the compound of Formula (II) can connect to the linker -L²-L³-L⁴-L⁵-L⁶-L⁷- via R³ or R⁴ (when defined to include X² and thus R⁹) or via R⁷ as described below.

In various embodiments, R¹ and R² in Formula (II) or Formula (II*) each individually can be selected from hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² can be taken together to form a ring. In an embodiment, at least one of R¹ and R² can be hydrogen. In an embodiment, at least one of R¹ and R² can be halogen. For example, in an embodiment, at least one of R¹ and R² can be fluoro. In an embodiment, R¹ and R² can be each fluoro. In another embodiment, R¹ can be each fluoro and R² can be hydrogen. In still another embodiment, R¹ can be each hydrogen and R² can be fluoro. In an embodiment, at least one of R¹ and R² can be chloro. In one embodiment, one of R¹ and R² can be fluoro and the other of R¹ and R² can be chloro. In one embodiment, R¹ can be chloro and R² can be fluoro. In an embodiment, at least one of R¹ and R² can be —CN. In an embodiment, at least one of R¹ and R² can be —OR⁵, wherein R⁵ can be a substituted or an unsubstituted C₁-C₆ alkyl. For example, in an embodiment, at least one of R¹ and R² can be methoxy. In one embodiment, R¹ and R² can be a substituted or an unsubstituted —O—(CR⁵R⁶)ₘ—O— such that R¹ and R² can be taken together to form a ring. In an embodiment, one of R¹ and R² can be a substituted or an unsubstituted —O—(CH₂)—O— such that R¹ and R² can be taken together to form 1,3-dioxolane.

In an embodiment, at least one of R¹ and R² in Formula (II) or Formula (II*) can be —NR⁵R⁶, wherein R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl.

In an embodiment, at least one of R¹ and R² in Formula (II) or Formula (II*) can be a substituted or an unsubstituted C₁-C₆ alkyl. In an embodiment, at least one of R¹ and R² can be a C₁-C₃ alkyl. For example, in an embodiment, at least one of R¹ and R² can be methyl. In an embodiment, at least one of R¹ and R² can be a C₁-C₃ alkyl and the other can be a halogen. For example, in an embodiment, at least one of R¹ and R² can be methyl and the other can be fluoro.

In an embodiment, at least one of R¹ and R² in Formula (II) or Formula (II*) can be a substituted or an unsubstituted C₁-C₆ haloalkyl. For example, in an embodiment, at least one of R¹ and R² can be difluoromethyl. In an embodiment, at least one of R¹ and R² can be a substituted or an unsubstituted —O—(C₁-C₆ alkyl). For example, in an embodiment, at least one of R¹ and R² can be methoxy. In an embodiment, at least one of R¹ and R² can be —[(CY₂)ₚO(CY₂)_{q}]ₜCY₃. In an embodiment, R¹ and R² individually can be a substituted or an unsubstituted —O—(CR⁵R⁶)ₘ—O— such that R¹ and R² can be taken together to form a ring in which the ends of the —O—(CR⁵R⁶)ₘ—O— are covalently bonded to the phenyl ring at the R¹ and R² positions of Formula (II) or Formula (II*) to form a heterocyclic ring.

In an embodiment, one of R¹ and R² in Formula (II) or Formula (II*) can be hydrogen and the other of R¹ and R² can be a halogen. In an embodiment, one of R¹ and R² in Formula (II) or Formula (II*) can be hydrogen and the other of R¹ and R² can be fluoro. In an embodiment, R¹ in Formula (II) or Formula (II*) can be fluoro and R² can be hydrogen. In an embodiment, R¹ in Formula (II) or Formula (II*) can be hydrogen and R² can be fluoro. In an embodiment, one of R¹ and R² can be hydrogen and the other of R¹ and R² can be a substituted or an unsubstituted C₁-C₆ alkyl. In an embodiment, one of R¹ and R² can be hydrogen and the other of R¹ and R² can be a substituted or an unsubstituted C₁-C₆ haloalkyl. In an embodiment, one of R¹ and R² can be hydrogen and the other of R¹ and R² can be a substituted or an unsubstituted —O—(C₁-C₆ alkyl). In an embodiment, both R¹ and R² can be hydrogen. In an embodiment, neither R¹ nor R² can be hydrogen.

In an embodiment, one of R¹ and R² in Formula (II) or Formula (II*) can be halogen and the other of R¹ and R² can be a substituted or an unsubstituted C₁-C₆ alkyl. In an embodiment, one of R¹ and R² can be halogen and the other of R¹ and R² can be a substituted or an unsubstituted C₁-C₆ haloalkyl. In an embodiment, one of R¹ and R² can be halogen and the other of R¹ and R² can be a substituted or an unsubstituted —O—(C₁-C₆ alkyl). In an embodiment, both R¹ and R² independently can be halogen. In an embodiment, neither R¹ nor R² can be halogen.

In an embodiment, one of R¹ and R² in Formula (II) or Formula (II*) can be a substituted or an unsubstituted C₁-C₆ alkyl and the other of R¹ and R² can be a substituted or an unsubstituted C₁-C₆ haloalkyl. In an embodiment, one of R¹ and R² can be a substituted or an unsubstituted C₁-C₆ alkyl and the other of R¹ and R² can be a substituted or an unsubstituted —O—(C₁-C₆ alkyl). In an embodiment, both R¹ and R² independently can be a substituted or an unsubstituted C₁-C₆ alkyl. In an embodiment, neither R¹ nor R² can be a substituted or an unsubstituted C₁-C₆ alkyl.

In an embodiment, one of R¹ and R² in Formula (II) or Formula (II*) can be a substituted or an unsubstituted C₁-C₆ haloalkyl and the other of R¹ and R² can be a substituted or an unsubstituted —O—(C₁-C₆ alkyl). In an embodiment, both R¹ and R² independently can be a substituted or an unsubstituted C₁-C₆ haloalkyl. In an embodiment, neither R¹ nor R² can be a substituted or an unsubstituted C₁-C₆ haloalkyl.

In an embodiment, one of R¹ and R² in Formula (II) or Formula (II*) can be a substituted or an unsubstituted —O—(C₁-C₆ alkyl). In an embodiment, both R¹ and R² independently can be a substituted or an unsubstituted —O—(C₁-C₆ alkyl). In an embodiment, neither R¹ nor R² can be a substituted or an unsubstituted —O—(C₁-C₆ alkyl). In an embodiment, R¹ and R² can be a substituted or an unsubstituted —O—(CR⁵R⁶)ₘ—O— such that R¹ and R² can be taken together to form a ring. In various embodiments, R¹ and R² each individually can be selected from hydrogen, fluoro, methoxy, methyl, difluoromethyl, and —O—(CH₂)—O— such that R¹ and R² can be taken together to form a ring.

In various embodiments, R³ in Formula (II) can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R³ can be hydrogen. In an embodiment, R³ can be —OR⁵, such as —OCH₃. In an embodiment, R³ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R³ can be a substituted or an unsubstituted C₁-C₆ alkyl. For example, in an embodiment, R³ can be methyl, —CH₂OH or —CH₂CH₂OH. As another example, in an embodiment, R³ can be —(CH₂)₃OH or —CH₂O(CH₂)₂OH. In an embodiment, R³ can be a substituted or an unsubstituted C₂-C₆ alkenyl. For example, in an embodiment, R³ can be —CH═CH₂. In another embodiment, R³ can be —CH₂CH═CH₂. In an embodiment, R³ can be a substituted or an unsubstituted C₂-C₆ alkynyl. For example, in an embodiment, R³ can be —C≡CH. In an embodiment, R³ is —[(CY₂)ₚO(CY₂)_{q}]ₜCY₃, where each Y individually can be H or halogen.

In various embodiments, R⁴ in Formula (II) can be selected from hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R⁴ can be hydrogen. In an embodiment, R⁴ can be —OR⁵, such as —OCH₃. In an embodiment, R⁴ can be —NR⁵R⁶, where R⁵ and R⁶ each individually can be a substituted or an unsubstituted C₁-C₆ alkyl; or R⁵ and R⁶, can be taken together with the nitrogen atom to which they are attached to form a substituted or unsubstituted 4- or 5-membered heterocyclyl. In an embodiment, R⁴ can be a substituted or an unsubstituted C₁-C₆ alkyl. For example, in an embodiment, R⁴ can be methyl, —CH₂OH or —CH₂CH₂OH. As another example, in an embodiment, R⁴ can be —(CH₂)₃OH or —CH₂O(CH₂)₂OH. In an embodiment, R⁴ can be a substituted or an unsubstituted C₂-C₆ alkenyl. For example, in an embodiment, R⁴ can be —CH═CH₂. In another embodiment, R⁴ is —CH₂CH═CH₂. In an embodiment, R⁴ can be a substituted or an unsubstituted C₂-C₆ alkynyl. For example, in an embodiment, R⁴ can be —C≡CH. In an embodiment, R⁴ can be —[(CY₂)ₚO(CY₂)_{q}]ₜCY₃, where each Y individually can be H or halogen.

In various embodiments, R³ in Formula (II*) can be selected from hydrogen, —OR⁵, —NR⁵R⁶, a substituted or an unsubstituted C₁-C₆ alkyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R³ can be hydrogen. In an embodiment, R³ can be —OR⁵, such as —OCH₃. In an embodiment, R³ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R³ can be a substituted or an unsubstituted C₁-C₆ alkyl. For example, in an embodiment, R³ can be methyl, —CH₂OH, or —CH₂CH₂OH. As another example, in an embodiment, R³ can be —(CH₂)₃OH or —CH₂O(CH₂)₂OH. In an embodiment, R³ can be —[(CY₂)ₚO(CY₂)_{q}]ₜCY₃, where each Y individually can be H or halogen.

In various embodiments, R⁴ in Formula (II*) can be selected from hydrogen, —OR⁵, —NR⁵R⁶, a substituted or an unsubstituted C₁-C₆ alkyl, a substituted or unsubstituted 4- or 5-membered heterocyclyl, and —[(CY₂)ₚO(CY₂)_{q}]ₜCY₃, with the proviso that R³ and R⁴ cannot both be hydrogen. In an embodiment, R⁴ can be hydrogen. In an embodiment, R⁴ can be —OR⁵, such as —OCH₃. In an embodiment, R⁴ can be —NR⁵R⁶, where R⁵ and R⁶ each individually can be a substituted or an unsubstituted C₁-C₆ alkyl; or R⁵ and R⁶ can be taken together with the nitrogen atom to which they are attached to form a substituted or unsubstituted 4- or 5-membered heterocyclyl. In an embodiment, R⁴ can be a substituted or an unsubstituted C₁-C₆ alkyl. For example, in an embodiment, R⁴ can be methyl, —CH₂OH, or —CH₂CH₂OH. As another example, in an embodiment, R⁴ can be —(CH₂)₃OH or —CH₂O(CH₂)₂OH. In an embodiment, R⁴ can be —[(CY₂)ₚO(CY₂)_{q}]ₜCY₃, where each Y individually can be H or halogen.

In some embodiments, one of R³ and R⁴ in Formula (II) or Formula (II*) can be hydrogen and the other of R³ and R⁴ can be a substituted or an unsubstituted C₁-C₆ alkyl. In other embodiments, one of R³ and R⁴ can be hydrogen and the other of R³ and R⁴ can be —NR⁵R⁶. In some embodiments, one of R³ and R⁴ can be —CH₃, with the proviso that R³ and R⁴ cannot both be —CH₃. For example, in some embodiments, one of R³ and R⁴ can be —CH₃ and the other of R³ and R⁴ can be a substituted or an unsubstituted C₁-C₆ alkyl that cannot be —CH₃, such as —CH₂OH or —CH₂CH₂OH. In some embodiments, one of R³ and R⁴ can be CH₃, and the other of R³ and R⁴ can be —NR⁵R⁶.

In some embodiments, R³ and/or R⁴ in Formula (II) or Formula (II*) can be —NR⁵R⁶ as described herein. For example, in an embodiment, R⁵ and R⁶ each individually can be a substituted or an unsubstituted C₁-C₆ alkyl (such as —CH₃, —CH₂CH₂OH, or

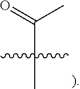 [SMILES: *C(C)=O]

In other embodiments of —NR⁵R⁶ described herein, R⁵ and R⁶ can be taken together with the nitrogen atom to which they are attached to form a substituted or unsubstituted 4- or 5-membered heterocyclyl. For example, in some embodiments, the —NR⁵R⁶ can be

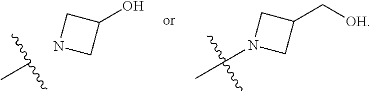

In other embodiments, the —NR⁵R⁶ can be

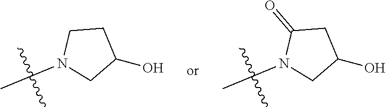 [SMILES: *N1CCC(O)C1 ; *N1CC(O)CC1=O]

In some embodiments, R³ and/or R⁴ in Formula (II) or Formula (II*) can be —[(CY₂)ₚO(CY₂)_{q}]ₜCY₃, such as —CH₂OCH₃. For example, in some embodiments, one of R³ and R⁴ can be —CH₂OH or —CH₂CH₂OH, and the other of R³ and R⁴ can be —CH₂OCH₃.

In some embodiments, R³ and R⁴ in Formula (II) or Formula (II*) can be taken together with the carbon atom to which they are attached to form an unsubstituted 4-, 5- or 6-membered heterocyclyl or a 4-, 5- or 6-membered heterocyclyl that can be substituted with an optionally substituted C₁-C₃ alkyl. For example, in some embodiments, R³ and R⁴, taken together with the carbon atom to which they are attached, can be selected from

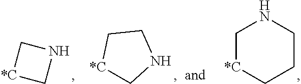

where * indicates the carbon atom to which R³ and R⁴ are attached. In other embodiments, R³ and R⁴, taken together with the carbon atom to which they are attached, can be selected from

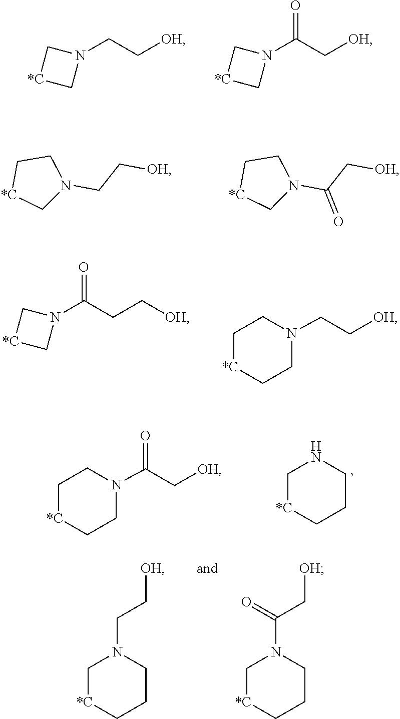

where * indicates the carbon atom to which R³ and R⁴ are attached.

In one embodiment, one of R³ and R⁴ in Formula (II) or Formula (II*) can be hydrogen and the other of R³ and R⁴ can be —CH₂OH. In one embodiment, one of R³ and R⁴ in Formula (II) or Formula (II*) can be —CH₃ and the other of R³ and R⁴ can be —CH₂OH. In one embodiment, one of R³ and R⁴ in Formula (IV) or Formula (IV*) can be —OCH₃ and the other of R³ and R⁴ can be —CH₂OH. In one embodiment, one of R³ and R⁴ in Formula (II) or Formula (II*) is —C₂H₅ and the other of R³ and R⁴ can be —CH₂OH. In one embodiment, one of R³ and R⁴ in Formula (II) can be —CH═CH₂ and the other of R³ and R⁴ can be —CH₂OH. In one embodiment, one of R³ and R⁴ in Formula (II) can be —C≡CH and the other of R³ and R⁴ can be —CH₂OH. In one embodiment, one of R³ and R⁴ in Formula (II) or Formula (II*) can be hydrogen and the other of R³ and R⁴ can be —(CH₂)₂OH. In one embodiment, one of R³ and R⁴ in Formula (II) or Formula (II) can be —CH₃ and the other of R³ and R⁴ can be —(CH₂)₂OH. In one embodiment, one of R³ and R⁴ in Formula (II) or Formula (II*) can be —OCH₃ and the other of R³ and R⁴ can be —(CH₂)₂OH. In one embodiment, one of R³ and R⁴ in Formula (II) or Formula (II*) can be —C₂H₅ and the other of R³ and R⁴ can be —(CH₂)₂OH. In one embodiment, one of R³ and R⁴ in Formula (IV) can be —CH═CH₂ and the other of R³ and R⁴ can be —(CH₂)₂OH. In one embodiment, one of R³ and R⁴ in Formula (II) can be —C≡CH and the other of R³ and R⁴ can be —(CH₂)₂OH. In one embodiment, one of R³ and R⁴ in Formula (II) or Formula (II*) can be hydrogen and the other of R³ and R⁴ can be —(CH₂)₃OH. In one embodiment, one of R³ and R⁴ in Formula (II) or Formula (II*) can be —CH₃ and the other of R³ and R⁴ can be —(CH₂)₃OH. In one embodiment, one of R³ and R⁴ in Formula (II) or Formula (II*) can be —OCH₃ and the other of R³ and R⁴ can be —(CH₂)₃OH. In one embodiment, one of R³ and R⁴ in Formula (II) or Formula (II*) can be —C₂H₅ and the other of R³ and R⁴ can be —(CH₂)₃OH. In one embodiment, one of R³ and R⁴ in Formula (II) can be —CH═CH₂ and the other of R³ and R⁴ can be —(CH₂)₃OH. In one embodiment, one of R³ and R⁴ in Formula (II) can be —C≡CH and the other of R³ and R⁴ can be —(CH₂)₃OH. In one embodiment, R³ and R⁴ both can be —CH₂OH. In one embodiment, one of R³ and R⁴ in Formula (II) or Formula (II*) can be hydrogen and the other of R³ and R⁴ can be —CH₂O(CH₂)₂OH. In one embodiment, one of R³ and R⁴ in Formula (II) or Formula (II*) can be —CH₃ and the other of R³ and R⁴ can be —CH₂O(CH₂)₂OH. In one embodiment, one of R³ and R⁴ in Formula (II) or Formula (II*) can be —OCH₃ and the other of R³ and R⁴ is —CH₂O(CH₂)₂OH. In one embodiment, one of R³ and R⁴ in Formula (II) or Formula (II*) can be —C₂H₅ and the other of R³ and R⁴ is —CH₂O(CH₂)₂OH. In one embodiment, one of R³ and R⁴ in Formula (II) can be —CH═CH₂ and the other of R³ and R⁴ can be —CH₂O(CH₂)₂OH. In one embodiment, one of R³ and R⁴ in Formula (IV) can be —C≡CH and the other of R³ and R⁴ can be —CH₂O(CH₂)₂OH. In one embodiment, one of R³ and R⁴ in Formula (II) or Formula (II*) can be hydrogen or —CH₃ and the other of R³ and R⁴ can be

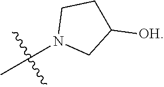 [SMILES: *N1CCC(O)C1]

In one embodiment, R³ and R⁴ both can be —CH₂OH. In one embodiment, one of R³ and R⁴ in Formula (II) or Formula (II*) can be —CH₂OH or —(CH₂)₂OH and the other of R³ and R⁴ can be —CH₂OCH₃. In one embodiment, one of R³ and R⁴ in Formula (II) or Formula (II*) can be hydrogen or —CH₃ and the other of R³ and R⁴ can be —N(CH₃)CH₂CH₂OH. In one embodiment, one of R³ and R⁴ in Formula (II) or Formula (II*) can be hydrogen or —CH₃ and the other of R³ and R⁴ can be —N(C═OCH₃)CH₂CH₂OH. In one embodiment, one of R³ and R⁴ in Formula (II) or Formula (II*) can be hydrogen or —CH₃ and the other of R³ and R⁴ can be

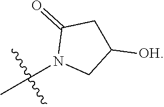 [SMILES: *N1CC(O)CC1=O]

In one embodiment, one of R³ and R⁴ in Formula (II) or Formula (II*) can be hydrogen or —CH₃ and the other of R³ and R⁴ can be

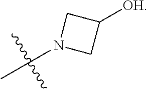 [SMILES: *N1CC(O)C1]

In one embodiment, one of R³ and R⁴ in Formula (II) or Formula (II*) can be hydrogen or —CH₃ and the other of R³ and R⁴ can be

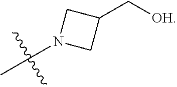 [SMILES: *N1CC(CO)C1]

In other embodiments, one of R³ and R⁴ in Formula (II) or Formula (II*) can be selected from a substituted or an unsubstituted —(C₁-C₆ alkyl)-X², a substituted or an unsubstituted —(C₁-C₆ haloalkyl)-X², a substituted or an unsubstituted —(C₁-C₆ alkenyl)-X², a substituted or an unsubstituted —(C₁-C₆ haloalkenyl)-X², a substituted or an unsubstituted —(C₁-C₆ alkynyl)-X², and a substituted or an unsubstituted —(C₁-C₆ haloalkynyl)-X². In various embodiments, X² can be —OR⁹, —SR⁹, or —NHR⁹, wherein R⁹ can be H, —COR⁸, —CO₂R⁸, —(CO)—NHR⁸, L⁴, L⁵, L⁶, or L⁷, with the proviso that exactly one of R⁷ and R⁹ can be L⁴, L⁵, L⁶, or L⁷. In such embodiments, the compound of Formula (II) can connect to the linker -L²-L³-L⁴-L⁵-L⁶-L⁷- via R³ or R⁴ when R³ or R⁴, respectively, includes X² and R⁹ can be L⁴, L⁵, L⁶, or L⁷. In an embodiment R³ or R⁴ can be a substituted or an unsubstituted —(C₁-C₆ alkyl)-X². In an embodiment, R³ or R⁴ can be a substituted or an unsubstituted —(C₁-C₆ haloalkyl)-X². In an embodiment, R³ or R⁴ can be a substituted or an unsubstituted —(C₁-C₆ alkenyl)-X². In an embodiment, R³ or R⁴ can be a substituted or an unsubstituted —(C₁-C₆ haloalkenyl)-X². In an embodiment, R³ or R⁴ can be a substituted or an unsubstituted —(C₁-C₆ alkynyl)-X². In an embodiment, R³ or R⁴ can be a substituted or an unsubstituted —(C₁-C₆ haloalkynyl)-X². In each such embodiment in which R³ or R⁴ includes X², the option for R⁹ to be L⁴, L⁵, L⁶, or L⁷ is provided, thus providing the option to thereby connect the compound of Formula (II) to the linker -L²-L³-L⁴-L⁵-L⁶-L⁷- via R³ or R⁴.

In various embodiments, R⁷ in Formula (II) or Formula (II*) can be H, —COR⁸, —CO₂R⁸, —(CO)—NHR⁸, L⁴, L⁵, L⁶, or L⁷, where each R⁸ individually can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³. In an embodiment, R⁷ can be H. In an embodiment, R⁷ can be —COR⁸. In an embodiment, R⁷ can be —CO₂R⁸. In an embodiment, R⁷ can be —(CO)—NHR⁸. Those skilled in the art will appreciate that when R⁷ can be H, —COR⁸, —CO₂R⁸, or —(CO)—NHR⁸, connection of the compound of Formula (II) or Formula (II*) to the linker -L²-L³-L⁴-L⁵-L⁶-L⁷- can be via R³ or R⁴ (and thus R⁹) as described elsewhere herein.

In various embodiments, each R⁸ in Formula (II) or Formula (II*) individually can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³, where X³ can be —H, —OH, —SH, or —NH₂. In an embodiment, each R⁸ individually can be a substituted or an unsubstituted C₁-C₆ alkyl-X³. In an embodiment, each R⁸ individually can be a substituted or an unsubstituted C₁-C₆ haloalkyl-X³. In an embodiment, each R⁸ individually can be —[(CY₂)ₚO(CY₂)_{q}]ₜCY₂—X³.

In various embodiments, X² in Formula (II) or Formula (II*) can be —OR⁹, —SR⁹, or —NHR⁹, where R⁹ can be H, —COR⁸, —CO₂R⁸, —(CO)—NHR⁸, L⁴, L⁵, L⁶, or L⁷. In an embodiment, X² can be —OR⁹. In an embodiment, X² can be —SR⁹. In an embodiment, X² can be —NHR⁹.

In various embodiments, R⁹ in Formula (II) or Formula (II*) can be H, —COR⁸, —CO₂R⁸, —(CO)—NHR⁸, L⁴, L⁵, L⁶, or L⁷, where R⁸ can be a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³. In an embodiment, R⁹ can be H. In an embodiment, R⁹ can be —COR⁸. In an embodiment, R⁹ can be —CO₂R⁸. In an embodiment, R⁹ can be —(CO)—NHR⁸. Those skilled in the art will appreciate that when R⁹ can be H, —COR⁸, —CO₂R⁸, or —(CO)—NHR⁸, connection of the compound of Formula (II) to the linker -L²-L³-L⁴-L⁵-L⁶-L⁷- can be via R⁷ as described elsewhere herein.

In various embodiments, R⁹ in Formula (II) or Formula (II*) can be L⁴, L⁵, L⁶, or L⁷. In an embodiment, R⁹ can be L⁴. In an embodiment, R⁹ can be L⁵. In an embodiment, R⁹ can be L⁶. In an embodiment, R⁹ can be L⁷. Those skilled in the art will appreciate that when R⁹ can be L⁴, L⁵, L⁶, or L⁷, connection of the compound of Formula (II) or Formula (II*) to the linker -L²-L³-L⁴-L⁵-L⁶-L⁷- can be via R³ or R⁴ as described elsewhere herein. In an embodiment, exactly one of R⁷ and R⁹ can be L⁴, L⁵, L⁶, or L⁷, in which case a covalent bond can link the drug D to the linker -L²-L³-L⁴-L⁵-L⁶-L⁷- and thereby to Mi.

In various embodiments, each X³ in Formula (II) or Formula (II*) individually can be —H, —OH, —SH, or —NH₂. In an embodiment, X³ can be —H. In an embodiment, X³ can be —OH. In an embodiment, X³ can be —SH. In an embodiment, X³ can be —NH₂.

In various embodiments, m in Formula (II) or Formula (II*) can be 1 or 2. In an embodiment, m can be t. In another embodiment, m can be 2.

In various embodiments, n⁴ and n⁵ in Formula (II) or Formula (II*) each individually can be 0, 1 or 2, with the proviso that n⁴ and n⁵ cannot both be 0. In an embodiment, n⁴ and n⁵ can both be 1. In an embodiment, n⁴ can be 0 and n⁵ can be 1. In an embodiment, n⁴ can be 0 and n⁵ can be 2. In an embodiment, n⁴ can be 1 and n⁵ can be 0. In an embodiment, n⁴ can be 2 and n⁵ can be 0.

In various embodiments, each Y in Formula (II) or Formula (II*) individually can be H or halogen. In an embodiment, each Y can be hydrogen. In an embodiment, —CY₂ can be —CH₂. In an embodiment, —CY₃ can be —CH₃. In an embodiment, —CY₃ can be —CHF₂. In an embodiment, —CY₃ can be —CH₂F. In an embodiment, —CY₃ can be —CF₃.

In various embodiments, each p in Formula (II) or Formula (II*) individually can be 1, 2, 3, 4, 5, or 6. In an embodiment, p can be 1. In an embodiment, p can be 2.

In various embodiments, each q in Formula (II) or Formula (II*) can be individually 0, 1, 2, 3, 4, 5, or 6. In an embodiment, q can be 1. In an embodiment, q can be 2.

In various embodiments, each t in Formula (II) or Formula (II*) individually can be 1, 2, 3, 4, 5, or 6. In an embodiment, t can be 1. In an embodiment, p can be t.

In various embodiments, R¹ and R² can be each individually selected from hydrogen, halogen, and an unsubstituted C₁-C₆ alkyl, or a substituted or an unsubstituted —O—(CR⁵R⁶)ₘ—O— such that R¹ and R² can be taken together form a ring; R³ and R⁴ can be each individually selected from hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ are not both hydrogen; or R³ and R⁴, can be taken together with the carbon atom to which they are attached, form an unsubstituted 4-, 5- or 6-membered heterocyclyl or a 4-, 5- or 6-membered heterocyclyl that is substituted with an unsubstituted C₁-C₃ alkyl; or one of R³ and R⁴ can be an unsubstituted —(C₁-C₆ alkyl)-X² or an unsubstituted —(C₁-C₆ alkenyl)-X²; X² can be OR⁹; R⁵ and R⁶ can be each individually an unsubstituted C₁-C₆ alkyl, or R⁵ and R⁶, can be taken together with the nitrogen atom to which they are attached, form a substituted or unsubstituted 4- or 5-membered heterocyclyl; n⁴ can be 2; n⁵ can be 0; each Y can be H; each m can be 1; p can be 1; q can be 2; t can be 1; R⁷ can be H; and R⁹ can be H, with the proviso that exactly one of R⁷ and R⁹ is L⁴, L⁵, L⁶, or L⁷. In various embodiments, R¹ and R² can be each individually selected from the group consisting of hydrogen, halogen and an unsubstituted C₁-C₆ alkyl; R³ and R⁴ can be each individually selected from the group consisting of hydrogen, —OR⁵, —NR⁵R⁶, a substituted or an unsubstituted C₁-C₆ alkyl, a substituted or unsubstituted 4- or 5-membered heterocyclyl, and [(CY₂)ₚO(CY₂)_{q}]ₜCY₃, with the proviso that R³ and R⁴ are not both hydrogen; or R³ and R⁴, can be taken together with the carbon atom to which they are attached, form an unsubstituted 4-, 5- or 6-membered heterocyclyl or a 4-, 5- or 6-membered heterocyclyl that is substituted with an unsubstituted C₁-C₃ alkyl; or one of R³ and R⁴ can be an unsubstituted —(C₁-C₆ alkyl)-X² or an unsubstituted —(C₁-C₆ alkenyl)-X²; X² can be —OR⁹; R⁵ and R⁶ can be each individually an unsubstituted C₁-C₆ alkyl, or R⁵ and R⁶, can be taken together with the nitrogen atom to which they are attached, form a substituted or unsubstituted 4- or 5-membered heterocyclyl; n⁴ can be 2; n⁵ can be 0; each Y can be H; each m can be 1; p can be 1; q can be 2; t can be 1; R⁷ can be H; and R⁹ can be H, with the proviso that exactly one of R⁷ and R⁹ is L⁴, L⁵, L⁶, or L⁷. In some embodiments, including those of this paragraphs, L² can be absent; L³ can be —(CH₂)n¹-C(═O)—; n¹ can be independently integers of 0 to 5; L⁴ can be a tetrapeptide residue; L⁵ can be —[NH(CH₂)n²]n³-; n² can be 1; n³ can be 1; L⁶ can be absent; L⁷ can be absent; and n can be 1. In other embodiments, including those of this paragraphs, L² can be

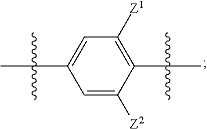

Z¹ and Z² can be each hydrogen; L³ can be —(CH₂)n¹-C(═O)—; n¹ is an integer of 0 to 5; L⁴ can be a tetrapeptide residue; L⁵ can be —[NH(CH₂)n²]n³-; n² can be 1; n³ can be 1; L⁶ can be absent; L⁷ can be absent; and n can be 1. In still other embodiments, including those of this paragraphs, L² can be

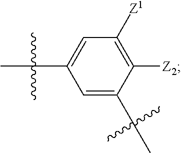

Z¹ and Z² can be each hydrogen; L³ can be —(CH₂)n¹-C(═O)—; n¹ can be an integer of 0 to 5; L⁴ can be a tetrapeptide residue; L⁵ can be —[NH(CH₂)n²]n³-; n² can be 1; n³ can be 1; L⁶ can be absent; L⁷ can be absent; and n can be 1.

In some embodiments, a portion of a drug moiety can connect to L⁴, L⁵, L⁶, or L⁷ as described herein. As an example, the portion of the drug moiety that connects to L⁴, L⁵, L⁶, or L⁷ can be R³ or R⁴ as described herein. In some embodiments, a portion of a drug moiety that connects to L⁴, L⁵, L⁶, or L⁷ can be —CH₂—O— or —CH₂CH₂—O—. In other embodiments, a portion of a drug moiety that connects to L⁴, L⁵, L⁶, or L⁷ can be a monocyclic amine with a —O— substitution where the —O— can be connected directly to the cyclic amine or via an alkylene. Examples of monocyclic amines with a hydroxy connected directly to the cyclic amine include

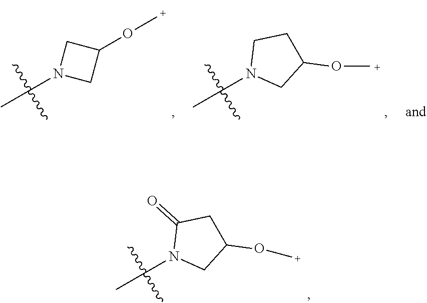

wherein “ [symbol] ” indicates the connection to the remaining portion of the drug moiety and “+” indicates the connection to L⁴, L⁵, L⁶, or L⁷. An example of monocyclic amine with a hydroxy connected via an alkylene is

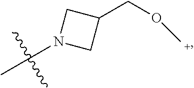 [SMILES: *N1CC(COC)C1]

wherein “ [symbol] ” indicates the connection to the remaining portion of the drug moiety and “+” indicates the connection to L⁴, L⁵, L⁶, or L⁷. In still other embodiments, a portion of a drug moiety that connects to L⁴, L⁵, L⁶, or L⁷ can be disubstituted amine, such as

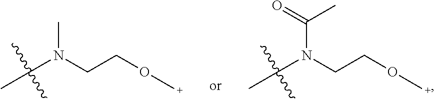

wherein “ [symbol] ” indicates the connection to the remaining portion of the drug moiety and “+” indicates the connection to L⁴, L⁵, L⁶, or L⁷.

In still other embodiments, a portion of a drug moiety that connects to L⁴, L⁵, L⁶, or L⁷ can be a monocyclic amine where a carbon from the monocyclic amine is connected in a spiro-fashion to the remaining portion of the drug moiety, for example, to a ring of the drug moiety. Exemplary monocyclic amines that are spiro-connected to the remaining portion of the drug moiety are

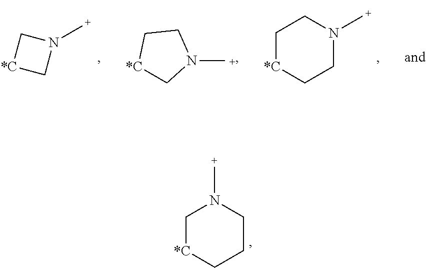

wherein * indicates the carbon atom of the monocyclic amine that attached to the remaining portion of the drug moiety and “+” indicates the connection to L⁴, L⁵, L⁶, or L⁷. In some embodiments, the monocyclic amine that is connected in a spiro-fashion to the remaining portion of the drug moiety can be substituted with a moiety that includes a terminal oxygen

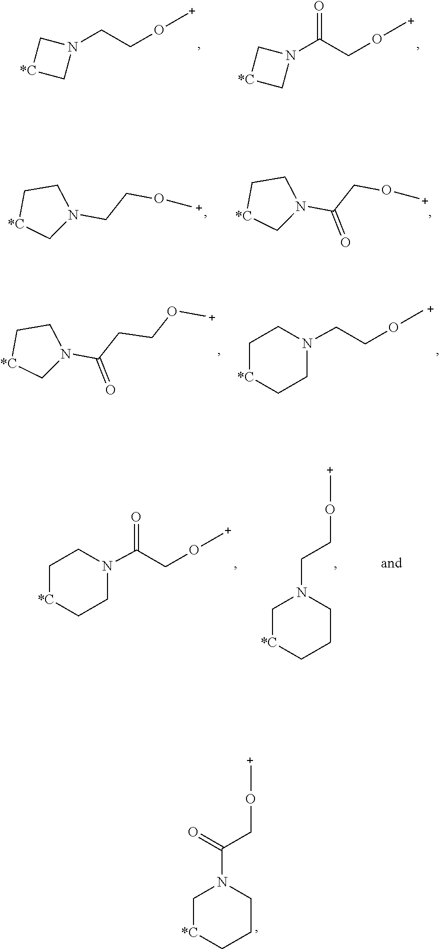

wherein * indicates the carbon atom of the monocyclic amine that attached to the remaining portion of the drug moiety and “+” indicates the connection to L⁴, L⁵, L⁶, or L⁷. In some embodiments, a portion of a drug moiety that connects to L⁴, L⁵, L⁶, or L⁷ cannot include a linear amide. For example, a portion of a drug moiety that connects to L⁴, L⁵, L⁶, or L⁷ cannot include —NH—C(═O)—.

### Immunoconjugates (Antibody-Drug Conjugates)

Various embodiments disclosed herein relate to an immunoconjugate of Formula (I), having the structure:

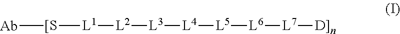

In various embodiments, L¹ in Formula (III) can be

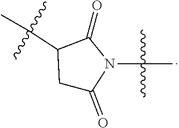 [SMILES: *C1CC(=O)N(*)C1=O ; "."]

In various embodiments, L² in Formula (III) can be absent,

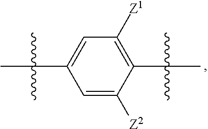

or

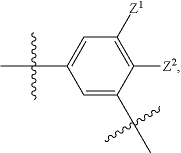

where Z¹ and Z² each individually can be hydrogen, halogen, NO₂, —O—(C₁-C₆ alkyl), or C₁-C₆ alkyl. In an embodiment, L² in Formula (III) can be absent. In an embodiment, L² in Formula (III) can be

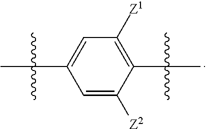

In an embodiment, L² in Formula (III) can be

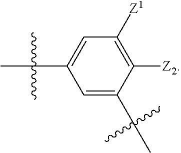

In various embodiments, Z¹ and Z² in Formula (III) each individually can be hydrogen, halogen, NO₂, —O—(C₁-C₆ alkyl), or C₁-C₆ alkyl. In an embodiment, at least one of Z¹ and Z² can be hydrogen. In an embodiment, at least one of Z¹ and Z² can be halogen. In an embodiment, at least one of Z¹ and Z² can be NO₂. In an embodiment, at least one of Z¹ and Z² can be —O—(C₁-C₆ alkyl). For example, in an embodiment, at least one of Z¹ and Z² can be methoxy. In an embodiment, at least one of Z¹ and Z² can be C₁-C₆ alkyl. For example, in an embodiment, at least one of Z¹ and Z² can be methyl.

In various embodiments, L³ in Formula (III) can be —(CH₂)n¹-C(═O)— or —(CH₂CH₂O)n¹-(CH₂)n¹C(═O)—, where each n¹ independently can be integers of 0 to 12. In an embodiment, L³ can be —(CH₂)n¹-C(═O)—. For example, in an embodiment, L³ can be —C(═O)—. In an embodiment, L³ can be —(CH₂CH₂O)n¹-(CH₂)n¹C(═O)—. For example, in an embodiment, L³ can be —CH₂C(═O)—. In embodiment, n¹ can be an integer of 1 to 12, such as 1 to 6 or 1 to 3.

In various embodiments, L⁴ in Formula (III) can be a tetrapeptide residue. For example, in an embodiment, L⁴ can be a tetrapeptide residue selected from SEQ ID NO: 43 GGFG (gly-gly-phe-gly), SEQ ID NO: 44 EGGF (glu-gly-gly-phe), SEQ ID NO: 45 SGGF (ser-gly-gly-phe), and SEQ ID NO: 46 KGGF (lys-gly-gly-phe).

In various embodiments, L⁵ in Formula (III) can be absent or —[NH(CH₂)n²]n³-, where n² can be an integer of 0 to 6 and n³ can be an integer of 0 to 2. In an embodiment, L⁵ can be absent. In an embodiment, L⁵ can be —[NH(CH₂)n²]n³-. For example, in an embodiment, L⁵ can be —NH—. In another embodiment, L⁵ can be —NHCH₂—.

In various embodiments, L⁶ in Formula (III) can be absent or

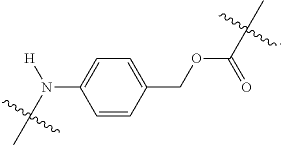 [SMILES: *Nc1ccc(COC(*)=O)cc1]

In an embodiment, L⁶ can be absent. In another embodiment, L⁶ can

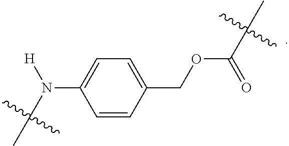 [SMILES: *Nc1ccc(COC(*)=O)cc1]

In various embodiments, L⁷ in Formula (III) can be absent,

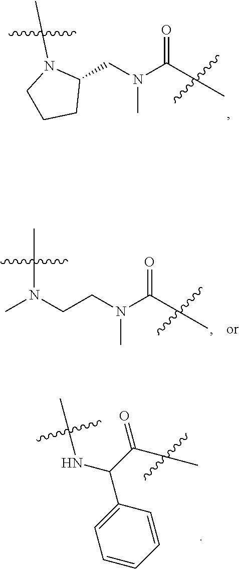

In an embodiment, L⁷ can be absent. In an embodiment, L⁷ can be

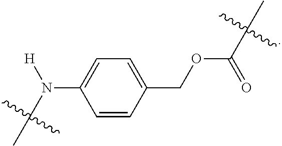 [SMILES: *Nc1ccc(COC(*)=O)cc1]

In an embodiment, L⁷ can be

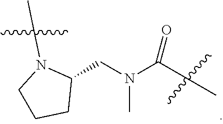 [SMILES: *C(=O)N(C)C[C@@H]1CCCN1*]

In an embodiment, L⁷ can be

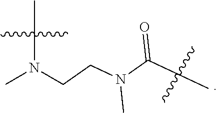 [SMILES: *C(=O)N(C)CCN(*)C]

In an embodiment, L⁷ can be

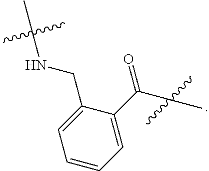 [SMILES: *NCc1ccccc1C(*)=O]

In various embodiments, D in the immunoconjugate of Formula (I) can be a drug moiety as described herein (e.g., under the heading “Drug Moieties” above or under the heading “Compounds” above). In various embodiments, D in the conjugate of Formula (III) can be a compound of Formula (II). In various embodiments. D in the conjugate of Formula (III) can be a compound of Formula (II*). In various embodiments, D in the conjugate of Formula (III) can be a compound of Formula (IV). In various embodiments, D in the conjugate of Formula (III) can be a compound of Formula (IV*). In an embodiment, D can be a cytotoxic anti-cancer drug moiety. In an embodiment, the drug moiety can be exatecan.

In various embodiments, Ab in Formula (I) can be an antibody or an antigen-binding fragment thereof. In an embodiment, Ab can specifically bind to human receptor tyrosine kinase like orphan receptor 1 (ROR1), Her2, TROP2, or Her3. In an embodiment, Ab can bind to a cancer cell surface. In an embodiment, Ab can be an anti-HER2 antibody or an antigen-binding fragment thereof.

In various embodiments, the immunoconjugate of Formula (I) can be selected from:

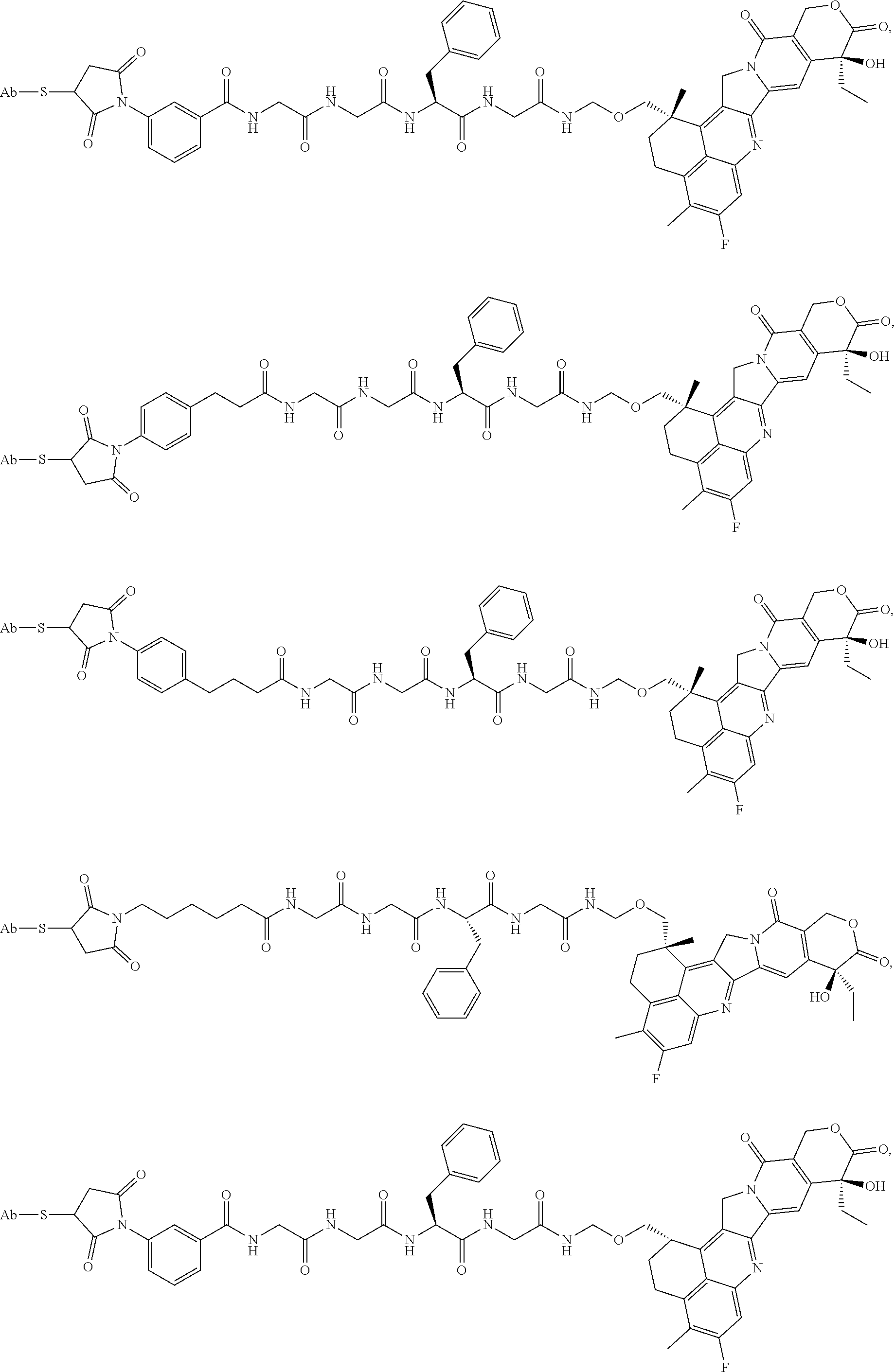

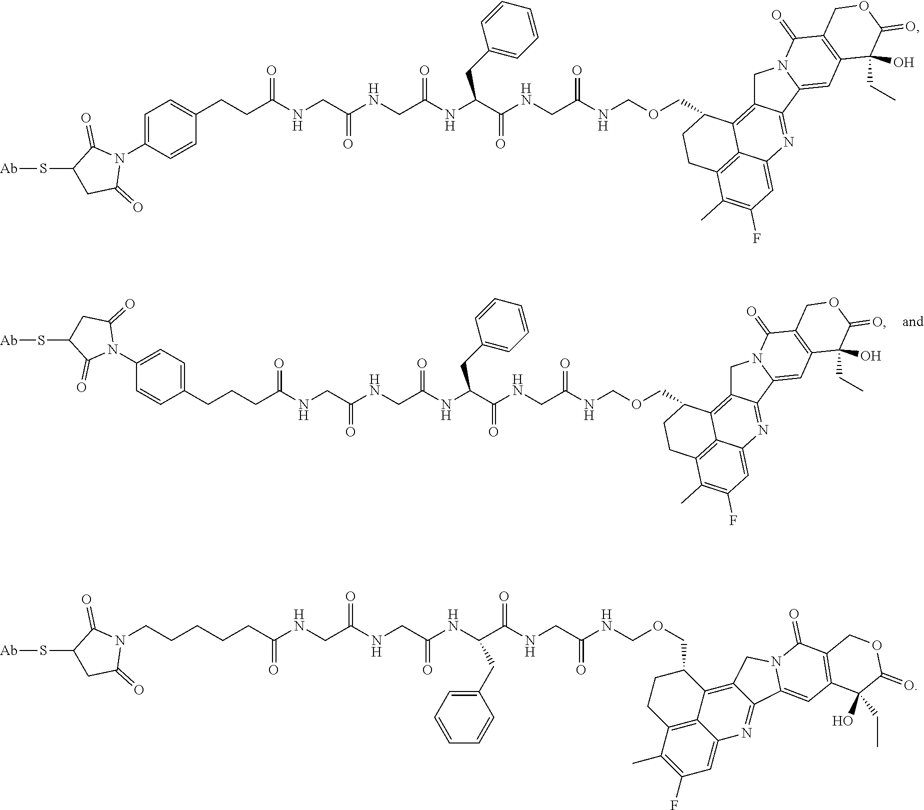

In various embodiments, the immunoconjugate of Formula (I) can be selected from:

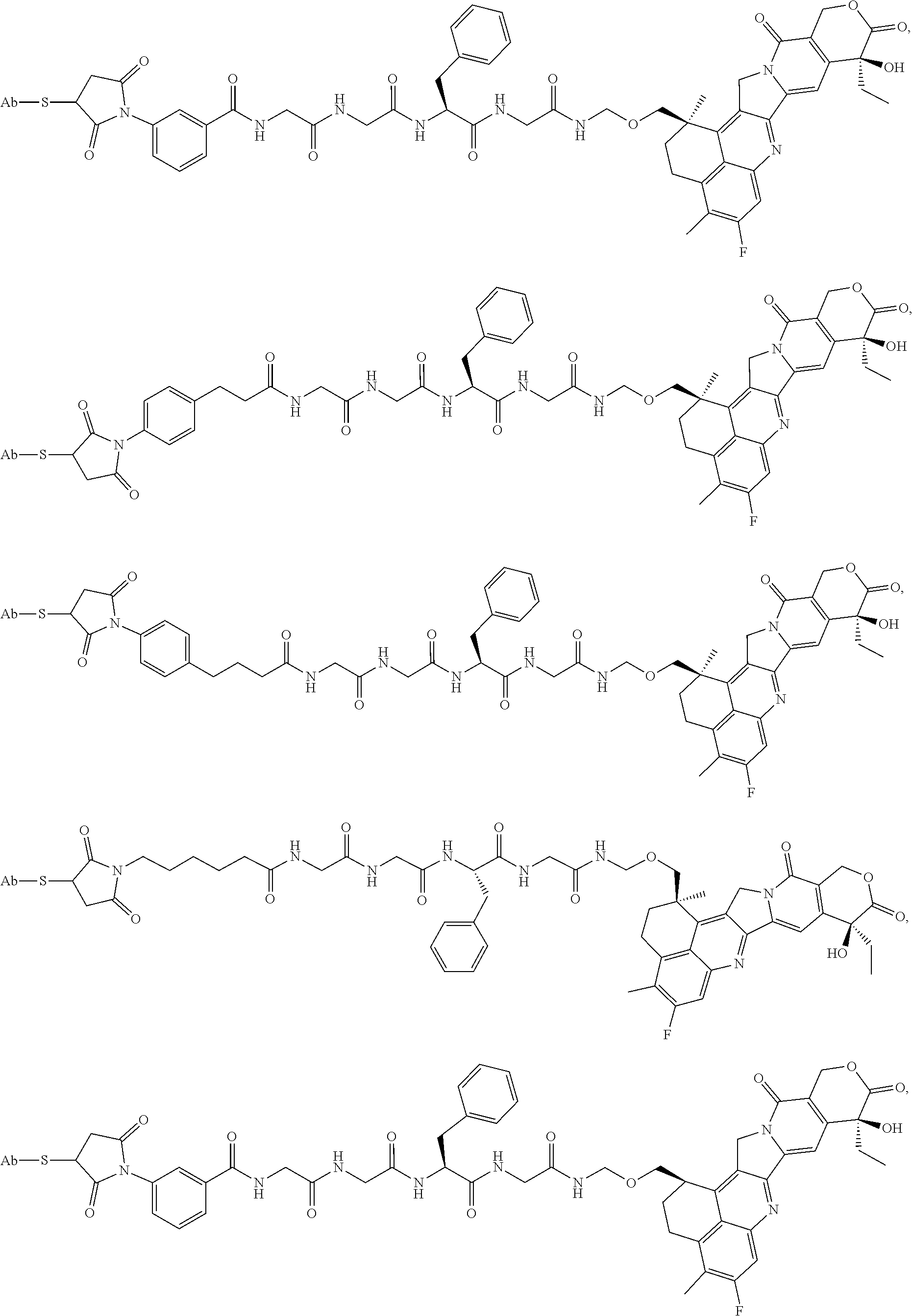

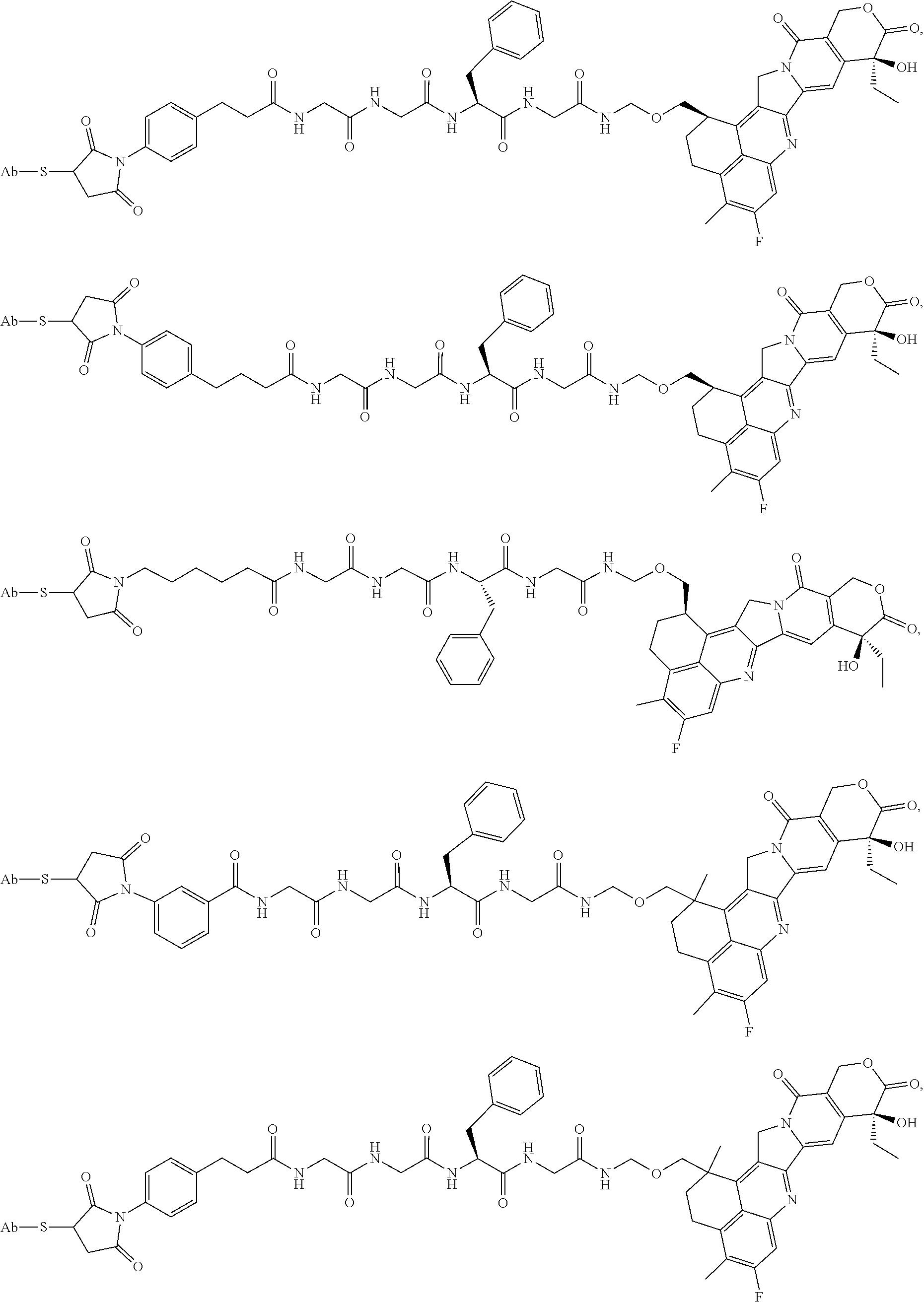

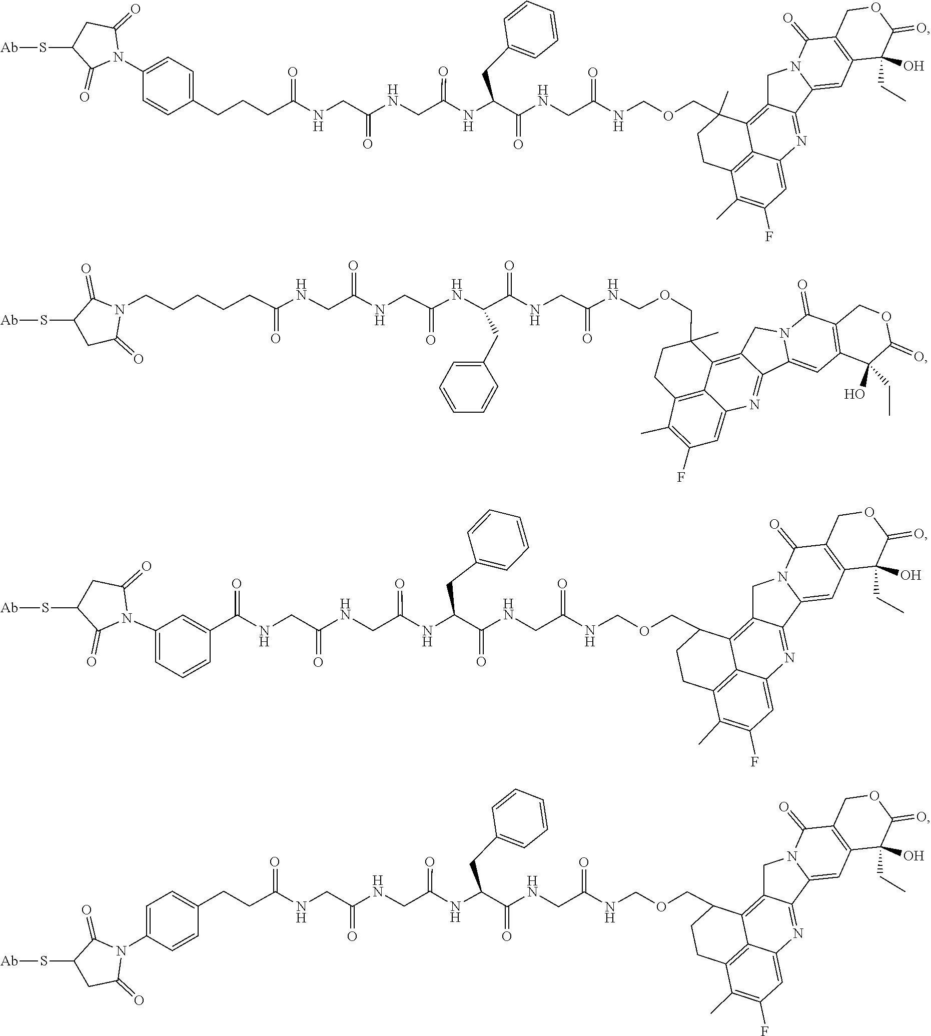

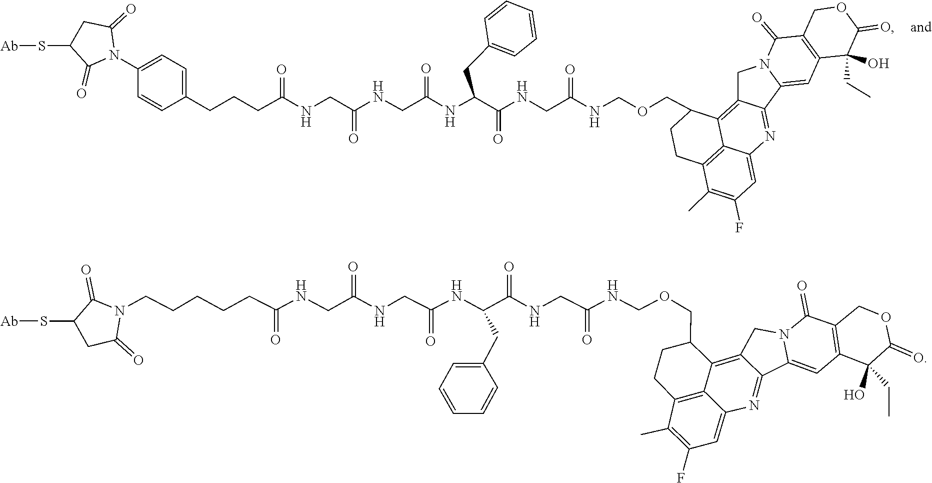

In various embodiments, the immunoconjugate of Formula (I) can be selected from:

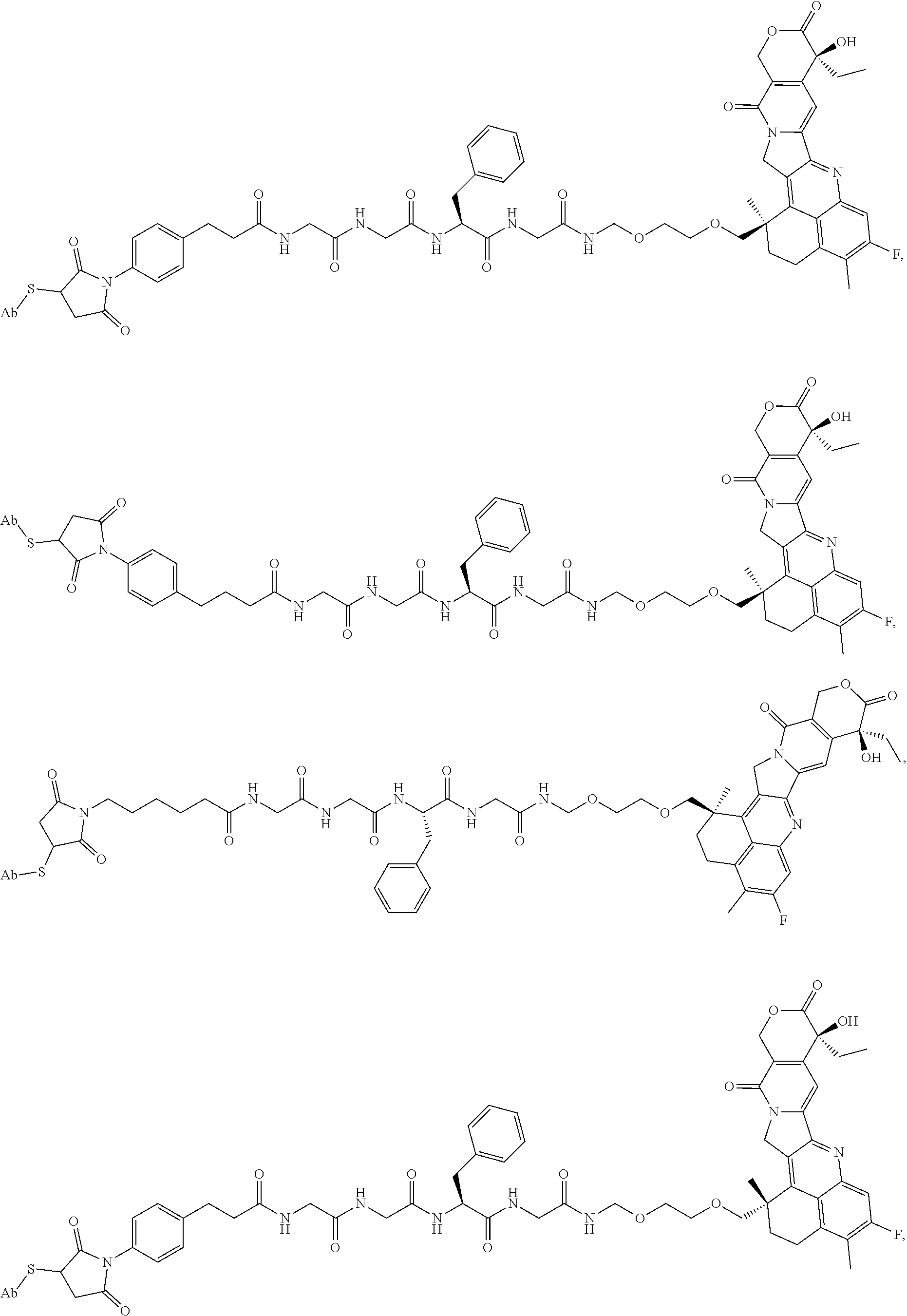

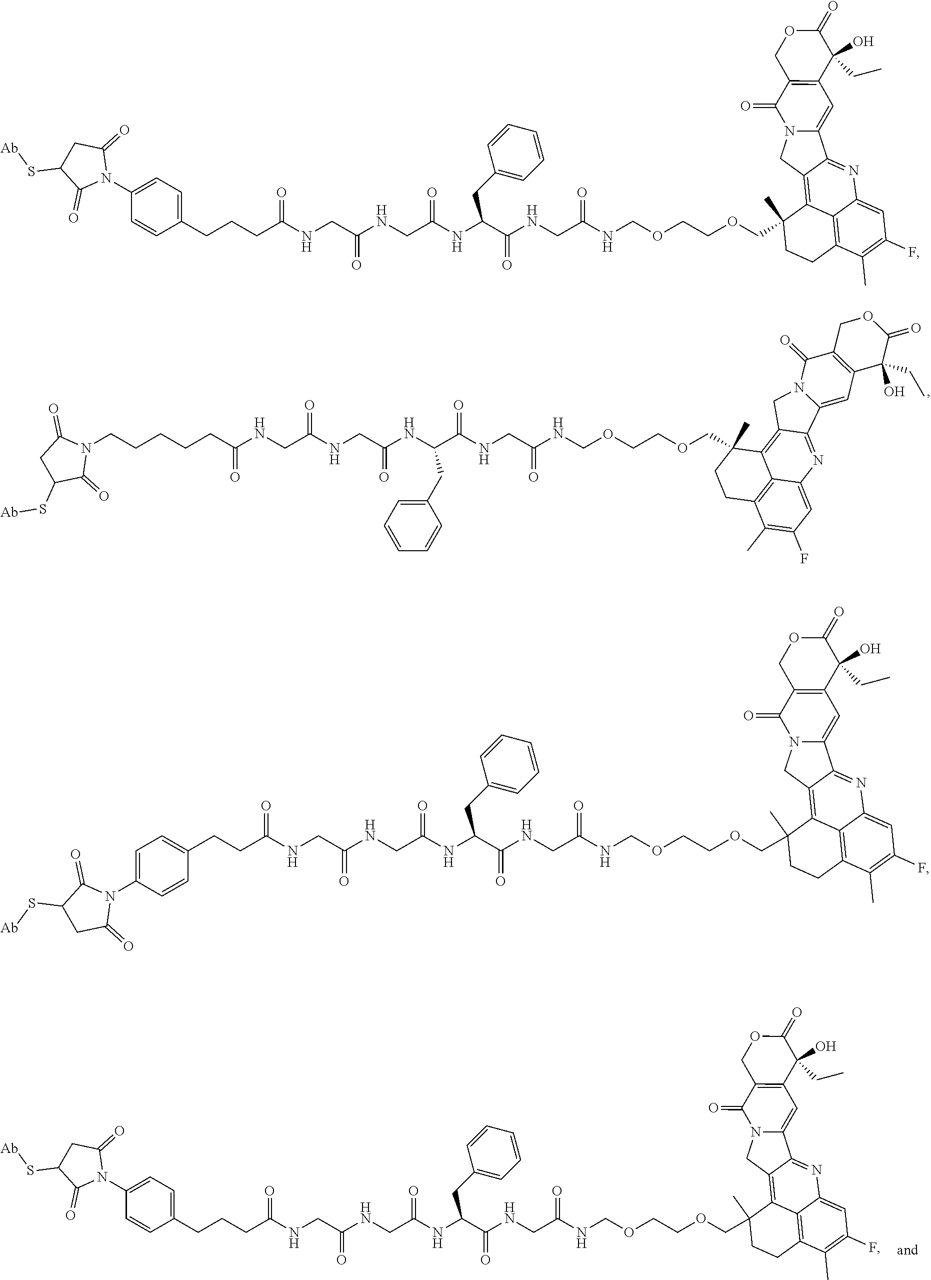

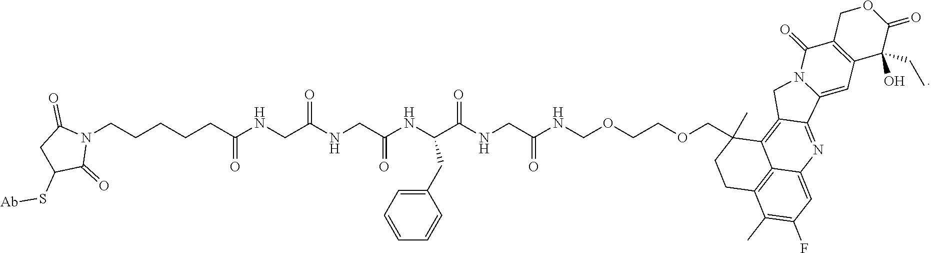

In one embodiment, Ab in any of the foregoing immunoconjugates of Formula (I) can be an antibody or an antibody binding fragment thereof comprising:
VHCDR 1 comprising an amino acid sequence: (i) having at least 95% sequence identity to the amino acid sequence of SEQ ID NO:1 or (ii) of SEQ ID NO: 1;
VHCDR 2 comprising an amino acid sequence: (iii) having at least 95% sequence identity to the amino acid sequence of SEQ ID NO:2 or (iv) of SEQ ID NO:2; and
VHCDR 3 comprising an amino acid sequence: (v) having at least 95% sequence identity to the amino acid sequence of SEQ ID NO:3 or (vi) of SEQ ID NO:3; and
b) a light chain comprising:
VLCDR 1 comprising an amino acid sequence: (vii) having at least 95% sequence identity to the amino acid sequence of SEQ ID NO:8 or (viii) of SEQ ID NO:8;
VLCDR 2 comprising an amino acid sequence: (ix) having at least 95% sequence identity to the amino acid sequence of AAS or (x) of AAS; and
VLCDR 3 comprising an amino acid sequence: (xi) having at least 95% sequence identity to the amino acid sequence of SEQ ID NO:10 or (xii) of SEQ ID NO:10;
wherein the antibody or an antigen-binding fragment thereof specifically binds to the extracellular domain of human receptor tyrosine kinase like orphan receptor 1 (ROR1).

In one embodiment, Ab in any of the foregoing immunoconjugates of Formula (I) can be an antibody or an antibody binding fragment thereof comprising an amino acid sequence: (a) having at least 95% sequence identity to the amino acid sequence of any one of SEQ ID NOs: 5, 7, 12, or 14 or (b) SEQ ID NOs: 5, 7, 12, or 14.

In one embodiment, Ab in any of the foregoing immunoconjugates of Formula (I) can be an antibody or an antibody binding fragment thereof comprising an amino acid sequence: (a) having at least 98% sequence identity to the amino acid sequence of any one of SEQ ID NOs: 5, 7, 12, or 14 or (b) SEQ ID NOs: 5, 7, 12, or 14.

In one embodiment, Ab in any of the foregoing immunoconjugates of Formula (I) can be an antibody or an antibody binding fragment thereof comprising an amino acid sequence of any one of (a) SEQ ID NOs: 5, 7, 12, or 14 or (b) SEQ ID NOs: 5, 7, 12, or 14.

In one embodiment, Ab in any of the foregoing immunoconjugates of Formula (I) can be an antibody or an antibody binding fragment thereof comprising:
VHCDR 1 comprising an amino acid sequence: (i) having at least 95% sequence identity to the amino acid sequence of SEQ ID NO:15 or (ii) of SEQ ID NO:15;
VHCDR 2 comprising an amino acid sequence: (iii) having at least 95% sequence identity to the amino acid sequence of SEQ ID NO:16 or (iv) of SEQ ID NO:16; and
VHCDR 3 comprising an amino acid sequence: (v) having at least 95% sequence identity to the amino acid sequence of SEQ ID NO:17 or (vi) of SEQ ID NO:17; and
b) a light chain comprising:
VLCDR 1 comprising an amino acid sequence: (vii) having at least 95% sequence identity to the amino acid sequence of SEQ ID NO:22 or (viii) of SEQ ID NO:22;
VLCDR 2 comprising an amino acid sequence: (ix) having at least 95% sequence identity to the amino acid sequence of DAY or (x) of DAY; and
VLCDR 3 comprising an amino acid sequence: (xi) having at least 95% sequence identity to the amino acid sequence of SEQ ID NO:24 or (xii) of SEQ ID NO:24;
wherein the antibody or an antigen-binding fragment thereof specifically binds to the extracellular domain of human receptor tyrosine kinase like orphan receptor 1 (ROR1).

In one embodiment, Ab in any of the foregoing immunoconjugates of Formula (I) can be an antibody or an antibody binding fragment thereof comprising an amino acid sequence: (a) having at least 95% sequence identity to the amino acid sequence of any one of SEQ ID NOs: 19, 21, 26 or 28 or (b) SEQ ID NOs: 19, 21, 26 or 28.

In one embodiment, Ab in any of the foregoing immunoconjugates of Formula (I) can be an antibody or an antibody binding fragment thereof comprising an amino acid sequence: (a) having at least 98% sequence identity to the amino acid sequence of any one of SEQ ID NOs: 19, 21, 26 or 28 or (b) SEQ ID NOs: 19, 21, 26 or 28.

In one embodiment, Ab in any of the foregoing immunoconjugates of Formula (I) can be an antibody or an antibody binding fragment thereof comprising an amino acid sequence of any one of (a) SEQ ID NOs: 19, 21, 26 or 28 or (b) SEQ ID NOs: 19, 21, 26 or 28.

In one embodiment, Ab in any of the foregoing immunoconjugates of Formula (I) can be an antibody or an antibody binding fragment thereof comprising:
VHCDR 1 comprising an amino acid sequence: (i) having at least 95% sequence identity to the amino acid sequence of SEQ ID NO:29 or (ii) of SEQ ID NO:29;
VHCDR 2 comprising an amino acid sequence: (iii) having at least 95% sequence identity to the amino acid sequence of SEQ ID NO:30 or (iv) of SEQ ID NO:30; and
VHCDR 3 comprising an amino acid sequence: (v) having at least 95% sequence identity to the amino acid sequence of SEQ ID NO:31 or (vi) of SEQ ID NO:31; and
b) a light chain comprising:
VLCDR 1 comprising an amino acid sequence: (vii) having at least 95% sequence identity to the amino acid sequence of SEQ ID NO:36 or (viii) of SEQ ID NO:36;
VLCDR 2 comprising an amino acid sequence: (ix) having at least 95% sequence identity to the amino acid sequence of DAS or (x) of DAS; and
VLCDR 3 comprising an amino acid sequence: (xi) having at least 95% sequence identity to the amino acid sequence of SEQ ID NO:38 or (xii) of SEQ ID NO:38
wherein the antibody or an antigen-binding fragment thereof specifically binds to the extracellular domain of human receptor tyrosine kinase like orphan receptor 1 (ROR1).

In one embodiment, Ab in any of the foregoing immunoconjugates of Formula (I) can be an antibody or an antibody binding fragment thereof comprising an amino acid sequence: (a) having at least 95% sequence identity to the amino acid sequence of any one of SEQ ID NOs: 33, 35, 40 or 42 or (b) SEQ ID NOs: 33, 35, 40 or 42.

In one embodiment, Ab in any of the foregoing immunoconjugates of Formula (I) can be an antibody or an antibody binding fragment thereof comprising an amino acid sequence: (a) having at least 98% sequence identity to the amino acid sequence of any one of SEQ ID NOs: 33, 35, 40 or 42 or (b) SEQ ID NOs: 33, 35, 40 or 42.

In one embodiment, Ab in any of the foregoing immunoconjugates of Formula (I) can be an antibody or an antibody binding fragment thereof comprising an amino acid sequence of any one of (a) SEQ ID NOs: 33, 35, 40 or 42 or (b) SEQ ID NOs: 33, 35, 40 or 42.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. Even a single variable domain (or half of an Fv comprising only three CDRs specific for an antigen) can have the ability to recognize and bind antigen, although in some instances at a lower affinity than the entire binding site. Similarly, as used herein with reference to use of the term CDR, in some alternatives any one or more of the CDRs may differ by one amino acid from the CDR sequence set forth in the sequence listing, e.g., by a conservative amino acid substitution.

### Pharmaceutical Compositions

Some embodiments described herein relate to a pharmaceutical composition, which can include an effective amount of one or more compounds described herein (for example, an immunoconjugate compound of Formula (I), a drug compound of the Formula (IV) or Formula (IV*), or a pharmaceutically acceptable salt thereof) and a pharmaceutically acceptable carrier, diluent, excipient or combination thereof.

The term “pharmaceutical composition” refers to a mixture of one or more compounds and/or salts disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration.

The term “physiologically acceptable” defines a carrier, diluent or excipient that does not abrogate the biological activity and properties of the compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound, salt and/or composition exist in the art including, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, a compound of Formula (I), or a pharmaceutically acceptable salt thereof, can be administered orally.

One may also administer the compound, salt and/or composition in a local rather than systemic manner, for example, via injection or implantation of the compound directly into the affected area, often in a depot or sustained release formulation. Furthermore, one may administer the compound in a targeted drug delivery system, for example, in a liposome coated with a targeting ligand to a specific cell or tissue type. The liposomes will be targeted to and taken up selectively by the targeted cell or tissue.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound and/or salt described herein formulated in a compatible pharmaceutical carrier may also be prepared, placed in an appropriate container and labeled for treatment of an indicated condition.

### Uses and Methods of Treatment

Some embodiments described herein relate to a method for treating a cancer or a tumor described herein that can include administering an effective amount of a compound described herein (for example, an immunoconjugate compound of Formula (I), a drug compound of the Formula (IV) or Formula (IV*), or a pharmaceutically acceptable salt thereof) or a pharmaceutical composition that includes a compound described herein (for example, an immunoconjugate compound of Formula (I), a drug compound of the Formula (IV) or Formula (IV*), or a pharmaceutically acceptable salt thereof) to a subject having the cancer or tumor. Other embodiments described herein relate to the use of an effective amount of a compound described herein (for example, an immunoconjugate compound of Formula (I), a drug compound of the Formula (IV) or Formula (IV*), or a pharmaceutically acceptable salt thereof) or a pharmaceutical composition that includes a compound described herein (for example, an immunoconjugate compound of Formula (I), a drug compound of the Formula (IV) or Formula (IV*), or a pharmaceutically acceptable salt thereof) in the manufacture of a medicament for treating a cancer or a tumor described herein. Still other embodiments described herein relate to an effective amount of a compound described herein (for example, an immunoconjugate compound of Formula (I), a drug compound of the Formula (IV) or Formula (IV*), or a pharmaceutically acceptable salt thereof) or a pharmaceutical composition that includes a compound described herein (for example, an immunoconjugate compound of Formula (I), a drug compound of the Formula (IV) or Formula (IV*), or a pharmaceutically acceptable salt thereof) for treating a cancer or a tumor described herein.

Examples of cancers and tumors include but are not limited to: lung cancer, urothelial cancer, colorectal cancer, prostate cancer, ovarian cancer, pancreatic cancer, breast cancer, bladder cancer, gastric cancer, gastrointestinal stromal tumor, uterine cervix cancer, esophageal cancer, squamous cell carcinoma, peritoneal cancer, liver cancer, hepatocellular cancer, colon cancer, rectal cancer, colorectal cancer, endometrial cancer, uterine cancer, salivary gland cancer, kidney cancer, vulval cancer, thyroid cancer, penis cancer, leukemia, malignant lymphoma, plasmacytoma, myeloma, or sarcoma.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant, for example, a child or infant with a fever. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound, or pharmaceutical agent, which elicits the biological or medicinal response indicated. For example, a therapeutically effective amount of compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the compounds disclosed herein required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a compound is the amount that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. In the treatment of lung cancer (such as non-small cell lung cancer), a therapeutically effective amount is that amount that alleviates or eliminates cough, shortness of breath and/or pain.

The amount of the inununoconjugate compound of Formula (I), drug compound of the Formula (IV) or Formula (IV*), or pharmaceutically acceptable salt thereof, required for use in treatment will vary not only with the particular compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the dosage ranges described herein in order to effectively and aggressively treat particularly aggressive diseases or conditions.

In general, however, a suitable dose will often be in the range of from about 0.05 mg/kg to about 10 mg/kg. For example, a suitable dose may be in the range from about 0.10 mg/kg to about 7.5 mg/kg of body weight per day, such as about 0.15 mg/kg to about 5.0 mg/kg of body weight of the recipient per day, about 0.2 mg/kg to 4.0 mg/kg of body weight of the recipient per day, or any amount in between. The compound may be administered in unit dosage form; for example, containing 1 to 500 mg, 10 to 100 mg, 5 to 50 mg or any amount in between, of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular compounds employed and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies. For example, useful dosages of an immunoconjugate compound of Formula (I), a drug compound of the Formula (IV) or Formula (IV*), or a pharmaceutically acceptable salt thereof, can be determined by comparing their in vitro activity and in vivo activity in animal models. Such comparison can be done by comparison against an established drug, such as cisplatin and/or gemcitabine.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

Compounds, salts and compositions disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Synthesis

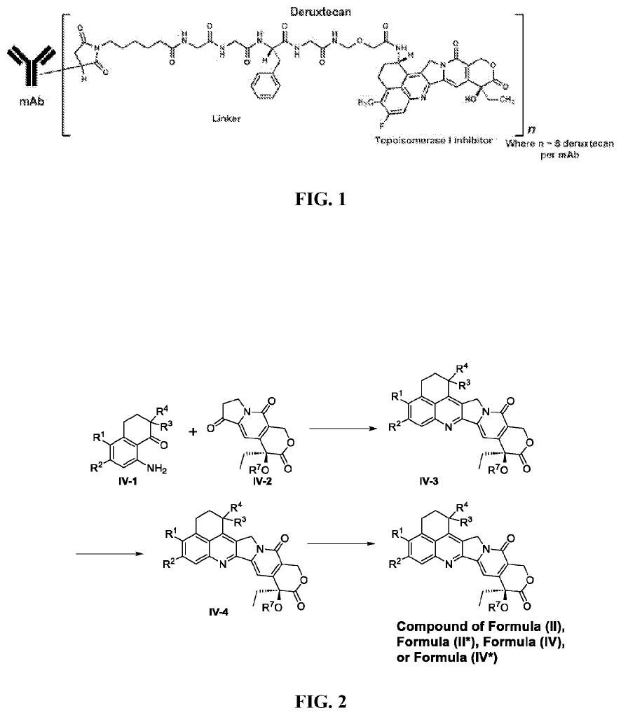

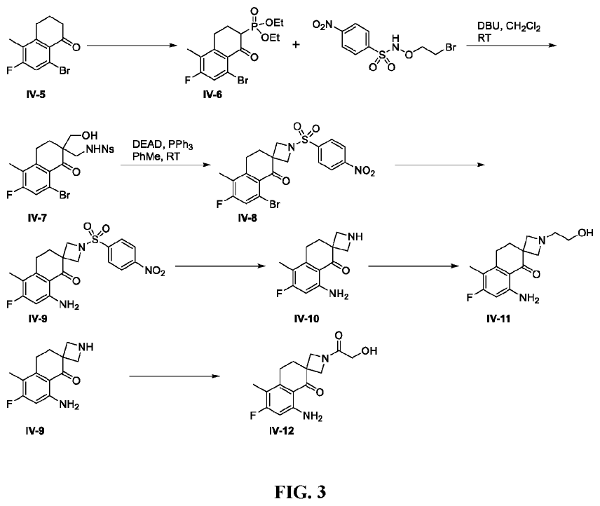

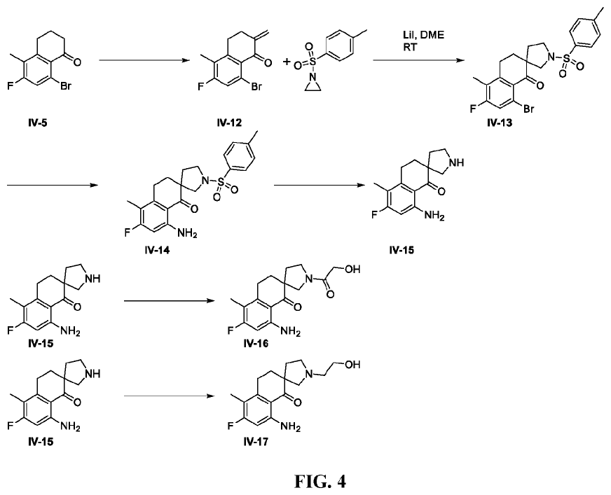

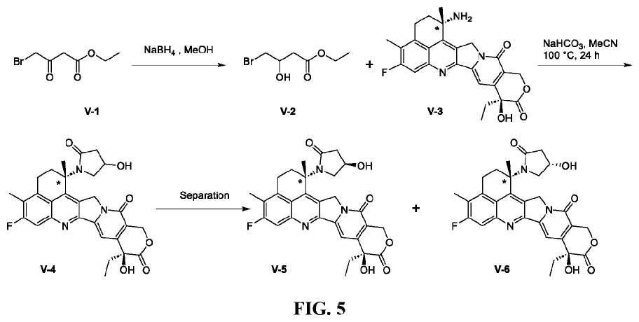

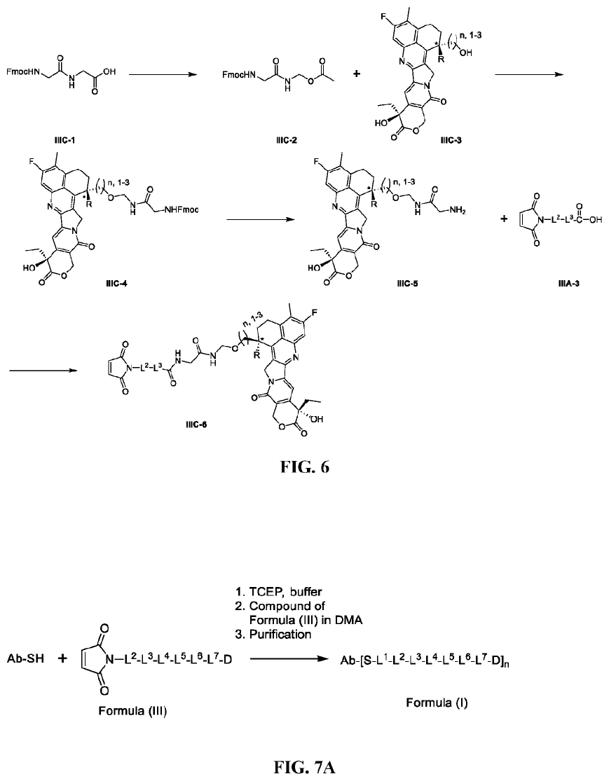

Drug compounds of the Formula (II), Formula (II*), Formula (IV), or Formula (IV*), or pharmaceutically acceptable salts thereof, can be made in various ways by those skilled using known techniques as guided by the detailed teachings provided herein. For example, in an embodiment, drug compounds of the Formula (II), Formula (II*), Formula (IV), or Formula (IV*) are prepared in accordance with the general schemes illustrated in FIGS. 2-4.

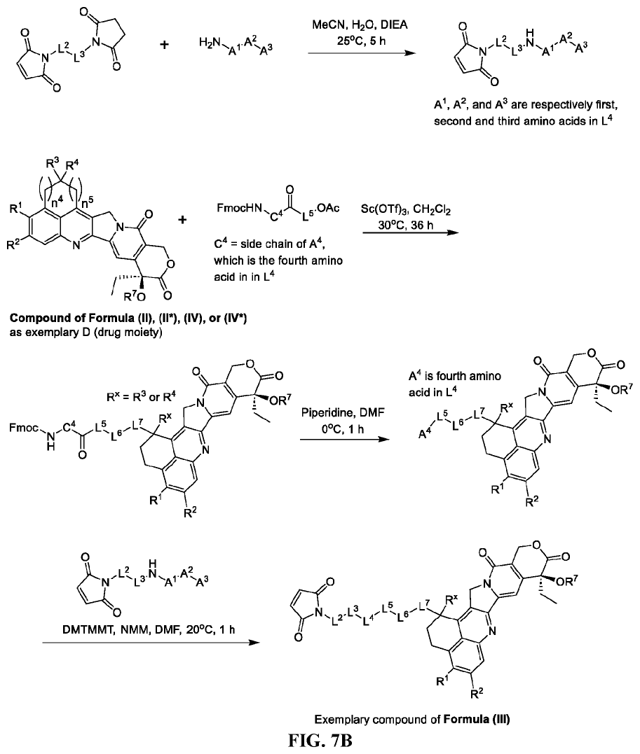

Conjugates of the Formula (III) can be made in various ways by those skilled using known techniques as guided by the detailed teachings provided herein. A general scheme for making a conjugate of Formula (III) is provided in FIG. 7B, whereby the variables L², L³, L⁴, L⁵, L⁶, and L⁷ are as defined herein for Formula (III); R¹, R², R 4, R, n⁴, and n⁵ are as defined herein for Formula (II), Formula (II*), Formula (IV), or Formula (IV*); and A¹, A², A³, A⁴, and R^{x} are defined within FIG. 7B itself.

Immunoconjugates of the Formula (I) can be made in various ways by those skilled using known techniques as guided by the detailed teachings provided herein. For example, in an embodiment, immunoconjugates of the Formula (I) are prepared in accordance with the general scheme illustrated in FIG. 7A. Briefly, the antibody is prepared for reduction (to form the cysteine residues Cys-SH) by the addition of Tris buffer, mM EDTA, at pH 8.5, followed by the addition of TCEP. After DMA is added and gently mixed with the reduced antibody solution to achieve a final 10% v/v during conjugation, a stock solution containing the toxin-linker, i.e., Compound of Formula (III) in DMA is added and gently mixed. The bioconjugation is allowed to proceed for approximately 16-20 h overnight at 20° C. The process of preparing immunoconjugates of Formula (I) is described in greater detail in Example 30. In an embodiment, a process of producing an immunoconjugate as described herein comprises reacting an effective amount of a thiol-functionalized antibody or antigen-binding fragment with a conjugate as described herein under reaction conditions effective to form the immunoconjugate. In an embodiment, the process comprises: (i) reducing an antibody or an antigen-binding fragment thereof to form the thiol-functionalized antibody or an antigen-binding fragment thereof; and (ii) adding the conjugate to the thiol-functionalized antibody or an antigen-binding fragment thereof. In one embodiment, step (i) of said process comprises reducing the antibody or an antigen-binding fragment thereof in the presence of (2-carboxyethyl) phosphine (TCEP) and ethylenediaminetetraacetic acid (EDTA) and at about pH 8.5. In one embodiment, step (ii) of said process comprises adding the conjugate to the thiol-functionalized antibody or an antigen-binding fragment thereof in the presence of dimethylacetamide (DMA) to incubate for at least 16 hours at about 20° C.

### EXAMPLES

Additional embodiments are disclosed in further detail in the following examples, which are not in any way intended to limit the scope of the claims.

### Example 1

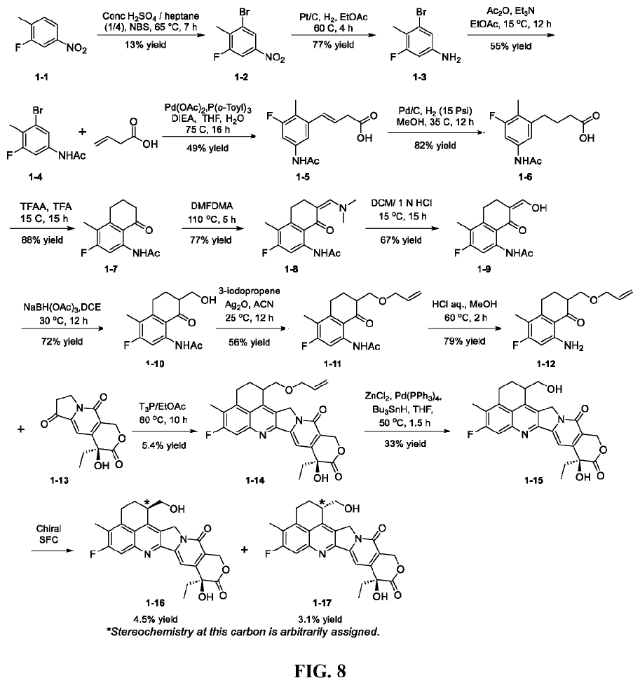

### (1R,9S)-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (1-16) and (1S,9S)-9-ethyl-5-fluoro-9-hydroxy-1-(hydroxymethyl)-4-methyl-1,2,3,9,12,15-hexahydro-10H,13H-benzo[de]pyrano[3′,4′:6,7]indolizino[1,2-b]quinoline-10,13-dione (1-17) (FIG. 8

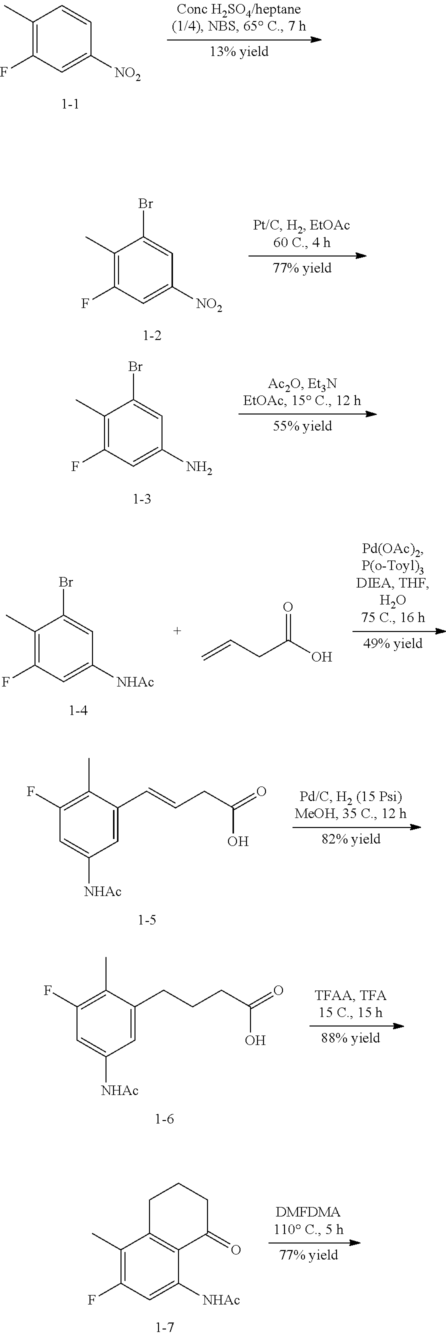

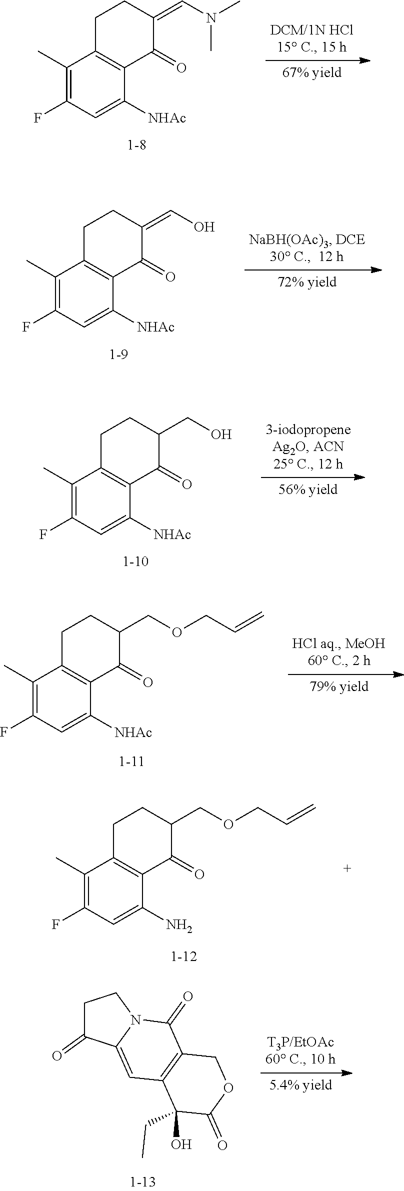

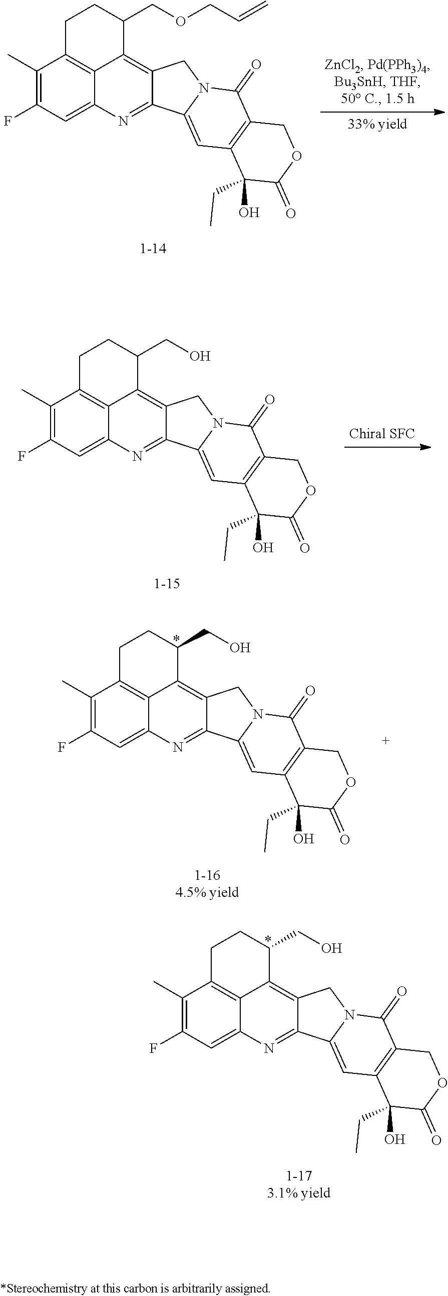 [SMILES: C=CCOCC1CCc2c(C)c(F)cc3nc4c(c1c23)Cn1c-4cc2c(c1=O)COC(=O)[C@]2(O)CC ; CC[C@@]1(O)C(=O)OCc2c1cc1n(c2=O)Cc2c-1nc1cc(F)c(C)c3c1c2C(CO)CC3 ; CC[C@@]1(O)C(=O)OCc2c1cc1n(c2=O)Cc2c-1nc1cc(F)c(C)c3c1c2[C@H](CO)CC3 ; CC[C@@]1(O)C(=O)OCc2c1cc1n(c2=O)Cc2c-1nc1cc(F)c(C)c3c1c2[C@@H](CO)CC3]

### 1-Bromo-3-fluoro-2-methyl-5-nitro-benzene (1-2)

To a stirred solution of 2-fluoro-1-methyl-4-nitro-benzene (1-1) (1.00 kg, 6.45 mol) in H₂SO₄ (1.25 L) and heptane (5.00 L) was added 1-bromopyrrolidine-2,5-dione (2.29 kg, 12.9 mol) at 65° C. portion wise over 6 h. After stirring at 65° C. for 1 h, it was quenched with 3.1 kg of ice, extracted with ethyl acetate (2×650 mL), washed with 10% Na₂SO₃ (3×650 mL), sat NaHCO₃ (2×650 mL), dried over Na₂SO₄, filtered and concentrated. Upon standing for 16 h at room temperature, yellow solids were formed; they were filtered, washed with cold heptane (1.20 L) and dried under vacuum to afford 1-bromo-3-fluoro-2-methyl-5-nitro-benzene (1-2) (238 g, 16% yield). ¹H NMR (400 MHz, CDCl₃) δ ppm 8.25 (t, J=1.7 Hz, 1H), 7.87 (dd, J=8.8, 2.2 Hz, 1H), 2.43 (d, J=2.4 Hz, 3H).

### 3-Bromo-5-fluoro-4-methyl-aniline (1-3)

To a stirred solution of 1-bromo-3-fluoro-2-methyl-5-nitro-benzene (1-2) (100 g, 427 mmol) in ethyl acetate (1.50 L) was added Pt/C (10 wt %, 10.0 g) under argon atmosphere, purged with H₂ three times, and stirred under H₂ (15 psi) at 60° C. for 4 h. After the H₂ atmosphere was exchanged with argon, it was filtered through a pad of celite, filter cake washed with ethyl acetate (500 mL), combined filtrates concentrated and dried under vacuum to afford 3-bromo-5-fluoro-4-methyl-aniline (1-3) (94.7 g, purity 71%, 77% yield). ¹H NMR (400 MHz, DMSO-d₆) δ ppm 6.79 (s, 1H), 6.50 (dd, J=11.7, 2.1 Hz, 1H), 2.10 (d, J=2.1 Hz, 3H). ¹⁹F NMR (376 MHz, CD₃OD) δ ppm −112.13. LCMS (ESI+) m/z: [MH]⁺, 205.8.

### N-(3-Bromo-5-fluoro-4-methyl-phenyl)acetamide (1-4)

To a solution of 3-bromo-5-fluoro-4-methyl-aniline (1-3) (300 g, 1.47 mol) in ethyl acetate (4.5 L) at 15° C. were added triethylamine (420, 3.01 mol) and acetic anhydride (179 mL, 1.91 mol). After stirring at 15° C. for 12 h, it was quenched with sat NH₄Cl (2.5 L), extracted with ethyl acetate (3×850 mL), combined organic layers washed with brine, dried over Na₂SO₄, concentrated and the residue purified by silica gel column chromatography eluting with 15% of ethyl acetate in petroleum ether to afford N-(3-bromo-5-fluoro-4-methyl-phenyl)acetamide (1-4) (200 g, 55% yield). ¹H NMR (400 MHz, CD₃OD) δ ppm 7.59 (t, J=1.5 Hz, 1H), 7.41 (dd, J=11.6, 2.0 Hz, 1H), 2.26 (d, J=2.2 Hz, 3H), 2.11 (s, 3H). ¹⁹F NMR (376 MHz, CD₃OD) δ ppm −112.95. LCMS (ESI+) m/z: [MH]⁺ 247.8.

### (E)-4-(5-Acetamido-3-fluoro-2-methyl-phenyl)but-3-enoic acid (1-5)

To a stirred solution of N-(3-bromo-5-fluoro-4-methyl-phenyl)acetamide (1-4) (200 g, 813 mmol) in tetrahydrofuran (1.00 L) and water (200 mL) were added diisopropylethyl amine (566 mL, 3.25 mol), tris-o-tolylphosphane (49.5 g, 163 mmol), but-3-enoic acid (168 g, 1.95 mol) and Pd(OAc)₂ (18.2 g, 81.3 mmol) under N₂. After stirring at 75° C. for 16 h, it was diluted with water (350 mL), pH adjusted to 2 with 3 N HCl, and filtered through a pad of Celite. The mixture was extracted with ethyl acetate (650 mL) and water (650 mL); after separation, the aqueous layer was extracted with ethyl acetate (3×300 mL). The combined organic layers were washed with brine, dried over Na₂SO₄, filtered, concentrated and the residue purified by silica gel column chromatography eluting with 70% ethyl acetate in petroleum ether to give (E)-4-(5-acetamido-3-fluoro-2-methyl-phenyl)but-3-enoic acid (1-5) (100 g, 49% yield). ¹H NMR (400 MHz, DMSO-d₆) δ ppm 12.32 (br s, 1H), 10.02 (s, 1H), 7.48 (ddd, J=12.0, 8.3, 1.6 Hz, 1H), 7.39 (s, 1H), 7.08-6.98 (m, 1H), 6.94-6.83 (m, 1H), 6.75-6.50 (m, 1H), 6.13 (td, J=15.7, 7.2 Hz, 1H), 5.69 (d, J=15.5 Hz, 1H), 3.59 (ddd, J=6.5, 4.1, 2.5 Hz, 1H), 3.56-3.46 (m, 1H), 3.31-3.21 (m, 1H), 2.16-2.06 (m, 3H), 2.05-1.99 (m, 3H). ¹⁹F NMR (376 MHz, DMSO-d₆) δ ppm −115.38. LCMS (ESI+) m/z: [MH]⁺ 252.0.

### 4-(5-Acetamido-3-fluoro-2-methyl-phenyl)butanoic acid (1-6)

To a stirred solution of (E)-4-(5-acetamido-3-fluoro-2-methyl-phenyl)but-3-enoic acid (1-5) (100 g, 398 mmol) in methanol (1.5 L) was added Pd/C (30.0 g, 39.8 mmol, 10 wt %, 0.10 eq.) under argon atmosphere, purged with H₂ three times, and stirred under H₂ (15 psi) at 35° C. for 12 h. After the H₂ atmosphere was exchanged with argon, it was filtered through a pad of celite, filter cake washed with methanol (2 L), combined filtrates concentrated and dried under vacuum to afford 4-(5-acetamido-3-fluoro-2-methyl-phenyl)butanoic acid (1-6) (82.5 g, 82% yield), which was used in the next step without further purification. ¹H NMR (400 MHz, DMSO-d₆) δ ppm 9.98 (s, 1H), 7.42 (dd, J=12.2, 1.7 Hz, 1H), 7.04 (s, 1H), 2.59-2.53 (m, 2H), 2.27 (t, J=7.2 Hz, 2H), 2.09 (d, J=1.8 Hz, 3H), 2.01 (s, 3H), 1.73-1.67 (m, 2H). ¹⁹F NMR (376 MHz, DMSO-d₆) δ ppm −115.64. LCMS (ESI+) m/z: [MH]⁺ 254.0.

### N-(3-fluoro-4-methyl-8-oxo-5,6,7,8-tetrahydronaphthalen-1-yl)acetamide (1-7)

To a stirred solution of 4-(5-acetamido-3-fluoro-2-methyl-phenyl)butanoic acid (1-6) (110 g, 434 mmol) in trifluoroacetic acid (330 mL) at 0° C. was added trifluoroacetic anhydride (121 mL, 869 mmol). After stirring at 15° C. for 15 h, it was poured into 50% acetonitrile aqueous solution (2 L) at 0° C. and stirred at 0° C. for 0.5 h. The resulting yellow suspension was adjusted to pH 7 with 25% NaOH at 0° C., resulting solids filtered, washed with water (350 mL), methyl tert-butyl ester (700 mL), dried, triturated with methyl tert-butyl ester (200 mL), filtered and dried to afford N-(3-fluoro-4-methyl-8-oxo-5,6,7,8-tetrahydronaphthalen-1-yl)acetamide (1-7) (89.9 g, 88% yield). ¹H NMR (400 MHz, DMSO-d₆) δ ppm 12.18 (s, 1H), 8.28 (d, J=13.2 Hz, 1H), 2.89 (t, J=6.1 Hz, 2H), 2.68-2.60 (m, 2H), 2.18-2.08 (m, 6H), 1.99 (quin, J=6.4 Hz, 2H). ¹⁹F NMR (376 MHz, DMSO-d₆) δ ppm −103.89. LCMS (ESI+) m/z: [MH]⁺ 236.0.

### N-(7-((Dimethylamino)methylene)-3-fluoro-4-methyl-8-oxo-5,6,7,8-tetrahydronaphthalen-1-yl)acetamide (1-8)

After a stirred solution of N-(3-fluoro-4-methyl-8-oxo-5,6,7,8-tetrahydronaphthalen-1-yl)acetamide (1-7) (100 mg, 0.426 mmol) in 1,1-dimethoxy-N,N-dimethylmethanamine (1 mL) was heated at 110° C. for 5 h, it was cooled to 15° C., concentrated and the residue purified by silica gel column chromatography eluting with 70% ethyl acetate in petroleum ether to afford N-(7-((dimethylamino)methylene)-3-fluoro-4-methyl-8-oxo-5,6,7,8-tetrahydronaphthalen-1-yl)acetamide (1-8) (96 mg, 77% yield). ¹H NMR (400 MHz, CDCl₃) δ ppm 12.88 (br s, 1H), 8.35 (d, J=13.0 Hz, 1H), 7.73 (s, 1H), 3.17 (s, 6H), 2.75-2.84 (m, 4H), 2.20 (s, 3H), 2.16 (d, J=1.8 Hz, 3H). ¹⁹F NMR (376 MHz, CDCl₃) δ ppm −106.40. LCMS (ESI+) m/z: [MH]⁺ 264.1. Note: 1-8 was converted to 1-9 under LCMS conditions.

### N-(3-Fluoro-7-(hydroxymethylene)-4-methyl-8-oxo-5,6,7,8-tetrahydronaphthalen-1-yl) acetamide (1-9)

To a stirred solution of N-(7-((dimethylamino)methylene)-3-fluoro-4-methyl-8-oxo-5,6,7,8-tetrahydro-naphthalen-1-yl)acetamide (1-8) (49.0 mg, 0.168 mmol) in dichloromethane (1 mL) at 15° C. was added 1 N HCl (1 mL). After stirring at 15° C. for 15 h, it was extracted with dichloromethane (3×2 mL), combined organic layers washed with brine, dried over Na₂SO₄, filtered, concentrated and dried under vacuum to afford N-(3-fluoro-7-(hydroxymethylene)-4-methyl-8-oxo-5,6,7,8-tetrahydronaphthalen-1-yl)acetamide (1-9) (30.0 mg, 67% yield). ¹H NMR (400 MHz, CDCl₃) δ ppm 13.99 (d, J=11.1 Hz, 1H), 11.85 (s, 1H), 8.40 (d, J=12.8 Hz, 1H), 7.45 (d, J=11.1 Hz, 1H), 2.81-2.87 (m, 2H), 2.48 (t, J=6.8 Hz, 2H), 2.24 (s, 3H), 2.18 (d, J=1.8 Hz, 3H). ¹⁹F NMR (376 MHz, CDCl₃) δ ppm −101.81. LCMS (ESI+) m/z: [MH]⁺ 264.1.

### N-(3-Fluoro-7-(hydroxymethyl)-4-methyl-8-oxo-5,6,7,8-tetrahydronaphthalen-1-yl) acetamide (1-10)

To a stirred solution of N-(3-fluoro-7-(hydroxymethylene)-4-methyl-8-oxo-5,6,7,8-tetrahydronaphthalen -1-yl)acetamide (1-9) (10.0 g, 38.0 mmol) in dichloroethane (100 mL) and acetic acid (1 mL) at 20° C. was added sodium triacetoxyhydroborate (9.66 g, 45.5 mmol) portion wise. After stirring at 30° C. for 12 h, it was quenched with water (100 mL), extracted with dichloromethane (3×150 mL), combined organic layers washed with water, dried over Na₂SO₄, filtered, concentrated and the residue purified by silica gel column chromatography eluting with 33% to 66% ethyl acetate in petroleum ether to afford N-(3-fluoro-7-(hydroxymethyl)-4-methyl-8-oxo-5,6,7,8-tetrahydronaphthalen-1-yl)acetamide (1-10) (7.25 g, 72% yield). ¹H NMR (400 MHz, CDCl₃) δ ppm 12.13 (br s, 1H), 8.44 (d, J=12.9 Hz, 1H), 3.81-3.98 (m, 2H), 3.07-3.09 (m, 1H), 2.67-2.90 (m, 3H), 2.24 (s, 3H), 2.10-2.18 (m, 4H), 1.83-1.97 (m, 1H). ¹⁹F NMR (376 MHz, CDCl₃) δ ppm −101.14. LCMS (ESI+) m/z: [MH]⁺ 266.1.

### N-(7-((Allyloxy)methyl)-3-fluoro-4-methyl-8-oxo-5,6,7,8-tetrahydronaphthalen-1-yl) acetamide (1-11)

To a stirred solution of N-(3-fluoro-7-(hydroxymethyl)-4-methyl-8-oxo-5,6,7,8-tetrahydronaphthalen-1-yl)acetamide (1-10) (11.8 g, 44.7 mmol) and 3-iodoprop-1-ene (20.3 mL, 222 mmol) in acetonitrile (236 mL) at 25° C. was added Ag₂O (30.9 g, 133 mmol). After stirring at 25° C. for 12 h, it was filtered, concentrated and the residue purified by silica gel column chromatography eluting with 7% ethyl acetate in petroleum ether to afford N-(7-((allyloxy)methyl)-3-fluoro-4-methyl-8-oxo-5,6,7,8-tetrahydronaphthalen-1-yl)acetamide (1-11) (3.80 g, 28% yield). ¹H NMR (400 MHz, CDCl₃) δ ppm 12.22 (s, 1H), 8.43 (d, J=12.9 Hz, 1H), 5.83-6.02 (m, 1H), 5.07-5.40 (m, 2H), 4.05 (d, J=5.6 Hz, 2H), 3.88 (dd, J=9.5, 4.5 Hz, 1H), 3.70 (dd, J=9.5, 7.1 Hz, 1H), 3.02-3.05 (m, 1H), 2.72-2.87 (m, 2H), 2.34-2.36 (m, 1H), 2.22 (s, 3H), 2.15 (d, J=1.6 Hz, 3H), 1.89-2.04 (m, 1H). ¹⁹F NMR (376 MHz, CDCl₃) δ ppm −101.93. LCMS (ESI+) m/z: [MH]⁺ 306.2.

### 2-((Allyloxy)methyl)-8-amino-6-fluoro-5-methyl-3,4-dihydronaphthalen-1(2H)-one (1-12)

To a stirred solution of N-(7-((allyloxy)methyl)-3-fluoro-4-methyl-8-oxo-5,6,7,8-tetrahydronaphthalen-1-yl)acetamide (1-11) (3.80 g, 12.5 mmol) in methanol (152 mL) at 20° C. was added 2 N HCl (152 mL). After stirring at 60° C. for 2 h, it was cooled to 0° C., pH adjusted to 7 with sat. NaHCO₃, extracted with ethyl acetate (3×300 mL), combined organic layers washed with brine, dried over Na₂SO₄, filtered, concentrated and the residue purified by silica gel column chromatography eluting with 2% ethyl acetate in petroleum ether to afford 2-((allyloxy)methyl)-8-amino-6-fluoro-5-methyl-3,4-dihydronaphthalen-1(2H)-one (1-12) (2.60 g, 79% yield). ¹H NMR (400 MHz, CDCl₃) δ ppm 6.49 (br s, 2H), 6.19 (d, J=11.6 Hz, 1H), 5.94 (m, 1H), 5.30 (dq, J=17.2, 1.6 Hz, 1H), 5.20 (dq, J=10.4, 1.3 Hz, 1H), 3.98-4.10 (m, 2H), 3.92 (dd, J=9.5, 4.4 Hz, 1H), 3.67 (dd, J=9.5, 7.8 Hz, 1H), 2.98 (dt, J=17.3, 4.6 Hz, 1H), 2.65-2.80 (m, 2H), 2.27-2.37 (m, 1H), 2.06 (d, J=1.6 Hz, 3H), 1.92-1.86 (m, 1H). ¹⁹F NMR (376 MHz, CDCl₃) δ ppm −106.56. LCMS (ESI+) m/z: [MH]⁺ 264.2.

### (9S)-1-((Allyloxy)methyl)-9-ethyl-5-fluoro-9-hydroxy-4-methyl-2,3,12,15-tetrahydrobenzo [de]pyrano[3′,4′:6,7]indolizino[1,2-b]quinoline-10,13(1H,9H)-dione (1-14)

A stirred mixture of 2-((allyloxy)methyl)-8-amino-6-fluoro-5-methyl-3,4-dihydronaphthalen-1(2H)-one (1-12) (100 mg, 0.380 mmol), (S)-4-ethyl-4-hydroxy-7,8-dihydro-1H-pyrano[3,4-f]indolizine-3,6,10(4H)-trione (1-13) (100 mg, 0.380 mmol) and 2,4,6-tripropyl-1,3,5,2,4,6-trioxatriphosphinane 2,4,6-trioxide (1.13 mL, 1.90 mmol, 50% wt in ethyl acetate) was heated at 60° C. for 10 h. Nineteen additional reactions were set up as described above and all twenty reaction mixtures were combined for work-up and purification when cooled down to room temperature. The combined reaction mixtures were diluted with water (200 mL), extracted with ethyl acetate (4×200 mL), combined organic layers washed with brine, dried over Na₂SO₄, filtered, concentrated and the residue purified by silica gel column chromatography eluting with 5% to 80% ethyl acetate in petroleum ether to afford (9S)-1-((allyloxy)methyl)-9-ethyl-5-fluoro-9-hydroxy-4-methyl-2,3,12,15-tetrahydrobenzo [de]pyrano [3′,4′:6,7]indolizino[1,2-b]quinoline-10,13(1H,9H)-dione (1-14) (310 mg, 5.4% yield). ¹H NMR (400 MHz, CDCl₃) δ ppm 7.71 (br d, J=10.6 Hz, 1H), 7.62 (s, 1H), 5.63-6.04 (m, 2H), 4.99-5.57 (m, 5H), 3.94 (br d, J=5.4 Hz, 2H), 3.66-3.79 (m, 2H), 3.47-3.64 (m, 2H), 3.14 (br d, J=3.1 Hz, 1H), 2.91-3.07 (m, 1H), 2.28-2.54 (m, 4H), 2.03-2.14 (m, 1H), 1.83-1.97 (m, 2H), 1.00-1.09 (m, 3H). ¹⁹F NMR (376 MHz, CDCl₃) δ ppm −110.59. LCMS (ESI+) m/z: [MH]⁺ 491.0.

### (9S)-9-Ethyl-5-fluoro-9-hydroxy-1-(hydroxymethyl)-4-methyl-2,3,12,15-tetrahydrobenzo[de]pyrano[3′,4′:6,7]indolizino[1,2-b]quinoline-10,13(1H,9H)-dione (1-15)

To a stirred solution of (9S)-1-((allyloxy)methyl)-9-ethyl-5-fluoro-9-hydroxy-4-methyl-2,3,12,15-tetrahydrobenzo[de]pyrano[3′,4′:6,7]indolizino[1,2-b]quinoline-10,13(1H,9H)-dione (1-14) (360 mg, 0.734 mmol) in tetrahydrofuran (18 mL) 25° C. was added zinc(II) chloride (130 mg, 0.954 mmol), stirred for 0.25 h at 25° C., followed by tetrakis(triphenylphosphine)palladium (212 mg, 0.183 mmol), stirred for 0.25 h, and followed by tributylstannane (3.88 mL, 14.7 mmol). After stirring at 50° C. for 1.5 h, it was cooled to 20° C., quenched with water (30 mL), extracted with ethyl acetate (4×30 mL), combined organic layers washed with brine, dried over Na₂SO₄, filtered, concentrated and dried under vacuum to afford (9S)-9-ethyl-5-fluoro-9-hydroxy-1-(hydroxymethyl)-4-methyl-2,3,12,15-tetrahydrobenzo[de]pyrano [3′,4′:6,7]indolizino[1,2-b]quinoline-10,13(1H,9H)-dione (1-15) (110 mg, 33% yield). LCMS (ESI+) m/z: [MH]⁺ 451.1.

### (1R,9S)-9-Ethyl-5-fluoro-9-hydroxy-1-(hydroxymethyl)-4-methyl-2,3,12,15-tetrahydro-benzo[de]pyrano[3′,4′:6,7]indolizino[1,2-b]quinoline-10,13(1H,9H)-dione (1-16) and (1S,9S)-9-ethyl-5-fluoro-9-hydroxy-1-(hydroxymethyl)-4-methyl-2,3,12,15-tetrahydro-benzo[de]pyrano [3′,4′:6,7]indolizino[1,2-b]quinoline-10,13(1H,9H)-dione (1-17)

(9S)-9-Ethyl-5-fluoro-9-hydroxy-1-(hydroxymethyl)-4-methyl-2,3,12,15-tetrahydrobenzo[de]pyrano [3′,4′:6,7]indolizino[1,2-b]quinoline-10,13(1H,9H)-dione (1-15) (110 mg, 0.244 mmol) was subjected to SFC, (Instrument: Waters SFC80 preparative SFC; Column: Phenomenex-Cellulose-2 (250 mm*30 mm,10 um); Mobile phase: A for CO₂ and B for EtOH; Gradient: B %=55% isocratic elution mode; Flow rate: 80 g/min; Wavelength: 220 nm; Column temperature: 40° C.; System back pressure: 100 bar), to afford (1R,9S)-9-ethyl-5-fluoro-9-hydroxy-1-(hydroxymethyl)-4-methyl-2,3,12,15-tetrahydrobenzo[de]pyrano [3′,4′:6,7]indolizino[1,2-b]quinoline-10,13(1H,9H)-dione (1-16) (15.0 mg, 4.5% yield) (compound 1-16 may be the opposite stereoisomer of that depicted) and (1S,9S)-9-ethyl-5-fluoro-9-hydroxy-1-(hydroxymethyl)-4-methyl-2,3,12,15-tetrahydrobenzo[de]pyrano[3′,4′:6,7]indolizino[1,2-h]quinoline-10,13(1H,9H)-dione (1-17) (10.1 mg, 3.1% yield) (compound 1-17 may be the opposite stereoisomer of that depicted). Note: The stereochemistry at the F-ring is arbitrarily assigned.

1-16: ¹H NMR (400 MHz, DMSO-D₆) δ ppm 7.75 (br d, J=11.1 Hz, 1H), 7.30 (s, 1H), 6.53 (s, 1H), 5.27-5.52 (m, 4H), 5.02 (br t, J=5.4 Hz, 1H), 3.67 (t, J=6.1 Hz, 2H), 3.46 (br d, J=0.9 Hz, 1H), 2.92-3.19 (m, 2H), 2.33-2.43 (m, 4H), 1.75-2.02 (m, 3H), 0.87 (br t, J=7.2 Hz, 3H). ¹⁹F NMR (376 MHz, DMSO-D₆) δ ppm −111.87. LCMS (ESI+) m/z: [MH]⁺ 451.1.

1-17: ¹H NMR (400 MHz, DMSO-D₆) δ ppm 7.75 (d, J=11.1 Hz, 1H), 7.30 (s, 1H), 6.52 (s, 1H), 5.27-5.50 (m, 4H), 5.02 (t, J=5.7 Hz, 1H), 3.66 (t, J=6.4 Hz, 2H), 3.46 (br d, J=3.5 Hz, 1H), 2.94-3.18 (m, 2H), 2.37 (s, 4H), 1.78-1.99 (m, 3H), 0.87 (t, J=7.3 Hz, 3H). ¹⁹F NMR (376 MHz, DMSO-D₆) δ ppm −111.86. LCMS (ESI+) m/z: [MH]⁺ calcd for C₂₅H₂₄FN₂O₅⁺: 451.1, found: 451.1.

### Example 2

### (S)-9-ethyl-5-fluoro-9-hydroxy-1,1-bis(hydroxymethyl)-4-methyl-2,3,12,15-tetrahydrobenzo [de]pyrano[3′,4′:6,7]indolizino[1,2-b]quinoline-10,13(1H,9H)-dione (2-20) (FIG. 9)

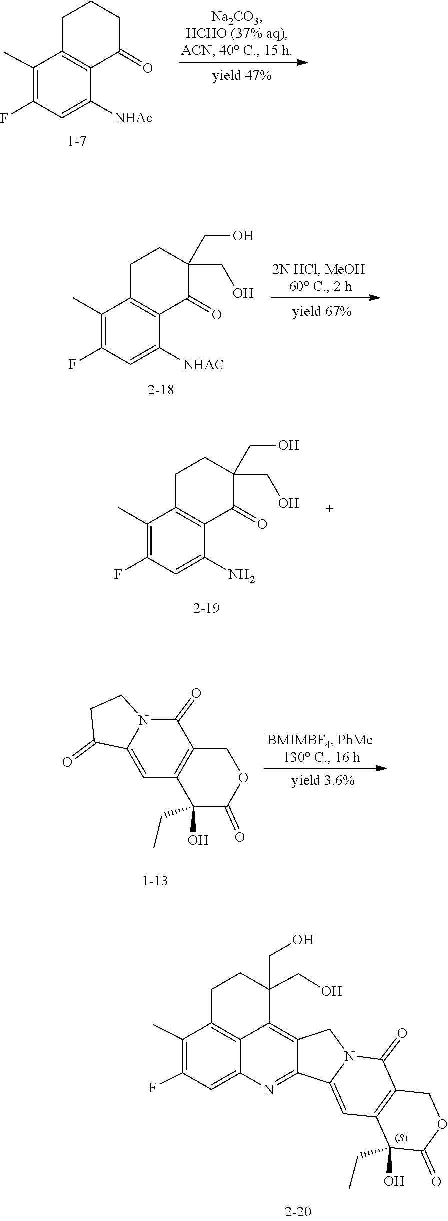

### N-(3-Fluoro-7,7-bis(hydroxymethyl)-4-methyl-8-oxo-5,6,7,8-tetrahydronaphthalen-1-yl) acetamide (2-18)

To a stirred solution of N-(3-fluoro-4-methyl-8-oxo-5,6,7,8-tetrahydronaphthalen-1-yl)acetamide (1-7) (5.00 g, 21.3 mmol) in acetonitrile (80 mL) were added sodium carbonate (451 mg, 4.25 mmol) and aqueous formaldehyde (10.4 g, 128 mmol). After stirring at 40° C. for 15 h, it was quenched with water (30 mL), extracted with dichloromethane (3×30 mL), combined organic layers washed with water, dried over Na₂SO₄, filtered, concentrated and the residue purified by silica gel column chromatography eluting with 5% to 70% ethyl acetate in petroleum ether to give a solid, which was triturated with tert-butyl methyl ether (10 mL) at 20° C. for 30 min to afford N-(3-fluoro-7,7-bis(hydroxymethyl)-4-methyl-8-oxo-5,6,7,8-tetrahydronaphthalen-1-yl)acetamide (2-18) (3.00 g, 47% yield). ¹H NMR (400 MHz, DMSO-D₆) δ ppm 11.98-12.25 (m, 1H), 8.29 (dd, J=13.2, 3.6 Hz, 1H), 4.70 (t, J=5.2 Hz, 1H), 4.54 (br d, J=7.6 Hz, 1H), 3.65-3.81 (m, 2H), 3.40-3.56 (m, 2H), 2.93 (br t, J=5.6 Hz, 2H), 2.05-2.19 (m, 8H). ¹⁹F NMR (400 MHz, DMSO-D₆) δ ppm −104.42. LCMS (ESI+) m/z: 296.1.

### 8-Amino-6-fluoro-2,2-bis(hydroxymethyl)-5-methyl-3,4-dihydronaphthalen-1(2H)-one (2-19)

To a stirred solution of N-(3-fluoro-7,7-bis(hydroxymethyl)-4-methyl-8-oxo-5,6,7,8-tetrahydro-naphthalen-1-yl)acetamide (2-18) (1.00 g, 3.39 mmol) in methanol (40 mL) was added 2N HCl (40 mL). After stirring at 60° C. for 2 h, it was cooled to 0° C., quenched with ice water (10 mL), pH adjusted to 7 with sat. sodium bicarbonate, extracted with ethyl acetate (3×100 mL), combined organic layers washed with brine, dried over Na₂SO₄, filtered, concentrated and the residue purified by silica gel column chromatography eluting with 5%˜33% ethyl acetate in petroleum ether to afford 8-amino-6-fluoro-2,2-bis(hydroxymethyl)-5-methyl-3,4-dihydronaphthalen-1(2H)-one (2-19) (0.50 g, 58% yield). ¹H NMR (400 MHz, CDCl₃) δ ppm 6.21 (d, J=11.6 Hz, 1H), 3.97 (d, J=11.2 Hz, 2H), 3.74 (d, J=11.2 Hz, 2H), 2.86 (t, J=6.4 Hz, 2H), 2.06 (d, J=1.6 Hz, 3H), 1.89 (t, J=6.4 Hz, 2H). ¹⁹F NMR (400 MHz, CDCl₃) δ ppm −105.09. LCMS (ESI+) m/z: [MH]⁺ 254.1.

### (S)-9-Ethyl-5-fluoro-9-hydroxy-1,1-bis(hydroxymethyl)-4-methyl-2,3,12,15-tetrahydro-benzo[de]pyrano[3′,4′:6,7]indolizino[1,2-b]quinoline-10,13(1H,9H)-dione (2-20)

To a stirred mixture of 8-amino-6-fluoro-2,2-bis(hydroxymethyl)-5-methyl-3,4-dihydronaphthalen-1(2H)-one (2-19) (100 mg, 0.394 mmol) and (S)-4-ethyl-4-hydroxy-7,8-dihydro-1H-pyrano[3,4-f]indolizine-3,6,10(4H)-trione (1-13) (208 mg, 0.790 mmol) in toluene (2 mL), was added 1-butyl-3-methyl-1H-imidazol-3-ium tetrafluoroborate (2 mL). (One additional reaction was set up as described above and two reaction mixtures were combined when cooled down to room temperature.) After stirring at 130° C. for 16 h, it was quenched with water (5 mL), extracted with ethyl acetate (3×5 mL), combined organic layers dried over Na₂SO₄, filtered, concentrated, the residue purified by prep-TLC (SiO₂, ethyl acetate/methanol=10/1) to give a solid, which was triturated with water (0.5 mL) at 20° C. for 10 min, filtered and dried under vacuum to afford (S)-9-ethyl-5-fluoro-9-hydroxy-1,1-bis(hydroxymethyl)-4-methyl-2,3,12,15-tetrahydrobenzo[de]pyrano[3′, 4′:6,7]indolizino[1,2-b]quinoline-10,13(1H,9H)-dione (2-20) (7.0 mg, 3.6% yield). ¹H NMR (400 MHz, CD₃OD) δ ppm 7.55-7.78 (m, 2H), 5.53-5.69 (m, 3H), 5.40 (d, J=16.0 Hz, 1H), 4.15 (d, J=11.6 Hz, 2H), 3.86 (d, J=11.6 Hz, 2H), 3.19 (t, J=6.4 Hz, 2H), 2.43 (s, 3H), 2.30 (t, J=6.8 Hz, 2H), 1.97 (dd, J=7.2, 5.2 Hz, 2H), 1.01 (t, J=7.2 Hz, 3H). ¹⁹F NMR (400 MHz, CD₃OD) δ ppm −113.70. LCMS (ESI+) m/z: [MH]⁺ 481.1.

### Example 3

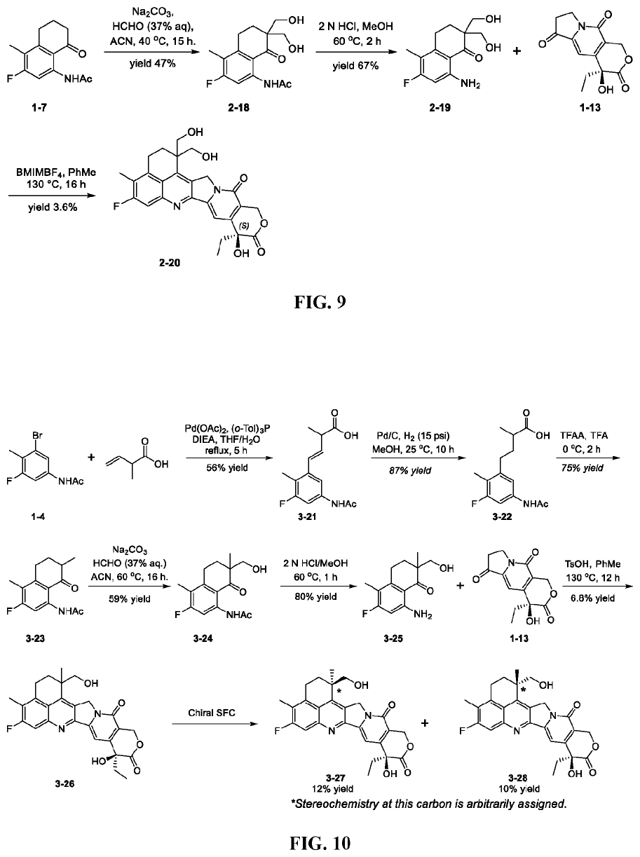

### (1R,9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-27) and (1S,9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-28) (FIG. 10)

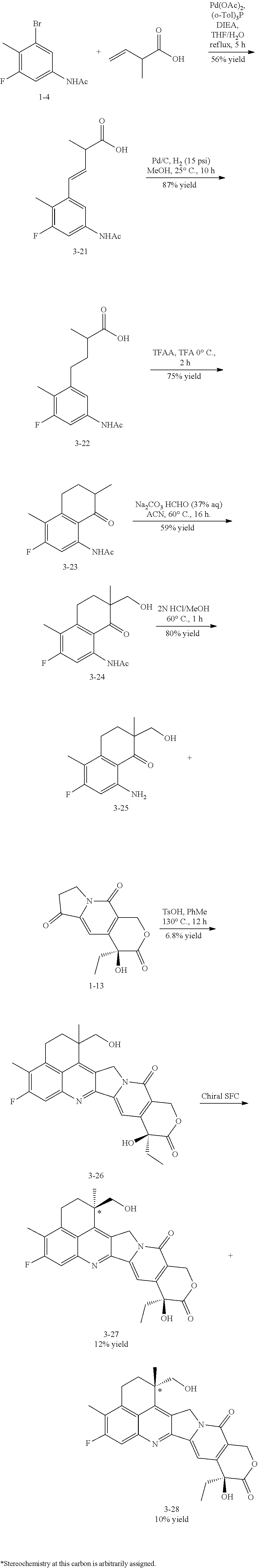

### 4-(5-Acetamido-3-fluoro-2-methylphenyl)-2-methylbut-3-enoic acid (3-21)

To a stirred mixture of N-(3-bromo-5-fluoro-4-methyl-phenyl)acetamide (1-4) (10.0 g, 40.6 mmol) and 2-methylbut-3-enoic acid (13.0 g, 130 mmol) in tetrahydrofuran (40 mL) and water (10 mL), were added N-ethyl-N,N-diisopropylamine (38.2 mL, 219 mmol), tris-o-tolylphosphane (2.47 g, 8.13 mmol) and diacetoxypalladium (912 mg, 4.06 mmol). After stirring at 75° C. for 5 h, it was cooled down to 0° C., quenched with water (50 mL), pH adjusted to 3 with 3 N HCl, mixture filtered through a pad of Celite and filter cake washed with ethyl acetate. The combined filtrates were extracted with ethyl acetate (3×150 mL), combined organic layers washed with brine, dried over Na₂SO₄, filtered, concentrated and the residue purified by silica gel column chromatography eluting with 50% ethyl acetate in petroleum ether to afford 4-(5-acetamido-3-fluoro-2-methylphenyl)-2-methylbut-3-enoic acid (3-21) (6.00 g, 56% yield). ¹H NMR (400 MHz, DMSO-D₆) δ ppm 12.38 (s, 1H), 10.02 (d, J=4.0 Hz, 1H), 7.46-7.52 (m, 1H), 6.99-7.38 (m, 1H), 6.65-6.69 (m, 1H), 6.17 (dd, J=15.6, 8.0 Hz, 1H), 3.49 (d, J=7.2 Hz, 1H), 2.11 (dd, J=13.2, 1.6 Hz, 3H), 2.02 (d, J=4.8 Hz, 3H), 1.24-1.88 (m, 3H). ¹⁹F NMR (376 MHz, DMSO-D₆) δ ppm −115.68. LCMS (ESI+) m/z: [MH]⁺ 266.1.

### 4-(5-Acetamido-3-fluoro-2-methylphenyl)-2-methylbutanoic acid (3-22)

To a degassed stirred mixture of 4-(5-acetamido-3-fluoro-2-methylphenyl)-2-methylbut-3-enoic acid (3-21) (5.00 g, 18.8 mmol) in methanol (20 mL), was added Pd/C (10 wt %) (2.40 g), purged with H₂ three times, and stirred under H₂ (15 psi) at 25° C. for 10 h. After H₂ atmosphere was replaced with argon, it was filtered through a pad of Celite and filter cake washed with methanol (300 mL), combined filtrates concentrated and dried under vacuum to afford 4-(5-acetamido-3-fluoro-2- methylphenyl)-2-methylbutanoic acid (3-22) (4.4 g, 87% yield), which was used directly for the next step. ¹H NMR (400 MHz, DMSO-D₆) δ ppm 10.0 (s, 1H), 7.42 (dd, J=12.4, 1.6 Hz, 1H), 7.04 (s, 1H), 2.52-2.58 (m, 2H), 2.24 (t, J=6.8 Hz, 2H), 2.08 (d, J=1.6 Hz. 3H), 2.01 (s, 3H), 1.40-1.65 (m, 4H). ¹9F NMR (376 MHz, DMSO-D₆) δ ppm −125.77. LCMS (ESI+) m/z: [MH]⁺ 268.1.

### N-(7-Fluoro-3,8-dimethyl-4-oxotetralin-5-yl)acetamide (3-23)

To a mixture of 4-(5-acetamido-3-fluoro-2-methylphenyl)-2-methylbutanoic acid (3-22) (5.00 g, 18.7 mmol) in trifluoroacetic acid (10 mL) was added trifluoroacetic anhydride (5.20 mL, 37.4 mmol) at 0° C. After stirred at 0° C. for 2 h, the reaction mixture was quenched with ice water (100 mL), pH adjusted to 7 with 25% aq. NaOH at 0° C., extracted with ethyl acetate (3×200 mL), combined organic layers washed with brine, dried over Na₂SO₄, filtered, concentrated, and residue purified by silica gel flash column chromatography eluting with 8% ethyl acetate in petroleum ether to give N-(7-fluoro-3,8-dimethyl-4-oxotetralin-5-yl)acetamide (3-23) (3.50 g, 75% yield). ¹H NMR (400 MHz, DMSO-D₆) δ ppm 12.13 (s, 1H), 8.28 (d, J=13.2 Hz, 1H), 2.93-3.04 (m, 1H), 2.81-2.92 (m, 1H), 2.63-2.75 (m, 1H), 2.07-2.18 (m, 7H), 1.74 (qd, J=12.0, 4.8 Hz, 1H), 1.15 (d, J=6.4 Hz, 3H). ¹⁹F NMR (376 MHz, DMSO-D₆) δ ppm −104.64. LCMS (ESI+) m/z: [MH]⁺ 250.1.

### N-(3-Fluoro-7-(hydroxymethyl)-4,7-dimethyl-8-oxo-5,6,7,8-tetrahydrona-phthalen-1-yl)acetamide (3-24)

To a stirred solution of N-(3-fluoro-4,7-dimethyl-8-oxo-5,6,7,8-tetrahydronaphthalen-1-yl)acetamide (3-23) (2.00 g, 8.04 mmol) in acetonitrile (32 mL) were added sodium carbonate (106 mg, 0.802 mmol) and aqueous formaldehyde (723 mg, 24.1 mmol). After stirring at 60° C. for 16 h, the reaction mixture was quenched with water (20 mL), extracted with dichloromethane (3×20 mL), combined organic layers washed with water, dried over Na₂SO₄, filtered, concentrated and residue purified by silica gel eluting with 30% ethyl acetate in petroleum ether to afford N-(3-fluoro-7-(hydroxymethyl)-4,7-dimethyl-8-oxo-5,6,7,8-tetrahydronaphthalen -1-yl)acetamide (3-24) (1.40 g, 59% yield). ¹H NMR (400 MHz, DMSO-D₆) δ ppm 12.17 (s, 1H), 8.26-8.34 (m, 1H), 4.81 (t, J=5.6 Hz, 1H), 3.69 (dd, J=10.4, 5.6 Hz, 1H), 2.78-3.01 (m, 2H), 2.06-2.32 (m, 8H), 1.73-1.90 (m, 1H), 1.07 (s, 3H). LCMS (ESI+) m/z: [MH]⁺ 280.1.

### 8-Amino-6-fluoro-2-(hydroxymethyl)-2,5-dimethyl-3,4-dihydronaphthalen-1(2H) -one (3-25)

To a stirred solution of N-(3-fluoro-7-(hydroxymethyl)-4,7-dimethyl-8-oxo-5,6,7,8-tetrahydro-naphthalen-1-yl)acetamide (3-24) (1.40 g, 5.00 mmol) in methanol (56 mL) was added HCl/methanol (56 mL, 2 M) at 15° C. After stirring at 60° C. for 1 h, the reaction mixture was quenched with ice water (10 mL), pH adjusted to 7 with sat. sodium bicarbonate at 0° C., extracted with ethyl acetate (6×100 mL), combined organic layers washed with brine, dried over Na₂SO₄, filtered, concentrated, and residue purified by silica gel column chromatography eluting with 15% ethyl acetate in petroleum ether to afford 8-amino-6-fluoro-2-(hydroxymethyl)-2,5-dimethyl-3,4-dihydronaphthalen-1(2H) -one (3-25) (1.00 g, 80% yield). ¹H NMR (400 MHz, DMSO-D₆) δ ppm 7.43 (br s, 2H), 6.34 (d, J=12.6 Hz, 1H), 4.65 (t, J=5.6 Hz, 1H), 3.65 (dd, J=10.4, 5.6 Hz, 1H), 3.25 (dd, J=10.4, 5.6 Hz, 1H), 2.64-2.87 (m, 2H), 2.06-2.08 (m, 1H), 1.98 (d, J=1.2 Hz, 3H), 1.62-1.76 (m, 1H), 1.01 (s, 3H). LCMS (ESI+) m/z: [MH]⁺ 238.1.

### (9S)-9-Ethyl-5-fluoro-9-hydroxy-1-(hydroxymethyl)-1,4-dimethyl-2,3,12,15-tetra-hydrobenzo[de]pyrano[3′,4′:6,7]indolizino[1,2-b]quinoline-10,13(1H,9H)-dione (3-26)

To a stirred clear solution of 8-amino-6-fluoro-2-(hydroxymethyl)-2,5-dimethyl-3,4-dihydro-naphthalen-1(2H)-one (3-25) (200 mg, 0.842 mmol) and (S)-4-ethyl-4-hydroxy-7,8-dihydro-1H-pyrano[3,4-f]indolizine-3,6,10(4H)-trione (1-13) (332 mg, 1.26 mmol) in toluene (10 mL) at 130° C., was added 4-methylbenzenesulfonic acid (58.1 mg, 0.337 mmol). Four additional reactions were set up as described above and all five reaction mixtures were combined for work-up and purification when cooled down to room temperature.) After stirring at 130° C. for 12 h, the reaction mixtures were concentrated and residue purified by silica gel column chromatography eluting with ethyl acetate to afford (9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-26) (157 mg, 6.8% yield). ¹H NMR (400 MHz, DMSO-D₆) δ ppm 7.74 (d, J=10.8 Hz, 1H), 7.31 (d, J=2.0 Hz, 1H), 6.46-6.56 (m, 1H), 5.35-5.58 (m, 4H), 5.04 (br d, J=6.8 Hz, 1H), 3.69-3.89 (m, 1H), 3.60 (br d, J=8.8 Hz, 1H), 2.96-3.16 (m, 2H), 2.26-2.41 (m, 3H), 2.17-2.19 (m, 1H), 1.87-1.92 (m, 3H), 1.32-1.48 (m, 3H), 0.87 (td, J=7.2, 2.0 Hz, 3H). LCMS (ESI+) m/z: [MH]⁺ 465.1.

### (1R,9S)-9-Ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-27) and (1S,9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-28)

(9S)-9-Ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-26) (157 mg, 0.323 mmol) was dissolved in methanol and separated by SFC (Instrument: Waters SFC150AP preparative SFC. Column: REGIS(s,s) WHELK-01 (250 mm*30 mm, 10 um); Mobile phase: A for CO₂ and B for EtOH; Gradient: B %=50% isocratic elution mode; Flow rate: 70 g/min; Wavelength:220 nm; Column temperature: 35 degrees centigrade; System back pressure: 120 bar) to afford (1R,9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-27) (18.0 mg, 12% yield) (compound 3-27 may be the opposite stereoisomer of that depicted) and (1S,9S)-9-ethyl-5-fluoro-9-hydroxy-1-(hydroxymethyl)-1,4-dimethyl-2,3,12,15-tetrahydrobenzo[de]pyrano[3′,4′:6,7]indolizino[1,2-b]quinoline-10,13(1H,9H)-dione (3-28) (15.0 mg, 10% yield) (compound 3-28 may be the opposite stereoisomer of that depicted). Note: the stereochemistry at the F-ring is arbitrarily assigned.

3-27: ¹H NMR (400 MHz, DMSO-D₆) δ ppm 7.76 (d, J=11.2 Hz, 1H), 7.31 (s, 1H), 6.51 (s, 1H), 5.34-5.63 (m, 4H), 5.03 (t, J=5.2 Hz, 1H), 3.79 (dd, J=11.3, 5.6 Hz, 1H), 3.56-3.68 (m, 1H), 3.10 (br d, J=5.6 Hz, 2H), 2.38 (s, 3H), 2.18-2.30 (m, 1H), 1.75-1.94 (m, 3H), 1.42 (s, 3H), 0.87 (t, J=7.2 Hz, 3H). ¹⁹F NMR (376 MHz, DMSO-D₆) δ ppm −112.58. LCMS (ESI+) m/z: [MH]⁺ 465.1.

3-28: ¹H NMR (400 MHz, DMSO-D₆) δ ppm 7.76 (d, J=10.8 Hz, 1H), 7.32 (s, 1H), 6.53 (s, 1H), 5.29-5.63 (m, 4H), 5.06 (t, J=5.2 Hz, 1H), 3.81 (br dd, J=11.2, 5.2 Hz, 1H), 3.61 (br dd, J=11.2, 5.2 Hz, 1H), 3.03-3.18 (m, 2H), 2.38 (s, 3H), 2.25 (m, 1H), 1.78-1.94 (m, 3H), 1.40 (s, 3H), 0.87 (t, J=7.2 Hz, 3H). ¹⁹F NMR (376 MHz, DMSO-D₆) δ ppm −112.57. LCMS (ESI+) m/z: [MH]⁺ 465.1.

### Example 4

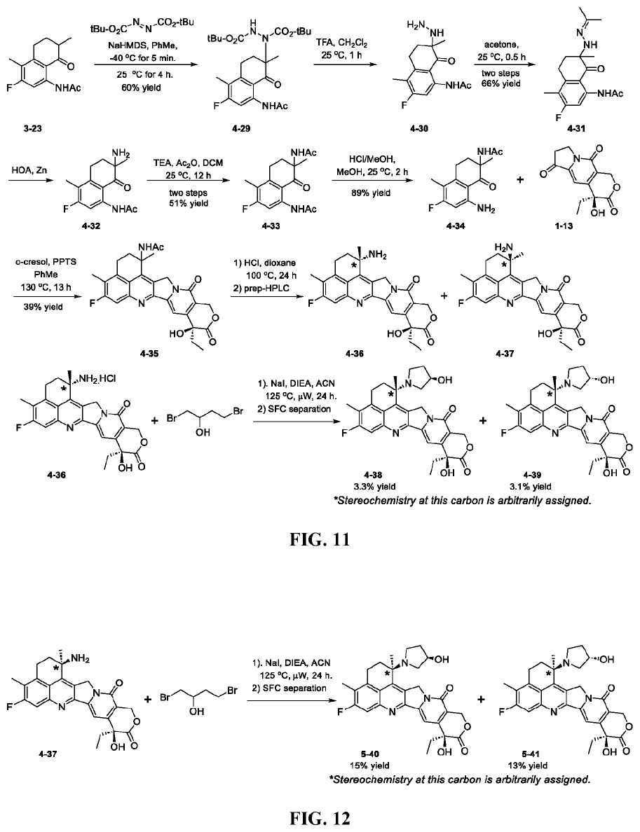

### (1S,9S)-9-ethyl-5-fluoro-9-hydroxy-1-((R)-3-hydroxypyrrolidin-1-yl)-1,4-dimethyl-2,3,12,15-tetrahydrobenzo[de]pyrano[3′,4′:6,7]indolizino[1,2-b]quinoline-10,13(1H,9H)-dione (4-38) and (1S,9S)-9-ethyl-5-fluoro-9-hydroxy-1-((S)-3-hydroxypyrrolidin-1-yl)-1,4-dimethyl-2,3,12,15-tetrahydrobenzo[de]pyrano[3′,4′:6,7]indolizino[1,2-b]quinoline-10,13(1H,9H)-dione (4-39) (FIG. 11)

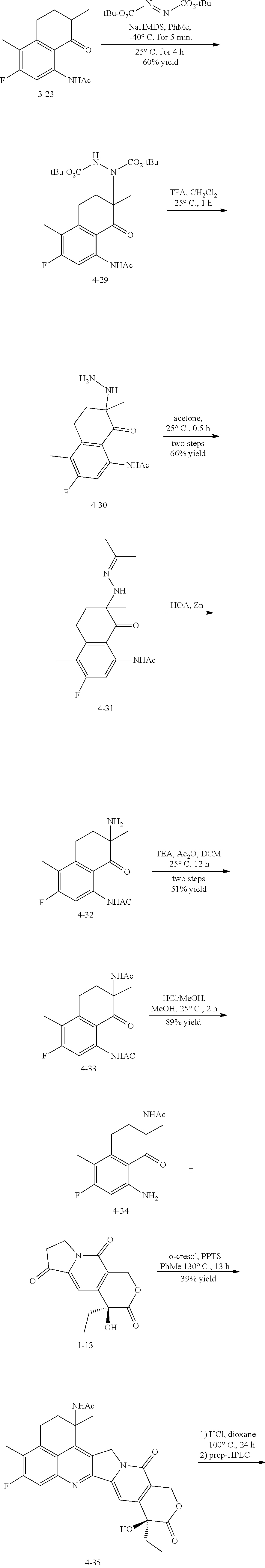

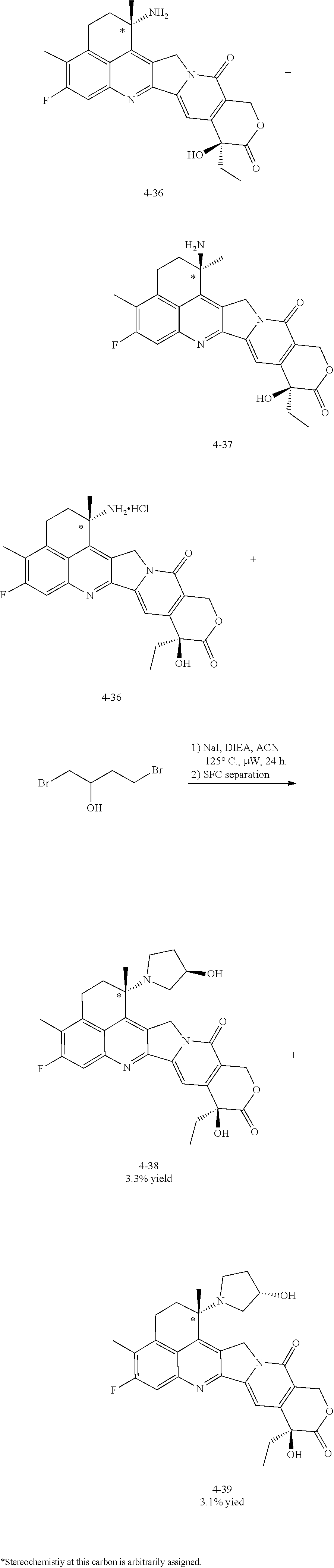

### Di-tert-Butyl 1-(8-acetamido-6-fluoro-2,5-dimethyl-1-oxo-1,2,3,4-tetrahydronaphthalen-2-yl)hydrazine-1,2-dicarboxylate (4-29)

To a stirred mixture of N-(7-fluoro-3,8-dimethyl-4-oxotetralin-5-yl)acetamide (3-23) (35.1 g, 140 mmol) in toluene (700 mL) was added sodium bis(trimethylsilyl)amide (309 mL, 1 M) dropwise at 0° C. under nitrogen, cooled down to −40° C., followed by a solution of di-tert-butyl diazene-1,2-dicarboxylate (42.1 g, 183 mmol) in toluene (350 mL). After stirring at 25° C. for 4 h, the reaction mixture was cooled to 0° C., diluted with water (1 L), extracted with ethyl acetate (3×500 mL), combined organic layers washed with brine, dried over Na₂SO₄, filtered, concentrated, and residue purified by silica gel column chromatography eluting with 20% of ethyl acetate in petroleum ether to afford di-tert-butyl 1-(8-acetamido-6-fluoro-2,5-dimethyl-1-oxo-1,2,3,4-tetrahydronaphthalen-2-yl) hydrazine-1,2-dicarboxylate (4-29) (41.0 g, 60% yield). ¹H NMR (400 MHz, DMSO-D₆) δ ppm 11.61-11.84 (m, 1H), 8.26 (d, J=12.8 Hz, 1H), 7.96-8.15 (m, 1H), 2.96-3.16 (m, 2H), 2.68-2.84 (m, 1H), 2.12 (s, 4H), 2.08 (s, 3H), 1.35-1.46 (m, 21H). ¹⁹F NMR (376 MHz, DMSO-D₆) δ ppm −105.29. LCMS (ESI+) m/z: [MH]⁺ calcd for C₂₄H₃₅FN₃O₆⁺: 480.2, found: 502 [MS+Na].

### N-(3-Fluoro-4,7-dimethyl-8-oxo-7-(2-(propan-2-ylidene)hydrazinyl)-5,6,7,8-tetrahydrona-phthalen-1-yl)acetamide (4-31)

To a solution of di-tert-butyl 1-(8-acetamido-6-fluoro-2,5-dimethyl-1-oxo-1,2,3,4-tetrahydronaphthalen-2-yl)hydrazine-1,2-dicarboxylate (4-29) (41.0 g, 85.5 mmol) in dichloromethane (820 mL) was added trifluoroacetic acid (410 mL) at 25° C., stirred at 25° C. for 1 h, followed by addition of acetone (480 mL). After stirring at 25° C. for 0.5 h, it was concentrated to afford N-(3-fluoro-4,7-dimethyl-8-oxo-7-(2-(propan-2-ylidene)hydrazinyl)-5,6,7,8-tetrahydronaphthalen-1-yl)acetamide (4-31) (18.1 g, 66% yield over 2 steps). ¹H NMR (400 MHz, DMSO-D₆) δ ppm 11.90 (s, 1H), 8.31 (d, J=12.8 Hz, 1H), 3.05-3.14 (m, 1H), 2.90-3.02 (m, 1H), 2.11-2.19 (m, 7H), 2.05-2.08 (m, 2H), 1.98 (d, J=2.0 Hz, 6H), 1.34 (s, 3H). ¹⁹F NMR (376 MHz, DMSO-D₆) δ ppm −75.04. LCMS (ESI+) m/z: [MH]⁺ calcd for C₁₇H₂₃FN₃O₂⁺: 320.1, found: 320.1.

### N-(7-Amino-3-fluoro-4,7-dimethyl-8-oxo-5,6,7,8-tetrahydronaphthalen-1-yl)acetamide (4-32)

To a mixture of N-(3-fluoro-4,7-dimethyl-8-oxo-7-(2-(propan-2-ylidene)hydrazinyl)-5,6,7,8-tetrahydronaphthalen-1-yl)acetamide (4-31) (15.1 g, 47.3 mmol) in acetic acid (302 mL) was added zinc powder (40.8 g, 624 mmol) portion wise. After stirring at 20° C. for 2 h, the reaction mixture was filtered and filtrate concentrated and dried under vacuum to afford N-(7-amino-3-fluoro-4,7-dimethyl-8-oxo-5,6,7,8-tetrahydronaphthalen-1-yl)acetamide (4-32) (23.8 g, crude), which was used directly in the next step without further purification. ¹H NMR (400 MHz, DMSO-D₆) δ ppm 11.56 (s, 1H), 8.26-8.30 (m, 1H), 3.01-3.09 (m, 2H), 2.12-2.19 (m, 10H), 1.43 (s, 3H). ¹⁹F NMR (376 MHz, DMSO-D₆) δ ppm −73.57. LCMS (ESI+) m/z: [MH]⁺ calcd for C₁₄H₁₈FN₂O₂⁺: 265.1, found: 265.1.

### N,N′-(3-Fluoro-4,7-dimethyl-8-oxo-5,6,7,8-tetrahydronaphthalene-1,7-diyl)diacetamide (4-33)

To a stirred mixture of N-(7-amino-3-fluoro-4,7-dimethyl-8-oxo-5,6,7,8-tetrahydronaphthalen-1-yl)acetamide (4-32) (23.8 g, 73.3 mmol) in dichloromethane (414 mL) were added acetic anhydride (8.28 mL, 88.0 mmol) and triethylamine (30.6 mL, 220 mmol). After stirring at 25° C. for 12 h, the reaction mixture was quenched with water (500 mL), extracted with dichloromethane (3×200 mL), combined organic layers washed with brine, dried over Na₂SO₄, filtered, concentrated, and residue purified by silica gel column chromatography eluting with 70% ethyl acetate in petroleum ether to afford N,N-(3-fluoro-4,7-dimethyl-8-oxo-5,6,7,8-tetrahydronaphthalene-1,7-diyl)diacetamide (4-33) (7.5 g, 51% yield). ¹H NMR (400 MHz, DMSO-D₆) δ ppm 11.81 (s, 1H), 8.20-8.37 (m, 2H), 2.94-3.03 (m, 1H), 2.77-2.88 (m, 1H), 2.65 (m, 1H), 2.06-2.18 (m, 6H), 1.85 (m, 1H), 1.79 (s, 3H), 1.32 (s, 3H). ¹⁹F NMR (376 MHz, DMSO-D₆) δ ppm −105.03.

### N-(8-Amino-6-fluoro-2,5-dimethyl-1-oxo-1,2,3,4-tetrahydronaphthalen-2-yl)acetamide (4-34)

To a stirred mixture of N,N-(3-fluoro-4,7-dimethyl-8-oxo-5,6,7,8-tetrahydronaphthalene-1,7-diyl)diacetamide (4-33) (7.50 g, 24.4 mmol) in methanol (105 mL) was added HCl/MeOH (105 mL, 4 M). After stirring at 25° C. for 2 h, the reaction mixture was concentrated, diluted with dichloromethane (300 mL), extracted with saturated aqueous NaHCO₃ (2×200 mL), washed with brine, dried over Na₂SO₄, filtered, concentrated and dried under vacuum to afford N-(8-amino-6-fluoro-2,5-dimethyl-1-oxo-1,2,3,4-tetrahydronaphthalen-2-yl)acetamide (4-34) (5.50 g, 89% yield). ¹H NMR (400 MHz, DMSO-D₆) δ ppm 7.91 (s, 1H), 7.39 (s, 2H), 6.36 (d, J=12.4 Hz, 1H), 2.78-2.89 (m, 1H), 2.67-2.75 (m, 2H), 1.97 (d, J=1.20 Hz, 3H), 1.83-1.88 (m, 1H), 1.80 (s, 3H), 1.27 (s, 3H). ¹⁹F NMR (376 MHz, DMSO-D₆) δ ppm −108.38. LCMS (ESI+) m/z: [MH]⁺ calcd for C₁₄H₁₈FN₂O₂⁺: 265.1, found: 265.1.

### N-((9S)-9-Ethyl-5-fluoro-9-hydroxy-1,4-dimethyl-10,13-dioxo-1,2,3,9,10,12,13,15-octahydro-benzo[de]pyrano[3′,4′:6,7]indolizino[1,2-b]quinolin-1-yl)acetamide (4-35)

To a stirred mixture of N-(8-amino-6-fluoro-2,5-dimethyl-1-oxo-1,2,3,4-tetrahydronaphthalen-2-yl)acetamide (500 mg, 1.89 mmol) (4-34) and (S)-4-ethyl-4-hydroxy-7,8-dihydro-1H-pyrano[3,4-f]indolizine-3,6,10(4H)-trione (1-13) (547 mg, 2.08 mmol) in toluene (25 mL) at 120° C. were added pyridine 4-methylbenzenesulfonate (71.3 mg, 0.283 mmol) and o-cresol (1.44 mL, 13.8 mmol) under argon. After stirring at 130° C. for 13 h with Dean-Stark trap to remove formed water, it was concentrated, and residue purified by silica gel column chromatography eluting with 7% methanol in dichloromethane to afford N-((9S)-9-ethyl-5-fluoro-9-hydroxy-1,4-dimethyl-10,13-dioxo-1,2,3,9,10,12,13,15-octahydrobenzo[de]py-rano[3′,4′:6,7]indolizino[1,2-b]quinolin-1-yl)acetamide (4-35) (362 mg, 39% yield). ¹H NMR (400 MHz, DMSO-D₆) δ ppm 7.78 (d, J=11.2 Hz, 1H), 7.30 (s, 1H), 6.53 (d, J=10.0 Hz, 1H), 5.25-5.54 (m, 4H), 4.81-4.90 (m, 1H), 3.24-3.29 (m, 1H), 2.86-3.11 (m, 3H), 2.39 (s, 3H), 1.95 (d, J=3.6 Hz, 3H), 1.84-1.89 (m, 2H), 1.50 (d, J=4.8 Hz, 3H), 0.87 (d, J=5.2 Hz, 3H). ¹⁹F NMR (376 MHz, DMSO-D₆) δ ppm −111.94. LCMS (ESI+) m/z: [MH]⁺ calcd for C₂₇H₂₇FN₃O₅⁺: 492.1, found: 492.2.

### (1S,9S)-1-Amino-9-ethyl-5-fluoro-9-hydroxy-1,4-dimethyl-2,3,12,15-tetrahydrobenzo[de]py-rano[3′,4′:6,7]indolizino[1,2-b]quinoline-10,13(1H,9H)-dione hydrochloride (4-36) and (1R,9S)-1-amino-9-ethyl-5-fluoro-9-hydroxy-1,4-dimethyl-2,3,12,15-tetrahydrobenzo[de]pyrano[3′,4′:6,7]indolizino[1,2-b]quinoline-10,13(1H,9H)-dione hydrochloride (4-37)

A stirred mixture of N-((9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)acet-amide(4-35) (600 mg, 1.22 mmol) in dioxane (6 mL) and concentrated hydrochloric acid (6 mL, 12 M) was heated at 100° C. for 24 h in a sealed tube under argon. The reaction mixture was concentrated and purified by prep-HPLC to afford (1S,9S)-1-amino-9-ethyl-5-fluoro-9-hydroxy-1,4-dimethyl-2,3,12,15-tetrahydrobenzo[de]pyrano[3′,4′:6,7]indolizino[1,2-b]quinoline-10,13(1H, 9H)-dione hydrochloride (4-36) (61.0 mg, 10% yield) (compound 4-36 may be the opposite stereoisomer of that depicted), and (1R,9S)-1-amino-9-ethyl-5-fluoro-9-hydroxy-1,4-dimethyl-2,3,12,15-tetrahydrobenzo[de]pyrano[3′,4′:6,7]indoli-zino[1,2-b]quinoline-10,13(1H,9H)-dione hydrochloride (4-37) (85.0 mg, 14% yield) (compound 4-37 may be the opposite stereoisomer of that depicted). Note: the stereochemistry at the F-ring is arbitrarily assigned.

4-36: ¹H NMR (400 MHz, DMSO-D₆) δ ppm 8.97 (s, 3H), 7.89 (d, J=10.8 Hz, 1H), 7.36 (s, 1H), 5.73-5.81 (m, 1H), 5.56-5.65 (m, 1H), 5.41-5.49 (m, 2H), 3.22 (d, J=4.4 Hz, 2H), 2.34-2.44 (m, 5H), 1.82-1.94 (m, 5H), 0.88 (t, J=7.2 Hz, 3H). ¹⁹F NMR (376 MHz, DMSO-D₆) δ ppm −111.24. LCMS (ESI+) m/z: [MH]⁺ calcd for C₂₅H₂₅FN₃O₄⁺: 450.1, found: 450.1

4-37: ¹H NMR (400 MHz, DMSO-D₆) δ ppm 8.98 (s, 3H), 7.89 (d, J=10.8 Hz, 1H), 7.36 (s, 1H), 5.71-5.79 (m, 1H), 5.57-5.64 (m, 1H), 5.46 (s, 2H), 3.18-3.26 (m, 2H), 2.36-2.44 (m, 5H), 1.81-1.94 (m, 5H), 0.87 (t, J=7.2 Hz, 3H). ¹⁹F NMR (376 MHz, DMSO-D₆) δ ppm −111.22. LCMS (ESI+) m/z: [MH]⁺ calcd for C₂₅H₂₅FN₃O₄⁺: 450.1, found: 450.1

### (1S,9S)-9-Ethyl-5-fluoro-9-hydroxy-1-((R)-3-hydroxypyrrolidin-1-yl)-1,4-dimethyl-2,3,12,15-tetrahydrobenzo[de]pyrano[3′,4′:6,7]indolizino[1,2-b]quinoline-10,13(1H,9H)-dione (4-38) and (1S,9S)-9-ethyl-5-fluoro-9-hydroxy-1-((S)-3-hydroxypyrrolidin-1-yl)-1,4-dimethyl-2,3,12,15-tetrahydrobenzo[de]pyrano[3′,4′:6,7]indolizino[1,2-b]quinoline-10,13 (1H,9H)-dione (4-39)

To a stirred mixture of (1S,9S)-1-amino-9-ethyl-5-fluoro-9-hydroxy-1,4-dimethyl-2,3,12,15-tetrahydro-benzo[de]pyrano[3′,4′:6,7]indolizino[1,2-b]quinoline-10,13(1H,9H)-dione hydrochloride (4-36) (50.0 mg, 0.102 mmol) in acetonitrile (10 mL) was added diisopropylethylamine (0.286 mL, 1.65 mmol), stirred at 20° C. for 0.5 h, followed by 1,4-dibromobutan-2-ol (0.0954 mL, 0.823 mmol) and sodium iodide (123 mg, 0.823 mmol). After stirring at 125° C. for 24 h under microwave, the reaction mixture was concentrated and residue purified by silica gel chromatography eluting with 0% to 10% methanol in ethyl acetate, followed by chiral SFC (Instrument: Waters SFC 80 preparative SFC; Column: DAICEL CHIRALPAK IG (250 mm*30 mm, 10 um); Mobile phase: A for CO₂ and B for IPA; Gradient: B %=60% isocratic elution mode; Flow rate: 80 g/min; Wavelength:220 nm; Column temperature: 40 degrees centigrade; System back pressure: 100 bar) to afford (1S,9S)-9-ethyl-5-fluoro-9-hydroxy-1-((R)-3-hydroxypyrrolidin-1-yl)-1,4-dimethyl-2,3,12,15-tetrahydrobenzo[de]pyrano[3′,4′:6,7]indolizino [1,2-b]quinoline-10,13(1H,9H)-dione (4-38) (1.80 mg, 3.3% yield) (compound 4-38 may be the opposite stereoisomer of that depicted) and (1S,9S)-9-ethyl-5-fluoro-9-hydroxy-1-((S)-3-hydroxypyrrolidin-1-yl)-1,4-dimethyl-2,3,12,15-tetrahydrobenzo[de]pyrano[3′,4′:6,7]indolizino[1,2-b]quinoline-10,13(1H,9H)-dione (4-39) (1.70 mg, 3.1% yield) (compound 4-39 may be the opposite stereoisomer of that depicted). Note: the stereochemistry at F-ring and pyrrolidine ring is arbitrarily assigned.

4-38: ¹H NMR (400 MHz, DMSO-D₆) δ ppm 7.75 (d, J=10.8 Hz, 1H), 7.29 (s, 1H), 6.49 (s, 1H), 5.34-5.50 (m, 3H), 5.23 (d, J=20.0 Hz, 1H), 4.74 (d, J=3.6 Hz, 1H), 4.22 (br s, 1H), 3.38 (br s, 1H), 2.88-2.98 (m, 1H), 2.66-2.82 (m, 3H), 2.48 (br s, 1H), 2.39 (s, 3H), 2.07-2.17 (m, 2H), 1.92-2.02 (m, 1H), 1.78-1.92 (m, 2H), 1.58-1.68 (m, 1H), 1.48 (s, 3H), 0.88 (t, J=7.2 Hz, 3H). ¹⁹F NMR (376 MHz, DMSO-D₆) δ ppm −112.14. LCMS (ESI+) m/z: [MH]⁺ calcd for C₂₉H₃₁FN₃O₅⁺: 520.2, found: 520.3.

4-39: ¹H NMR (400 MHz, DMSO-D₆) δ ppm 7.75 (d, J=10.8 Hz, 1H), 7.29 (s, 1H), 6.49 (s, 1H), 5.56-5.65 (m, 1H), 5.34-5.45 (m, 3H), 4.73 (d, J=3.2 Hz, 1H), 4.24 (br s, 1H), 3.38 (br s, 1H), 2.85-3.01 (m, 3H), 2.43-2.49 (m, 1H), 2.31-2.41 (m, 4H), 2.07-2.22 (m, 2H), 1.92-1.98 (m, 1H), 1.77-1.92 (m, 2H), 1.62-1.67 (m, 1H), 1.49 (s, 3H), 0.88 (t, J=7.2 Hz, 3H). ¹⁹F NMR (376 MHz, DMSO-D₆) δ ppm −112.15. LCMS (ESI+) m/z: [MH]⁺ calcd for C₂₉H₃₁FN₃O₅⁺: 520.2, found: 520.2.

### Example 5

### (1R,9S)-9-ethyl-5-fluoro-9-hydroxy-1-((R)-3-hydroxypyrrolidin-1-yl)-1,4-dimethyl-2,3,12,15-tetrahydrobenzo[de]pyrano[3′,4′:6,7]indolizino [1,2-b]quinoline-10,13(1H,9H)-dione (5-40) and (1R,9S)-9-ethyl-5-fluoro-9-hydroxy-1-((S)-3-hydroxypyrrolidin-1-yl)-1,4-dimethyl-2,3,12,15-tetrahydrobenzo[de]pyrano[3′,4′:6,7]indolizino[1,2-b]quinoline-10,13(1H,9H)-dione (5-41) (FIG. 12)

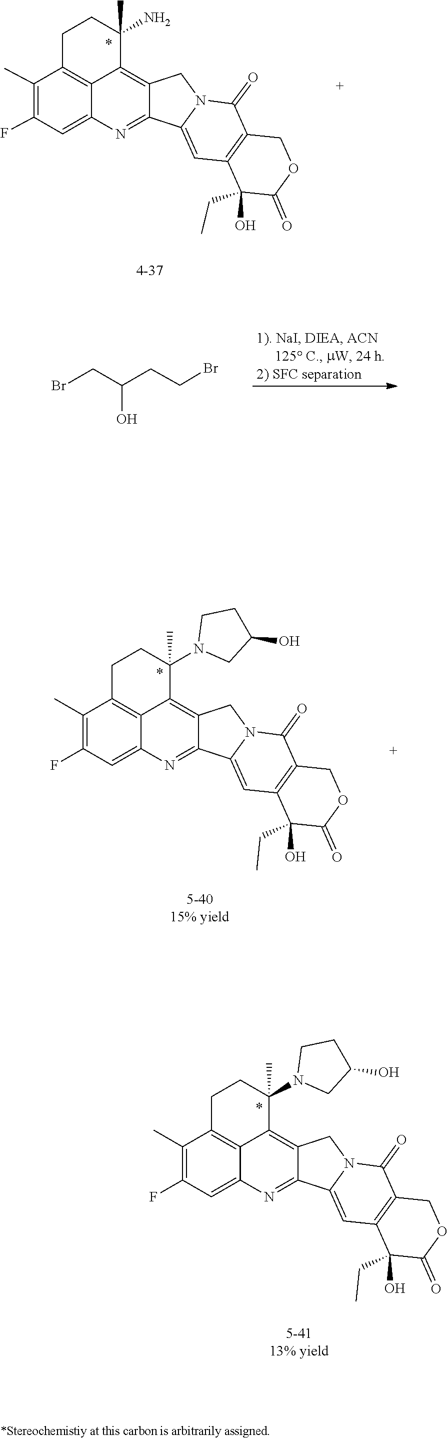 [SMILES: CC[C@@]1(O)C(=O)OCc2c1cc1n(c2=O)Cc2c-1nc1cc(F)c(C)c3c1c2[C@@](C)(N)CC3 ; OC(CBr)CCBr ; CC[C@@]1(O)C(=O)OCc2c1cc1n(c2=O)Cc2c-1nc1cc(F)c(C)c3c1c2[C@](C)(N1CC[C@@H](O)C1)CC3 ; CC[C@@]1(O)C(=O)OCc2c1cc1n(c2=O)Cc2c-1nc1cc(F)c(C)c3c1c2[C@](C)(N1CC[C@H](O)C1)CC3]

5-40 and 5-41 were made in a similar fashion to that of 4-38 and 4-39 using 4-37 instead of 4-36. Compounds 5-40 and 5-41 may be the opposite stereoisomers of those depicted. Note: the stereochemistry at F-ring and pyrrolidine ring is arbitrarily assigned.

5-40: ¹H NMR (400 MHz, DMSO-D₆) δ ppm 7.71 (d, J=10.8 Hz, 1H), 7.28 (s, 1H), 6.49 (s, 1H), 5.54-5.64 (m, 1H), 5.32-5.44 (m, 3H), 4.74 (d, J=3.2 Hz, 1H), 4.24 (br s, 1H), 3.37 (br s, 1H), 2.83-2.99 (m, 3H), 2.41-2.49 (m, 1H), 2.31-2.40 (m, 4H), 2.07-2.20 (m, 2H), 1.93-2.03 (m, 1H), 1.82-1.87 (m, 2H), 1.61-1.70 (m, 1H), 1.48 (s, 3H), 0.87 (t, J=7.2 Hz, 3H). ¹⁹F NMR (376 MHz, DMSO-D₆) δ ppm −112.15. LCMS (ESI+) m/z: [MH]⁺ calcd for C₂₉H₃₁FN₃O₅⁺: 520.2, found: 520.2.

5-41: ¹H NMR (400 MHz, DMSO-D₆) δ ppm 7.71 (d, J=10.8 Hz, 1H), 7.29 (s, 1H), 6.49 (s, 1H), 5.38-5.47 (m, 3H), 5.22 (d, J=20.0 Hz, 1H), 4.75 (d, J=3.6 Hz, 1H), 4.19-4.27 (m, 1H), 3.37 (br s, 1H), 2.86-2.98 (m, 1H), 2.64-2.83 (m, 3H), 2.50-2.52 (m, 1H), 2.38 (s, 3H), 2.05-2.22 (m, 2H), 1.92-2.02 (m, 1H), 1.79-1.92 (m, 2H), 1.57-1.70 (m, 1H), 1.47 (s, 3H), 0.87 (t, J=7.2 Hz, 3H). ¹⁹F NMR (376 MHz, DMSO-D₆) δ ppm −112.13. LCMS (ESI+) m/z: [MH]⁺ calcd for C₂₉H₃₁FN₃O₅⁺: 520.2, found: 520.2.

### Example 6

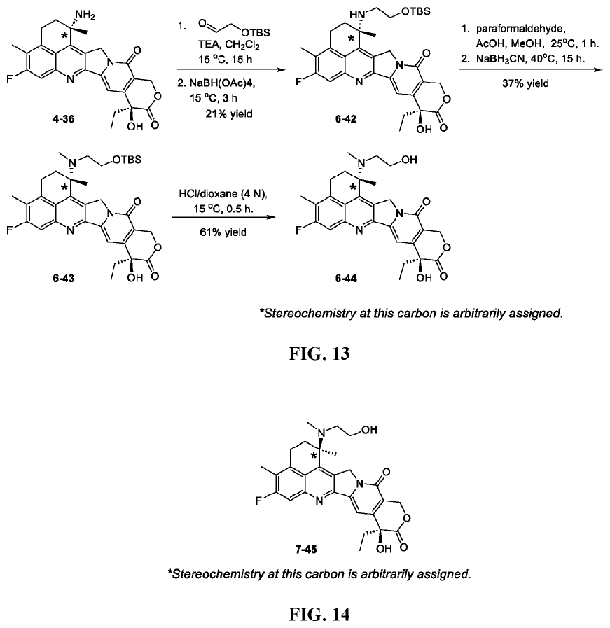

### (1S,9S)-9-ethyl-5-fluoro-9-hydroxy-1-((2-hydroxyethyl) (methyl)amino)-1,4-dimethyl-2,3,12,15-tetrahydrobenzo[de]pyrano[3′,4′:6,7]indolizino[1,2-b]quinoline-10,13(1H,9H)-dione hydrochloride (6-44) (FIG. 13)

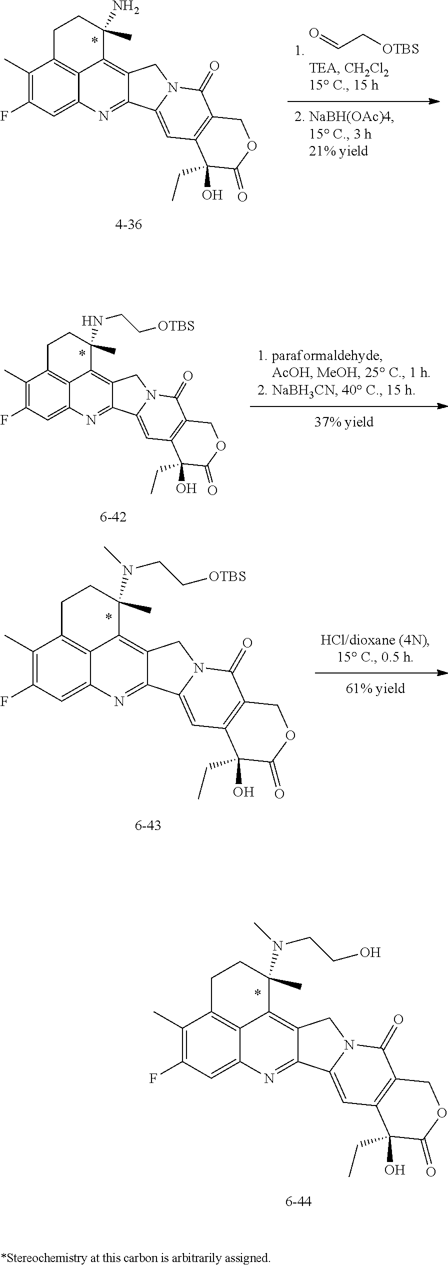 [SMILES: CC[C@@]1(O)C(=O)OCc2c1cc1n(c2=O)Cc2c-1nc1cc(F)c(C)c3c1c2[C@@](C)(N)CC3 ; CC(C)(C)[Si](C)(C)OCC=O ; CC[C@@]1(O)C(=O)OCc2c1cc1n(c2=O)Cc2c-1nc1cc(F)c(C)c3c1c2[C@@](C)(NCCO[Si](C)(C)C(C)(C)C)CC3 ; CC[C@@]1(O)C(=O)OCc2c1cc1n(c2=O)Cc2c-1nc1cc(F)c(C)c3c1c2[C@@](C)(N(C)CCO[Si](C)(C)C(C)(C)C)CC3 ; CC[C@@]1(O)C(=O)OCc2c1cc1n(c2=O)Cc2c-1nc1cc(F)c(C)c3c1c2[C@@](C)(N(C)CCO)CC3]

### (1S,9S)-1-((2-((tert-Butyldimethylsilyl)oxy)ethyl)amino)-9-ethyl-5-fluoro-9-hydroxy-1,4-dimethyl-2,3,12,15-tetrahydrobenzo[de]pyrano[3′,4′:6,7]indolizino[1,2-b]quinoline-10,13 (1H,9H)-dione (6-42)

To a suspension of (1S,9S)-1-amino-9-ethyl-5-fluoro-9-hydroxy-1,4-dimethyl-2,3,12,15-tetra-hydrobenzo[de]pyrano[3′,4′:6,7]indolizino[1,2-b]quinoline-10,13(1H,9H)-dione hydrochloride (4-36) (200 mg, 0.411 mmol) in dichloroethane (8 mL) was added triethylamine (57.2 mL, 0.411 mmol), stirred at 15° C. for 0.5 h, followed by 2-((tert-butyldimethylsilyl)oxy)acetaldehyde (156 mL, 0.823 mmol). After stirring at 15° C. for 15 h, sodium triacetoxyhydroborate (95.9 mg, 0. 457 mmol) was added, and stirred at 15° C. for another 3 h. Then, the reaction mixture was quenched with water (20 mL), extracted with dichloroethane (3×20 mL), combined organic layers dried over Na₂SO₄, filtered, concentrated and residue purified by silica gel chromatography eluting with 35% ethyl acetate in petroleum ether to afford (1S, 9S)-1-((2-((tert-butyldimethylsilyl)oxy)ethyl)amino)-9-ethyl-5-fluoro-9-hydroxy-1,4-dimethyl-2,3,12,15-tetra-hydrobenzo[de]pyrano[3′,4′:6,7]indolizino[1,2-b]quinoline-10,13(1H,9H)-dione (6-42) (60.0 mg, 21% yield). ¹H NMR (400 MHz, CDCl₃) δ ppm 7.69 (d, J=10.5 Hz, 1H), 7.60 (s, 1H), 5.77 (d, J=16.3 Hz, 1H), 5.42-5.66 (m, 2H), 5.31 (d, J=16.3 Hz, 1H), 3.65-3.93 (m, 3H), 3.26-3.38 (m, 1H), 2.95-3.11 (m, 1H), 2.89 (m, 1H), 2.30-2.50 (m, 5H), 2.03-2.14 (m, 1H), 1.77-2.00 (m, 3H), 1.49 (s, 3H), 1.05 (t, J=7.3 Hz, 3H), 0.89 (s, 9H), 0.08 (s, 3H), 0.10 (s, 3H). ¹⁹F NMR (376 MHz, CDCl₃) δ ppm −111.20. LCMS (ESI+) m/z: [MH]⁺ calcd for C₃₃H₄₃FN₃O₅Si⁺: 608.3, found: 608.2.

### (1S,9S)-1-((2-((tert-Butyldimethylsilyl)oxy)ethyl)(methyl)amino)-9-ethyl-5-fluoro-9-hydroxy-1,4-dimethyl-2,3,12,15-tetrahydrobenzo[de]pyrano[3′,4′:6,7]indolizino[1,2-b]quinoline-10,13(1H,9H)-dione (6-43)

To a stirred solution of (1S, 9S)-1-((2-((tert-butyldimethylsilyl)oxy)ethyl)amino)-9-ethyl-5-fluoro-9-hydroxy-1,4-dimethyl-2,3,12,15-tetrahydrobenzo[de]pyrano[3′,4′:6,7]indolizino[1,2-h]quinoline-10,13(1H,9H)-dione (6-42) (50.0 mg, 0.0822 mmol) in methanol (2.0 mL), were added acetic acid (0.00941 mL, 0.164 mmol) and paraformaldehyde (12.3 mg, 0.409 mmol), stirred at 25° C. for 1 h, followed by sodium cyanoborohydride (5.16 mg, 0.0822 mmol). After stirring at 40° C. for 15 h, another batch of paraformaldehyde (12.3 mg, 0.409 mmol) was added, stirred at 40° C. for 1 h, followed by sodium cyanoborohydride (20.6 mg, 0.329 mmol). After stirring at 40° C. for another 15 h, the reaction mixture was quenched with water (1.0 mL), cooled to 0° C., pH adjusted to 8 with aqueous NaHCO₃, extracted with ethyl acetate (3×5 mL), combined organic layers washed with brine, dried over Na₂SO₄, filtered, concentrated and residue purified by silica gel chromatography eluting with 10% ethyl acetate in dichloromethane to afford (1S,9S)-1-((2-((tert-butyldimethylsilyl)oxy)ethyl)(methyl)amino)-9-ethyl-5-fluoro-9-hydroxy-1,4-dimethyl-2,3,12,15-tetrahydrobenzo[de]pyrano[3′,4′:6,7]indolizino[1,2-b]quinoline-10,13(1H,9H)-dione (6-43) (19.0 mg, 37% yield). ¹H NMR (400 MHz, CDCl₃) δ ppm 7.70 (d, J=10.5 Hz, 1H), 7.61 (s, 1H), 5.77 (d, J=16.3 Hz, 1H), 5.50-5.62 (m, 1H), 5.36-5.47 (m, 1H), 5.31 (d, J=16.3 Hz, 1H), 3.77-3.91 (m, 2H), 3.70 (s, 1H), 3.36 (br d, J=15.9 Hz, 1H), 2.82-3.07 (m, 2H), 2.48-2.62 (m, 1H), 2.39-2.46 (m, 3H), 2.30 (br s, 4H), 2.05-2.17 (m, 1H), 1.79-2.01 (m, 2H), 1.53 (s, 3H), 1.06 (t, J=7.3 Hz, 3H), 0.87 (s, 9H), 0.06 (s, 3H), 0.10 (s, 3H). ¹⁹F NMR (376 MHz, CDCl₃) δ ppm −111.00. LCMS (ESI+) m/z: [MH]⁺ calcd for C₃₄H₄₅FN₃O₅Si⁺: 622.3, found: 622.2.

### (1S,9S)-9-Ethyl-5-fluoro-9-hydroxy-1-((2-hydroxyethyl)(methyl)amino)-1,4-dimethyl-2,3,12,15-tetrahydrobenzo[de]pyrano[3′,4′:6,7]indolizino[1,2-b]quinoline-10,13(1H,9H)-dione hydrochloride (6-44)

To a stirred solution of (1S,9S)-1-((2-((tert-butyldimethylsilyl)oxy)ethyl)(methyl)amino)-9-ethyl-5-fluoro-9-hydroxy-1,4-dimethyl-2,3,12,15-tetrahydrobenzo[de]pyrano[3′,4′:6,7]indolizino[1,2-b]quinoline-10,13(1H,9H)-dione (6-43) (19.0 mg, 0.0305 mmol) in dioxane (0.45 mL) was added HCl/dioxane (0.45 mL, 4 M). After stirring at 15° C. for 1 h, the reaction mixture was filtered, filter cake washed with tert-butyl methyl ether (3×1 mL), resulting solids dissolved in water (0.5 mL), and lyophilized to give (1S,9S)-9-ethyl-5-fluoro-9-hydroxy-1-((2-hydroxyethyl)(methyl)amino)-1,4-dimethyl-2,3,12,15-tetrahydrobenzo[de]pyrano[3′,4′:6,7]indolizino[1,2-b]quinoline-10,13(1H,9H) -dione hydrochloride (6-44) (10.2 mg, 61% yield) (compound 6-44 may be the opposite stereoisomer of that depicted). Note: the stereochemistry at the F-ring is arbitrarily assigned. ¹H NMR (400 MHz, D₂O) δ ppm 7.03-7.37 (m, 2H), 5.30-5.57 (m, 4H), 3.67-3.94 (m, 2H), 3.44-3.64 (m, 1H), 3.12-3.35 (m, 3H), 2.87-2.98 (m, 1H), 2.42-2.79 (m, 4H), 2.12-2.33 (m, 6H), 1.89 (q, J=7.3 Hz, 2H), 0.88 (t, J=7.4 Hz, 3H). ¹⁹F NMR (376 MHz, D₂O) δ ppm −108.73. LCMS (ESI+) m/z: [MH]⁺ calcd for C₂₈H₃₁FN₃O₅⁺: 508.2, found: 508.1.

### Example 7

### (1R,9S)-9-ethyl-5-fluoro-9-hydroxy-1-((2-hydroxyethyl)(methyl)amino)-1,4-dimethyl-2,3,12,15-tetrahydrobenzo[de]pyrano[3′,4′:6,7]indolizino[1,2-b]quinoline-10,13(1H,9H)-dione hydrochloride (7-45) (FIG. 14)

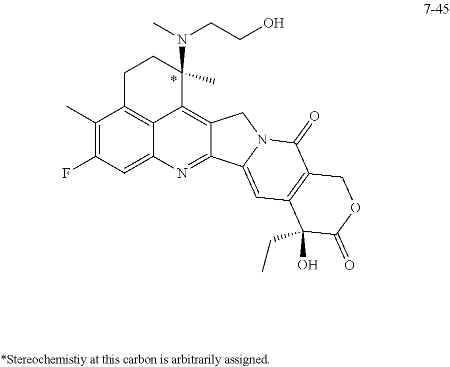 [SMILES: CC[C@@]1(O)C(=O)OCc2c1cc1n(c2=O)Cc2c-1nc1cc(F)c(C)c3c1c2[C@](C)(N(C)CCO)CC3]

7-45 was made in a similar fashion to that of 6-44 using 4-37 instead of 4-36. Compound 7-45 may be the opposite stereoisomer of that depicted. Note: the stereochemistry at the F-ring is arbitrarily assigned.

7-45: ¹H NMR (400 MHz, CD₃OD) δ ppm 7.51-7.93 (m, 2H), 5.33-5.73 (m, 4H), 3.79-3.83 (m, 2H), 3.40-3.44 (m, 1H), 3.38-3.50 (m, 2H), 2.79-3.19 (m, 2H), 2.28-2.55 (m, 6H), 2.06-2.27 (m, 2H), 1.91-2.03 (m, 2H), 1.41-1.72 (m, 2H), 1.01 (t, J=7.2 Hz, 3H). ¹⁹F NMR (400 MHz, CD₃OD) δ ppm −113.03. LCMS (ESI+) m/z: [MH]⁺ calcd for C₂₈H₃₁FN₃O₅⁺: 508.2, found: 508.2.

### Example 8

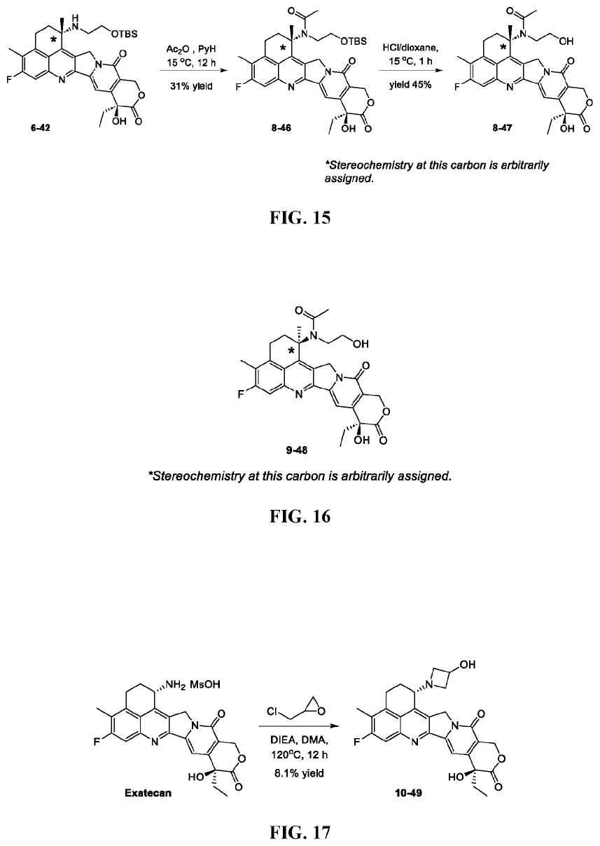

### Synthesis of N-((1S,9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)-N-(2-hydroxyethyl)acetamide (8-47) (FIG. 15)

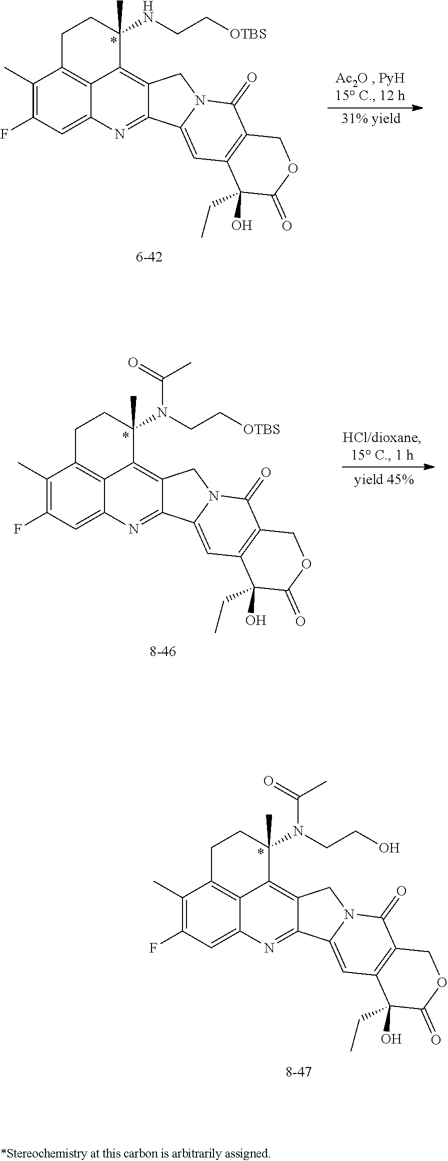 [SMILES: CC[C@@]1(O)C(=O)OCc2c1cc1n(c2=O)Cc2c-1nc1cc(F)c(C)c3c1c2[C@@](C)(NCCO[Si](C)(C)C(C)(C)C)CC3 ; CC[C@@]1(O)C(=O)OCc2c1cc1n(c2=O)Cc2c-1nc1cc(F)c(C)c3c1c2[C@@](C)(N(CCO[Si](C)(C)C(C)(C)C)C(C)=O)CC3 ; CC[C@@]1(O)C(=O)OCc2c1cc1n(c2=O)Cc2c-1nc1cc(F)c(C)c3c1c2[C@@](C)(N(CCO)C(C)=O)CC3]

### N-(2-((tert-Butyldimethylsilyl)oxy)ethyl)-N-((1S,9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)acetamide (8-46)

To a stirred solution of (1S, 9S)-1-((2-((tert-butyldimethylsilyl)oxy)ethyl)amino)-9-ethyl-5-fluoro-9-hydroxy-1,4-dimethyl-2,3,12,15-tetrahydrobenzo[de]pyrano[3′,4′:6,7]indolizino [1,2-b]quinoline-10,13(1H,9H)-dione (6-42) (60.0 mg, 0.0987 mmol) in pyridine (0.6 mL) was added acetic anhydride (100 mg, 0. 987 mmol). After stirring at 15° C. for 12 h, the reaction mixture was directly purified by silica gel chromatography eluting with 30% ethyl acetate in dichloromethane to afford N-(2-((tert-butyldimethylsilyl)oxy)ethyl)-N-((1S,9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)acetamide (8-46) (26.0 mg, 31% yield). ¹H NMR (400 MHz, DMSO-D₆) δ ppm 7.74 (d, J=10.8 Hz, 1H), 6.98 (s, 1H), 5.40-5.59 (m, 4H), 3.70 (q, J=5.2 Hz, 2H), 3.29 (br s, 1H), 2.95-3.09 (m, 1H), 2.74-2.85 (m, 1H), 2.38 (s, 4H), 2.20 (s, 3H), 2.10-2.17 (m, 3H), 1.95 (br d, J=1.6 Hz, 1H), 1.41 (s, 3H), 0.90 (br t, J=7.2 Hz, 3H), 0.81 (s, 9H), 0.02 (s, 6H). ¹⁹F NMR (376 MHz, DMSO-D₆) δ ppm −112.09. LCMS (ESI+) m/z: [MH]⁺ calcd for C₃₅H₄₅FN₃O₆Si⁺: 650.3, found: 650.2.

### N-((1S,9S)-9-Ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)-N-(2-hydroxyethyl)acetamide (8-47)

To a stirred solution of N-(2-((tert-butyldimethylsilyl)oxy)ethyl)-N-((1S,9S)-9-ethyl-5-fluoro-9-hydroxy-1,4-dimethyl-10,13-dioxo-1,2,3,9,10,12,13,15-octahydrobenzo[de]pyrano [3′,4′:6,7]indolizino[1,2-b]quinolin-1-yl)acetamide (8-46) (28.0 mg, 0.0431 mmol) in dioxane (1.12 mL) was added HCl/dioxane (215 mL, 2 M). After stirring at 15° C. for 1 h, the reaction mixture was concentrated and the residue triturated with tert-butyl methyl ether (5 mL) at 20° C. for 5 min, filtered and resulting solids further purified by prep-TLC (ethyl acetate, R_{f}=0.5) to afford N-((1S,9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)-N-(2-hydroxyethyl)acetamide (8-47) (10.0 mg, 45% yield) (compound 8-47 may be the opposite stereoisomer of that depicted). Note: the stereochemistry at the F-ring is arbitrarily assigned. ¹H NMR (400 MHz, DMSO-D₆) δ ppm 7.70 (d, J=10.8 Hz, 1H), 7.08 (s, 1H), 5.33-5.63 (m, 4H), 3.52 (td, J=5.6, 2.0 Hz, 2H), 3.29 (br d, J=17.2 Hz, 1H), 2.99 (br t, J=13.6 Hz, 1H), 2.70-2.79 (m, 1H), 2.36 (s, 3H), 2.25-2.33 (m, 1H), 2.14-2.18 (m, 4H), 2.04-2.12 (m, 2H), 1.92-2.00 (m, 1H), 1.37 (s, 3H), 0.89 (t, J=7.2 Hz, 3H). ¹⁹F NMR (376 MHz, DMSO-D₆) δ ppm −111.54. LCMS (ESI+) m/z: [MH]⁺ calcd for C₂₉H₃₁FN₃O₆^{+z}: 536.2, found: 536.2.

### Example 9

### N-((1R,9S)-9-ethyl-5-fluoro-9-hydroxy-1,4-dimethyl-10,13-dioxo-1,2,3,9,10,12,13,15-octahydrobenzo[de]pyrano[3′,4′:6,7]indolizino[1,2-b]quinolin-1-yl)-N-(2-hydroxyethyl)acetamide hydrochloride (9-48) (FIG. 16)

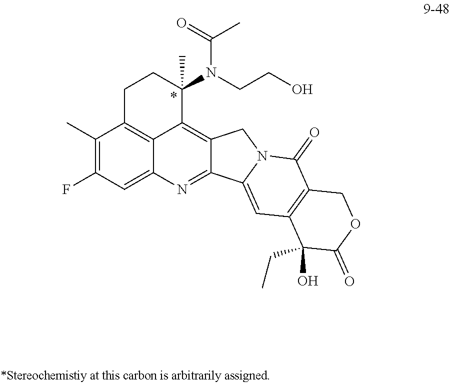 [SMILES: CC[C@@]1(O)C(=O)OCc2c1cc1n(c2=O)Cc2c-1nc1cc(F)c(C)c3c1c2[C@](C)(N(CCO)C(C)=O)CC3]

9-48 was made in a similar fashion to that of 8-47 using 4-37 instead of 4-36. Compound 9-48 may be the opposite stereoisomer of that depicted. Note: the stereochemistry at the F-ring is arbitrarily assigned.

9-48: ¹H NMR (400 MHz, DMSO-D₆) δ ppm 9.50-9.72 (m, 1H), 8.81-9.03 (m, 1H), 7.84-7.91 (m, 1H), 7.06 (s, 1H), 5.77-5.88 (m, 1H), 5.45-5.60 (m, 3H), 5.13-5.40 (m, 1H), 3.64 (br d, J=4.0 Hz, 2H), 3.20 (br d, J=5.2 Hz, 2H), 3.08 (br s, 2H), 2.62-2.66 (m, 1H), 2.41 (s, 3H), 2.26-2.31 (m, 1H), 2.22 (s, 3H), 2.14 (dt, J=10.4, 7.2 Hz, 2H), 1.96 (br s, 3H), 0.90 (t, J=7.2 Hz, 3H). ¹⁹F NMR (376 MHz, DMSO-D₆) δ ppm −111.19. LCMS (ESI+) m/z: [MH]⁺ calcd for C₂₉H₃₁FN₃O₆⁺: 536.2, found: 536.2.

### Example 10

### (1S,9S)-9-ethyl-5-fluoro-9-hydroxy-1-(3-hydroxyazetidin-1-yl)-4-methyl-2,3,12,15-tetrahydrobenzo[de]pyrano[3′,4′:6,7]indolizino[1,2-b]quinoline-10,13(1H,9H)-dione 2,2,2-trifluoroacetate (10-49) (FIG. 17)

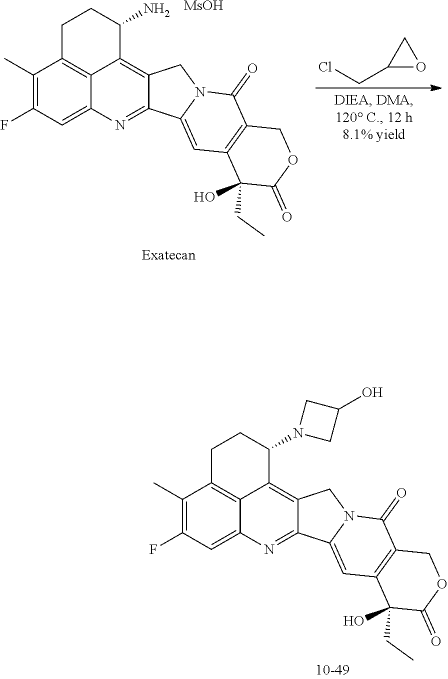 [SMILES: "MsOH" ; CC[C@@]1(O)C(=O)OCc2c1cc1n(c2=O)Cc2c-1nc1cc(F)c(C)c3c1c2[C@@H](N)CC3 ; ClCC1CO1 ; CC[C@@]1(O)C(=O)OCc2c1cc1n(c2=O)Cc2c-1nc1cc(F)c(C)c3c1c2[C@@H](N1CC(O)C1)CC3]

To a stirred mixture of (1S,9S)-1-amino-9-ethyl-5-fluoro-9-hydroxy-4-methyl-2,3,12,15-tetrahydro-benzo[de]pyrano[3′,4′:6,7]indolizino[1,2-b]quinoline-10,13(1H,9H)-dione methanesulfonate (100 mg, 0.188 mmol) and diisopropylethylamine (131 mL, 0.752 mmol) in N,N-dimethylacetamide (2 mL) was added 2-(chloromethyl)oxirane (29.5 mL, 0.376 mmol) at 25° C. After stirring at 120° C. for 10 h, the reaction mixture was concentrated and residue purified by silica gel chromatography eluting with 10% methanol in dichloromethane, further purified by pre-HPLC (acidic condition under TFA buffer) to afford (1S,9S)-9-ethyl-5-fluoro-9-hydroxy-1-(3-hydroxyazetidin-1-yl)-4-methyl-2,3,12,15-tetrahydrobenzo[de]pyrano[3′,4′:6,7]indolizino[1,2-b]quinoline-10,13(1H,9H)-dione 2,2,2-trifluoroacetate (10-49) (9.25 mg, 8.1% yield) (compound 10-49 may be the opposite stereoisomer of that depicted). Note: the stereochemistry at the F-ring is arbitrarily assigned. ¹H NMR (400 MHz, CD₃OD) δ ppm 7.78 (d, J=10.5 Hz, 1H), 7.64 (s, 1H), 5.53-5.65 (m, 2H), 5.36-5.49 (m, 2H), 5.15 (br s, 1H), 4.58 (br s, 2H), 4.06-4.46 (m, 3H), 3.36 (br s, 1H), 2.96-3.09 (m, 1H), 2.73 (br d, J=15.6 Hz, 1H), 2.46 (s, 3H), 2.28-2.40 (m, 1H), 1.88-2.03 (m, 2H), 1.01 (t, J=7.4 Hz, 3H). ¹⁹F NMR (376 MHz, CD₃OD) δ ppm −77.05 (3 F), −111.31 (1 F). LCMS (ESI+) m/z: [MH]⁺ calcd for C₂₇H₂₇FN₃O₅⁺: 492.1, found: 492.1.

### Example 11

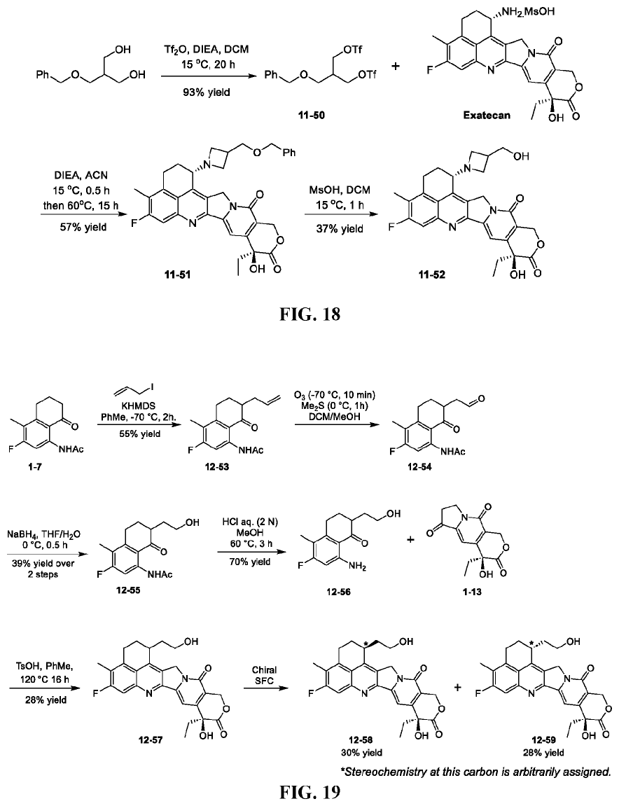

### (1S,9S)-9-ethyl-5-fluoro-9-hydroxy-1-(3-(hydroxymethyl)azetidin-1-yl)-4-methyl-2,3,12,15-tetrahydrobenzo[de]pyrano[3′,4′:6,7]indolizino[1,2-b]quinoline-10,13(1H,9H)-dione 2,2,2-trifluoroacetate (11-52) (FIG. 18)

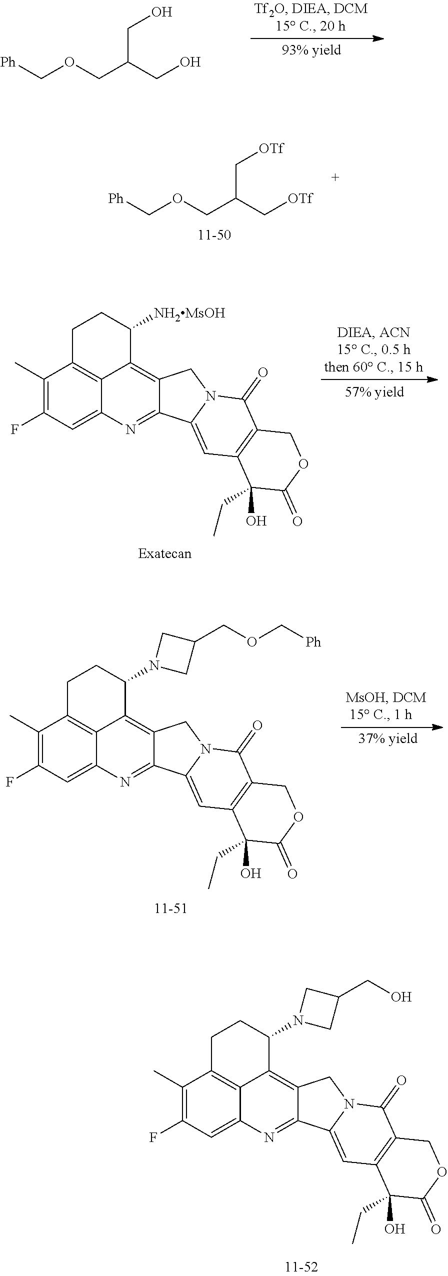

### 2-((Benzyloxy)methyl)propane-1,3-diyl bis(trifluoromethanesulfonate) (11-50)

To a stirred mixture of 2-((benzyloxy)methyl)propane-1,3-diol (200 mg, 1.02 mmol) in dichloromethane (2 mL) were added diisopropylethylamine (0.390 mL, 2.24 mmol) and trifluoromethanesulfonic anhydride (0.369 mL, 2.24 mmol) at 0° C. After stirring at 15° C. for 20 h, the reaction mixture was quenched with water (5 mL), extracted with dichloromethane (3×10 mL), combined organic layers washed with brine, dried over Na₂SO₄, filtered, concentrated and residue purified by silica gel chromatography eluting with 2% ethyl acetate in petroleum ether to afford 2-((benzyloxy)methyl)propane-1,3-diyl bis(trifluoromethanesulfonate) (11-50) (437 mg, 93% yield). ¹H NMR (400 MHz, CDCl₃) δ ppm 7.29-7.42 (m, 5H), 4.58-4.70 (m, 4H), 4.54 (s, 2H), 3.59 (d, J=5.50 Hz, 2H), 2.64 (m, 1H). ¹⁹F NMR (376 MHz, CDCl₃) δ ppm −74.30.

### (1S,9S)-1-(3-((Benzyloxy)methyl)azetidin-1-yl)-9-ethyl-5-fluoro-9-hydroxy-4-methyl-2,3,12,15-tetrahydrobenzo[de]pyrano[3′,4′:6,7]indolizino[1,2-b]quinoline-10,13(1H,9H)-dione (11-51)

To a stirred mixture of (1S,9S)-1-amino-9-ethyl-5-fluoro-9-hydroxy-4-methyl-2,3,12,15-tetrahydro-benzo[de]pyrano[3′,4′:6,7]indolizino[1,2-b]quinoline-10,13(1H,9H)-dione methanesulfonate (Exatecan) (50.0 mg, 0.094 mmol) in acetonitrile (10 mL) was added diisopropylethylamine (81.9 mL, 0.470 mmol) at 15° C., stirred at 15° C. for 0.5 h, followed by a solution of 2-((benzyloxy)methyl)propane-1,3-diyl bis(trifluoromethanesulfonate) (11-50) (86.6 mg, 0.188 mmol) in acetonitrile (2.5 mL). After stirring at 60° C. for 15 h, the reaction mixture was cooled to 15° C., concentrated, and residue purified by silica gel chromatography eluting with 7% methanol in dichloromethane to afford (1S,9S)-1-(3-((benzyloxy)methyl)azetidin-1-yl)-9-ethyl-5-fluoro-9-hydroxy-4-methyl-2,3,12,15-tetrahydrobenzo[de]pyrano[3′,4′:6,7]indolizino[1,2-b]quinoline-10,13(1H,9H)-dione (11-51) (32.0 mg, 57% yield). ¹H NMR (400 MHz, CDCl₃) δ ppm 7.55-7.70 (m, 2H), 7.29-7.39 (m, 5H), 5.76 (d, J=16.4 Hz, 1H), 5.60 (dd, J=6.0, 1.6 Hz, 1H), 5.38 (s, 2H), 5.28-5.34 (m, 1H), 4.51 (s, 2H), 3.91-4.01 (m, 2H), 3.78 (br s, 1H), 3.55 (br d, J=6.4 Hz, 2H), 3.03-3.31 (m, 4H), 2.75 (m, 1H), 2.40 (s, 3H), 1.87-1.98 (m, 4H), 1.05 (t, J=7.2 Hz, 3H). ¹⁹F NMR (376 MHz, CDCl₃) δ ppm −110.71. LCMS (ESI+) m/z: [MH]⁺ calcd for C₃₅H₃₅FN₃O₅⁺: 596.2, found: 596.3.

### (1S,9S)-9-Ethyl-5-fluoro-9-hydroxy-1-(3-(hydroxymethyl)azetidin-1-yl)-4-methyl-2,3,12,15-tetrahydrobenzo[de]pyrano[3′,4′:6,7]indolizino[1,2-b]quinoline-10,13(1H,9H)-dione 2,2,2-trifluoroacetate (11-52)

To a stirred mixture of (1S,9S)-1-(3-((benzyloxy)methyl)azetidin-1-yl)-9-ethyl-5-fluoro-9-hydroxy-4-methyl-2,3,12,15-tetrahydrobenzo[de]pyrano[3′,4′:6,7]indolizino[1,2-b]quinoline-10,13(1H,9H)-dione (11-51) (32.0 mg, 0.0537 mmol) in dichloromethane (4 mL) was added methanesulfonic acid (0.8 mL) at 0° C. After stirring at 15° C. for 1 h, the reaction mixture was concentrated, residue dissolved in dimethyl sulfoxide (3 mL), and purified by prep-HPLC (acidic condition under TFA buffer) to afford (1S, 9S)-9-ethyl-5-fluoro-9-hydroxy-1-(3-(hydroxymethyl)azetidin-1-yl)-4-methyl-2,3,12,15-tetrahydrobenzo[de]pyrano[3′,4′:6,7]indolizino[1,2-b]quinoline-10,13(1H,9H)-dione methanesulfonate 2,2,2-trifluoroacetate (11-52) (12.5 mg, 37% yield). ¹H NMR (400 MHz, D₂O) δ ppm 7.26-7.36 (m, 2H), 5.40-5.52 (m, 2H), 5.27-5.38 (m, 2H), 5.20 (br s, 1H), 4.69-4.72 (m, 2H), 4.33-4.41 (m, 1H), 4.15 (br t, J=8.8 Hz, 1H), 3.61-3.72 (m, 2H), 3.31 (br dd, J=18.4, 6.0 Hz, 1H), 3.02 (m, 1H), 2.75-2.97 (m, 2H), 2.40-2.54 (m, 1H), 2.28 (s, 3H), 1.90 (q, J=7.2 Hz, 2H), 0.88 (t, J=7.2 Hz, 3H). ¹⁹F NMR (376 MHz, D₂O) δ ppm −75.60, −108.10. LCMS (ESI+) m/z: [MH]⁺ calcd for C₂₈H₂₉FN₃O₅⁺: 506.2, found: 506.1.

### Example 12

### (1S,9S)-9-Ethyl-5-fluoro-9-hydroxy-1-(2-hydroxyethyl)-4-methyl-1,2,3,9,12,15-hexahydro-10H,13H benzo[de]pyrano[3′,4′:6,7]indolizino[1,2-b]quinoline-10,13-dione (12-58) and (1R,9S)-9-Ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (12-59) (FIG. 19)

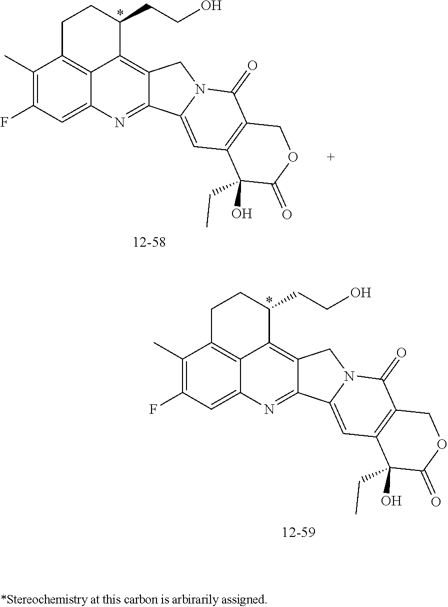 [SMILES: CC[C@@]1(O)C(=O)OCc2c1cc1n(c2=O)Cc2c-1nc1cc(F)c(C)c3c1c2[C@H](CCO)CC3 ; CC[C@@]1(O)C(=O)OCc2c1cc1n(c2=O)Cc2c-1nc1cc(F)c(C)c3c1c2[C@@H](CCO)CC3]

N-(7-Allyl-3-fluoro-4-methyl-8-oxo-5,6,7,8-tetrahydronaphthalen-1-yl)acetamide (12-53): To a solution of N-(3-fluoro-4-methyl-8-oxo-5,6,7,8-tetrahydronaphthalen-1-yl)acetamide (1-7) (7.50 g, 31.9 mmol, 1.0 eq.) in PhMe (150 mL), was added potassium bis(trimethylsilyl)amide) (1.0 M, 63.8 mL, 2.0 eq.) at −70° C., stirred at −70° C. for 1 h, followed by a solution of 3-iodoprop-1-ene (5.36 g, 31.9 mmol, 2.91 mL, 1.0 eq.) in PhMe (75 mL). After stirring at −70° C. for 1 h, it was quenched with H₂O (100 mL) at −70° C. and slowly warmed up to 25° C. Another reaction on the same scale was set up and worked up in the same way described above. The combined mixture was extracted with ethyl acetate (3×200 mL), washed with brine, dried over Na₂SO₄, filtered, and concentrated. The residue was subjected to silica gel flash chromatography (petroleum ether/ethyl acetate=100/1 to 94/6) to afford N-(7-allyl-3-fluoro-4-methyl-8-oxo-5,6,7,8-tetrahydronaphthalen-1-yl)acetamide (12-53) (9.7 g, 55% yield). ¹H NMR (400 MHz, DMSO-d₆) δ ppm 12.09 (br s, 1H) 8.29 (d, J=13.13 Hz, 1H) 5.69-5.97 (m, 1H) 4.98-5.21 (m, 2H) 2.93-3.07 (m, 1H) 2.77-2.90 (m, 1H) 2.64-2.74 (m, 1H) 2.54-2.63 (m, 1H) 2.05-2.26 (m, 8H) 1.65-1.78 (m, 1H). ¹⁹F NMR (376 MHz, DMSO-D₆) δ ppm −104.42. LCMS (ESI+) m/z: [MH]⁺ calcd for C₁₆H₁₉FNO₂⁺: 276.1, found: 276.1.

N-(3-Fluoro-4-methyl-8-oxo-7-(2-oxoethyl)-5,6,7,8-tetrahydronaphthalen-1-yl)acetamide (12-54): After a solution of N-(7-allyl-3-fluoro-4-methyl-8-oxo-5,6,7,8-tetrahydronaphthalen-1-yl)acetamide (12-53) (1.50 g, 5.45 mmol, 1.0 eq.) in dichloromethane (30 mL) and methanol (15 mL) was cooled down to −70° C., ozone (generated rate: 30 g/h) was bubbled at −70° C. into the mixture for 10 min to result in a light blue solution. Then, O₂ was bubbled into the reaction mixture at −70° C. for 15 min to remove excess ozone, Me₂S (846 mg, 13.6 mmol, 1.00 mL, 2.5 eq.) was added and the mixture warmed up to 0° C. and stirred at 0° C. for 1 h. It was quenched with H₂O (50 mL), extracted with ethyl acetate (3×50.0 mL), combined organic layers washed with brine, dried over Na₂SO₄, filtered and concentrated to give N-(3-fluoro-4-methyl-8-oxo-7-(2-oxoethyl)-5,6,7,8-tetrahydronaphthalen-1-yl)acetamide (12-54) as a crude product (1.60 g), and it was used directly without further purification. LCMS (ESI+) m/z: [MH]⁺ calcd for C₁₅H₁₆FNO₃⁺: 278.1, found: 278.1.

N-(3-Fluoro-7-(2-hydroxyethyl)-4-methyl-8-oxo-5,6,7,8-tetrahydronaphthalen-1-yl) acetamide (12-55): To a solution of crude N-(3-fluoro-4-methyl-8-oxo-7-(2-oxoethyl)-5,6,7,8-tetrahydronaphthalen-1-yl)acetamide (12-54) (1.60 g, crude) in THF (36 mL) and H₂O (18 mL), was NaBH₄ (65.5 mg, 1.73 mmol) at 25° C. After stirring at 0° C. for 0.5 h, it was quenched with H₂O (50 mL), extracted with ethyl acetate (3×100 mL), combined organic layers washed with brine, dried over Na₂SO₄, filtered, and concentrated. The residue was subjected to silica gel flash column chromatography (petroleum ether/ethyl acetate=100/1 to 7/3) to give N-(3-fluoro-7-(2-hydroxyethyl)-4-methyl-8-oxo-5,6,7,8-tetrahydronaphthalen-1-yl) acetamide (12-55) (600 mg, 39% yield over 2 steps). ¹H NMR (400 MHz, DMSO-D₆) δ ppm 12.09 (s, 1H) 8.29 (d, J=13.26 Hz, 1H) 4.50 (t, J=5.19 Hz, 1H) 3.47-3.58 (m, 2H) 2.93-3.04 (m, 1H) 2.78-2.90 (m, 1H) 2.62-2.75 (m, 1H) 2.10-2.22 (m, 7H) 1.98-2.06 (m, 1H) 1.68-1.83 (m, 1H) 1.42-1.59 (m, 1H). ¹⁹F NMR (376 MHz, DMSO-D₆) δ ppm −104.70. LCMS (ESI+) m/z: [MH−H₂O]⁺ calcd for C₁₅H₁₆FNO⁺: 262.1, found: 262.1.

8-Amino-6-fluoro-2-(2-hydroxyethyl)-5-methyl-3,4-dihydronaphthalen-1(2H)-one (12-56): To a solution of N-(3-fluoro-7-(2-hydroxyethyl)-4-methyl-8-oxo-5,6,7,8-tetrahydronaphthalen-1-yl)acetamide (12-55) (600 mg, 2.15 mmol, 1.0 eq.) in methanol (24.0 mL) was added HCl (2 M, 24.0 mL, 22 eq.). After stirring at 60° C. for 3 h, the reaction mixture was adjusted to pH 7 by saturated aq. NaHCO₃ at 0° C., extracted with ethyl acetate (3×30.0 mL), combined organic layers dried over Na₂SO₄, filtered, and concentrated. The residue was subjected to silica gel flash column chromatography (petroleum ether/ethyl acetate=100/1 to 4/1) to give 8-amino-6-fluoro-2-(2-hydroxyethyl)-5-methyl-3,4-dihydronaphthalen-1(2H)-one (12-56) (360 mg, 70% yield). ¹H NMR (400 MHz, DMSO-D₆) δ ppm 7.40 (br s, 2H) 6.35 (d, J=12.63 Hz, 1H) 4.46 (t, J=5.25 Hz, 1H) 3.44-3.56 (m, 2H) 2.85 (dt, J=17.32, 4.85 Hz, 1H) 2.60-2.74 (m, 1H) 2.44-2.50 (m, 1H) 1.89-2.15 (m, 5H) 1.59-1.74 (m, 1H) 1.36-1.51 (m, 1H) ¹⁹F NMR (376 MHz, DMSO-D₆) δ ppm −108.49. LCMS (ESI+) m/z: [MH]⁺ calcd for C₁₃H₁₇FNO₂⁺: 238.1, found: 238.1.

(1S,9S)-9-Ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (12-57): A mixture of 8-amino-6-fluoro-2-(2-hydroxyethyl)-5-methyl-3,4-dihydronaphthalen-1(2H)-one (12-56) (180 mg, 0.759 mmol, 1.0 eq.), (S)-4-ethyl-4-hydroxy-7,8-dihydro-1H-pyrano[3,4-f]indolizine-3,6,10(4H)-trione (1-13) (220 mg, 0.834 mmol, 1.1 eq.) and 4-methylbenzenesulfonic acid (52.3 mg, 0.303 mmol, 0.4 eq.) in PhMe (10 mL) was degassed and purged with argon for 3 times, and stirred at 120° C. for 16 h under argon atmosphere. Another reaction on the same scale was set up. The two combined reaction mixtures were quenched with H₂O (10 mL) at 25° C., extracted with ethyl acetate (3×10.0 mL), combined organic layers washed with brine, dried over Na₂SO₄, filtered, and concentrated. The residue was subjected to silica gel flash column chromatography (petroleum ether/ethyl acetate=100/1 to 0/1) to give (9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino [1,2-b]quinoline-10,13-dione (12-57) (200 mg, 28% yield). ¹1H NMR (400 MHz, DMSO-D₆) δ ppm 7.71 (br d, J=10.79 Hz, 1H) 7.29 (d, J=1.13 Hz, 1H) 6.50 (d, J=1.63 Hz, 1H) 5.43 (s, 2H) 5.27 (s, 2H) 4.73 (t, J=4.89 Hz, 1H) 3.58-3.67 (m, 1H) 3.53 (br d, J=3.64 Hz, 2H) 2.95-3.15 (m, 2H) 2.35 (s, 3H) 2.28 (br d, J=13.18 Hz, 1H) 1.81-1.98 (m, 3H) 1.71 (br d, J=4.52 Hz, 2H) 0.88 (br t, J=7.28 Hz, 3H). ¹⁹F NMR (376 MHz, DMSO-D₆) δ ppm −111.93. LCMS (ESI+) m/z: [MH]⁺ calcd for C₂₆H₂₆FN₂O₅⁺: 465.1, found: 465.3. SFC (retention time=1.656 min, 1.970 min).

(1S,9S)-9-Ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (12-58) and (1R,9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (12-59): (9S)-9-ethyl-5-fluoro-9-hydroxy-1-(2-hydroxyethyl)-4-methyl-1,2,3,9,12,15-hexahydro-10H,13H-benzo[de]pyrano[3′,4′:6,7]indolizino[1,2-b]quinoline-10,13-dione (12-57) (200 mg, 0.430 mmol, 1.0 eq.) was separated by SFC (Instrument: Waters SFC150AP preparative SFC; Column: DAICEL CHIRALPAK AD(250 mm*30 mm, 10 um); Mobile phase: A for CO₂ and B for ethyl alcohol; Gradient: B %=50% isocratic elution mode; Flow rate: 70 g/min; Wavelength:220 nm; Column temperature: 35 degrees centigrade; System back pressure: 120 bar.) to afford (1S,9S)-9-ethyl-5-fluoro-9-hydroxy-1-(2-hydroxyethyl)-4-methyl-1,2,3,9,12,15-hexahydro-10H,13H-benzo[de]pyrano [3′,4′:6,7]indolizino[1,2-b]quinoline-10,13-dione (12-58) (compound 12-58 may be the opposite enantiomer of that depicted) (60 mg, 30% yield) and (1R,9S)-9-ethyl-5-fluoro-9-hydroxy-1-(2-hydroxyethyl)-4-methyl-1,2,3,9,12,15-hexahydro-10H,13H-benzo[de]pyrano [3′,4′:6,7]indolizino[1,2-b]quinoline-10,13-dione (12-59) (compound 12-59 may be the opposite enantiomer of that depicted) (55 mg, 28% yield). Note: The stereochemistry at the F-ring carbon is arbitrarily assigned.

12-58: ¹H NMR (400 MHz, DMSO-D₆) δ ppm 7.73 (d, J=11.13 Hz, 1H) 7.30 (s, 1H) 6.51 (s, 1H) 5.43 (s, 2H) 5.29 (s, 2H) 4.74 (t, J=5.07 Hz, 1H) 3.58-3.67 (m, 1H) 3.49-3.57 (m, 2H) 2.98-3.17 (m, 2H) 2.37 (s, 3H) 2.29 (br d, J=13.38 Hz, 1H) 1.80-1.99 (m, 3H) 1.66-1.79 (m, 2H) 0.87 (t, J=7.32 Hz, 3H). ¹⁹F NMR (376 MHz, DMSO-D₆) δ ppm −111.91. LCMS (ESI+) m/z: [MH]⁺ calcd for C₂₆H₂₆FN₂O₅⁺: 465.1, found: 465.3. SFC (retention time=1.659 min)

12-59: ¹H NMR (400 MHz, DMSO-D₆) δ ppm 7.73 (d, J=11.01 Hz, 1H) 7.30 (s, 1H) 6.50 (s, 1H) 5.43 (s, 2H) 5.29 (s, 2H) 4.74 (t, J=5.13 Hz, 1H) 3.58-3.66 (m, 1H) 3.53 (dt, J=10.60, 5.27 Hz, 2H) 2.98-3.17 (m, 2H) 2.37 (s, 3H) 2.25-2.33 (m, 1H) 1.80-2.00 (m, 3H) 1.66-1.78 (m, 2H) 0.88 (t, J=7.32 Hz, 3H). ¹⁹F NMR (376 MHz, DMSO-D₆) δ ppm −111.91. LCMS (ESI+) m/z: [MH]⁺ calcd for C₂₆H₂₆FN₂O₅⁺: 465.1, found: 465.3. SFC (retention time=1.976 min).

### Example 13

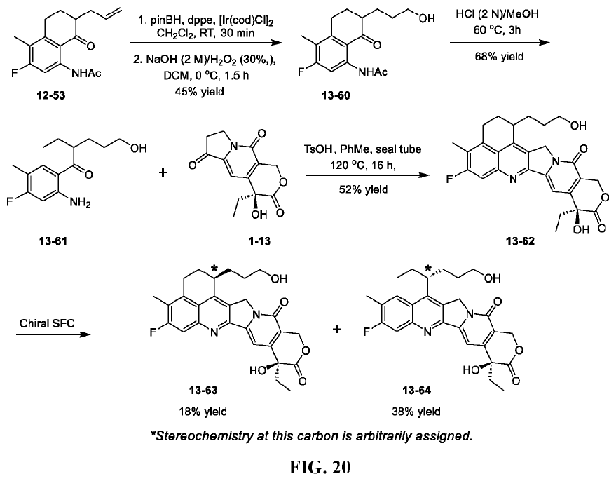

### (1S,9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-63) and (1R,9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-64) (FIG. 20)

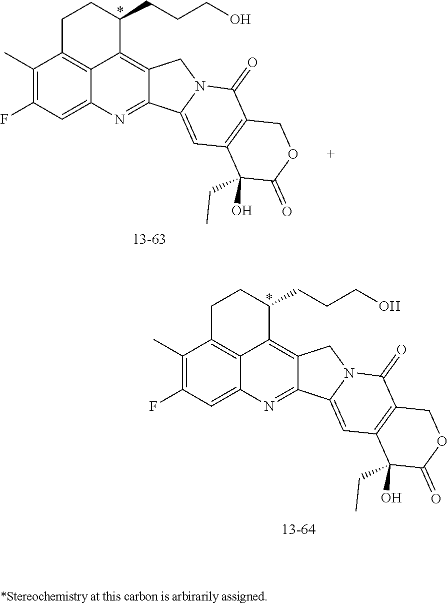 [SMILES: CC[C@@]1(O)C(=O)OCc2c1cc1n(c2=O)Cc2c-1nc1cc(F)c(C)c3c1c2[C@H](CCCO)CC3 ; CC[C@@]1(O)C(=O)OCc2c1cc1n(c2=O)Cc2c-1nc1cc(F)c(C)c3c1c2[C@@H](CCCO)CC3]

N-(3-Fluoro-7-(3-hydroxypropyl)-4-methyl-8-oxo-5,6,7,8-tetrahydronaphthalen-1-yl) acetamide (13-60): To a solution of N-(7-allyl-3-fluoro-4-methyl-8-oxo-5,6,7,8-tetrahydronaphthalen-1-yl)acetamide (12-53) (700 mg, 2.54 mmol, 1.0 eq.) in dichloromethane (30 mL) at room temperature, was added chloroiridium(1Z,5Z)-cycloocta-1,5-diene (85.3 mg, 0.127 mmol, 0.05 eq.), 1,2-bis-(diphenylphosphino)ethane (101 mg, 0.254 mmol, 0.1 eq.), stirred for 10 min, followed by 4,4,5,5-tetramethyl-1,3,2-dioxaborolane (389 mg, 3.05 mmol, 0.442 mL, 1.2 eq.). After stirring for 25 min, it was cooled to 0° C., 2 M NaOH (12 mL) and 30% aqueous H₂O₂(27.5 g, 242 mmol, 23 mL, 30%, 95 eq.) were added subsequently under vigorous stirring. Then, it was stirred for 1.5 h at 0° C., quenched with saturated aqueous Na₂S₂O₃ (50 mL), extracted with dichloromethane (3×100 mL), combined organic layers washed with brine (200 mL), dried over Na₂SO₄, filtered and concentrated. The residue was subjected to silica gel flash column chromatography (petroleum ether/ethyl acetate=100/1 to 7/3) to afford N-(3-fluoro-7-(3-hydroxypropyl)-4-methyl-8-oxo-5,6,7,8-tetrahydronaphthalen-1-yl) acetamide (13-60) (340 mg, 45% yield). ¹H NMR (400 MHz, DMSO-D₆) δ ppm 12.12 (s, 1H) 8.29 (d, J=13.13 Hz, 1H) 4.41 (t, J=5.19 Hz, 1H) 3.42 (q, J=6.09 Hz, 2H) 2.94-3.04 (m, 1H) 2.79-2.91 (m, 1H) 2.53-2.64 (m, 1H) 2.06-2.22 (m, 7H) 1.69-1.91 (m, 2H) 1.36-1.60 (m, 3H). ¹⁹F NMR (376 MHz, DMSO-D₆) δ ppm −104.66. LCMS (ESI+) m/z: [MH−H₂O]⁺ calcd for C₁₆H₁₉FNO₂⁺: 276.1, found: 276.1.

8-Amino-6-fluoro-2-(3-hydroxypropyl)-5-methyl-3,4-dihydronaphthalen-1(2H)-one (13-61): To a solution of N-(3-fluoro-7-(3-hydroxypropyl)-4-methyl-8-oxo-5,6,7,8-tetrahydronaphthalen-1-yl) acetamide (13-60) (340 mg, 1.16 mmol, 1.0 eq.) in methanol (15 mL) was added HCl (2 M, 13 mL, 23 eq.). After stirring at 60° C. for 3 h, the reaction mixture was adjusted to pH=7 by saturated aq. NaHCO₃ at 0° C., extracted with ethyl acetate (3×30 mL), combined organic layers washed with brine, dried over Na₂SO₄, filtered, and concentrated. The residue was subjected to silica gel flash column chromatography (petroleum ether/ethyl acetate=100/1 to 7/3) to give 8-amino-6-fluoro-2-(3-hydroxypropyl)-5-methyl-3,4-dihydronaphthalen-1(2H)-one (13-61) (200 mg, 68% yield). ¹H NMR (400 MHz, DMSO-D₆) δ ppm 7.42 (brs, 2H) 6.35 (d, J=12.64 Hz, 1H) 4.39 (t, J=5.19 Hz, 1H) 3.37-3.43 (m, 2H) 2.85 (dt, J=17.52, 5.13 Hz, 1H) 2.62-2.76 (m, 1H) 2.31-2.44 (m, 1H) 2.01-2.14 (m, 1H) 1.97 (d, J=1.19 Hz, 3H) 1.62-1.85 (m, 2H) 1.30-1.58 (m, 3H). ¹⁹F NMR (376 MHz, DMSO-D₆) δ ppm −108.52. LCMS (ESI+) m/z: [MH]⁺ calcd for C₁₄H₁₉FNO₂⁺: 252.1, found: 252.1.

(9S)-9-Ethyl-5-fluoro-9-hydroxy-1-(3-hydroxypropyl)-4-methyl-1,2,3,9,12,15-hexahydro-10H,13H-benzo[de]pyrano [3′,4′:6,7]indolizino[1,2-b]quinoline-10,13-dione (13-62): A mixture of 8-amino-6-fluoro-2-(3-hydroxypropyl)-5-methyl-3,4-dihydronaphthalen-1(2H)-one (13-61) (200 mg, 0.795 mmol, 1.0 eq.), (S)-4-ethyl-4-hydroxy-7,8-dihydro-1H-pyrano[3,4-f]indolizine-3,6,10(4H)-trione (1-13) (230 mg, 0.872 mmol, 1.1 eq.) and toluene-4-sulfonic acid (54.7 mg, 0.316 mmol, 0.4 eq.) in PhMe (3.5 mL) was degassed and purged with argon 3 times, and stirred at 120° C. for 16 h under argon atmosphere. It was cooled to 25° C., quenched with H₂O (10 mL), extracted with ethyl acetate (3×10 mL), combined organic layers washed with brine, dried over Na₂SO₄, filtered, and concentrated. The residue was subjected to silica gel flash column chromatography (petroleum ether/ethyl acetate=100/1 to 0/1) to give (9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino [1,2-b]quinoline-10,13-dione (13-62) (200 mg, 52% yield). ¹H NMR (400 MHz, DMSO-D₆) δ ppm 7.74 (d, J=11.13 Hz, 1H) 7.30 (d, J=1.22 Hz, 1H) 6.52 (d, J=2.08 Hz, 1H) 5.44 (s, 2H) 5.31-5.40 (m, 1H) 5.25 (br d, J=3.42 Hz, 1H) 4.39-4.47 (m, 1H) 3.40-3.51 (m, 2H) 3.35-3.39 (m, 1H) 3.10 (br d, J=3.30 Hz, 2H) 2.38 (s, 3H) 2.30 (br d, J=13.20 Hz, 1H) 1.81-1.99 (m, 3H) 1.51-1.73 (m, 4H) 0.88 (t, J=7.34 Hz, 3H). ¹⁹F NMR (376 MHz, DMSO-D₆) δ ppm −111.87. LCMS (ESI+) m/z: [MH]⁺ calcd for C₂₇H₂₈FN₂O₅⁺: 479.2, found: 479.4. SFC (retention time=0.842 min, 1.830 min).

(1S,9S)-9-Ethyl-5-fluoro-9-hydroxy-1-(3-hydroxypropyl)-4-methyl-1,2,3,9,12-15-hexahydro-10H,13H-benzo[de]pyrano[3′,4′:6,7]indolizino[1,2-b]quinoline-10,13-dione (13-63) and (1R,9S)-9-Ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-64): (9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-62) (200 mg, 0.42 mmol) was separated by chiral SFC (Instrument: Waters SFC150AP preparative SFC; Column: DAICEL CHIRALPAK IG(250 mm*30 mm, 10 um); Mobile phase: A for CO₂ and B for ethyl alcohol (0.1% NH₃H₂O); Gradient: B %=37% isocratic elution mode; Flow rate: 70 g/min; Wavelength:220 nm; Column temperature: 35 degrees centigrade; System back pressure: 120 bar) to afford (1S,9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-63) (compound 13-63 may be the opposite enantiomer of that depicted) (36.8 mg, 18% yield) and (1R,9S)-9-ethyl-5-fluoro-9-hydroxy-1-(3-hydroxypropyl)-4-methyl-1,2,3,9,12,15-hexahydro-10H,13H-benzo[de]pyrano[3′,4′:6,7]indolizino[1,2-b]quinoline-10,13-dione (13-64) (compound 13-64 may be the opposite enantiomer of that depicted) (76.2 mg, 38% yield). Note: The stereochemistry at the F-ring carbon is arbitrarily assigned.

13-63: ¹H NMR (400 MHz, DMSO-d₆) δ ppm 7.74 (d, J=11.13 Hz, 1H) 7.30 (s, 1H) 6.51 (s, 1H) 5.43 (s, 2H) 5.32-5.39 (m, 1H) 5.18-5.26 (m, 1H) 4.42 (t, J=5.13 Hz, 1H) 3.43-3.49 (m, 2H) 3.35 (br s, 1H) 2.97-3.17 (m, 2H) 2.37 (s, 3H) 2.29 (br d, J=12.63 Hz, 1H) 1.79-2.00 (m, 3H) 1.51-1.77 (m, 4H) 0.87 (t, J=7.25 Hz, 3H). ¹⁹F NMR (376 MHz, DMSO-D₆) δ ppm −111.87. LCMS (ESI+) m/z: [MH]⁺ calcd for C₂₇H₂₈FN₂O₅⁺: 479.2, found: 479.5. Chiral SFC (RT=0.837 min)

13-64: ¹H NMR (400 MHz, DMSO-D₆) δ ppm 7.73 (d, J=11.04 Hz, 1H) 7.30 (s, 1H) 6.52 (s, 1H) 5.43 (s, 2H) 5.31-5.40 (m, 1H) 5.18-5.28 (m, 1H) 4.43 (t, J=5.14 Hz, 1H) 3.42-3.51 (m, 2H) 3.33-3.39 (m, 1H) 2.98-3.16 (m, 2H) 2.37 (s, 3H) 2.24-2.34 (m, 1H) 1.80-1.99 (m, 3H) 1.51-1.77 (m, 4H) 0.87 (t, J=7.34 Hz, 3H). ¹⁹F NMR (376 MHz, DMSO-D₆) δ ppm −111.87. LCMS (ESI+) m/z: [MH]⁺ calcd for C₂₇H₂₈FN₂O₅⁺: 479.2, found: 479.5. Chiral SFC (RT=1.803 min).

### Example 14

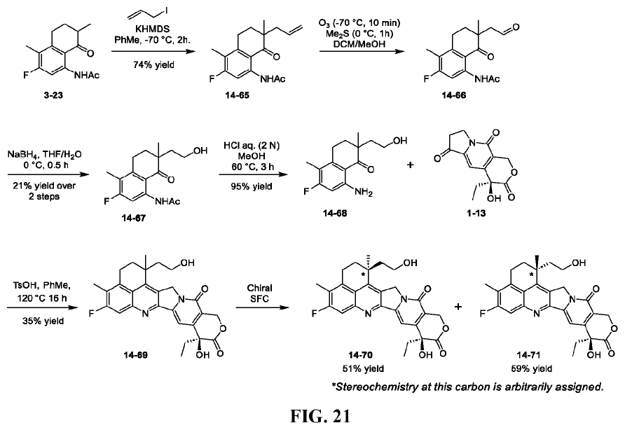

### (1S,9S)-9-ethyl-5-fluoro-9-hydroxy-1-(2-hydroxyethyl)-1,4-dimethyl-1,2,3,9,12,15-hexahydro-10H,13H-benzo[de]pyrano [3′,4′:6,7]indolizino[1,2-b]quinoline-10,13-dione (14-70) and (1R,9S)-9-ethyl-5-fluoro-9-hydroxy-1-(2-hydroxyethyl)-1,4-dimethyl-1,2,3,9,12,15-hexahydro-10H,13H-benzo[de]pyrano[3′,4′:6,7]indolizino[1,2-b]quinoline-10,13-dione (14-71) (FIG. 21)

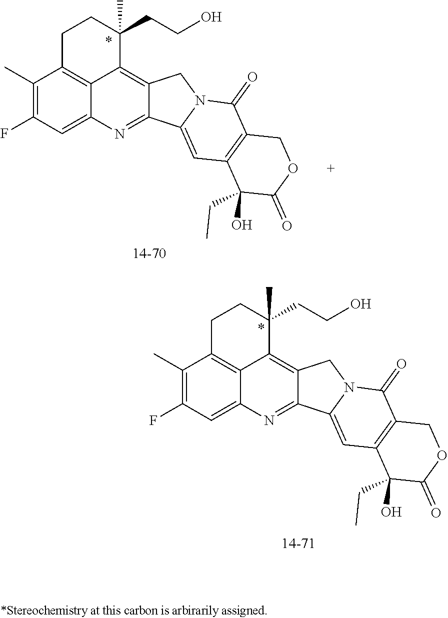 [SMILES: CC[C@@]1(O)C(=O)OCc2c1cc1n(c2=O)Cc2c-1nc1cc(F)c(C)c3c1c2[C@](C)(CCO)CC3 ; CC[C@@]1(O)C(=O)OCc2c1cc1n(c2=O)Cc2c-1nc1cc(F)c(C)c3c1c2[C@@](C)(CCO)CC3]

N-(7-Allyl-3-fluoro-4,7-dimethyl-8-oxo-5,6,7,8-tetrahydronaphthalen-1-yl)acetamide (14-65): To a solution of N-(3-Fluoro-4,7-dimethyl-8-oxo-5,6,7,8-tetrahydronaphthalen-1-yl)acetamide (3-23) (12.0 g, 48.1 mmol, 1.0 eq.) in PhMe (240 mL) was added dropwise potassium bis(trimethylsilyl)amide (1.0 M, 96.2 mL, 96.2 mmol, 2.0 eq.) at −70° C., stirred at −70° C. for 1 h, followed by 3-iodoprop-1-ene (8.09 g, 48.1 mmol, 4.39 mL, 1.0 eq.). After stirring at −70° C. for 1 h, it was quenched with H₂O (200 mL) at 25° C., extracted with ethyl acetate (3×100 mL), combined organic layers washed with brine, dried over Na₂SO₄, filtered, and concentrated. The residue was subjected to silica gel flash column chromatography (petroleum ether/ethyl acetate=100/1 to 96/4) to give N-(7-allyl-3-fluoro-4,7-dimethyl-8-oxo-5,6,7,8-tetrahydronaphthalen-1-yl) acetamide (14-65) (10.3 g, 74% yield). ¹H NMR (400 MHz, DMSO-D₆) δ ppm 12.12 (s, 1H), 8.31 (d, J=13.26 Hz, 1H), 5.69-5.85 (m, 1H), 5.04-5.15 (m, 2H), 2.80-3.00 (m, 2H), 2.30-2.40 (m, 1H), 2.10-2.25 (m, 7H), 1.92-2.02 (m, 1H), 1.77-1.91 (m, 1H), 1.11 (s, 3H). ¹⁹F NMR (376 MHz, DMSO-D₆) δ ppm −104.43. LCMS (ESI+) m/z: [MH]⁺ calcd for C₁₇H₂₁FNO₂⁺: 290.1, found: 290.1.

N-(3-Fluoro-4,7-dimethyl-8-oxo-7-(2-oxoethyl)-5,6,7,8-tetrahydronaphthalen-1yl)acetamide (14-66): After a solution of N-(7-allyl-3-fluoro-4,7-dimethyl-8-oxo-5,6,7,8-tetrahydronaphthalen-1-yl) acetamide (14-65) (1.50 g, 5.18 mmol, 1.0 eq.) in dichloromethane (30 mL) was cooled to −70° C., ozone (generated rate: 30 g/h) was bubbled into the mixture for 10 min to result in a light blue solution. Then, O₂ was bubbled into the mixture at −70° C. for 15 min to remove excess ozone, followed by Me₂S (805 mg, 12.9 mmol, 0.951 mL, 2.5 eq.). After it was warmed up to 25° C. and stirred at 25° C. for 0.5 h, it was quenched with H₂O (15 mL), extracted with ethyl acetate (3×40 mL), combined organic layers washed with brine, dried over Na₂SO₄, filtered, concentrated to give N-(3-fluoro-4,7-dimethyl-8-oxo-7-(2-oxoethyl)-5,6,7,8-tetrahydronaphthalen-1-yl)acetamide (14-66) (1.40 g, crude). LCMS (ESI+) m/z: [MH]⁺ calcd for C₁₆H₁₉FNO₃⁺: 292.1, found: 292.1.

N-(3-Fluoro-7-(2-hydroxyethyl)-4,7-dimethyl-8-oxo-5,6,7,8-tetrahydronaphthalen-1-yl)acetamide (14-67): To a solution of N-(3-fluoro-4,7-dimethyl-8-oxo-7-(2-oxoethyl)-5,6,7,8-tetrahydronaphthalen-1-yl)acetamide (14-66) (1.40 g, crude) in THF (28 mL) and water (14 mL) was added NaBH₄ (36.3 mg, 0.961 mmol, 0.5 eq.). After stirring at 0° C. for 0.5 h, it was quenched with H₂O (7 mL), extracted with ethyl acetate (3×10 mL), combined organic layers washed with brine, dried over Na₂SO₄, filtered, and concentrated. The residue was subjected to silica gel flash column chromatography (petroleum ether/ethyl acetate=100/1 to 7/3) to give N-(3-fluoro-7-(2-hydroxyethyl)-4,7-dimethyl-8-oxo-5,6,7,8-tetrahydronaphthalen-1-yl) acetamide (14-67) (330 mg, 21% yield). ¹H NMR (400 MHz, DMSO-D₆) δ ppm 11.99 (s, 1H), 8.30 (d, J=13.13 Hz, 1H), 4.39 (t, J=5.07 Hz. 1H), 3.35-3.53 (m, 2H), 2.80-3.00 (m, 2H), 2.10-2.17 (m, 6H), 1.97-2.08 (m, 1H), 1.81-1.91 (m, 1H), 1.70-1.80 (m, 1H), 1.60-1.69 (m, 1H), 1.13 (s, 3H). ¹⁹F NMR (376 MHz, DMSO-D₆) δ ppm −105.12. LCMS (EST+) m/z: [MH−H₂O]⁺ calcd for C₁₆H₁₉FNO₂⁺: 276.1, found: 276.1.

8-Amino-6-fluoro-2-(2-hydroxyethyl)-2,5-dimethyl-3,4-dihydronaphthalen-1(2H)-one (14-68): To a solution of N-(3-fluoro-7-(2-hydroxyethyl)-4,7-dimethyl-8-oxo-5,6,7,8-tetrahydronaphthalen-1-yl) acetamide (14-67) (330 mg, 1.13 mmol, 1.0 eq.) in MeOH (13.2 mL) was added HCl (2 M, 13.2 mL, 23.4 eq.). After stirring at 60° C. for 3 h, it was cooled down to 25° C., quenched with saturated NaHCO₃ to adjust the pH to 7, extracted with ethyl acetate (3×30.0 mL), combined organic layers washed with brine, dried over Na₂SO₄, filtered and concentrated. The residue was subjected to silica gel flash column chromatography (petroleum ether/ethyl acetate=100/1 to 7/3) to give 8-amino-6-fluoro-2-(2-hydroxyethyl)-2,5-dimethyl-3,4-dihydronaphthalen-1(2H)-one (14-68) (270 mg, 95% yield). ¹H NMR (400 MHz, DMSO-D₆) δ ppm 7.40 (br s, 2H), 6.36 (d, J=12.63 Hz, 1H), 4.34 (t, J=5.19 Hz, 1H), 3.37-3.51 (m, 2H), 2.64-2.89 (m, 2H), 1.89-2.02 (m, 4H), 1.66-1.84 (m, 2H), 1.52-1.65 (m, 1H), 1.08 (s, 3H). ¹⁹F NMR (376 MHz, DMSO-D₆) δ ppm −108.69. LCMS (ESI+) m/z: [MH−H₂O]⁺ calcd for C₁₄H₁₇FNO⁺: 234.1, found: 234.1.

(9S)-9-Ethyl-5-fluoro-9-hydroxy-1-(2-hydroxyethyl)-1,4-dimethyl-1,2,3,9,12,15-hexahydro-10H,13H-benzo[de]pyrano[3′,4′:6,7]indolizino[1,2-b]quinoline-10,13-dione (14-69): A mixture of 8-amino-6-fluoro-2-(2-hydroxyethyl)-2,5-dimethyl-3,4-dihydronaphthalen-1(2H)-one (14-68) (270 mg, 1.07 mmol, 1.0 eq.), (S)-4-ethyl-4-hydroxy-7,8-dihydro-1H-pyrano[3,4-f]indolizine-3,6,10(4H)-trione (1-13) (311 mg, 1.18 mmol, 1.1 eq.) and 4-methylbenzenesulfonic acid (74.0 mg, 0.429 mmol, 0.4 eq.), in PhMe (13.5 mL) was degassed and purged with argon 3 times, and stirred at 120° C. for 16 h under argon atmosphere. Then, the reaction mixture was diluted with H₂O (10 mL), extracted with ethyl acetate (3×10 mL), combined organic layers washed with brine, dried over Na₂SO₄, filtered, and concentrated. The residue was subjected to silica gel flash column chromatography (ethyl acetate/methanol=100/1 to 98/2) to give (9S)-9-ethyl-5-fluoro-9-hydroxy-1-(2-hydroxyethyl)-1,4-dimethyl-1,2,3,9,12,15-hexahydro-10H,13H-benzo[de]pyrano [3′,4′:6,7]indolizino [1,2-b]quinolone-10,13-dione (KP-6363-5) as alight red solid (180 mg, 35% yield). 1H NMR (400 MHz, DMSO-D6) δ ppm 7.74 (d, J=10.67 Hz, 1H), 7.31 (s, 1H), 6.51 (d, J=2.01 Hz, 1H), 5.32-5.54 (m, 4H), 4.45-4.51 (m, 1H), 3.39-3.62 (m, 2H), 2.98-3.23 (m, 2H), 2.37 (s, 3H), 2.08-2.24 (m, 1H), 1.80-1.98 (m, 5H), 1.53 (d, J=4.14 Hz, 3H), 0.83-0.92 (m, 3H). ¹⁹F NMR (376 MHz, DMSO) δ ppm −112.52. LCMS (ESI+) m/z: [MH]⁺ calcd for C₂₇H₂₈FN₂O₅: 479.2, found: 479.3.

(1S,9S)-9-ethyl-5-fluoro-9-hydroxy-1-(2-hydroxyethyl)-1,4-dimethyl-1,2,3,9,12,15-hexahydro-10H,13H-benzo[de]pyrano [3′,4′:6,7]indolizino[1,2-b]quinoline-10,13-dione (14-70) and (1R,9S)-9-ethyl-5-fluoro-9-hydroxy-1-(2-hydroxyethyl)-1,4-dimethyl-1,2,3,9,12,15-hexahydro-10H,13H-benzo[de]pyrano[3′,4′:6,7]indolizino[1,2-b]quinoline-10,13-dione (14-71): (9S)-9-ethyl-5-fluoro-9-hydroxy-1-(2-hydroxyethyl)-1,4-dimethyl-1,2,3,9,12,15-hexahydro-10H,13H-benzo[de]pyrano[3′,4′:6,7]indolizino[1,2-b]quinoline-10,13-dione (14-69) (180 mg, 0.37 mmol) was separated by SFC (Instrument: Waters SFC150AP preparative SFC; Column: REGIS(s,s) WHELK-01 (250 mm*30 mm, 10 um); Mobile phase: A for CO₂ and B for ethyl alcohol; Gradient: B %=50% isocratic elution mode; Flow rate: 70 g/min; Wavelength:220 nm; Column temperature: 35 degrees centigrade; System back pressure: 120 bar) to give (1S,9S)-9-ethyl-5-fluoro-9-hydroxy-1-(2-hydroxyethyl)-1,4-dimethyl-1,2,3,9,12,15-hexahydro-10H,13H-benzo[de]pyrano[3′,4′:6,7]indolizino[1,2-b]quinoline-10,13-dione (14-70) (compound 14-70 may be the opposite enantiomer of that depicted) (40.3 mg, 22.4% yield) and (1R,9S)-9-ethyl-5-fluoro-9-hydroxy-1-(2-hydroxyethyl)-1,4-dimethyl-1,2,3,9,12,15-hexa-hydro-10H,13H-benzo[de]pyrano[3′,4′:6,7]indolizino[1,2-b]quinoline-10,13-dione (14-71) (compound 14-71 may be the opposite enantiomer of that depicted) (60.5 mg, 33.6% yield). Note: The stereochemistry at the F-ring carbon is arbitrarily assigned.

14-70: ¹H NMR (400 MHz, DMSO-D₆) δ ppm 7.75 (d, J=10.76 Hz, 1H), 7.30 (s, 1H), 6.52 (s, 1H), 5.31-5.54 (m, 4H), 4.48 (t, J=5.07 Hz, 1H), 3.40-3.59 (m, 2H), 3.00-3.21 (m, 2H), 2.37 (s, 3H), 2.08-2.22 (m, 1H), 1.77-2.02 (m, 5H), 1.54 (s, 3H), 0.88 (t, J=7.32 Hz, 3H). ¹⁹F NMR (376 MHz, DMSO-D₆) δ ppm −112.53. LCMS (EST+) m/z: [MH]⁺ calcd for C₂₇H₂₈FN₂O₅⁺: 479.2, found: 479.4. SFC (retention time=0.801 min).

14-71: ¹H NMR (400 MHz, DMSO-D₆) δ ppm 7.75 (d, J=10.76 Hz, 1H), 7.31 (s, 1H), 6.53 (s, 1H), 5.32-5.54 (m, 4H), 4.49 (t, J=5.00 Hz, 1H), 3.39-3.62 (m, 2H), 2.97-3.21 (m, 2H), 2.38 (s, 3H), 2.15 (dt, J=12.73, 6.21 Hz, 1H), 1.79-2.01 (m, 5H), 1.53 (s, 3H), 0.87 (t, J=7.32 Hz, 3H). ¹⁹F NMR (376 MHz, DMSO-D₆) δ ppm −112.51. LCMS (ESI+) m/z: [MH]⁺ calcd for C₂₇H₂₈FN₂O₅: 479.2, found: 479.3. SFC (retention time=1.223 min).

### Example 15

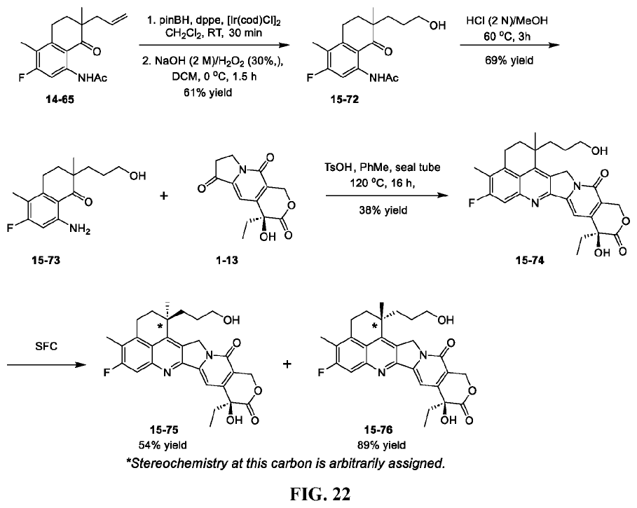

### (1S,9S)-9-Ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-75) and (1R,9S)-9-Ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-76) (FIG. 22)

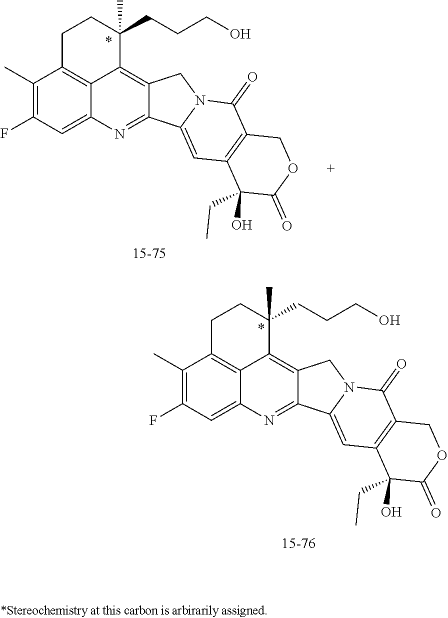 [SMILES: CC[C@@]1(O)C(=O)OCc2c1cc1n(c2=O)Cc2c-1nc1cc(F)c(C)c3c1c2[C@](C)(CCCO)CC3 ; CC[C@@]1(O)C(=O)OCc2c1cc1n(c2=O)Cc2c-1nc1cc(F)c(C)c3c1c2[C@@](C)(CCCO)CC3]

N-(3-Fluoro-7-(3-hydroxypropyl)-4,7-dimethyl-8-oxo-5,6,7,8-tetrahydronaphthalen-1-yl) acetamide (15-72): To a solution of N-(7-allyl-3-fluoro-4,7-dimethyl-8-oxo-5,6,7,8-tetrahydronaphthalen-1-yl)acetamide (14-65) (800 mg, 2.76 mmol, 1.0 eq.) in dichloromethane (40 mL) at room temperature, was added chloro(1,5-cyclooctadiene)iridium(I) dimer (92.9 mg, 0.138 mmol, 0.05 eq.), 1,2-bis(diphenylphosphino)ethane (110 mg, 0.276 mmol, 0.1 eq.), stirred for 10 min, followed by 4,4,5,5-tetramethyl-1,3,2-dioxaborolane (424 mg, 3.32 mmol, 0.481 mL, 1.2 eq.). After stirring at room temperature for 25 min, it was cooled to 0° C., and NaOH (2M, 8 mL) and H₂O₂(8 mL, 30 wt %) were added sequentially and stirred for 1.5 h. The reaction mixture was quenched with saturated Na₂S₂O₃ (16 mL) at 0° C., extracted with ethyl acetate (3×20 mL), combined organic layers washed with brine, dried over anhydrous Na₂SO₄, filtered and concentrated. The residue was subjected to silica gel flash column chromatography (petroleum ether/ethyl acetate=100/1 to 65/35), to give N-(3-fluoro-7-(3-hydroxypropyl)-4,7-dimethyl-8-oxo-5,6,7,8-tetrahydronaphthalen-1-yl) acetamide (15-72) (520 mg, 61% yield). ¹H NMR (400 MHz, DMSO-D₆) δ ppm 12.16 (s, 1H), 8.32 (d, J=13.20 Hz, 1H), 4.38 (t, J=5.26 Hz, 1H), 3.33-3.38 (m, 2H), 2.81-2.97 (m, 2H), 2.16 (s, 3H), 2.12 (d, J=1.59 Hz, 3H), 1.94-2.03 (m, 1H), 1.83-1.92 (m, 1H), 1.32-1.62 (m, 4H), 1.11 (s, 3H). ¹⁹F NMR (376 MHz, DMSO-D₆) δ ppm −104.67. LCMS (ESI+) m/z: [MH−H₂O]⁺ calcd for C₁₇H₂₁FNO₂⁺: 290.1, found: 290.1.

8-Amino-6-fluoro-2-(3-hydroxypropyl)-2,5-dimethyl-3,4-dihydronaphthalen-1(2H)-one (15-73): To a solution of N-(3-fluoro-7-(3-hydroxypropyl)-4,7-dimethyl-8-oxo-5,6,7,8-tetrahydro-naphthalen-1-yl) acetamide (15-72) (520 mg, 1.69 mmol, 1.0 eq.) in MeOH (26 mL) was added HCl (2 M, 26 mL, 24 eq.). After stirring at 60° C. for 3 h, it was cooled to 25° C., quenched with saturated NaHCO₃ to adjust pH to 7, extracted with ethyl acetate (3×30 mL), combined organic layers washed with brine, dried over anhydrous Na₂SO₄, filtered and concentrated. The residue was subjected to silica gel flash column chromatography (petroleum ether/ethyl acetate=100/1 to 65/35), to give 8-amino-6-fluoro-2-(3-hydroxypropyl)-2,5-dimethyl-3,4-dihydronaphthalen-1(2H)-one (15-73) (310 mg, 69% yield). ¹H NMR (400 MHz, DMSO-d₆) δ ppm 7.41 (br d, J=1.75 Hz, 2H), 6.35 (d, J=12.63 Hz, 1H), 4.36 (t, J=5.19 Hz, 1H), 3.35 (br s, 1H), 3.31 (br s, 1H), 2.64-2.84 (m, 2H), 1.95-2.05 (m, 3H), 1.86-1.94 (m, 1H), 1.72-1.82 (m, 1H), 1.28-1.55 (m, 4H), 1.07 (s, 3H). ¹⁹F NMR (376 MHz, DMSO) δ ppm −108.72. LCMS (ESI+) m/z: [MH−H₂O]⁺ calcd for C₁₅H₁₉NFO⁺: 248.1, found: 248.1.

(9S)-9-Ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-74): A mixture of 8-amino-6-fluoro-2-(3-hydroxypropyl)-2,5-dimethyl-3,4-dihydronaphthalen-1(2H)-one (15-73) (310 mg, 1.17 mmol, 1.0 eq.), (S)-4-ethyl-4-hydroxy-7,8-dihydro-1H-pyrano[3,4-f]indolizine-3,6,10(4H)-trione (1-13) (338 mg, 1.28 mmol, 1.1 eq.) and p-toluenesulfonic acid (80.6 mg, 0.468 mmol, 0.4 eq.) in PhMe (15 mL) was degassed and purged with argon 3 times, and stirred at 120° C. for 16 h under argon atmosphere. It was cooled down to 25° C., diluted with water (8 mL), extracted with ethyl acetate (3×10 mL), combined organic layers washed with brine, dried over anhydrous Na₂SO₄, filtered and concentrated. The residue was subjected to silica gel flash column chromatography (petroleum ether/ethyl acetate=100/0 to 15/85), to give (9S)-9-Ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-74) (220 mg, 38% yield). ¹H NMR (400 MHz, DMSO-D₆) δ ppm 7.74 (br d, J=10.63 Hz, 1H), 7.30 (s, 1H), 6.51 (d, J=2.13 Hz, 1H), 5.32-5.57 (m, 4H), 4.40 (q, J=4.79 Hz, 1H), 3.34-3.45 (m, 2H), 3.08 (br s, 2H), 2.38 (s, 3H), 2.06-2.20 (m, 1H), 1.75-2.02 (m, 4H), 1.27-1.73 (m, 6H), 0.87 (t, J=7.21 Hz, 3H). ¹⁹F NMR (376 MHz, DMSO-D₆) δ ppm −112.45. LCMS (ESI+) m/z: [MH]⁺ calcd for C₂₈H₃₀N₂O₅F⁺: 493.2, found: 493.4.

(1S,9S)-9-Ethyl-5-fluoro-9-hydroxy-1-(3-hydroxypropyl)-1,4-dimethyl-1,2,3,9,12,15-hexahydro-10H,13H-benzol[de]pyrano[3′,4′:6,7]indolizino [1,2-b]quinoline-10,13-dione (15-75) and (1R,9S)-9-Ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-76): (9S)-9-ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]indolizino[1,2-b]quinoline-10,13-dione (15-74) (220 mg, 0.447 mmol) was separated by SFC (Instrument: Waters SFC80Q preparative SFC; Column: REGIS(s,s) WHELK-01 (250 mm*30 mm,10 um); Mobile phase: A for CO₂ and B for ethyl alcohol; Gradient: B %=54% isocratic elution mode; Flow rate: 75 g/min; Wavelength: 220 nm; Column temperature: 40° C.; System back pressure: 100 bar) to give (1S,9S)-9-ethyl-5-fluoro-9-hydroxy-1-(3-hydroxypropyl)-1,4-dimethyl-1,2,3,9,12,15-hexahydro-10H,13H-benzo[de]pyrano[3′,4′:6,7]ind olizino[1,2-b]quinoline-10,13-dione (15-75) (compound 15-75 may be the opposite enantiomer of that depicted) (65.2 mg, 30% yield) and (1R,9S)-9-ethyl-5-fluoro-9-hydroxy-1-(3-hydroxypropyl)-1,4-dimethyl-1,2,3,9,12,15-hexahydro-10H,13H-benzo[de]pyra no[3′,4′:6,7]indolizino[1,2-b]quinoline-10,13-dione (15-76) (compound 15-76 may be the opposite enantiomer of that depicted) (90.5 mg, 41% yield). Note: The stereochemistry at the F-ring carbon is arbitrarily assigned.

15-75: ¹H NMR (400 MHz, DMSO-D₆) δ ppm 7.74 (br d, J=10.76 Hz, 1H), 7.30 (s, 1H), 6.51 (s, 1H), 5.29-5.59 (m, 4H), 4.40 (t, J=5.14 Hz, 1H), 3.32-3.40 (m, 2H), 3.08 (br s, 2H), 2.37 (s, 3H), 2.06-2.19 (m, 1H), 1.72-1.95 (m, 4H), 1.58-1.69 (m, 1H), 1.50 (s, 3H), 1.34-1.48 (m, 2H), 0.87 (br t, J=7.21 Hz, 3H). ¹⁹F NMR (376 MHz, DMSO-D₆) δ ppm −112.45. LCMS (ESI+) m/z: [MH]⁺ calcd for C₂₈H₃₀FN₂O₅: 493.2, found: 493.5. SFC (retention time=0.761 min).

15-76: ¹H NMR (400 MHz, DMSO-D₆) δ ppm 7.75 (d, J=10.76 Hz, 1H), 7.31 (s, 1H), 6.52 (s, 1H), 5.34-5.52 (m, 4H), 4.42 (t, J=5.14 Hz, 1H), 3.32-3.40 (m, 2H), 3.08 (br s, 2H), 2.37 (s, 3H), 2.07-2.21 (m, 1H), 1.76-1.96 (m, 4H), 1.57-1.70 (m, 1H), 1.49 (s, 3H), 1.32-1.45 (m, 2H), 0.87 (br t, J=7.21 Hz, 3H). ¹⁹F NMR (376 MHz, DMSO-D₆) δ ppm −112.44. LCMS (ESI+) m/z: [MH]J calcd for C₂₈H₃₀FN₂O₅⁺: 493.2, found: 493.4. SFC (retention time=1.165 min).

### Example 16

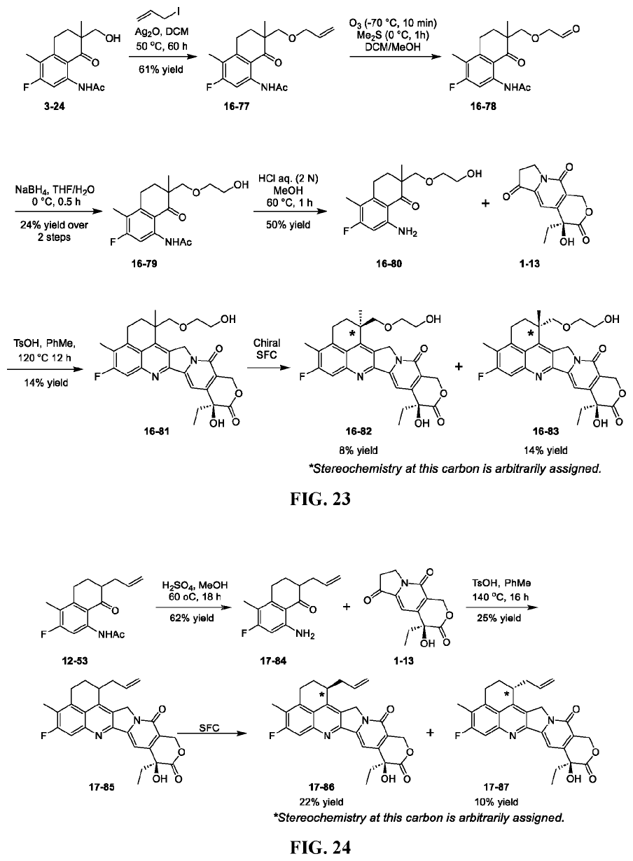

### (1R,9S)-9-Ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-82) and (1S,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-83) (FIG. 23)

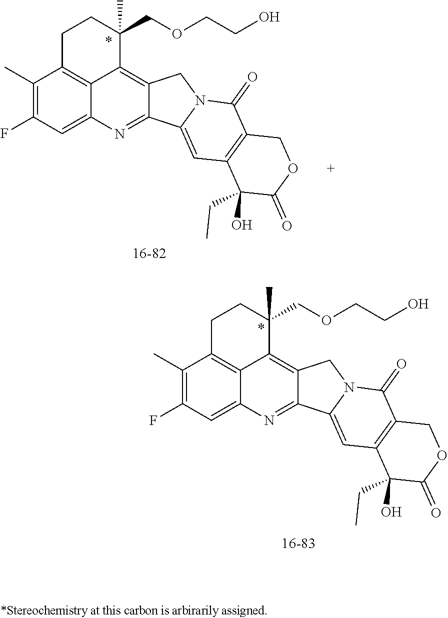 [SMILES: CC[C@@]1(O)C(=O)OCc2c1cc1n(c2=O)Cc2c-1nc1cc(F)c(C)c3c1c2[C@](C)(COCCO)CC3 ; CC[C@@]1(O)C(=O)OCc2c1cc1n(c2=O)Cc2c-1nc1cc(F)c(C)c3c1c2[C@@](C)(COCCO)CC3]

N-(7-((Allyloxy)methyl)-3-fluoro-4,7-dimethyl-8-oxo-5,6,7,8-tetrahydronaphthalen-1-yl) acetamide (16-77): To a solution of N-(3-fluoro-7-(hydroxymethyl)-4,7-dimethyl-8-oxo-5,6,7,8-tetrahydronaphthalen -1-yl) acetamide (3-24) (5.00 g, 17.9 mmol, 1.0 eq.) in CH₂Cl₂ (100 mL) were added Ag₂0 (41.4 g, 179 mmol, 10 eq.) and 3-iodoprop-1-ene (60.1 g, 358 mmol, 20 eq.). After stirring at 50° C. for 60 h under argon atmosphere, it was cooled down to room temperature, filtered through a pad of celite, the filter cake washed with CH₂Cl₂, and combined filtrates washed with brine, dried over anhydrous Na₂SO₄ and concentrated. The residue was subjected to silica gel flash column chromatography eluting with 20% ethyl acetate in petroleum ether to give N-(7-((allyloxy)methyl)-3-fluoro-4,7-dimethyl-8-oxo-5,6,7,8-tetrahydronaphthalen-1-yl)acetamide (16-77) (3.5 g, 61% yield). ¹H NMR (400 MHz, DMSO-D₆) δ ppm 12.09 (br d, J=3.4 Hz, 1H), 8.30 (d, J=13.2 Hz, 1H), 5.73-5.96 (m, 1H), 5.02-5.29 (m, 2H), 3.90-3.99 (m, 2H), 3.62-3.82 (m, 1H), 3.34 (s, 1H), 2.82-3.03 (m, 2H), 2.00-2.30 (m, 7H), 1.81-1.94 (m, 1H), 1.04-1.17 (m, 3H). LCMS (ESI+) m/z: [MH]⁺ calcd for C₁₈H₂₃FNO₃⁺: 320.1, found: 320.3.

N-(3-Fluoro-4,7-dimethyl-8-oxo-7-((2-oxoethoxy)methyl)-5,6,7,8-tetrahydronaphthalen-1-yl) acetamide (16-78): A solution of N-(7-((allyloxy)methyl)-3-fluoro-4,7-dimethyl-8-oxo-5,6,7,8-tetrahydronaphthalen-1-yl) acetamide (16-77) (3.50 g, 10.9 mmol, 1.0 eq.) in CH₂Cl₂ (68 mL) and MeOH (34 mL) was stirred at −78° C., followed by bubbling ozone into the mixture for 15 min to result in a light blue mixture, and addition of Me₂S (1.69 g, 27.3 mmol, 1.95 mL, 2.5 eq.) at −78° C. It was allowed to warm up to 25° C., stirred at 25° C. for 1 h, quenched with water (100 mL), extracted with dichloromethane (3×200 mL), combined organic layers washed with brine, dried over Na₂SO₄, filtered and concentrated to give N-(3-fluoro-4,7-dimethyl-8-oxo-7-((2-oxoethoxy)methyl)-5,6,7,8-tetrahydronaphthalen-1-yl)acetamide (16-78) (1.50 g, crude), which was used directly in the next step without further purification.

N-(3-Fluoro-7-((2-hydroxyethoxy)methyl)-4,7-dimethyl-8-oxo-5,6,7,8-tetrahydronaphthalen -1-yl) acetamide (16-79): To a solution of N-(3-fluoro-4,7-dimethyl-8-oxo-7-((2-oxoethoxy)methyl)-5,6,7,8-tetrahydro-naphthalen-1-yl)acetamide (16-78) (1.50 g, 3.73 mmol, 1.0 eq.) in THF (30 mL) and H₂O (15 mL) was added NaBH₄ (45.0 mg, 1.19 mmol, 0.32 eq.) portion wise at 0° C. After stirring at 0° C. for 30 min, it was quenched with H₂O (50 mL), extracted with ethyl acetate (3×100 mL), combined organic layers washed with brine, dried over anhydrous Na₂SO₄, filtered and concentrated. The residue was subjected to silica gel flash column chromatography eluting with 40% ethyl acetate in petroleum ether to give N-(3-fluoro-7-((2-hydroxyethoxy)methyl)-4,7-dimethyl-8-oxo-5,6,7,8-tetrahydro-naphthalen-1-yl)acetamide (16-79) (800 mg, 23% yield for two steps). ¹H NMR (400 MHz, DMSO-D₆) δ ppm 12.09 (br d, J=8.75 Hz, 1H), 8.30 (br d, J=13.13 Hz, 1H), 4.60 (br d, J=2.38 Hz, 1H), 3.66-3.80 (m, 1H), 3.33-3.52 (m, 4H), 3.14-3.24 (m, 1H), 2.79-3.03 (m, 2H), 2.06-2.25 (m, 7H), 1.86 (dt, J=8.04, 5.36 Hz, 1H), 1.11 (br d, J=9.63 Hz, 3H). LCMS (EST+) m/z: [MH]⁺ calcd for C₁₇H₂₃FNO₄⁺: 324.1, found: 324.3.

8-Amino-6-fluoro-2-((2-hydroxyethoxy)methyl)-2,5-dimethyl-3,4-dihydronaphthalen-1(2H) -one (16-80): To a solution of N-(3-fluoro-7-((2-hydroxyethoxy)methyl)-4,7-dimethyl-8-oxo-5,6,7,8-tetrahydronaphthalen-1-yl)acetamide (16-79) (800 mg, 2.47 mmol, 1.0 eq.) in MeOH (32 mL) was added 2 N hydrochloric acid solution (32 mL) at 20° C. After stirring at 60° C. for 1 h under argon protection, it was cooled down to 0° C., pH adjusted to 8 with saturated aqueous NaHCO₃, extracted with ethyl acetate (2×100 mL), combined organic layers washed with brine, dried over anhydrous Na₂SO₄, filtered and concentrated. The residue was subjected to silica gel flash column chromatography eluting with 35% ethyl acetate in petroleum ether to give 8-amino-6-fluoro-2-((2-hydroxyethoxy)methyl)-2,5-dimethyl-3,4-dihydronaphthalen-1(2H)-one (16-80) (350 mg, 50% yield). ¹H NMR (400 MHz, DMSO-D₆) δ ppm 7.45 (brs, 2H), 6.35 (d, J=12.59 Hz, 1H), 4.50 (t, J=5.38 Hz, 1H), 3.70 (d, J=9.05 Hz, 1H), 3.36-3.47 (m, 4H), 3.25 (d, J=9.05 Hz, 1H), 2.69-2.88 (m, 2H), 2.10-2.16 (dt, J=8.99, 4.55 Hz, 1H), 1.99 (s, 3H), 1.75 (dt, J=13.60, 5.55 Hz, 1H), 1.04 (s, 3H). LCMS (ESI+) m/z: [MH]⁺ calcd for C₁₅H₂₁FNO₃⁺: 282.1, found: 282.2.

(9S)-9-Ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-81): A mixture of 8-amino-6-fluoro-2-((2-hydroxyethoxy)methyl)-2,5-dimethyl-3,4-dihydro-naphthalen-1(2H)-one (16-80) (30.0 mg, 0.106 mmol, 1.0 eq.) and (S)-4-ethyl-4-hydroxy-7,8-dihydro-1H-pyrano[3,4-f]indolizine-3,6,10(4H)-trione (1-13) (83.8 mg, 0.319 mmol, 3.0 eq.) in PhMe (1.5 mL) was stirred at 120° C. until all solids dissolved, followed by addition of 4-methylbenzenesulfonic acid (11.0 mg, 0.064 mmol, 0.6 eq.) in one portion at 120° C. under argon protection. Ten more reactions were set up as described above and all eleven reaction mixtures were combined after stirring at 120° C. for 12 h. The combined mixtures were concentrated and the residue was subjected to silica gel flash column chromatography eluting with ethyl acetate to give (9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quino-line-10,13-dione (16-81) (85 mg, 14% yield). ¹H NMR (400 MHz, DMSO-D₆) δ ppm 7.76 (d, J=11.0 Hz, 1H), 7.31 (s, 1H), 6.51 (s, 1H), 5.28-5.46 (m, 4H), 3.35-3.44 (m, 6H), 3.08-3.19 (m, 1H), 2.19-2.29 (m, 5H), 2.18 (t, J=6.2 Hz, 2H), 1.22 (s, 3H), 0.79-0.90 (m, 3H). LCMS (ESI+) m/z: [MH]⁺ calcd for C₂₈H₃₀FN₂O₆⁺: 509.2, found: 509.2.

(1R,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-82) and (1S,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-83): (9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-81) (60.0 mg, 0.117 mmol) was dissolved in MeOH and separated by chiral SFC (Instrument: Waters SFC80 preparative SFC; Column: DAICEL CHIRALPAK AD(250 mm*30 mm, 10 um); Mobile phase: A for CO₂ and B for ethyl alcohol; Gradient: B %=50% isocratic elution mode; Flow rate: 80 g/min; Wavelength:220 nm; Column temperature: 40° C.; System back pressure: 100 bar), to afford (1R,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-82) (compound 16-82 may be the opposite enantiomer of that depicted) (5.0 mg, 8.3% yield), and (1S,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-83) (compound 16-83 may be the opposite enantiomer of that depicted) (9.0 mg, 15% yield). Note: the stereochemistry at the F-ring carbon is arbitrarily assigned.

16-82: ¹H NMR (400 MHz, CDCl₃) δ ppm 7.67 (br d, J=10.4 Hz, 1H), 7.61 (s, 1H), 5.75 (br d, J=16.3 Hz, 1H), 5.47 (br s, 2H), 5.30 (br d, J=15.8 Hz, 1H), 3.50-4.02 (m, 7H), 3.12 (br t, J=6.0 Hz, 2H), 2.41 (br s, 4H), 1.85-2.08 (m, 4H), 1.53 (s, 3H), 1.05 (br t, J=7.1 Hz, 3H). ¹⁹F NMR (376 MHz, CDCl₃) δ ppm −111.40. LCMS (ESI+) m/z: [MH]⁺ calcd for C₂₈H₃₀FN₂O₆⁺: 509.2, found: 509.5. SFC (RT=1.842 min).

16-83: ¹H NMR (400 MHz, CDCl₃) δ ppm 7.67 (br d, J=10.5 Hz, 1H), 7.61 (s, 1H), 5.75 (br d, J=16.4 Hz, 1H), 5.47 (br s, 2H), 5.32 (s, 1H), 3.47-4.06 (m, 7H), 3.12 (br t, J=5.9 Hz, 2H), 2.30-2.50 (m, 4H), 1.82-2.04 (m, 4H), 1.53 (s, 3H), 0.98-1.13 (m, 3H). ¹⁹F NMR (376 MHz, CDCl₃) δ ppm −111.40. LCMS (EST+) m/z: [MH]⁺ calcd for C₂₈H₃₀FN₂O₆⁺: 509.2, found: 509.5. SFC (RT=2.103 min).

### Example 17

### (1S,9S)-1-Allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-86) and (1R,9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-87) (FIG. 24)

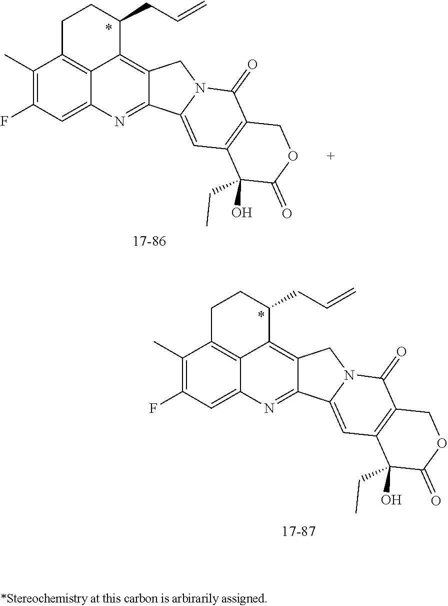 [SMILES: C=CC[C@@H]1CCc2c(C)c(F)cc3nc4c(c1c23)Cn1c-4cc2c(c1=O)COC(=O)[C@]2(O)CC ; C=CC[C@H]1CCc2c(C)c(F)cc3nc4c(c1c23)Cn1c-4cc2c(c1=O)COC(=O)[C@]2(O)CC]

2-Allyl-8-amino-6-fluoro-5-methyl-3,4-dihydronaphthalen-1(2H)-one (17-84): To a solution of N-(7-allyl-3-fluoro-4-methyl-8-oxo-5,6,7,8-tetrahydronaphthalen-1-yl)acetamide (12-53) (1.0 g, 3.63 mmol, 1.0 eq.) in MeOH (30 mL) was added sulfuric acid (2.0 mL), and stirred at 60° C. for 18 h under argon atmosphere. Five more reactions were set up as described above, and all six reaction mixtures were combined after cooling down to room temperature. They were quenched with water (150 mL), pH adjusted to 7 with saturated aqueous NaHCO₃ at 0° C., extracted with EtOAc (3×300 mL), combined organic layers washed with brine, dried over anhydrous Na₂SO₄, filtered and concentrated. The residue was subjected to silica gel flash column chromatography, eluting with 10% ethyl acetate in petroleum ether to give 2-allyl-8-amino-6-fluoro-5-methyl-3,4-dihydronaphthalen-1(2H)-one (17-84) (3.2 g, 63% yield). ¹H NMR (400 MHz, DMSO-D₆) δ ppm 7.42 (br s, 2H), 6.35 (d, J=12.7 Hz, 1H), 5.59-5.99 (m, 1H), 4.93-5.23 (m, 2H), 2.87 (dt, J=17.3, 4.5 Hz, 1H), 2.52-2.74 (m, 2H), 2.48 (br s, 1H), 1.93-2.21 (m, 5H), 1.54-1.70 (m, 1H). LCMS (ESI+) m/z: [MH]⁺ calcd for C₁₄H₁₇FNO⁺: 234.1, found: 234.4.

(9S)-1-Allyl-9-ethyl-5-fluoro-9-hydroxy-4-methyl-1,2,3,9,12,15-hexahydro-10H,13H benzo[de]pyrano[3′,4′:6,7]indolizino[1,2-b]quinoline-10,13-dione (17-85): To a suspension of 2-allyl-8-amino-6-fluoro-5-methyl-3,4-dihydronaphthalen-1(2H)-one (17-84) (200 mg, 0.857 mmol, 1.0 eq.) and (S)-4-ethyl-4-hydroxy-7,8-dihydro-1H-pyrano[3,4-f]indolizine-3,6,10(4H)-trione (10) (451 mg, 1.71 mmol, 2.0 eq.) in PhMe (10 mL) was added 4-methylbenzenesulfonic acid (59.1 mg, 0.342 mmol, 0.4 eq.) at 140° C., and stirred for 16 h with Dean-Stark trap to remove the water formed during reaction under. Two more reactions were set up as described above. The three reaction mixtures were combined, concentrated, and the residue subjected to silica gel flash chromatography, eluting with 50% ethyl acetate in petroleum ether to afford (9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-85) (300 mg, 25% yield). ¹H NMR (400 MHz, DMSO-D₆) δ ppm 7.74 (br d, J=10.9 Hz, 1H), 7.30 (s, 1H), 6.51 (s, 1H), 5.88-6.12 (m, 1H), 5.27-5.51 (m, 4H), 5.02-5.25 (m, 2H), 3.45 (br d, J=3.3 Hz, 1H), 3.08 (br s, 2H), 2.34-2.47 (m, 4H), 2.18-2.33 (m, 2H), 1.83-1.95 (m, 3H), 0.87 (br t, J=7.0 Hz, 3H). LCMS (ESI+) m/z: [MH]⁺ calcd for C₂₇H₂₆FN₂O₄⁺: 461.1, found: 461.4.

(1S,9S)-1-Allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-86) and (1R,9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-87): (9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-85) (150 mg, 0.326 mmol) was separated by SFC (Instrument: Waters SFC80 Preparative SFC System Column: REGIS(S,S)WHELK-O1(250 mm*25 mm, 10 um), Mobile phase: A for CO₂ and B for methanol, Gradient: B %=60.00% isocratic elution mode, Flow rate: 75.00 g/min, Monitor wavelength: 220&254 nm, Column temperature: 40° C., System back pressure: 100 bar) to afford (1S,9S)-1-allyl-9-ethyl-5-fluoro-9-hydroxy-4-methyl-1,2,3,9,12,15-hexahydro-10H,13H-benzo[de]pyrano [13′,4′:6,7]indolizino[1,2-b]quinoline-10,13-dione (17-86) (compound 17-86 may be the opposite enantiomer of that depicted) (25.2 mg, 17% yield) and (1R,9S)-1-allyl-9-ethyl-5-fluoro-9-hydroxy-4-methyl-1,2,3,9,12,15-hexahydro-10H,13H-benzo[de]pyrano[3′,4′:6,7]indolizino[1,2-b]quinoline-10,13-dione (17-87) (compound 17-87 may be the opposite enantiomer of that depicted) (11.9 mg, 8% yield). Note: the stereochemistry at the F-ring carbon is arbitrarily assigned.

17-86: ¹H NMR (400 MHz, DMSO-D₆) δ ppm 7.76 (d, J=11.1 Hz, 1H), 7.31 (s, 1H), 6.51 (s, 1H), 5.91-6.05 (m, 1H), 5.24-5.47 (m, 4H), 5.09-5.20 (m, 2H), 3.40-3.50 (m, 1H), 3.03-3.15 (m, 2H), 2.36-2.47 (m, 4H), 2.21-2.31 (m, 2H), 1.79-1.99 (m, 3H), 0.88 (t, J=7.3 Hz, 3H). ¹⁹F NMR (376 MHz, DMSO-D₆) δ ppm −111.77. LCMS (ESI+) m/z: [MH]⁺ calcd for C₂₇H₂₆FN₂O₄⁺: 461.1, found: 461.3. SFC (retention time=1.020 min).

17-87: ¹H NMR (400 MHz, DMSO-D₆) δ ppm 7.74 (d, J=11.0 Hz, 1H), 7.30 (s, 1H), 6.52 (s, 1H), 5.87-6.06 (m, 1H), 5.24-5.51 (m, 4H), 5.03-5.21 (m, 2H), 3.40-3.52 (m, 1H), 3.01-3.16 (m, 2H), 2.36-2.48 (m, 4H), 2.20-2.34 (m, 2H), 1.79-2.02 (m, 3H), 0.87 (t, J=7.3 Hz, 3H). ¹⁹F NMR (376 MHz, DMSO-D₆) δ ppm −111.78. LCMS (ESI+) m/z: [MH]⁺ calcd for C₂₇H₂₆FN₂O₄⁺: 461.1, found: 461.2. SFC (retention time=2.047 min).

### Example 18

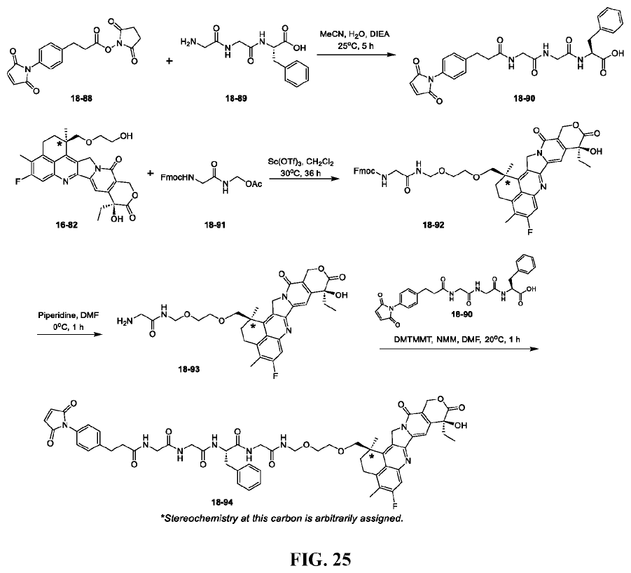

### (S)-2-(2-(2-(3-(4-(2,5-dioxo-2,5-dihydro-1H-pyrrol-1-yl)phenyl)propanamido)acetamido)acetamido)-N-(2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (18-94) (FIG. 25)

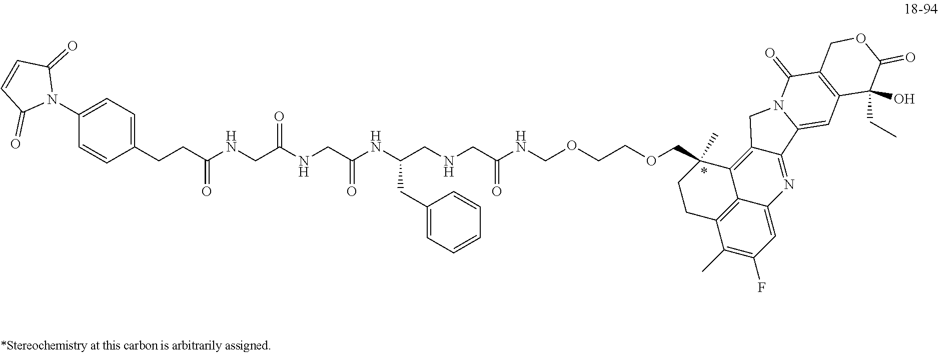 [SMILES: CC[C@@]1(O)C(=O)OCc2c1cc1n(c2=O)Cc2c-1nc1cc(F)c(C)c3c1c2[C@](C)(COCCOCNC(=O)CNC[C@H](Cc1ccccc1)NC(=O)CNC(=O)CNC(=O)CCc1ccc(N2C(=O)C=CC2=O)cc1)CC3]

### (3-(4-(2,5-dioxo-2,5-dihydro-1H-pyrrol-1-yl)phenyl)propanoyl)glycylglycyl-L-phenylalanine (18-90)

A mixture of (S)-2-(2-(2-aminoacetamido)acetamido)-3-phenylpropanoic acid (18-89) and N-ethyl-NN-diisopropylamine is added dropwise to a mixture of 2,5-dioxopyrrolidin-1-yl 3-(4-(2,5-dioxo-2,5-dihydro-1H-pyrrol-1-yl)phenyl)propanoate (18-88) in acetonitrile. The reaction mixture is stirred at 25° C. for 5 h. filtered and the filtrate is purified by prep-HPLC to give (3-(4-(2,5-dioxo-2,5-dihydro-1H-pyrrol-1-yl)phenyl)propanoyl)glycylglycyl-L-phenylalanine (18-90).

### (9H-fluoren-9-yl)methyl (2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)carbamate (18-92)

To a mixture of (1R,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-82)dichloromethane at ° C. is added Scandium(III) trifluoromethanesulfonate, and [[2-(9H-fluoren-9-ylmethoxycarbonylamino)acetyl]amino]methyl acetate (18-91) in three portions over 1.5 h. The reaction mixture is stirred at 30° C. for 36 h, quenched with water and extracted with dichloromethane. The combined organic phases are washed with brine, dried over Na₂SO₄, filtered, concentrated, and the residue purified by silica gel flash column chromatography eluting with 10%-100% of dichloromethane in ethyl acetate to give (9H-fluoren-9-yl)methyl (9H-fluoren-9-yl)methyl (2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano [3′,4′:6,7]indolizino[1,2-b]quinolin-1-yl)methoxy)ethoxy)methyl)amino)-2-oxoethyl)carbamate (18-92). 2-amino-N-((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)acetamide (18-93):

To a mixture of (9H-fluoren-9-yl)methyl (9H-fluoren-9-yl)methyl (2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)carbamate (18-92) in N,N-dimethylformamide was added piperidine. The reaction mixture is stirred at 0° C. for 1 h, filtered and the filtrate purified by prep-HPLC to give 2-amino-N-((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)acetamide (18-93).

### (S)-2-(2-(2-(3-(4-(2,5-dioxo-2,5-dihydro-1H-pyrrol-1-yl)phenyl)propanamido)acetamido)acetamido)-N-(2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (18-94)

A mixture of (2S)-2-[[2-[[2-[3-[4-(2,5-dioxopyrrol-1-yl)phenyl]propanoylamino]acetyl]amino]acetyl]amino]-3-phenyl-propanoic acid (18-90), 2-amino-N-((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)acetamide (18-93), 4-(4,6-dimethoxy-1,3,5-triazin-2-yl)-4-methylmorpholin-4-ium tetrafluoroborate and 4-methylmorpholine in N,N-dimethylformamide was stirred at 25° C. for 1 h, filtered and the filtrate purified by prep-HPLC to give (S)-2-(2-(2-(3-(4-(2,5-dioxo-2,5-dihydro-1H-pyrrol-1-yl)phenyl)propanamido)acetamido)acetamido)-N-(2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (18-94) (compound 18-94 may be the opposite enantiomer of that depicted). Note: the stereochemistry at the F-ring carbon is arbitrarily assigned.

### Example 19

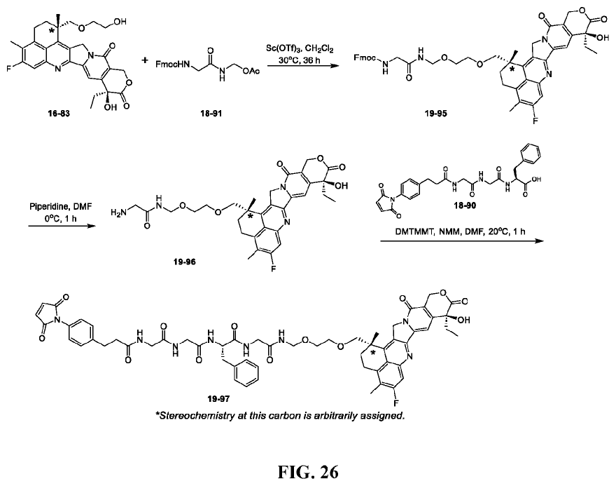

### (S)-2-(2-(2-(3-(4-(2,5-dioxo-2,5-dihydro-1H-pyrrol-1-yl)phenyl)propanamido)acetamido)acetamido)-N-(2-(((2-(((1S,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (19-97) (FIG. 26)

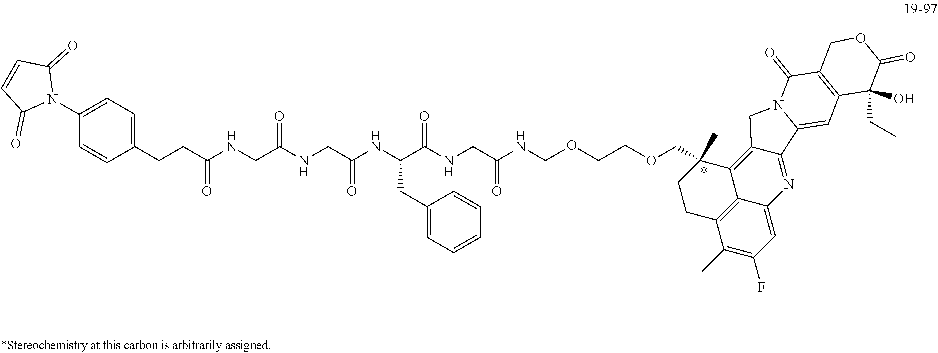 [SMILES: CC[C@@]1(O)C(=O)OCc2c1cc1n(c2=O)Cc2c-1nc1cc(F)c(C)c3c1c2[C@@](C)(COCCOCNC(=O)CNC(=O)[C@H](Cc1ccccc1)NC(=O)CNC(=O)CNC(=O)CCc1ccc(N2C(=O)C=CC2=O)cc1)CC3]

### (9H-fluoren-9-yl)methyl (2-(((2-(((1S,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)carbamate (19-95)

To a mixture of (1S,9S)-9-ethyl-5-fluoro-9-hydroxy-1-((2-hydroxyethoxy)methyl)-1,4-dimethyl-1,2,3,9,12,15-hexahydro-10H,13H-benzo[de]pyrano[3′,4′:6,7]indolizino[1,2-b]quinoline-10,13-dione (16-83)dichloromethane at ° C. is added Scandium(III) trifluoromethanesulfonate, and [[2-(9H-fluoren-9-ylmethoxycarbonylamino)acetyl]amino]methyl acetate (18-91) in three portions over 1.5 h. The reaction mixture is stirred at 30° C. for 36 h, quenched with water and extracted with dichloromethane. The combined organic phases are washed with brine, dried over Na₂SO₄, filtered, concentrated, and the residue purified by silica gel flash column chromatography eluting with 10%-100% of dichloromethane in ethyl acetate to give (9H-fluoren-9-yl)methyl (9H-fluoren-9-yl)methyl (2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)carbamate (19-95). 2-amino-N-((2-(((1S,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)acetamide (19-96):

To a mixture of (9H-fluoren-9-yl)methyl (9H-fluoren-9-yl)methyl (2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)carbamate (19-95) in N,N-dimethylformamide was added piperidine. The reaction mixture is stirred at 0° C. for 1 h, filtered and the filtrate purified by prep-HPLC to give 2-amino-N-((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)acetamide (19-96).

### (S)-2-(2-(2-(3-(4-(2,5-dioxo-2,5-dihydro-1H-pyrrol-1-yl)phenyl)propanamido)acetamido)acetamido)-N-(2-(((2-(((1R,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (19-97)

A mixture of (2S)-2-[[2-[[2-[3-[4-(2,5-dioxopyrrol-1-yl)phenyl]propanoylamino]acetyl]amino]acetyl]amino]-3-phenyl-propanoic acid (18-90) (see Example 18), 2-amino-N-((2-(((1S,9S)-9-ethyl-5-fluoro-9-hydroxy-1,4-dimethyl-10,13-dioxo-2,3,9,10,13,15-hexahydro-1H,12H-benzo[de]pyranol[3′,4′:6,7]indolizino[1,2-b]quinolin-1-yl)methoxy)ethoxy)methyl)acetamide (19-96), 4-(4,6-dimethoxy-1,3,5-triazin-2-yl)-4-methylmorpholin-4-ium tetrafluoroborate and 4-methylmorpholine in N,N-dimethylformamide was stirred at 25° C. for 1 h, filtered and the filtrate purified by prep-HPLC to give (S)-2-(2-(2-(3-(4-(2,5-dioxo-2,5-dihydro-1H-pyrrol-1-yl)phenyl)propanamido)acetamido)acetamido)-N-(2-(((2-(((1S,9S)-9-ethyl-5-fluoro-9-hydroxy-1,4-dimethyl-10,13-dioxo-2,3,9,10,13,15-hexahydro-1H,12H-benzo[de]pyrano[3′,4′:6,7]indolizino[1,2-b]quinolin-1-yl)methoxy)ethoxy)methyl)amino)-2-oxoethyl)-3-phenylpropanamide (19-97). (compound 19-97 may be the opposite enantiomer of that depicted). Note: the stereochemistry at the F-ring carbon is arbitrarily assigned.

### Example 20

### CTG Assays of Payloads (Jeko-1 and MDA-MB-468)

The CTG assay is a method of determining the number of viable cells in culture based on quantitation of the ATP present, an indicator of metabolically active cells. The cell assay requires the addition of a single reagent, Cell Titer Glo, in which cells are lysed and generation of a luminescent signal is produced. The luminescent signal is proportional to the amount of ATP present. The amount of ATP is directly proportional to the number of cells present in culture. For this assay, cells were ensured to be in log-phase for either Jeko-1 or MDA-MB-468. Cells were transferred to 96 wells and treated with compounds in three-fold serial dilution starting from 1 mM to 0.0000508 mM (10 points dilution), for 72 h. Cell viability was analyzed with CellTiter-Glo® Luminescent Cell Viability Assay (Promega) following manufactures' instruction. Percentage of viable cells in each compound concentration was determined by normalizing with the luminescence of vehicle control and plotted into percentage of viability versus dose response curve by nonlinear fit in GraphPad Prism software. Compound IC₅₀ was calculated as the concentration of compound killing 50% of cells. Representative assay results are summarized in Tables 1 and 2.

### Example 21

### Human Hepatocyte Clearance (HHEP CL)

Suspensions of human hepatocytes (from 10 mixed gender human donors, final concentration 0.5×10⁶ cell/mL) in Williams' E medium were incubated for 90 min with a test compound (0.90% acetonitrile and 0.10% DMSO, final concentration 1 mM) and positive controls (7-Ethoxycoumarin, 7-Hydroxycoumarin, 0.90% acetonitrile and 0.10% DMSO, final concentration 3 mM), with constant shaking at about 600 rpm at 37° C. in an incubator at 5% CO₂ and 95% humidity. The total volume of incubation was 200 μl. A sample (25 mL) was taken out at T0, 15, 30, 60 and 90 min, which was added intermediately to the ice-cold stop solution (acetonitrile with 200 ng/mL of to butamide and labetalol as internal standard) (125 μl), and vortexed at 500 rpm for 10 min, centrifuged at 3220×g for 20 min at 4° C. Analytical plates were sealed and stored at 4° C. until LCMS analysis. Viability of hepatocytes at pre-incubation was determined to be 84.5%. Representative assay results are summarized in Tables 1 and 2.

### Example 22

### Human Liver Microsome Clearance (HLM CL)

Working solution was prepared by adding 5 μL of compound and control stock solution (10 mM in dimethyl sulfoxide, DMSO) to 495 μL of acetonitrile (ACN) (intermediate solution concentration: 100 μM, 99% CAN and 1% DMSO. The appropriate concentrations of microsome working solutions were prepared in 100 mM potassium phosphate buffer. After reaction plates containing mixtures of compound and microsomes were pre-incubated at 37° C. for 10 min, 98 mL of 2 mM of NADPH and 2 mM of MgCl₂ solution was added to start the reaction. The final concentrations of incubation medium were as follows: microsome—0.5 mg protein/mL, test compound/control compound—1 mM, NADPH—1 mM, MgCl₂—1 mM, acetonitrile 0.99%, DMSO 0.01%. Incubations were performed at 37° C. for 60 min. Samples were taken out at T0, T5, T15, T30, T45 and T60, which was added intermediately to the ice-cold stop solution (acetonitrile with 200 ng/mL of to butamide and labetalol as internal standard) (125 μl), shaken for 10 min, centrifuged at 4000 rpm for 20 min at 4° C. Analytical plates were analyzed by LCMS. Representative assay results are summarized in Tables 1 and 2.

Human liver microsome clearance assay assess metabolism by the cytochrome P450 system (phase I enzymes). These enzymes oxidize substrates by incorporating oxygen atoms into hydrocarbons, thus causing the introduction of hydroxyl groups, or N- O- and S-dealkylation of substrates and forming more polar products easier to be cleared. Human hepatocyte clearance assay measures more broadly the overall cellular metabolism of the test compound (phase I and phase II enzyme pathways). Phase II enzymes catalyze the conjugation reaction of xenobiotic metabolites and charged species, such as glutathione, sulfate, glycine, or glucuronic acid to form even more polar compounds for easier clearance.

The payloads with higher intrinsic clearance may provide better therapeutic index due to their potential lower systemic plasma exposure. (Maderna, A.; Doroski, M; Subramanyam, C.; Porte, A.; Leverett, C. A.; Vetelino, B. C.; Chen, Z.; Risley, H.; Parris, K.; Pandit, J.; Varghese, A. H.; Shanker, S.; Song, C.; Sukuru, S. C. K.; Farley, K. A.; Wagenaar, M. M.; Shapiro, M. J.; Musto, S.; Lam, M-H.; Loganzo, F.; O'Donnell, C. J. “Discovery of cytotoxic dolastatin 10 analogues with N-terminal modifications” Journal Medicinal Chemistry, 2014, 57, 10527-10543). In Table 1, the payloads with higher intrinsic clearance likely have an improved safety profile because the payload, which is potentially toxic to healthy cells, is quickly removed from the plasma, decreasing its chance of interacting with healthy cells.

### Example 23

### PAMPA (Parallel Artificial Membrane Permeability Assay)

PAMPA is a method which determines the permeability of substances from a donor compartment, through a lipid-infused artificial membrane into an acceptor compartment. See Ottaviani, G.; Martel, S.; Carrupt, P-A. “Parallel Artificial Membrane Permeability Assay: A New Membrane for the Fast Prediction of Passive Human Skin Permeability”, Journal of Medicinal Chemistry, 2006, 49 (13), 3948-3954). A multi-well microtitre plate is used for the donor and a membrane/acceptor compartment is placed on top; the whole assembly is commonly referred to as a “sandwich”. At the beginning of the test, the drug is added to the donor compartment, and the acceptor compartment is drug-free. After an incubation period which may include stirring, the sandwich is separated, and the amount of drug is measured in each compartment. Mass balance allows calculation of drug that remains in the membrane.

The PAMPA was performed by Pion Inc using the GIT-0 lipid and 5 mM donor solution in pH 5.0 and pH 7.4 PRISMA buffer (containing 0.05% DMSO). The higher PAMPA data has been associated with better bystander killing. (Ogitani Y.; Hagihara K.; Oitate, M.; Naito, H.; Agatsuma T. “Bystander killing effect of DS-8201a, a novel anti-human epidermal growth factor receptor 2 antibody-drug conjugate, in tumors with human epidermal growth factor receptor 2 heterogeneity” Cancer Science, 2016, 107 (7), 1039-1046).

Representative assay results are summarized in Tables 1 and 2. Higher permeability is important because it implies greater potential for “bystander killing”. That is, once the payload has neutralized a tumor cell, a more permeable payload is more likely to escape the neutralized tumor cell, then imbed in a neighboring tumor cell. Once there, it can neutralize the tumor cell, escape, embed in another neighboring tumor cell, and repeat the process.

TABLE 1
Representative PAMPA assay, HHEP CL, HLM CL, CTG assay
with Jeko-1 and MDA-MB-468 cells results of Compounds
1-16, 1-17, 2-20, 3-27, 3-28, 5-40, 5-41, 6-44, and 7-45.

| | PAMPA Pe | | | | | | MDA-MB- |
|---|---|---|---|---|---|---|---|
| | (10⁻⁶ cm/s) | HHEP Cl | | HLM Cl | | JeKo-1 | 468 |
| Compound | pH 5.0/7.4 | (T_{1/2}) | | (T_{1/2}) | | IC₅₀ (nM) | IC₅₀ (nM) |
| Dxd | 24/4.0 | 17.8 | (217) | 8.6 | (>145) | 1.42 | 11.6 |
| Exatecan | 37.2/5.7 | 59.3 | (65.0) | 11.7 | (106) | 0.59 | 6.2 |
| 1-16 | 40.4/8.5 | 33.0 | (117) | 44.0 | (28) | 0.79 | 7.2 |
| 1-17 | 74.7/7.8 | 17.9 | (215) | 53.2 | (23.5) | 1.0 | 7.7 |
| 2-20 | —/— | 24.0 | (161) | 46.0 | (27) | 40.8 | 351 |
| 3-27 | 62.2/8.5 | 62.4 | (62) | 128 | (9.7) | 2.1 | 18.6 |
| 3-28 | 64.0/8.4 | 62.9 | (61) | 53.8 | (23) | 1.3 | 9.9 |
| 5-40 | 52.2/6.0 | 102 | (38) | 238 | (5.2) | 10.0 | 69.1 |
| 5-41 | 53.5/6.6 | 49.8 | (77) | 92.5 | (13.5) | 11.6 | 92.2 |
| 6-44 | 54.9/8.3 | 48.3 | (80) | 51.7 | (24) | 24.1 | 198 |
| 7-45 | 56.6/7.5 | 92.2 | (42) | 137 | (9) | 33.5 | 258 |

TABLE 2
Representative PAMPA assay, HHEP CL, HLM CL, CTG
assay with Jeko-1 cells results of Compounds 12-
58, 12-59, 13-63, 13-64, 14-70, 14-71, 15-75, 15-
76, 16-82, 16-83, 17-86, and 17-87 with JeKo-1

| | PAMPA Pe | | | |
|---|---|---|---|---|
| | (10⁻⁶ cm/s) | HHEP Cl | HLM Cl | JeKo-1 |
| Compound | pH 5.0/7.4 | (T_{1/2}) | (T_{1/2}) | IC₅₀ (nM) |
| Dxd | 10.6/4.7 | >217 | 52.2 | 0.52 |
| 12-58 | 2.9/3.8 | 68.1 | 20.1 | 0.24 |
| 12-59 | 46.9/33.4 | 64.3 | 20.1 | 0.32 |
| 13-63 | 34.7/32.9 | 141.4 | 27.1 | 0.44 |
| 13-64 | 14.6/10.6 | 79.4 | 22.0 | 0.28 |
| 14-70 | 44.2/28.5 | 53.6 | 18.4 | 0.79 |
| 14-71 | 25.0/17.3 | 47.0 | 10.8 | 0.18 |
| 15-75 | 16.5/11.5 | 76.5 | 22.4 | 0.88 |
| 15-76 | 45.5/33.2 | 40.8 | 5.2 | 0.27 |
| 16-82 | 54.8/25.5 | 45.0 | 5.6 | 0.64 |
| 16-83 | 47.7/25.1 | 113.4 | 25.7 | 1.1 |
| 17-86 | NA | 46 | 13 | 0.93 |
| 17-87 | NA | 31 | 5.4 | 0.58 |

Dxd: deruxtecan; HHEP Cl: human hepatocytes intrinsic clearance, mL/min/Kg; HHEP t_{1/2}, min. HLM: human liver microsome clearance, mL/min/Kg. —/—: low permeability. NA: not available.

### Example 24

### Development of Anti-ROR-1-Specific Monoclonal Antibodies

Novel and diversified anti-ROR-1-specific monoclonal antibodies were developed to bind to multiple regions of the ROR-1 extracellular domain (ECD) by employing an antibody development campaign using three strategies: (1) mice of cohort 1 were immunized using full length ROR-1 ECD; (2) mice of cohorts 2 and 3 were immunized with the ROR-1 IgG-like domain; and (3) mice of cohort 4 were immunized with a short region of the human IgG-like sequence of ROR-1. After immunization of the mice, monoclonal antibodies were generated using conventional approaches. Briefly, unique variable heavy and light chain pairs from hybridoma and phage display campaigns were cloned into vectors designed to express full length antibodies as IgGs in HEK293 cells under the control of a CMV promoter. Antibody expression vectors were complexed with polyethylenimine and transfected into HEK293 cultures. After 5 days of shaking at 37° C. in 293 cell culture media, antibodies were captured on agarose-based protein A resin. After several stringent washes, antibodies were eluted in glycine solution, pH 3, neutralized with Hepes, pH 9, and buffer exchanged into PBS.

Several monoclonal antibodies were developed using these approaches and the generated antibodies were subjected to additional screening to assess specific characteristics of the antibodies. To fully evaluate the characteristics of the novel antibodies several assays were performed. First, confirmation of antibody binding to the ROR-1 epitope was confirmed both biochemically, as well as, in ROR-1 positive cell lines. The specificity of binding was assessed biochemically by screening binding to human ROR-2 protein, rodent ROR-1 protein, as well as, in cell-based assays. Further screening parameters included analyses of antibody internalization, epitope binning against known anti-ROR-1 antibodies (UC961 and 4a5), binding to human ROR-1 Ig-like domain, thermal shift, and assessment for self-interaction with Affinity-Capture Self-Interaction Nanoparticle Spectroscopy (AC-SINS).

### Example 25

### Assay Evaluating Saturation Concentrations and Human ROR-1 Binding Affinities of Anti-ROR-1 Specific Monoclonal Antibodies

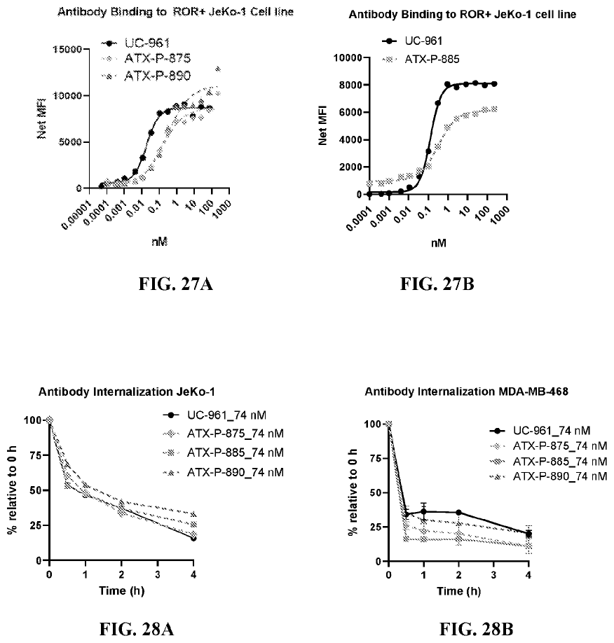

A cell binding saturation assay was developed to evaluate how well the anti-ROR-1 antibodies developed in Example 22 bound to endogenously expressed extracellular ROR-1 protein on cell lines. More specifically, the anti-ROR-1 monoclonal antibodies developed in Example 22, e.g., ATX-P-875, ATX-P-885, and ATX-P-890, were analyzed in a cellular binding assay. Briefly, two ROR-1 positive cell lines, JeKo-1 and MDA-MB-468, were incubated in a titration series concentration of each antibody construct. Cells were then washed and subjected to secondary antibody staining and detection by flow cytometry. Mean fluorescence (MFI) was determined by analysis on cytometer software. The binding of ATX-P-875, ATX-P-885, and ATX-P-890 was compared to cell binding saturation data for the monoclonal anti-ROR-1 antibody UC-961. (See FIGS. 27A-27B). As shown in FIGS. 27A-27B, the cell binding saturation for antibodies ATX-P-875, ATX-P-885, and ATX-P-890 were comparable to the cell binding saturation for UC-961 though a greater concentration of ATX-P-875 was needed to achieve saturation, as compared to UC-961. ATX-P-890 and ATX-P-885 were as good or improved, respectively, compared to UC-961 in concentrations needed to achieve binding saturation. Comparable saturation to UC-961 demonstrates that the anti-ROR-1 antibodies ATX-P-875, ATX-P-885, and ATX-P-890 have a similar affinity to the human ROR-1 target as a clinically approved antibody UC-961.

### Example 26

### Assay Evaluating Capacity of Anti-ROR-1 Specific Monoclonal Antibodies

### To Internalize ROR-1 on Human ROR-1-Positive Tumor Cells

After a saturating concentration (74 nM) was determined in the binding assay, the anti-ROR-1 antibodies developed herein (ATX-P-875, P-885, P-890) were evaluated for their capacity to internalize the ROR-1 receptor on human ROR-1 positive cells (JeKo-1 and MDA-MB-468). Briefly, the ROR-1-positive cell lines were incubated with antibody at super saturating conditions so as to bind all available ROR-1 receptors. Excess antibody was washed off and the cells were incubated at 37° C. for a designated amount of time over a four-hour time course. At the end of each time point, internalization was stopped by placing an aliquot of cells on ice. The antibody remaining on the surface was detected using a labeled secondary antibody and flow cytometry. Percent internalization was calculated based on time zero, and time zero was assumed that 100% of available receptors are on the cell surface. The results in FIGS. 28A-28B demonstrate that all antibodies internalize ROR-1 receptor on JeKo-1 and MDA-MB-468 cells by a reduction of at least 75% over 4 hours. Unexpectedly, in MDA-MB-468, internalization of two of the anti-ROR-1 antibodies (ATX-P-875 and ATX-P-890) was improved over the clinically used UC-961 anti-ROR-1 antibody providing evidence that the ATX-P-875 and ATX-P-890 antibodies have an improved ability to internalize the ROR-1 receptor from the surface of solid tumors.

### Example 27

### Epitope Binding Studies of Anti-ROR-1 Specific Monoclonal Antibodies

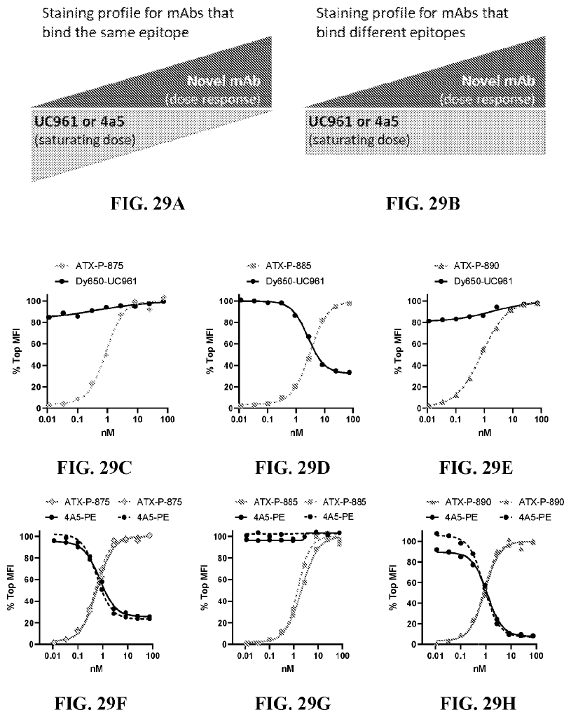

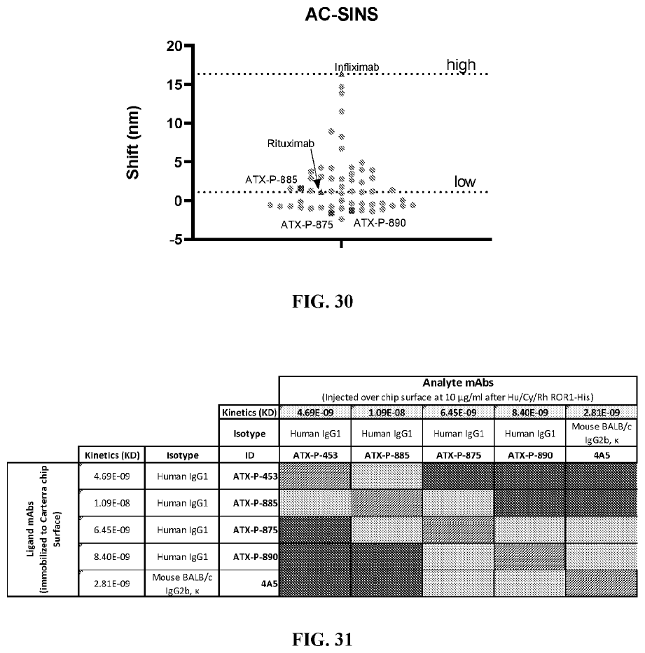

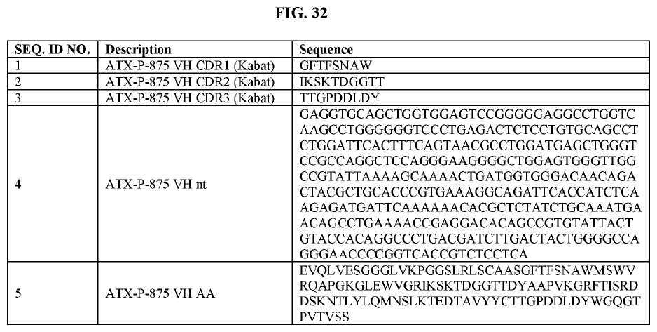

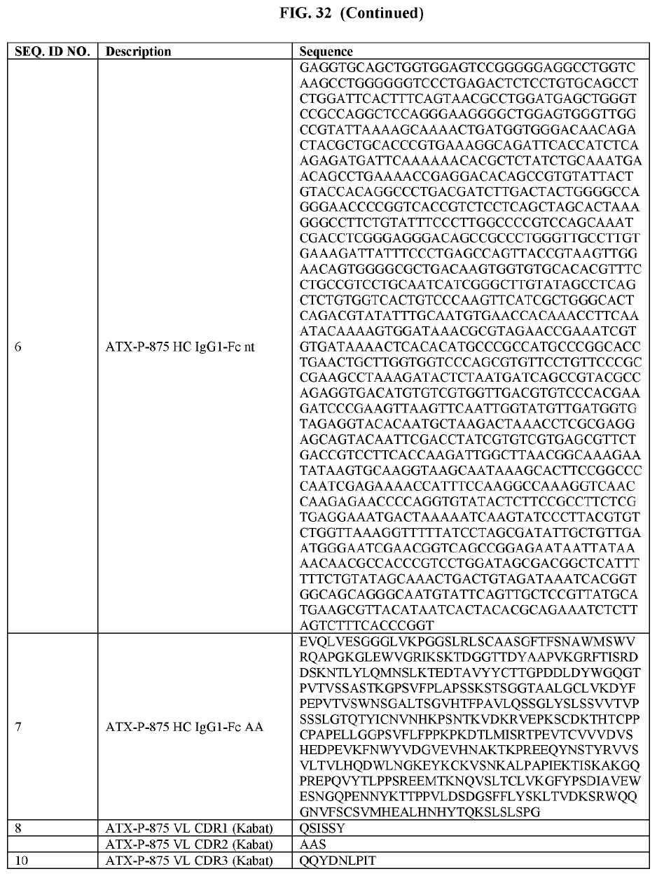

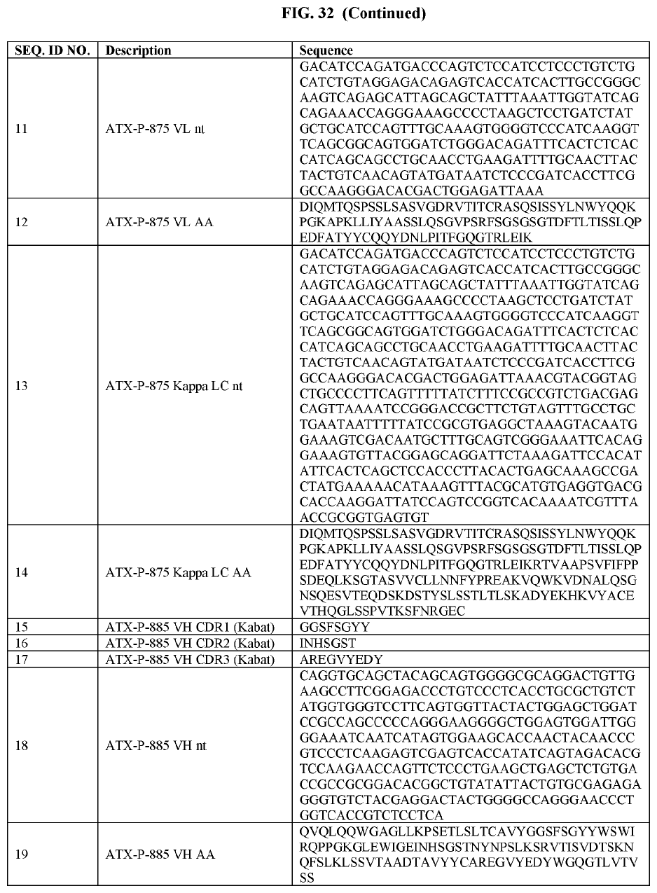

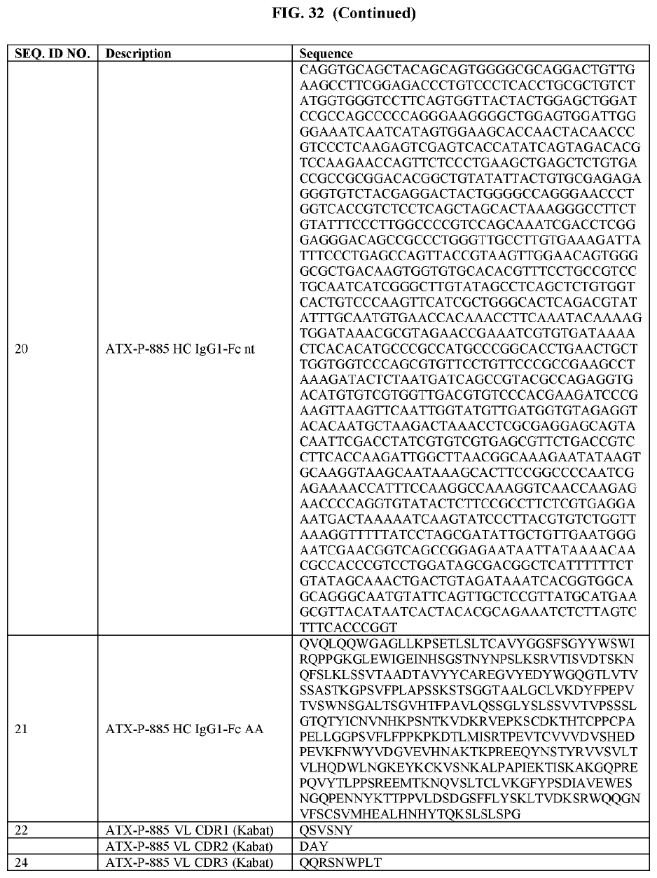

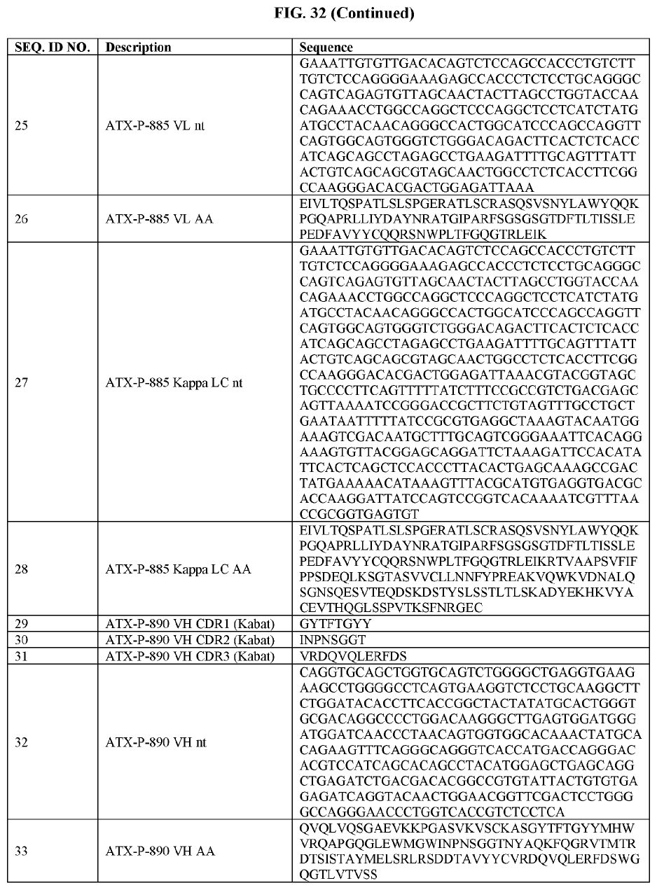

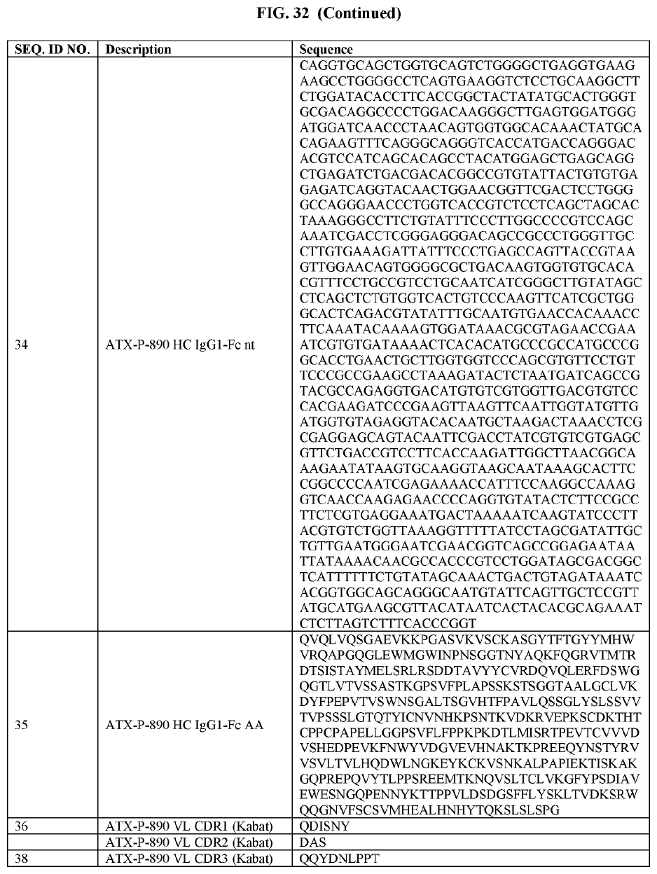

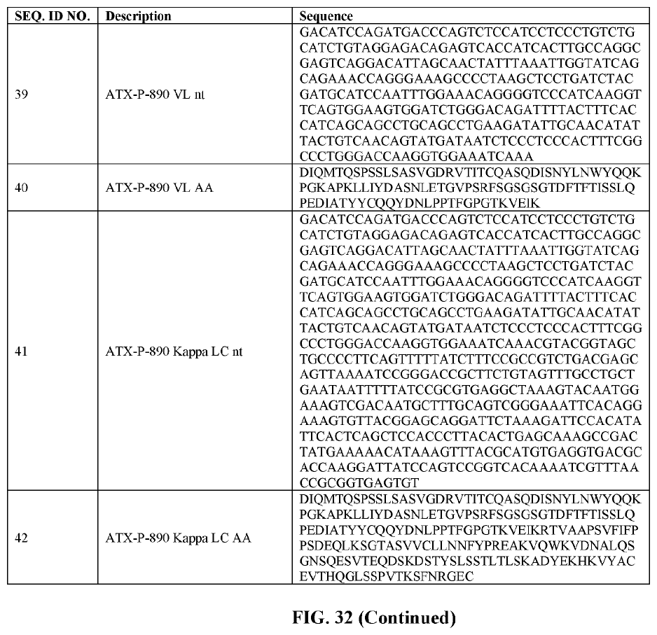

Cellular binning was also employed to determine if monoclonal antibodies ATX-P-875, ATX-P-885, and ATX-P-890 bound to the same epitopes as conventionally known anti-ROR-1 binding monoclonal antibodies UC-961 and 4A5 (controls). In Step 1 of the cellular binning experiments, ATX-P-875, ATX-P-885, and ATX-P-890 monoclonal antibodies were separately incubated with ROR-1 expressing cells (MDA-MB-468) at various amounts. In Step 2, a fluorescently labeled secondary antibody recognizing the novel antibodies was incubated with the samples. And finally in Step 3, the ROR-1 expressing cells coated with ATX-P-875, ATX-P-885, and ATX-P-890 were incubated with a saturating dose of labeled UC-961 (Dy650-UC 961) or 4A5 antibody (PE 4A5) and analyzed by flow cytometry. The UC-961 and 4A5 staining signal was then compared to the novel antibody staining signal to determine if the ATX-P-875, ATX-P-885, and ATX-P-890 antibodies bound the same epitope as the known ROR-1 binding antibodies UC961 and 4A5. FIG. 29A shows the staining profile expected if the ATX-P-875, ATX-P-885, and ATX-P-890 antibodies bound the same epitope as the UC-961 and 4A5 antibodies. FIG. 29B, shows the expected profile if the ATX-P-875, ATX-P-885, and ATX-P-890 antibodies bound to a separate epitope on ROR-1 than the UC-961 or 4A5 antibodies. Briefly, if binding the same epitope, increased novel antibody concentration would block the binding of prelabeled competitor antibody, thereby reducing the signal of the competitor at higher concentrations. In the case antibodies bound separate epitopes, each antibody, novel and competitor, would have increased staining with increased dose as there would be no competition for binding to the receptor. The cellular binning data obtained in MDA-MB-468 cells indicated that ATX-P-885 appreciably bound the same epitope as UC-961 and both ATX-P-875 and ATX-P-890 appreciably bound the same epitope as 4A5. (See FIGS. 29C-29H and FIG. 30). The ability of the antibodies developed herein to bind distinct ROR-1 epitopes provides the opportunity to regulate the target in a variety of ways.

### Example 28

### Biochemical Binning Studies of Anti-ROR-1 Specific Monoclonal Antibodies

Biochemical binning by SPR was also evaluated for the anti-ROR1 antibodies (ATX-P-875, P-885, P-890) as compared against control anti-ROR-1 antibodies UC-961 and 4A5. In these experiments 10 ug/ml of purified clonal protein of Hu/Cy/Rh ROR1-His was covalently coupled to the HC30M chip. Individual dilutions of each antibody at 10 μg/mL were injected over the chip and binding was evaluated by Carterra SPR. Unexpectedly the data demonstrate that there are 3 distinct binding epitopes between ATX-P-875, ATX-P-885, and ATX-P-890 with ATX-885 being the only antibody to impart partial blocking to the UC-961 antibody (See FIG. 31). Cellular binning only evaluated the ability of the anti-ROR1 antibodies (ATX-P-875, P-885, P-890) to block either UC-961 or 4A5, two clinically used ROR-1 antibodies. Biochemical SPR evaluation also tested the antibodies' ability to block each other and found that ATX-P-875 was able to block the binding of 4A5 as well as ATX-P-885, while still not being able to block UC-961.

### Example 29

### Antibody Characterization

Antibody characterization of ATX-P-875, ATX-P-885, and ATX-P-890, as compared to UC-961, are summarized in FIG. 31 and Tables 3-6. An initial assessment of antibody developability was performed by AC-SINS to evaluate the potential for self-interaction (FIG. 28). Control antibody Rituximab show expected low shift while control antibody Infliximab shows an expected high shift. The anti-ROR-1 antibodies developed herein, ATX-P-875, ATX-P-885, and ATX-P-890, are in line with control antibodies that do not show significant self-interaction and therefore are not likely to pose a significant developability risk. Additionally, the binding characteristics of monoclonal antibodies ATX-P-875, ATX-P-885, and ATX-P-890 were compared to the binding characteristics for UC961 in additional experiments. Tables 3 and 4 provide this antibody characterization data in comparison to the known ROR-1 binding antibody UC-961 including tabled results for biochemical binding to purified proteins and measured by SPR (Table 3-4), cellular binding to ROR-1 positive cell lines JeKo-1 and MDA-MB-468 (EC50) (Table 5), and cellular internalization (% internalized) (Table 6). Of particular note, it is believed that the reduced affinity of ATX-P-885 (KD: 1.09E-08) compared to UC-961 and other anti-ROR-1 antibodies (ATX-P-875 and ATX-P-890) can provide an unexpected therapeutic benefit. It is contemplated that by binding less tightly to the ROR-1 epitope, the ATX-P-885 antibody can penetrate further into the tumor to reach more distant cells expressing ROR-1 target.

TABLE 3
Human/Cyno/Rhesus ROR-1 binding
Hu/Cy/Rh ROR1 - His

| | kₐ | k_{d} | K_{D} | Rmax | Res | % |
|---|---|---|---|---|---|---|
| Name | (M−1 s−1) | (s−1) | (M) | (RU) | SD | Rmax |
| ATX-P- | 1.26E+06 | 5.91E−03 | 4.69E−09 | 529.0 | 25.6 | 4.85% |
| 453 | | | | | | |
| (UC961) | | | | | | |
| ATX-P- | 4.93E+05 | 3.16E−03 | 6.45E−09 | 593.6 | 22.3 | 3.76% |
| 875 | | | | | | |
| ATX-P- | 2.70E+05 | 2.95E−03 | 1.09E−08 | 341.6 | 10.2 | 2.99% |
| 885 | | | | | | |
| ATX-P- | 3.92E+05 | 3.14E−03 | 8.40E−09 | 504.2 | 15.0 | 2.97% |
| 890 | | | | | | |

TABLE 4
Mouse ROR-1 binding
Mouse ROR1 - His

| | kₐ | k_{d} | K_{D} | Rmax | Res | % |
|---|---|---|---|---|---|---|
| Name | (M−1 s−1) | (s−1) | (M) | (RU) | SD | Rmax |
| ATX-P- | N/A | N/A | N/A | N/A | N/A | N/A |
| 453 | | | | | | |
| (UC961) | | | | | | |
| ATX-P- | N/A | N/A | N/A | N/A | N/A | N/A |
| 875 | | | | | | |
| ATX-P- | 2.92E+04 | 1.32E−03 | 4.53E−08 | 73.5 | 8.9 | 12.14% |
| 885 | | | | | | |
| ATX-P- | N/A | N/A | N/A | N/A | N/A | N/A |
| 890 | | | | | | |

TABLE 5
JeKo-1 and MDA-MB-468 cellular binding summary
Cellular Binding Summary

| Name | JeKo-1 (Avg EC50) | MDA-MB-468 (Avg EC50) |
|---|---|---|
| ATX-P-453 (UC961) | 0.073 | 0.176 |
| ATX-P-875 | 0.145 | 0.708 |
| ATX-P-885 | 1.075 | 2.508 |
| ATX-P-890 | 0.174 | 1.016 |

TABLE 6
JeKo-1 and MDA-MB-468 cellular internalization summary
Cellular Internalization Summary

| Name | JeKo-1 (%) | MDA-MB-468(%) |
|---|---|---|
| ATX-P-453 (UC961) | 75 | 80 |
| ATX-P-875 | 75 | 89 |
| ATX-P-885 | 75 | 89 |
| ATX-P-890 | 70 | 80 |

### Example 30

### Preparation of Antibody-Drug Conjugates

The synthesis of the immunoconjugates is accomplished as set forth in this example. The antibodies are produced as described in Example 24 and are suspended in PBS pH 7.2 with protein concentrations ranging between 10-20 mg/mi. For the reduction and conjugation calculations, a molecular weight of 1500001 Da for all antibodies was used.

Each antibody was prepared for reduction by the addition of 5% v/v of 500 mM Tris, 25 mM EDTA, pH 8.5., followed by the addition of TCEP (6 equivalent, 10 mM stock of TCEP in water) and the mixture is maintained at 20° C. for 2 h. This reduction step forms the cysteine residues Cys-SH on the antibodies to facilitate bioconjugation with the toxin-linkers, i.e., compounds of Formula (III) as described herein.

After DMA is added and gently mixed with the above reduced protein solution to achieve a final 10% v/v during conjugation, a toxin-linker stock solution (12 equivalent, 50 mM in DMA) is added and gently mixed. The bioconjugation is allowed to proceed for approximately 16-20 h overnight at 20° C.; it is complete within 2 h with the extended time allowed for maleimide ring opening.

The crude conjugate is buffer exchanged to PBS pH 7.4 using a gravity fed NAP 25 (small scale) or a flow HiPrep G25 (large scale) with the columns prepared and operated according to manufacturer's (Cytivia) instructions. To remove residual toxin, a 100 mg/ml slurry of activated carbon (Sigma/C9157) in PBS pH 7.4 is prepared and added to achieve 1 mg carbon to 1 mg starting antibody mass. It is mixed gently for 2 h, sufficiently to maintain the carbon in suspension. Then, the carbon is removed by centrifugation at 4000 g. Polysorbate 20 (PS20) is added from a 10% w/v stock solution in PBS pH7.4 to achieve a final 0.02% PS20 w/v in the product. The antibody-drug conjugate (ADC) product is terminally filtered through a suitably sized 0.2 μm PES filter (chromatography direct/FIL-S-PES-022-13-100-S) under grade A laminar flow. The final product is analyzed as follows: monomer and [ADC] mg/ml by SEC HPLC, average DAR by PLRP, residual toxin by RP-HPLC, and endotoxin by Endosafe kinetic chromogenic.

Analytical processes are carried out on HPLC instruments Agilent 1100 or 1260.

### Example 31

### CTG Assays of Antibody-Drug Conjugates

Novel ROR-1 antibody-drug conjugates (ADCs) are evaluated by CTG assays in a similar manner as was described in Example 20 and Tables 1 and 2 for screening of payloads. By no means of limiting the scope of the antibody-drug conjugates of the present disclosure and by way of example only, a total of 3 unique antibodies ATX-P-875, ATX-P-885, and ATX-P-890 are conjugated to novel linker/payloads or compounds of Formula (III), including but not limited to compounds 18-94, 19-97 as well as any of the exemplary compounds of Formula (III) described in paragraphs [0121] through [0123], each of which begins with “In various embodiments, the conjugate of Formula (III) can be represented by a structure selected . . . ”. Briefly, ROR-positive (JeKo-1/MDA-MB-468) or ROR-negative (Ramos) cells are transferred to 96 wells and treated with the test ADCs in three-fold serial dilution starting from 1 mM to 0.0000508 mM (10 points dilution), for 72 h. Cell viability is analyzed with CellTiter-Glo® Luminescent Cell Viability Assay (Promega) following the manufacturer's instructions. The percentage of viable cells at each ADC concentration is determined by normalizing with the luminescence of vehicle control and plotted into percentage of viability versus dose response curve by nonlinear fit in GraphPad Prism software. The IC₅₀ for each test ADC is calculated as the concentration of compound killing 50% of cells and is benchmarked against UC-961. The ADCs described herein, including those prepared by conjugating antibodies ATX-P-875, ATX-P-885, and ATX-P-890 to compounds 18-94, 19-97 and any of the exemplary compounds of Formula (III), have an IC₅₀ value below 500 nM (for example, below 300 nM, below 100 nM, below 50 nM or below 30 nM) based on CTG assays with Jeko-1 or MDA-MB-468 cells.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

1. An immunoconjugate having Formula (I), 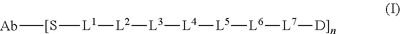
wherein:
Ab is an antibody or an antigen-binding fragment thereof;
L¹ is 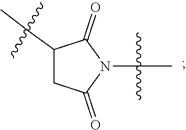 [SMILES: *C1CC(=O)N(*)C1=O]
L² is absent, 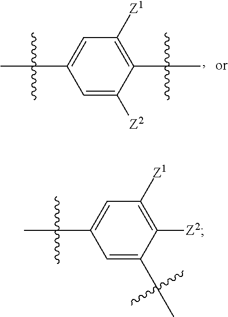
Z¹ and Z² are each individually hydrogen, halogen, NO₂, —O—(C₁-C₆ alkyl), or C₁-C₆alkyl;
L³ is —(CH₂)n¹-C(═O)— or —(CH₂CH₂O)n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or 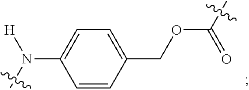
L⁷ is absent, 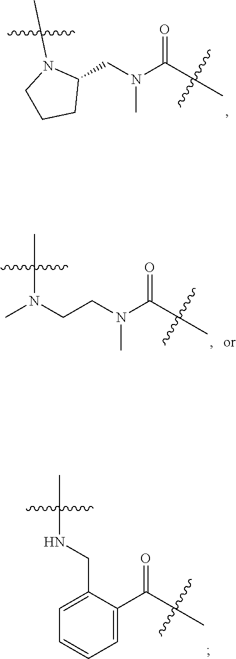
D is a drug moiety; and
n is an integer from 1 to 10;
wherein D is a drug moiety of Formula (II) having the structure: 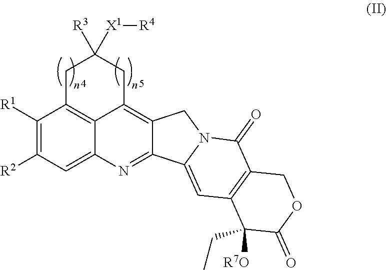
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² taken together form a ring;
R³ is selected from the group consisting of hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and [(CY₂)ₚO(CY₂)_{q}]ₜCY₃;
R⁴ is selected from the group consisting of substituted or unsubstituted —(C₁-C₆ alkyl)-X², substituted or unsubstituted —(C₁-C₆ haloalkyl)-X², substituted or unsubstituted —(C₁-C₆ alkenyl)-X², substituted or unsubstituted —(C₁-C₆ haloalkenyl)-X², substituted or unsubstituted —(C₁-C₆ alkynyl)-X², and substituted or unsubstituted —(C₁-C₆ haloalkynyl)-X²
X² is —OR⁹, —SR⁹, or —NHR⁹;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each t is individually 0, 1, 2, 3, 4, 5, or 6;
each t is individually 1, 2, 3, 4, 5, or 6;
R⁷ is H, —COR⁸, —CO₂R⁸, —(CO)—NHR⁸, L⁴, L⁵, L⁶, or L⁷;
R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ is H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
each X³ is individually —H, —OH, —SH, or —NH₂.

2-11. (canceled)

12. The immunoconjugate of claim 1, wherein L⁴ is gly-gly-phe-gly (GGFG).

13-21. (canceled)

22. The immunoconjugate of claim 21, wherein R¹ is C₁-C₃ alkyl and R² is a halogen.

23. The immunoconjugate of claim 21, wherein R¹ is methyl and R² is F.

24-56. (canceled)

57. The immunoconjugate of claim 1, wherein the immunoconjugate of Formula (I) is selected from the group consisting of:

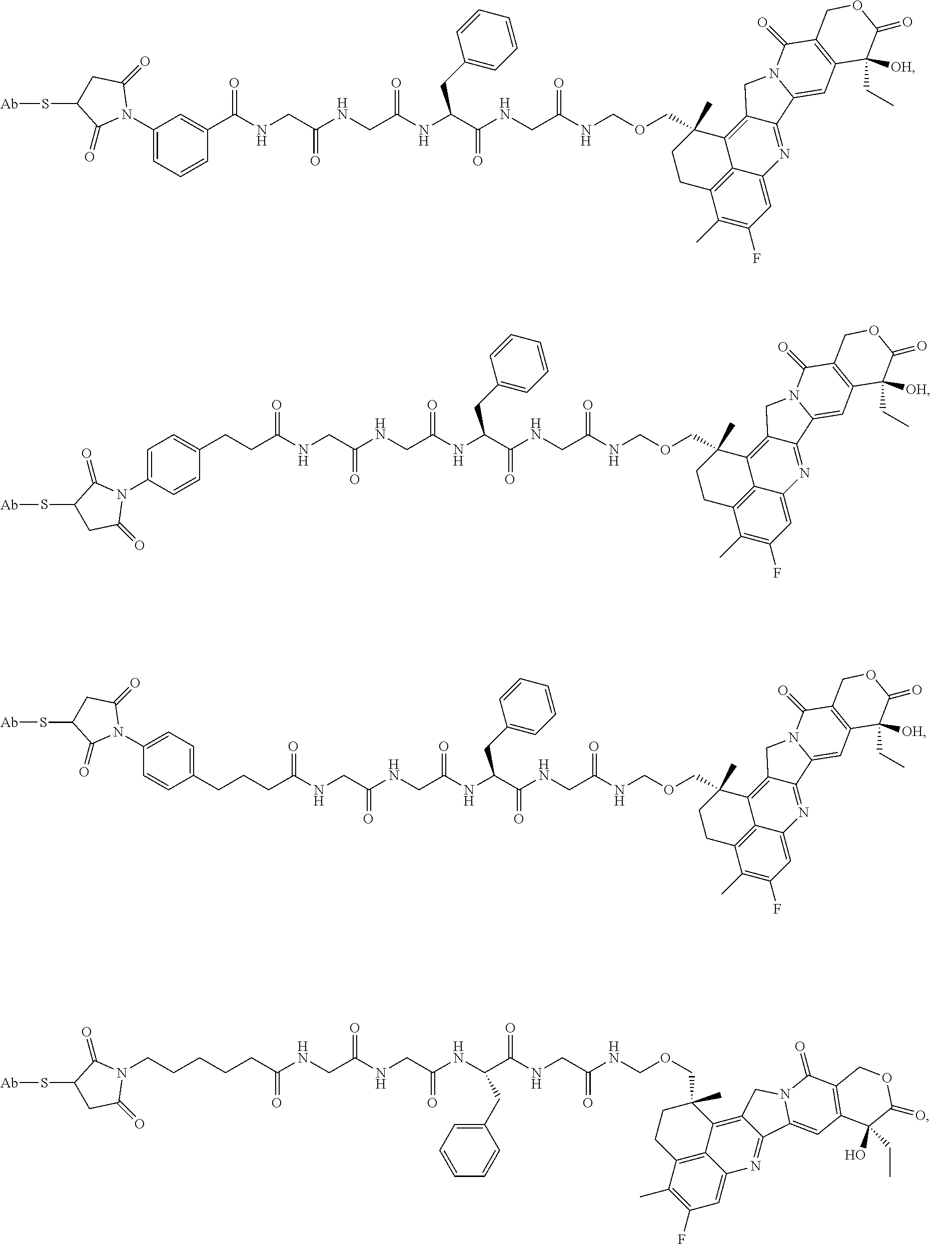

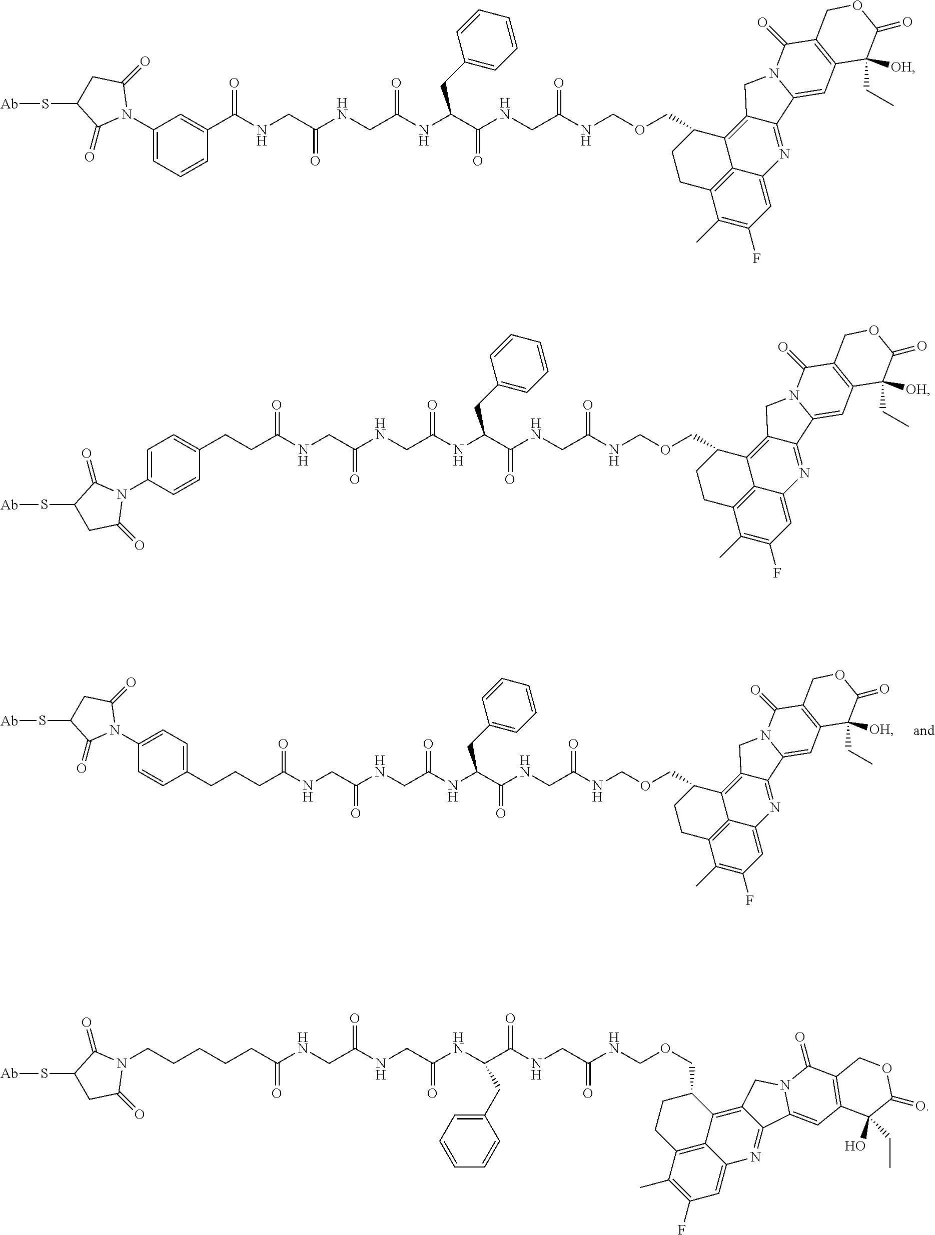

58. The immunoconjugate of claim 1, wherein the immunoconjugate of Formula (I) is selected from the group consisting of:

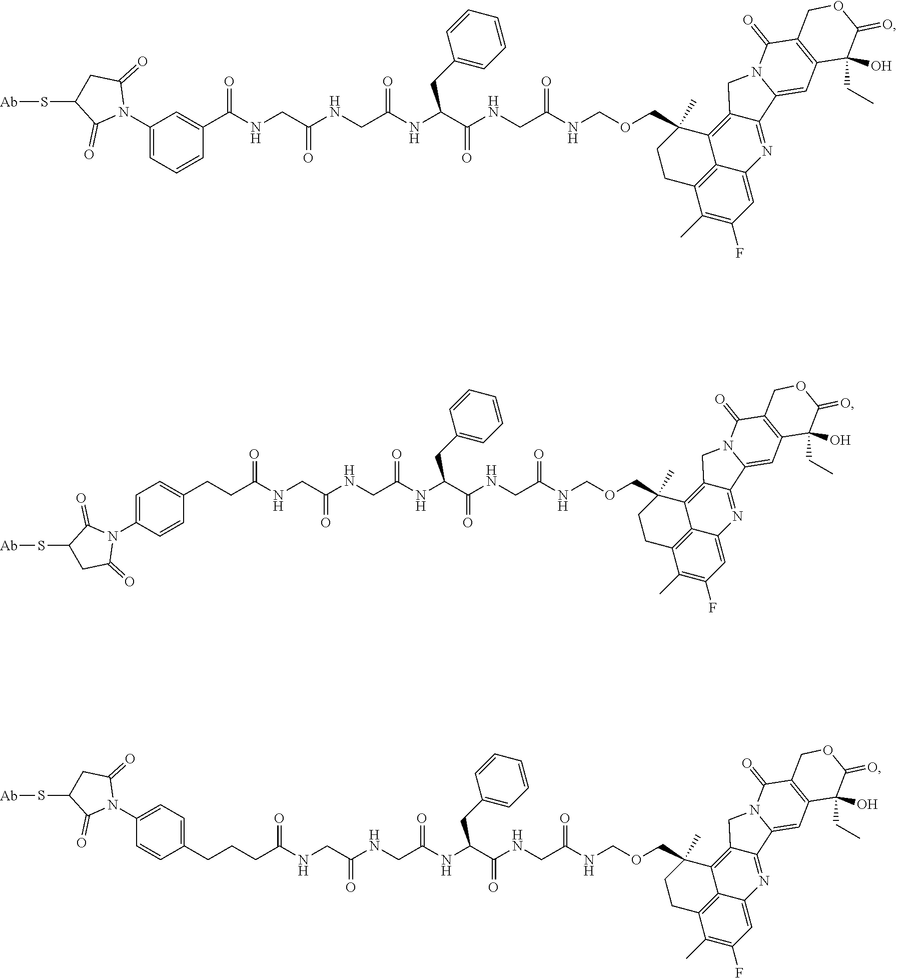

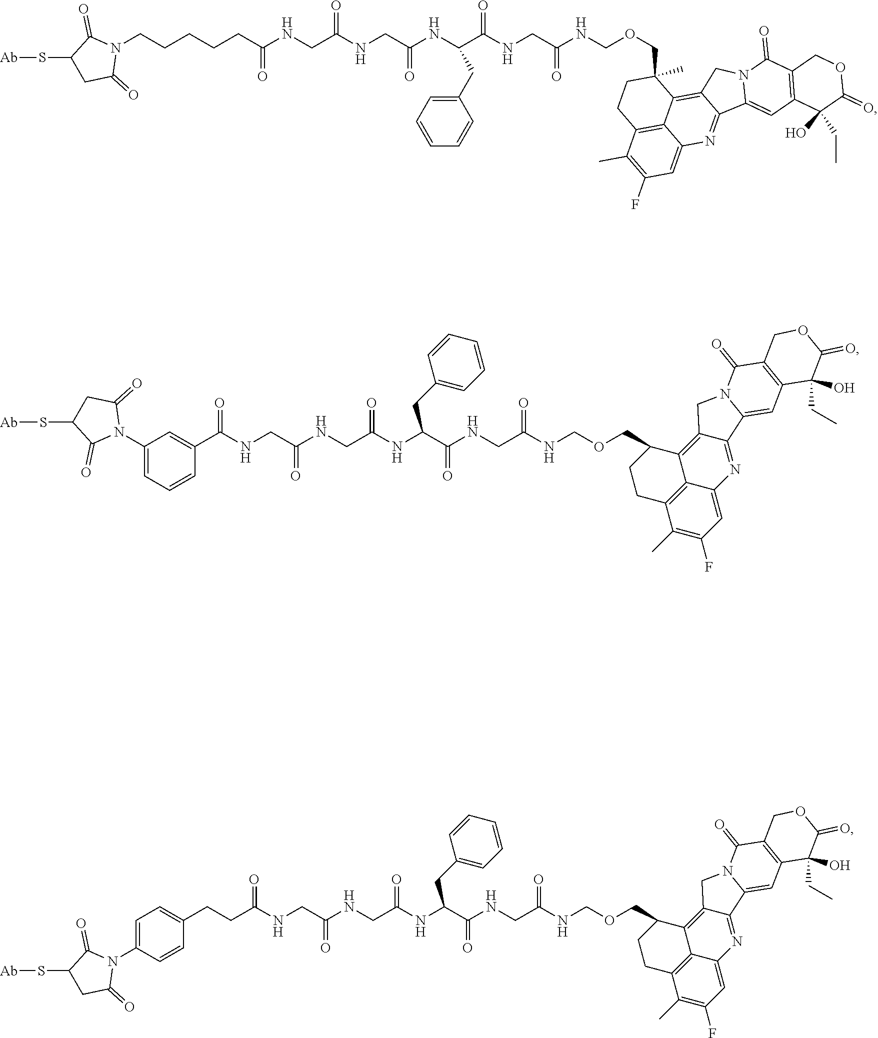

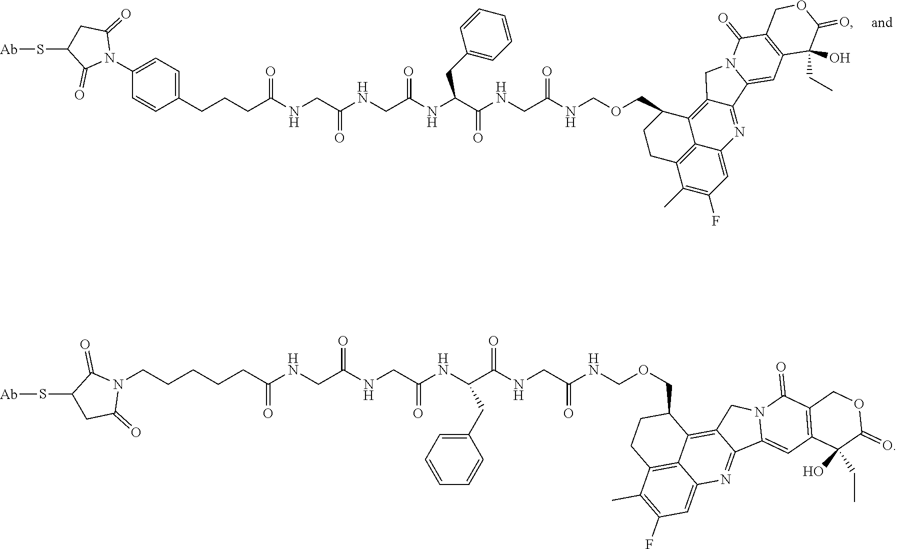

59. The immunoconjugate of claim 1, wherein the immunoconjugate of Formula (I) is selected from the group consisting of:

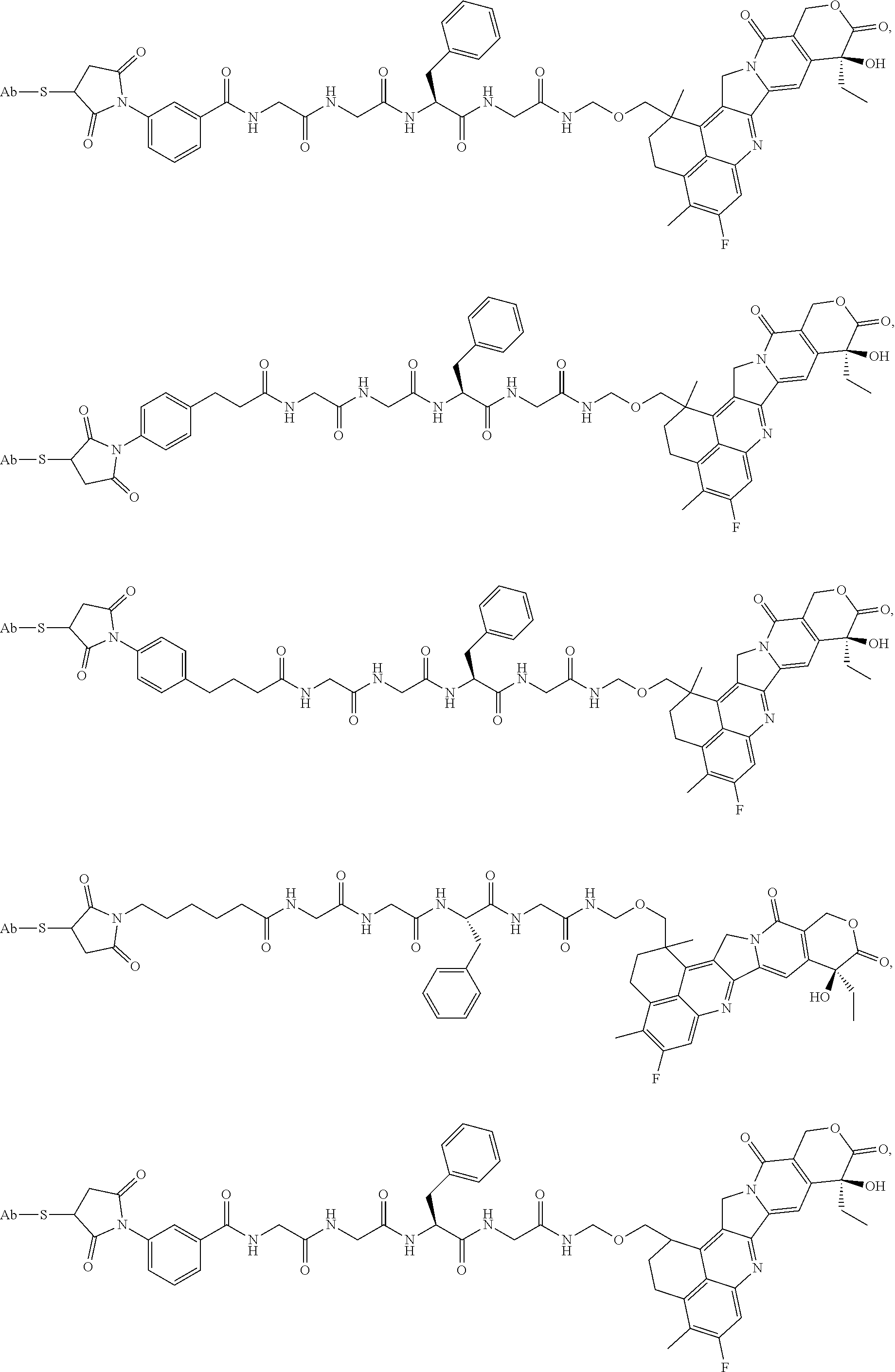

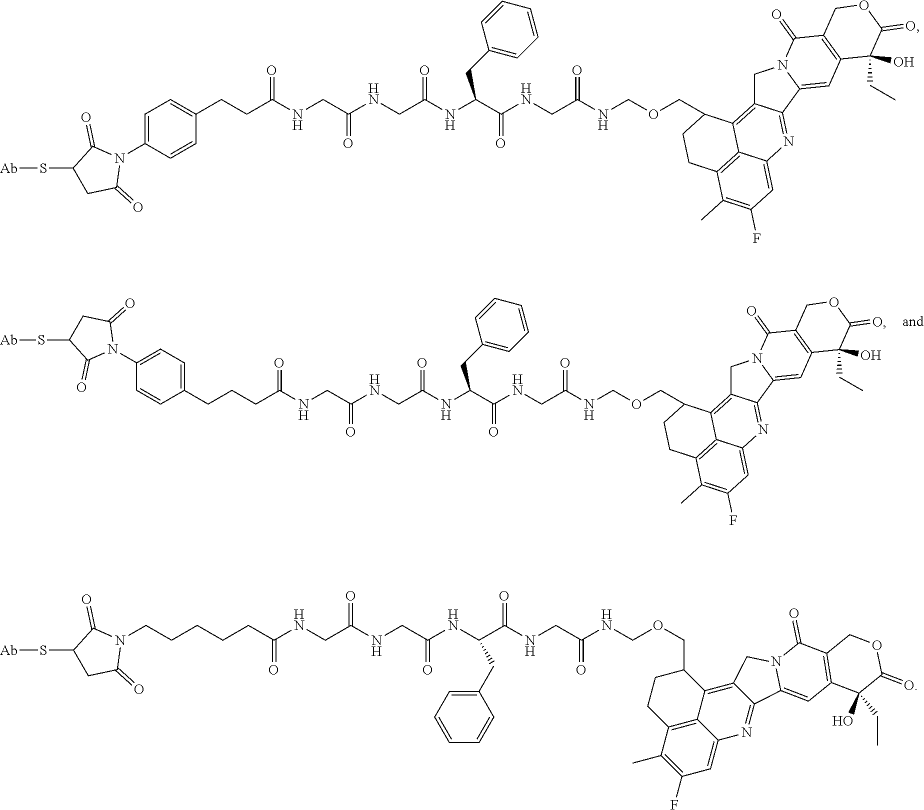

60. The immunoconjugate of claim 1, wherein the immunoconjugate of Formula (I) is selected from the group consisting of:

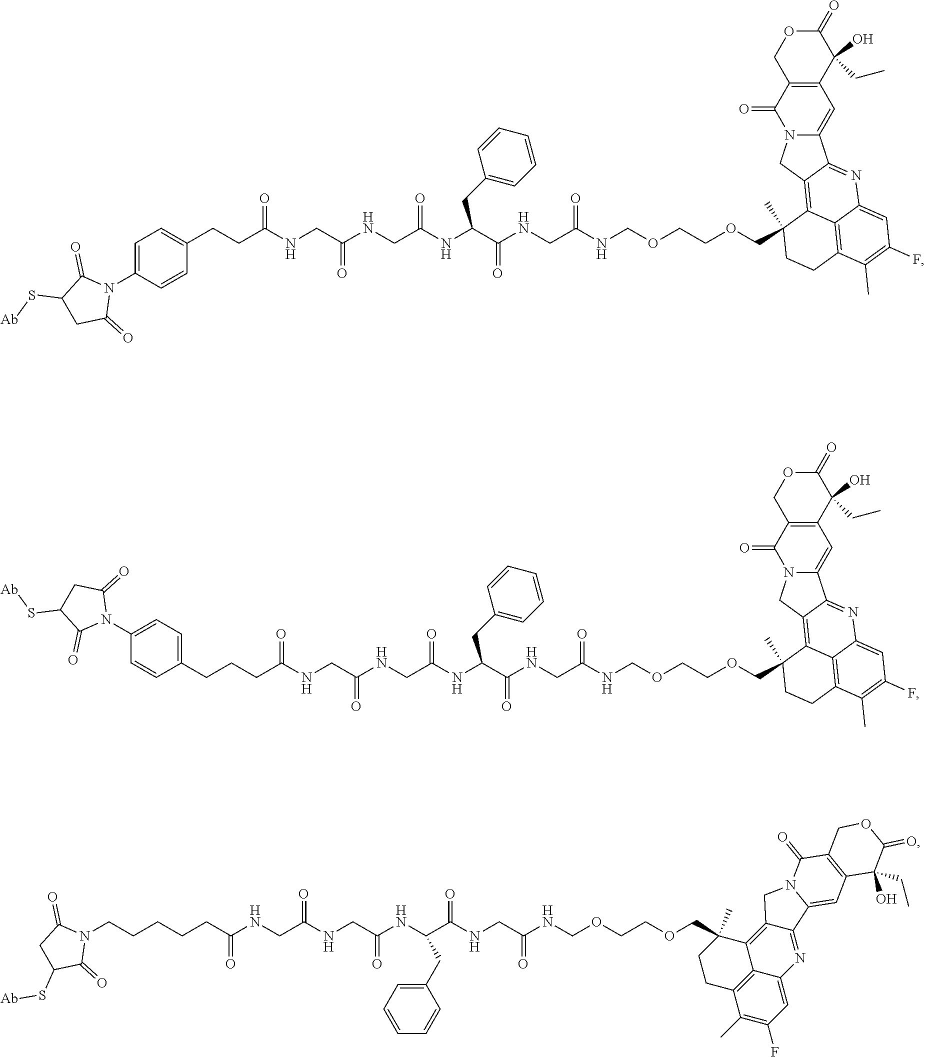

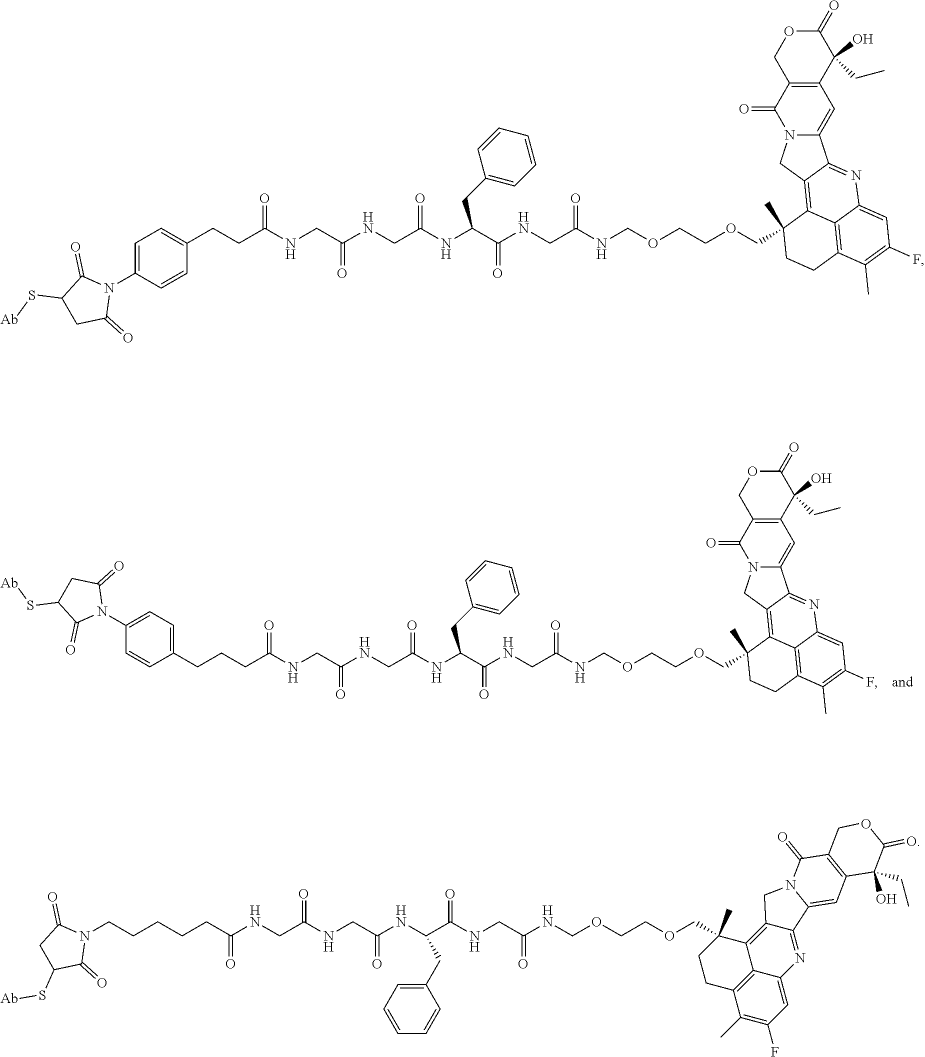

61. The immunoconjugate of claim 1, wherein the immunoconjugate of Formula (I) is selected from the group consisting of:

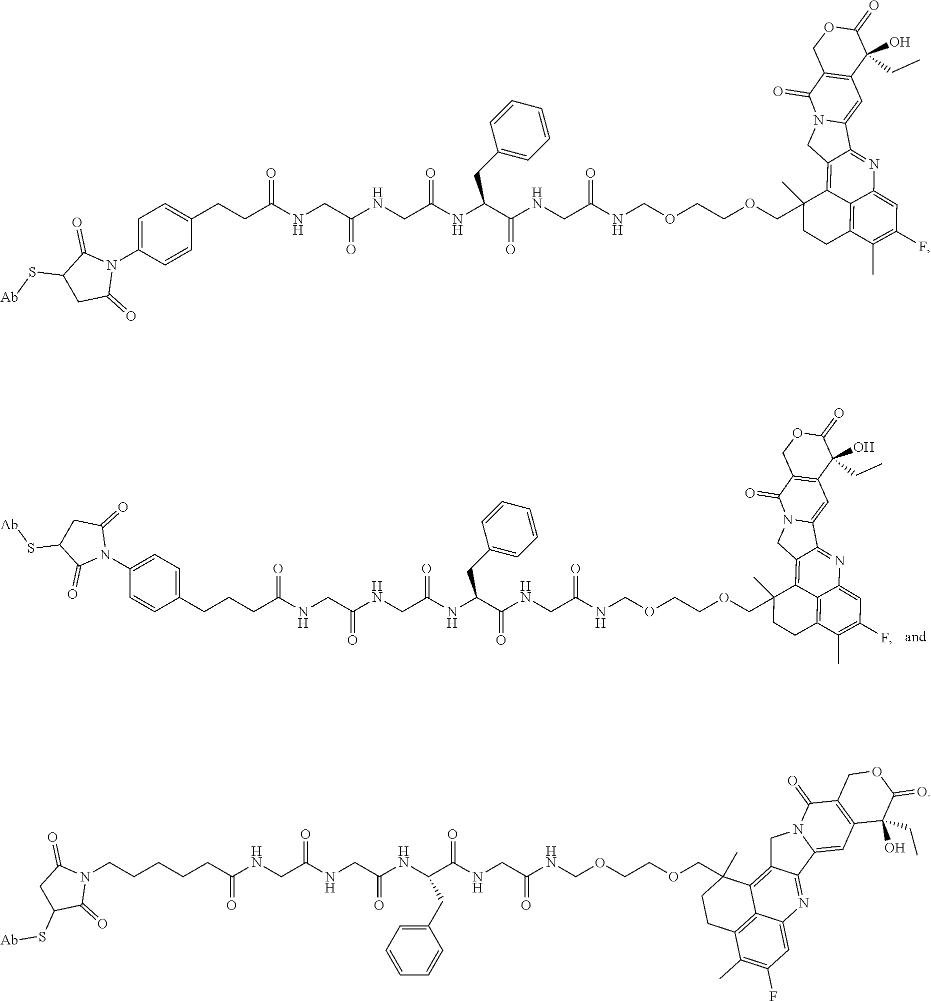

62. A compound of Formula (IV), or a pharmaceutically acceptable salt thereof, having the structure: 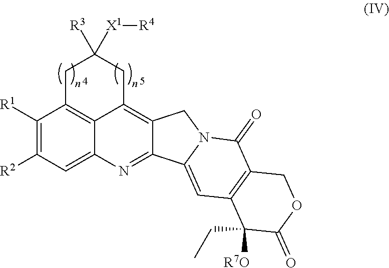
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² taken together form a ring;
R³ is selected from the group consisting of hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and —[(CY₂)ₚO(CY₂)_{q}]ₜCY₃;
R⁴ is selected from the group consisting of a substituted or an unsubstituted C₁-C₆ alkyl, a substituted or an unsubstituted C₂-C₆ alkenyl, a substituted or an unsubstituted C₂-C₆ alkynyl;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each q is individually 0, 1, 2, 3, 4, 5, or 6; and
each t is individually 1, 2, 3, 4, 5, or 6;
R⁷ is H, —COR⁸, —CO₂R⁸, or —(CO)—NHR⁸; and
R⁸ is a substituted or an unsubstituted C₁-C₆ alkyl, a substituted or an unsubstituted C₁-C₆ haloalkyl, or —[(CY₂)ₚO(CY₂)_{q}]ₜCY₃.

63. The compound of claim 62, or a pharmaceutically acceptable salt thereof, wherein R¹ is C₁-C₃ alkyl and R² is a halogen.

64. The compound of claim 62, or a pharmaceutically acceptable salt thereof, wherein R¹ is methyl and R² is F.

65-95. (canceled)

96. The compound of claim 62, or a pharmaceutically acceptable salt thereof, wherein the compound of Formula (IV) is selected from the group consisting of:

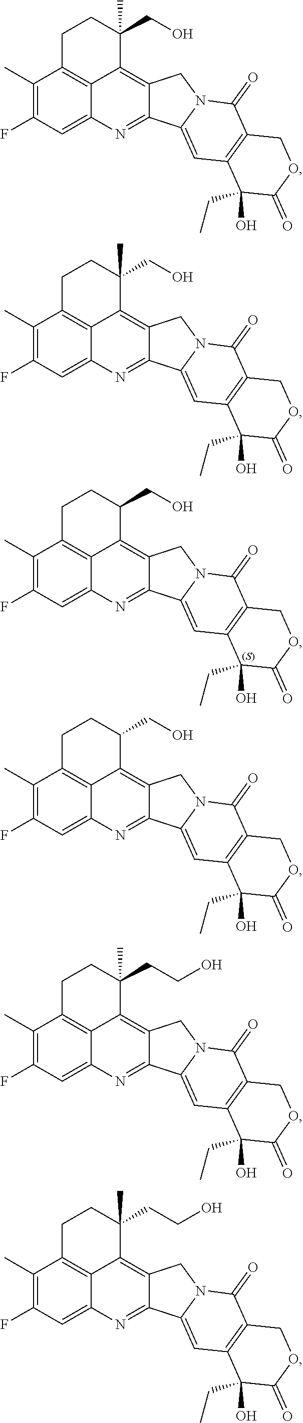

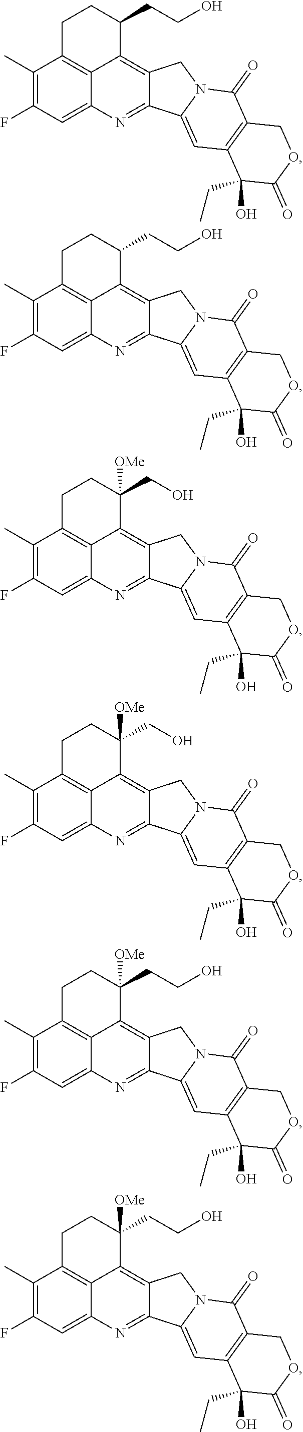

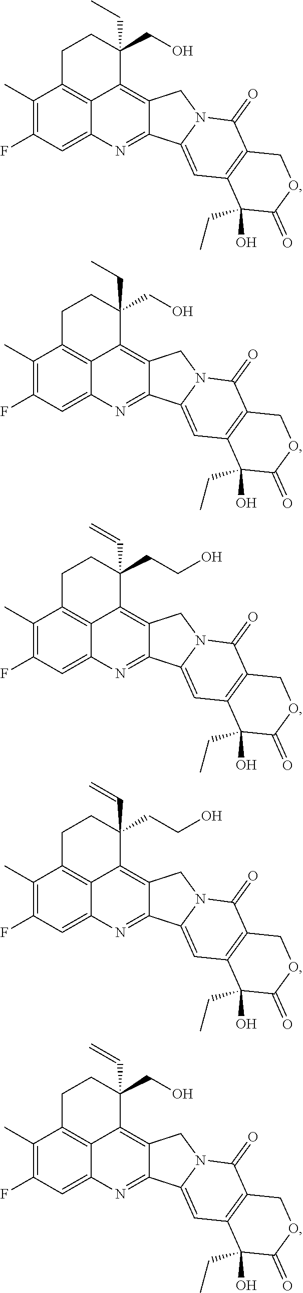

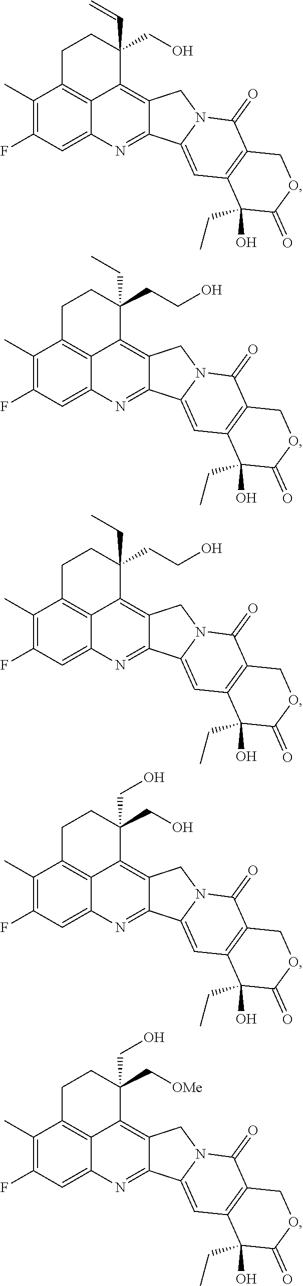

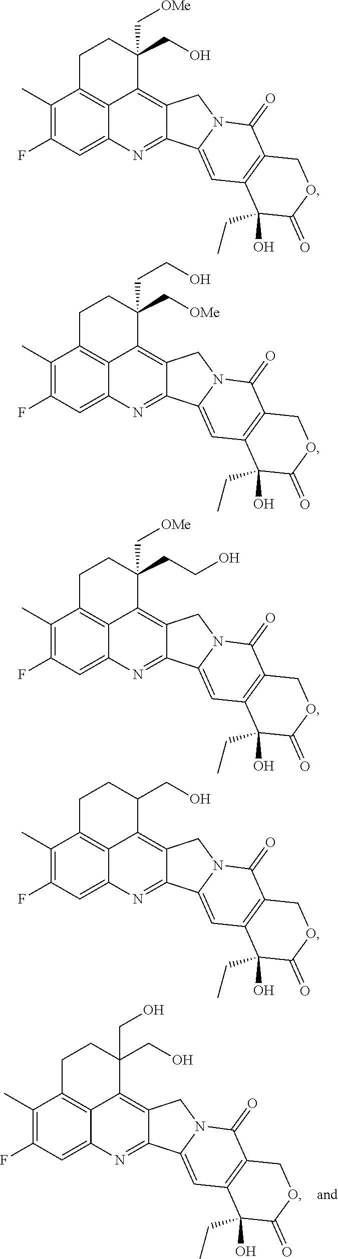

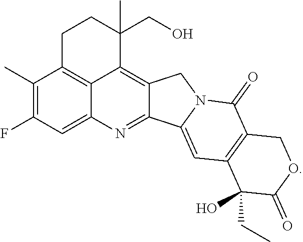 [SMILES: CC[C@@]1(O)C(=O)OCc2c1cc1n(c2=O)Cc2c-1nc1cc(F)c(C)c3c1c2C(C)(CO)CC3]

97. The compound of claim 62, or a pharmaceutically acceptable salt thereof, wherein the compound of Formula (IV) is selected from the group consisting of:

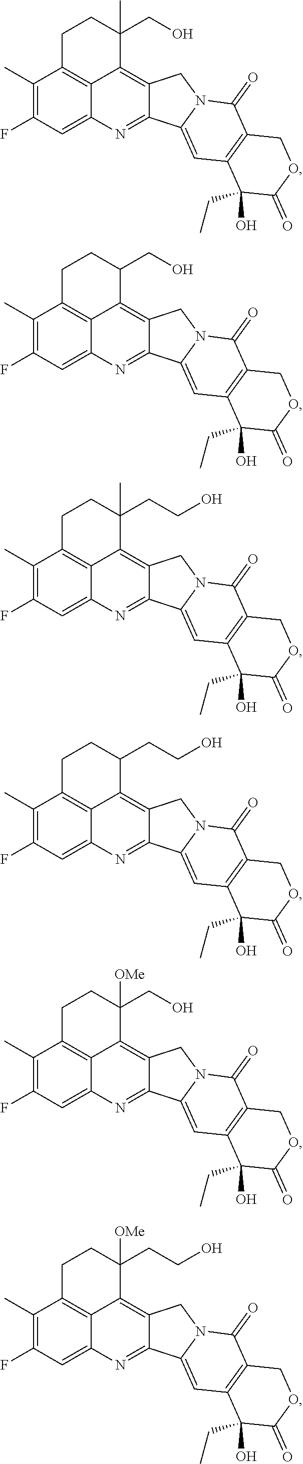

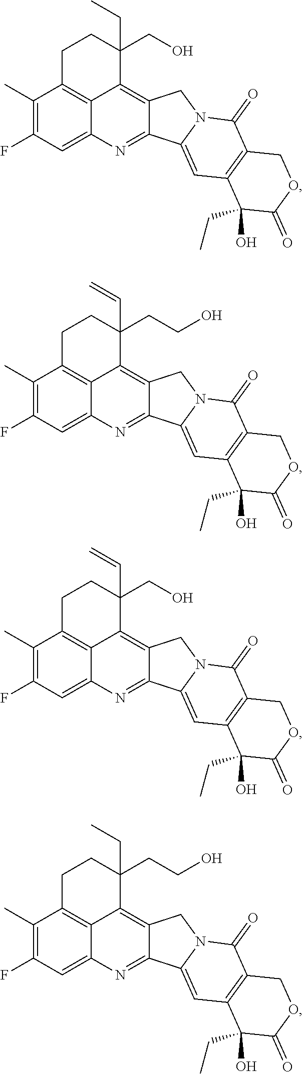 [SMILES: CCC1(CO)CCc2c(C)c(F)cc3nc4c(c1c23)Cn1c-4cc2c(c1=O)COC(=O)[C@]2(O)CC ; C=CC1(CCO)CCc2c(C)c(F)cc3nc4c(c1c23)Cn1c-4cc2c(c1=O)COC(=O)[C@]2(O)CC ; C=CC1(CO)CCc2c(C)c(F)cc3nc4c(c1c23)Cn1c-4cc2c(c1=O)COC(=O)[C@]2(O)CC ; CCC1(CCO)CCc2c(C)c(F)cc3nc4c(c1c23)Cn1c-4cc2c(c1=O)COC(=O)[C@]2(O)CC]

98. The compound of claim 62, or a pharmaceutically acceptable salt thereof, wherein the compound of Formula (IV) is selected from the group consisting of:

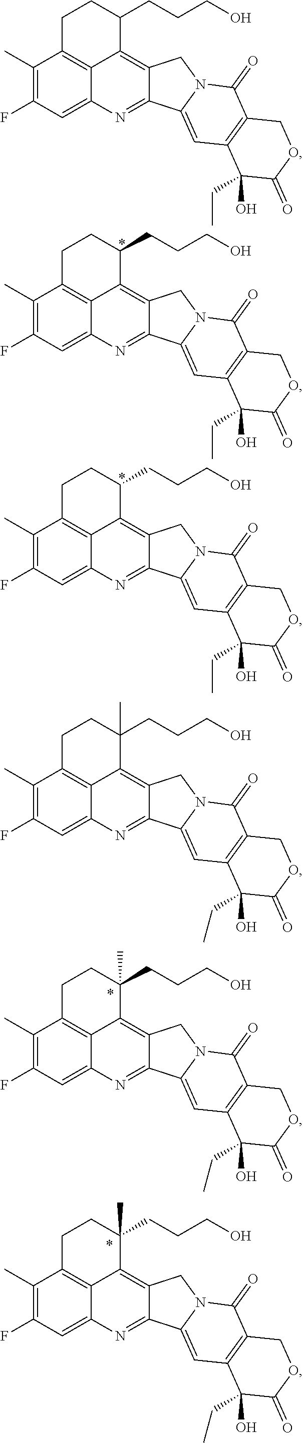

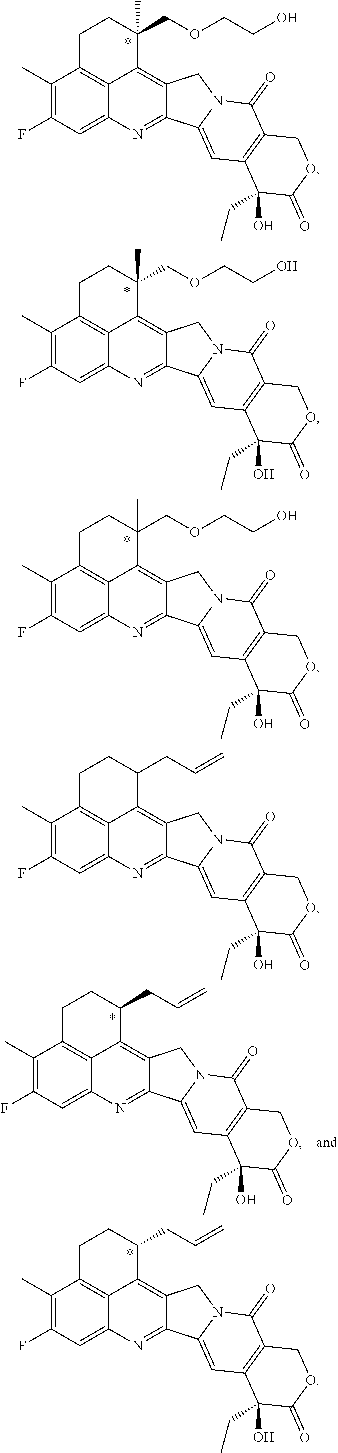

99. The compound of claim 62, or a pharmaceutically acceptable salt thereof, wherein the compound of Formula (IV) is selected from the group consisting of:

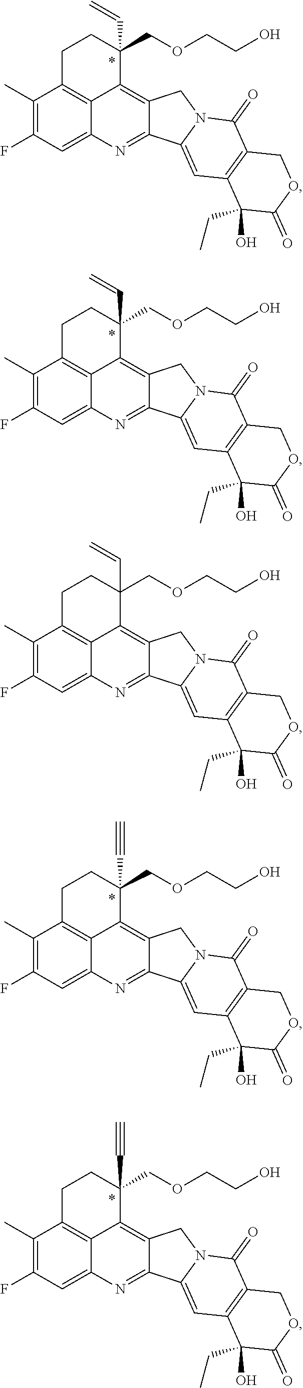 [SMILES: C=C[C@@]1(COCCO)CCc2c(C)c(F)cc3nc4c(c1c23)Cn1c-4cc2c(c1=O)COC(=O)[C@]2(O)CC ; C=C[C@]1(COCCO)CCc2c(C)c(F)cc3nc4c(c1c23)Cn1c-4cc2c(c1=O)COC(=O)[C@]2(O)CC ; C=CC1(COCCO)CCc2c(C)c(F)cc3nc4c(c1c23)Cn1c-4cc2c(c1=O)COC(=O)[C@]2(O)CC ; C#C[C@@]1(COCCO)CCc2c(C)c(F)cc3nc4c(c1c23)Cn1c-4cc2c(c1=O)COC(=O)[C@]2(O)CC ; C#C[C@]1(COCCO)CCc2c(C)c(F)cc3nc4c(c1c23)Cn1c-4cc2c(c1=O)COC(=O)[C@]2(O)CC]

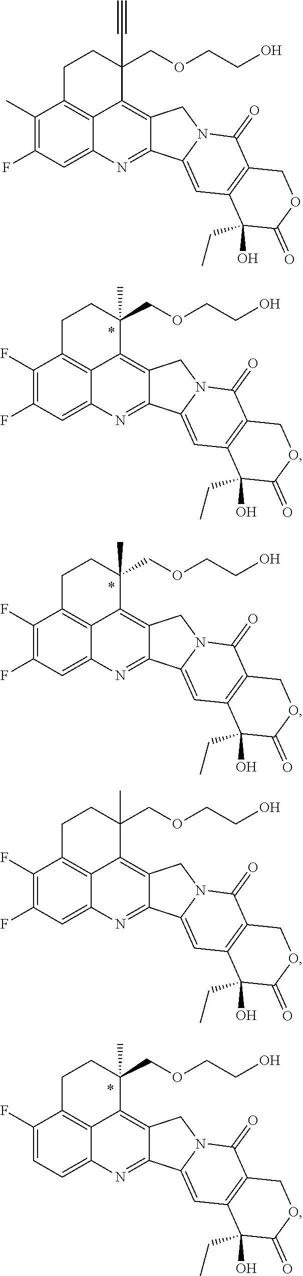

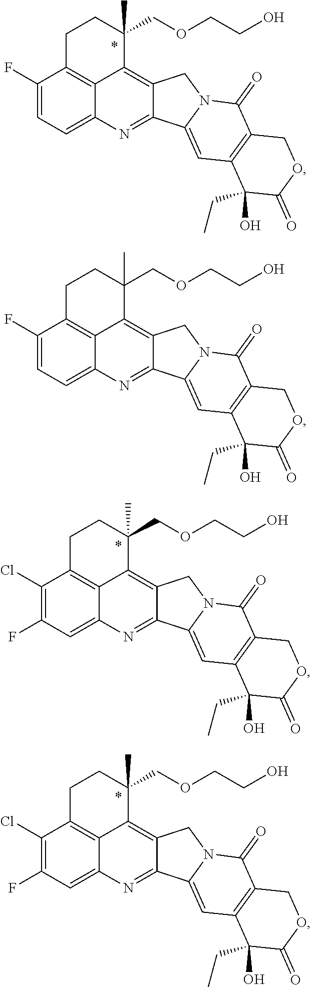

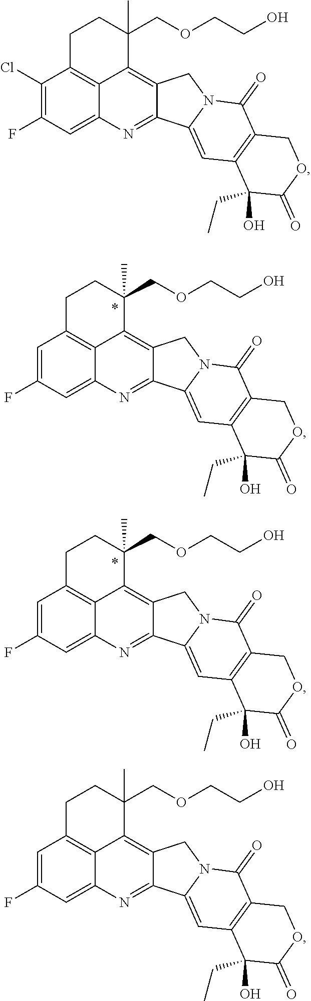

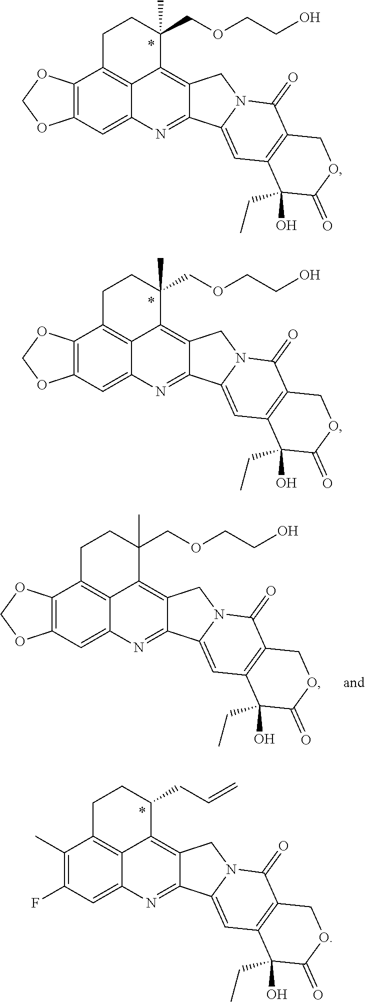

100. A pharmaceutical composition comprising an immunoconjugate of claim 1, or a pharmaceutically active salt thereof, and a pharmaceutically acceptable carrier, diluent, excipient or combination thereof.

101. A method for treating a cancer or a tumor comprising administering an effective amount of claim 1, or a pharmaceutically active salt thereof, to a subject having the cancer or the tumor.

102. The method of claim 101, wherein the cancer or the tumor is selected from lung cancer, urothelial cancer, colorectal cancer, prostate cancer, ovarian cancer, pancreatic cancer, breast cancer, bladder cancer, gastric cancer, gastrointestinal stromal tumor, uterine cervix cancer, esophageal cancer, squamous cell carcinoma, peritoneal cancer, liver cancer, hepatocellular cancer, colon cancer, rectal cancer, colorectal cancer, endometrial cancer, uterine cancer, salivary gland cancer, kidney cancer, vulval cancer, thyroid cancer, penis cancer, leukemia, malignant lymphoma, plasmacytoma, myeloma, or sarcoma.

103-104. (canceled)

105. A conjugate having Formula (III), 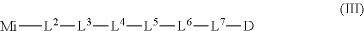
wherein:
Mi is 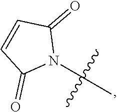 [SMILES: *N1C(=O)C=CC1=O]
L² is absent, 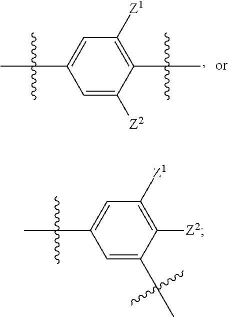
Z¹ and Z² are each individually hydrogen, halogen, NO₂, —O—(C₁-C₆ alkyl), or C₁-C₆alkyl;
L³ is —(CH₂)n¹-C(═O)— or —(CH₂CH₂O)n¹-(CH₂)n¹C(═O)—;
n¹ are independently integers of 0 to 12;
L⁴ is a tetrapeptide residue;
L⁵ is absent or —[NH(CH₂)n²]n³-;
n² is an integer of 0 to 6;
n³ is an integer of 0 to 2;
L⁶ is absent or 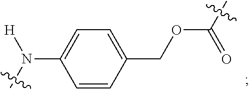
L⁷ is absent, 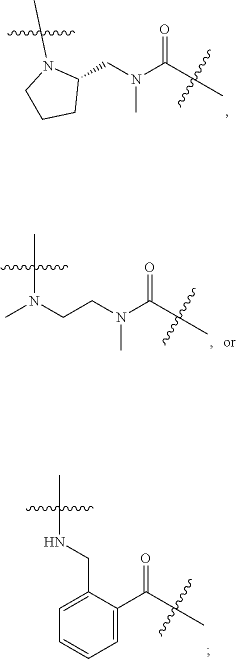
and
D is a drug moiety of Formula (II) having the structure: 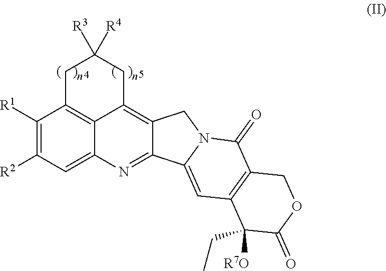
wherein:
R¹ and R² are each individually selected from the group consisting of hydrogen, halogen, —CN, —OR⁵, —NR⁵R⁶, a substituted or an unsubstituted C₁-C₆ alkyl, a substituted or an unsubstituted C₁-C₆ haloalkyl, a substituted or an unsubstituted —O—(C₁-C₆ alkyl), a substituted or an unsubstituted —O—(C₁-C₆ haloalkyl), and —[(CY₂)ₚO(CY₂)_{q}]ₜCY₃, or a substituted or an unsubstituted —O—(CR⁵R⁶)ₘ—O— such that R¹ and R² taken together form a ring;
R³ is selected from the group consisting of hydrogen, —OR⁵, —NR⁵R⁶, a substituted or an unsubstituted C₁-C₆ alkyl, a substituted or an unsubstituted C₂-C₆ alkenyl, a substituted or an unsubstituted C₂-C₆ alkynyl, a substituted or unsubstituted 4- or 5-membered heterocyclyl, and [(CY₂)ₚO(CY₂)_{q}]ₜCY₃;
R⁴ is selected from the group consisting of substituted or unsubstituted —(C₁-C₆ alkyl)-X², substituted or unsubstituted —(C₁-C₆ haloalkyl)-X², substituted or unsubstituted —(C₁-C₆ alkenyl)-X², substituted or unsubstituted —(C₁-C₆ haloalkenyl)-X², substituted or unsubstituted —(C₁-C₆ alkynyl)-X², and substituted or unsubstituted —(C₁-C₆ haloalkynyl)-X²
X² is —OR⁹, —SR⁹, or —NHR⁹;
R⁵ and R⁶ are each individually a substituted or an unsubstituted C₁-C₆ alkyl; or R⁵ and R⁶, taken together with the nitrogen atom to which they are attached, form a substituted or unsubstituted 4- or 5-membered heterocyclyl;
n⁴ and n⁵ are each individually 0, 1 or 2, with the proviso that n⁴ and n⁵ are not both 0;
each Y is individually H or halogen;
each m is individually 1 or 2;
each p is individually 1, 2, 3, 4, 5, or 6;
each t is individually 0, 1, 2, 3, 4, 5, or 6;
each t is individually 1, 2, 3, 4, 5, or 6;
R⁷ is H, —COR⁸, —CO₂R⁸, —(CO)—NHR⁸, L⁴, L⁵, L⁶, or L⁷;
R⁸ is a substituted or an unsubstituted C₁-C₆ alkyl-X³, a substituted or an unsubstituted C₁-C₆ haloalkyl-X³, or —[(CY₂)ₚO(CY₂)_{q}]ₜCY₂—X³;
R⁹ is H, —COR⁸, —CO₂R⁸, —(CO)—NHR⁸, L⁴, L⁵, L⁶, or L⁷, with the proviso that exactly one of R⁷ and R⁹ is L⁴, L⁵, L⁶, or L⁷; and
each X³ is individually —H, —OH, —SH, or —NH₂.

106-115. (canceled)

116. The conjugate of claim 105, wherein L⁴ is gly-gly-phe-gly (GGFG).

117-125. (canceled)

126. The conjugate of claim 125, wherein R¹ is C₁-C₃ alkyl and R² is a halogen.

127. The conjugate of claim 125 or 126, wherein R¹ is methyl and R² is F.

128-158. (canceled)

159. The conjugate of claim 105, wherein the conjugate having Formula (III) is selected from the group consisting of:

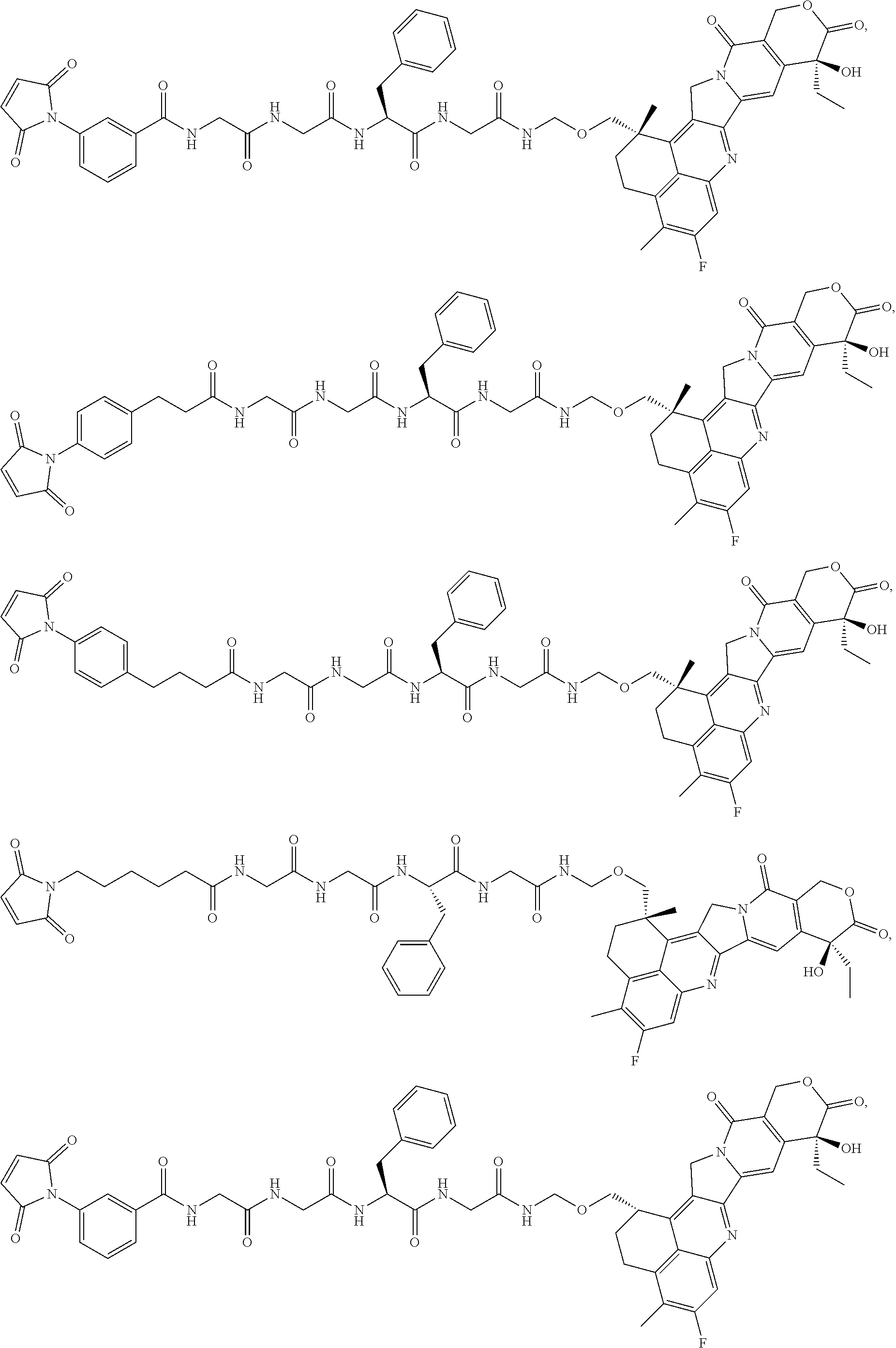

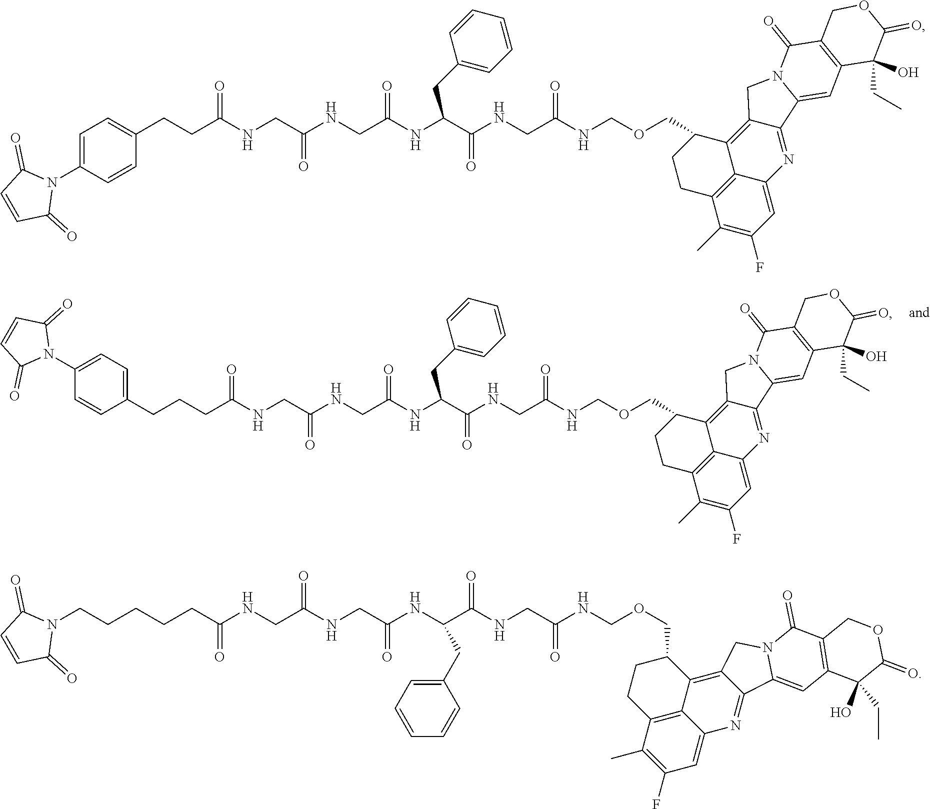

160. The conjugate of claim 105, wherein the conjugate having Formula (III) is selected from the group consisting of:

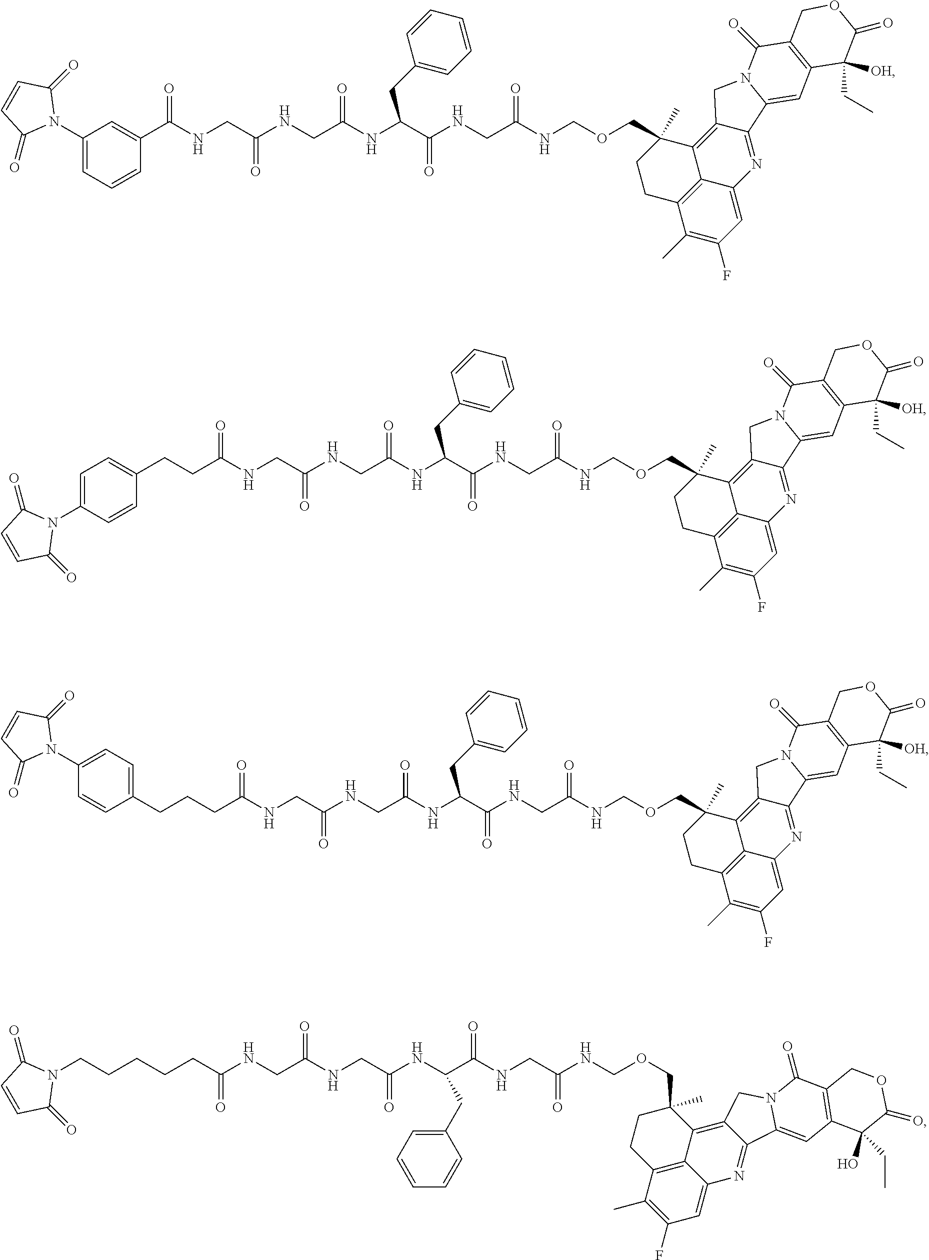

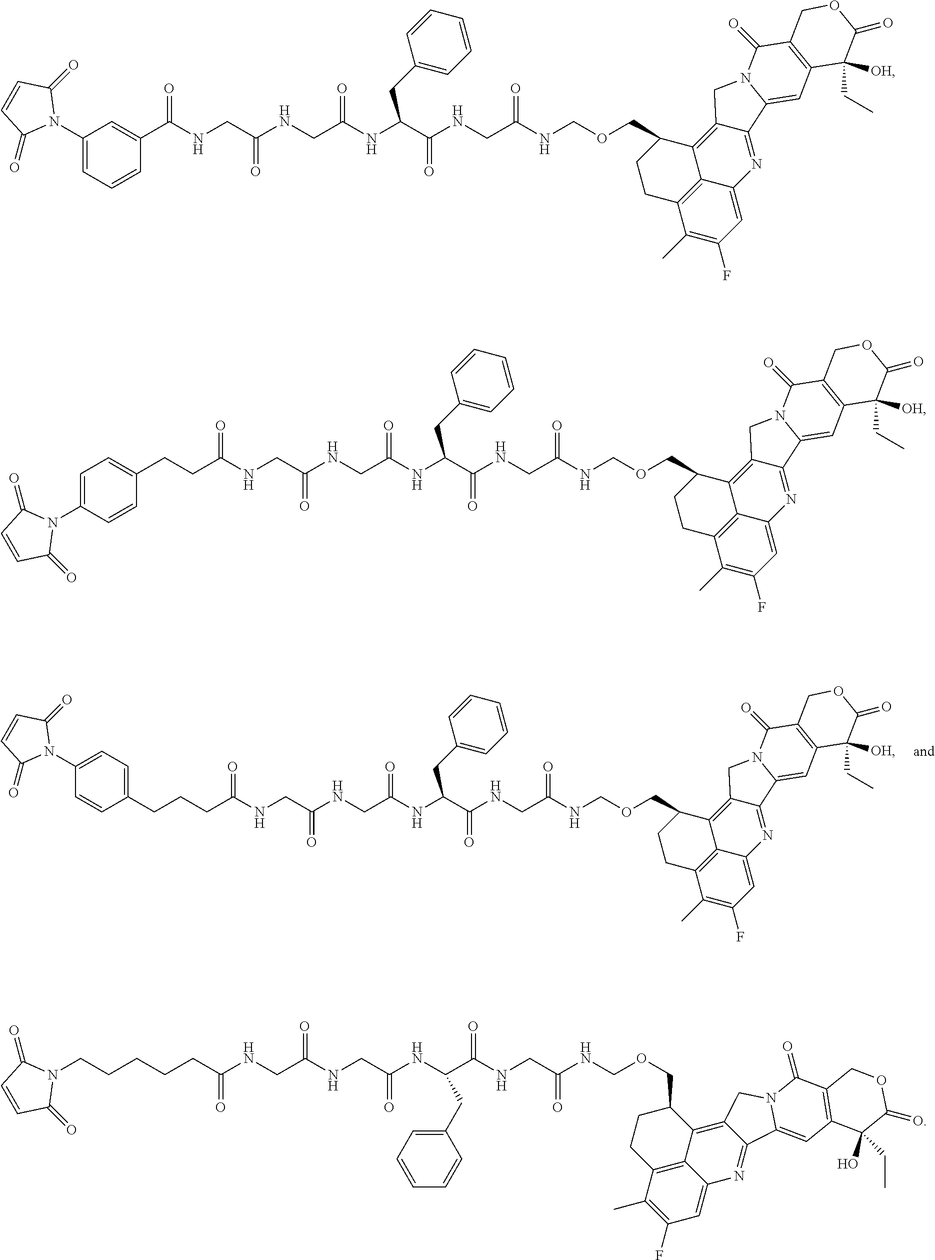

161. The conjugate of claim 105, wherein the conjugate having Formula (III) is selected from the group consisting of:

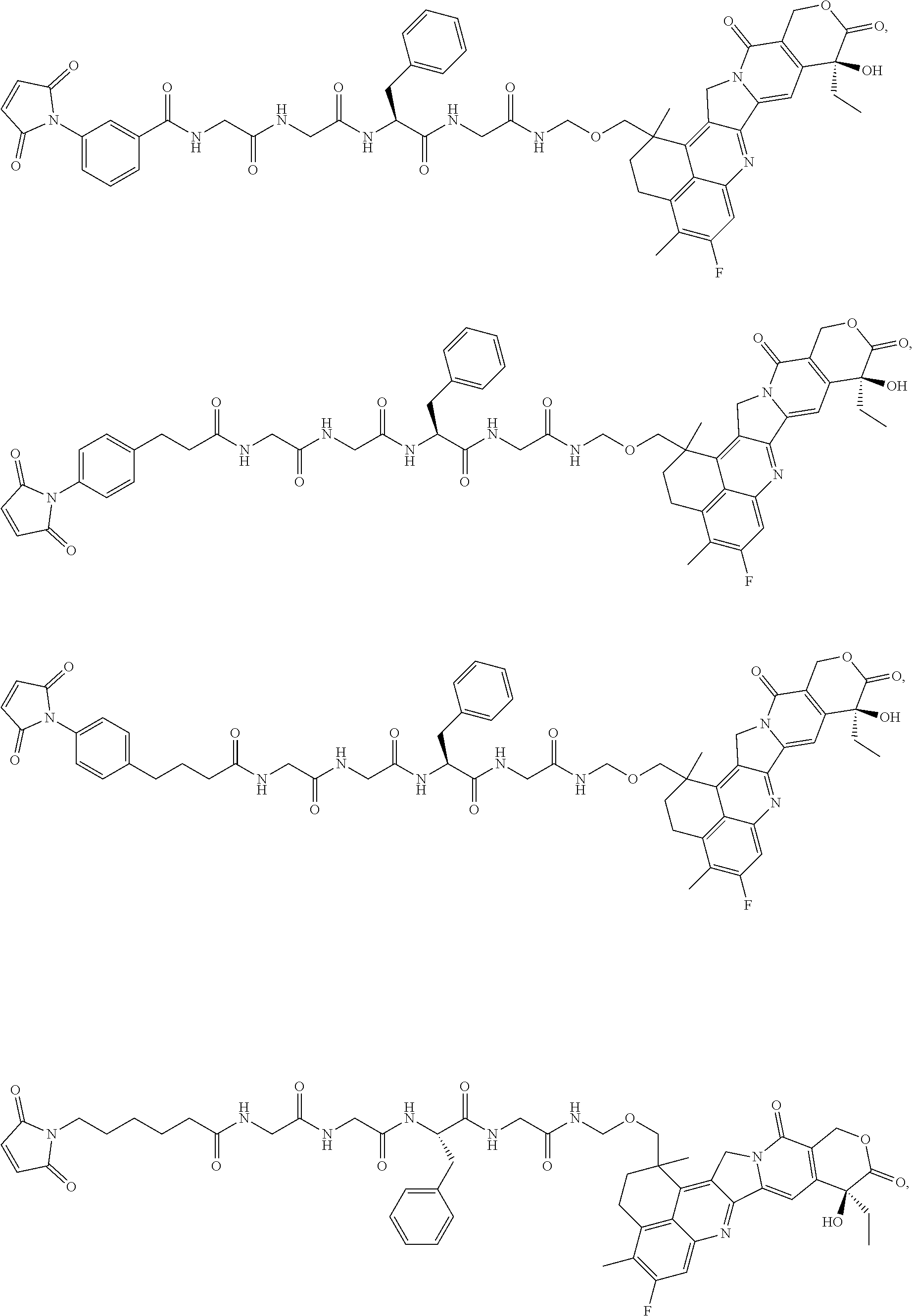

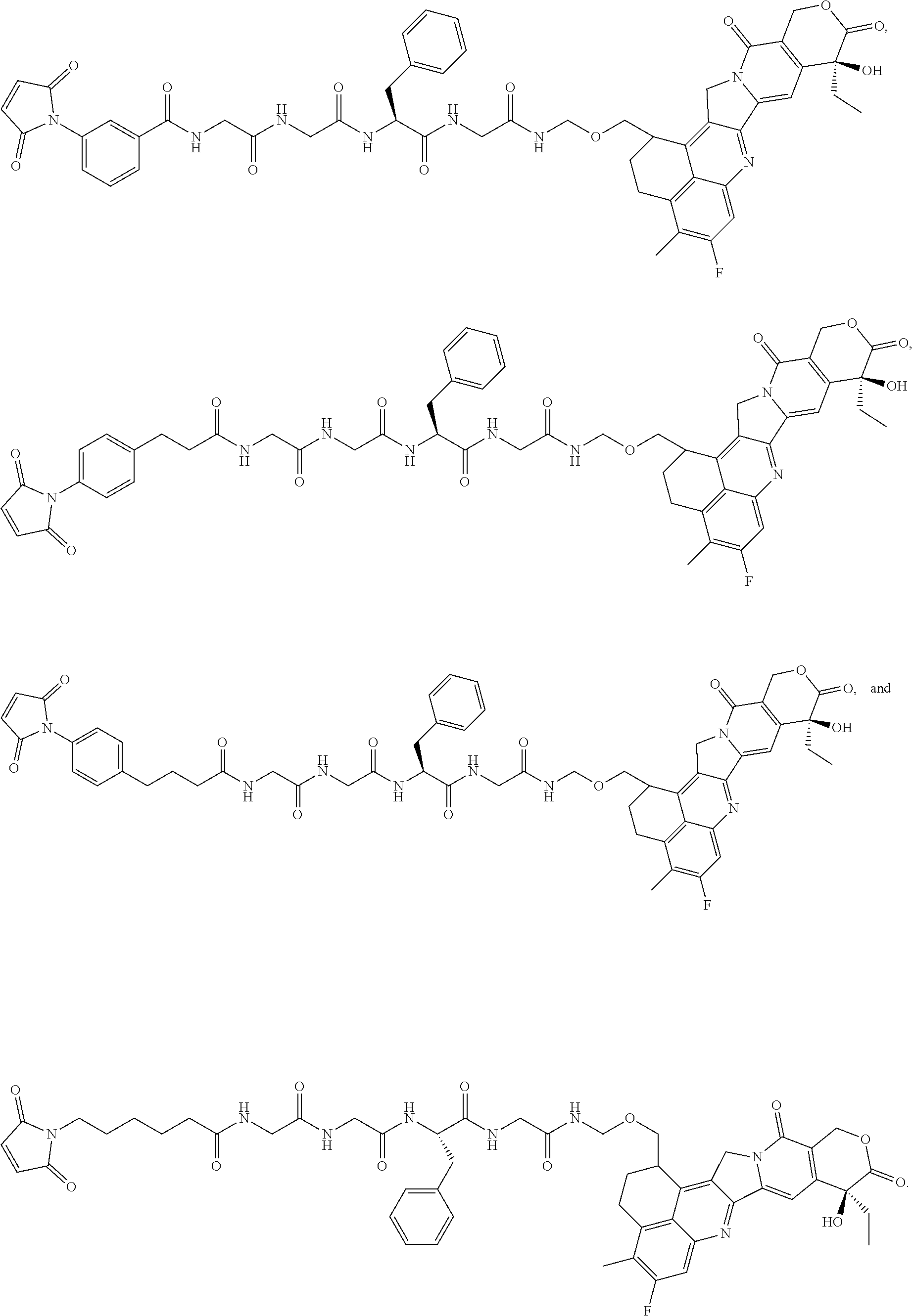

162. The conjugate of claim 105, wherein the conjugate having Formula (III) is selected from the group consisting of:

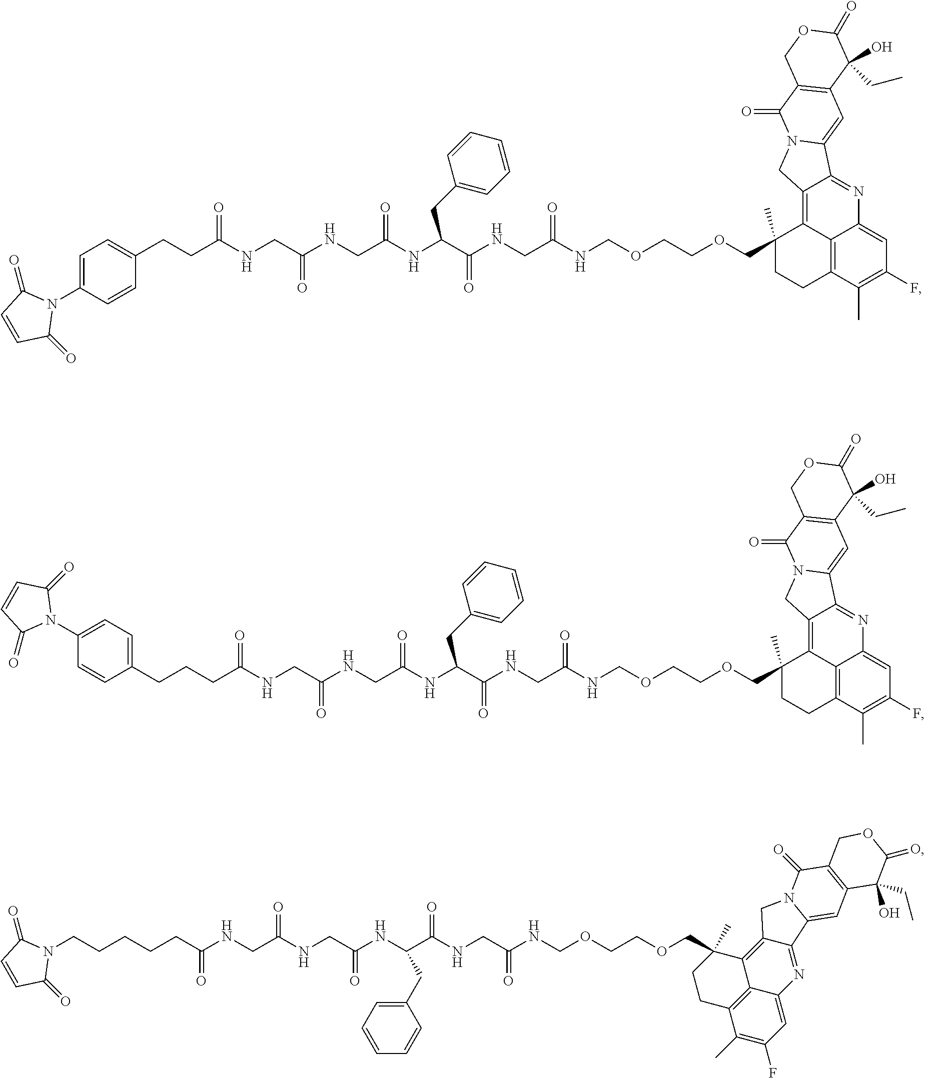 [SMILES: CC[C@@]1(O)C(=O)OCc2c1cc1n(c2=O)Cc2c-1nc1cc(F)c(C)c3c1c2[C@](C)(COCCOCNC(=O)CNC(=O)[C@H](Cc1ccccc1)NC(=O)CNC(=O)CNC(=O)CCc1ccc(N2C(=O)C=CC2=O)cc1)CC3 ; CC[C@@]1(O)C(=O)OCc2c1cc1n(c2=O)Cc2c-1nc1cc(F)c(C)c3c1c2[C@](C)(COCCOCNC(=O)CNC(=O)[C@H](Cc1ccccc1)NC(=O)CNC(=O)CNC(=O)CCCc1ccc(N2C(=O)C=CC2=O)cc1)CC3 ; CC[C@@]1(O)C(=O)OCc2c1cc1n(c2=O)Cc2c-1nc1cc(F)c(C)c3c1c2[C@](C)(COCCOCNC(=O)CNC(=O)[C@H](Cc1ccccc1)NC(=O)CNC(=O)CNC(=O)CCCCCN1C(=O)C=CC1=O)CC3]

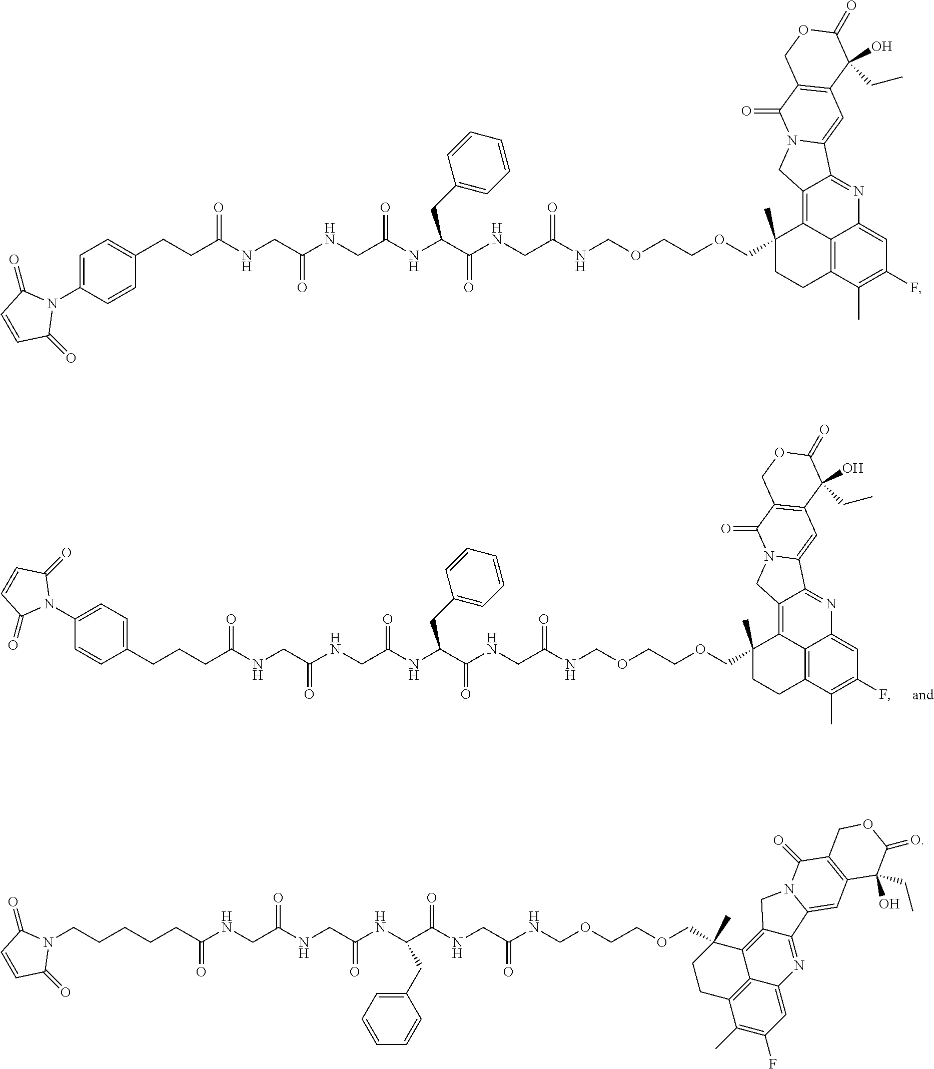 [SMILES: CC[C@@]1(O)C(=O)OCc2c1cc1n(c2=O)Cc2c-1nc1cc(F)c(C)c3c1c2[C@@](C)(COCCOCNC(=O)CNC(=O)[C@H](Cc1ccccc1)NC(=O)CNC(=O)CNC(=O)CCc1ccc(N2C(=O)C=CC2=O)cc1)CC3 ; CC[C@@]1(O)C(=O)OCc2c1cc1n(c2=O)Cc2c-1nc1cc(F)c(C)c3c1c2[C@@](C)(COCCOCNC(=O)CNC(=O)[C@H](Cc1ccccc1)NC(=O)CNC(=O)CNC(=O)CCCc1ccc(N2C(=O)C=CC2=O)cc1)CC3 ; CC[C@@]1(O)C(=O)OCc2c1cc1n(c2=O)Cc2c-1nc1cc(F)c(C)c3c1c2[C@@](C)(COCCOCNC(=O)CNC(=O)[C@H](Cc1ccccc1)NC(=O)CNC(=O)CNC(=O)CCCCCN1C(=O)C=CC1=O)CC3]

163. The conjugate of claim 105, wherein the conjugate having Formula (III) is selected from the group consisting of:

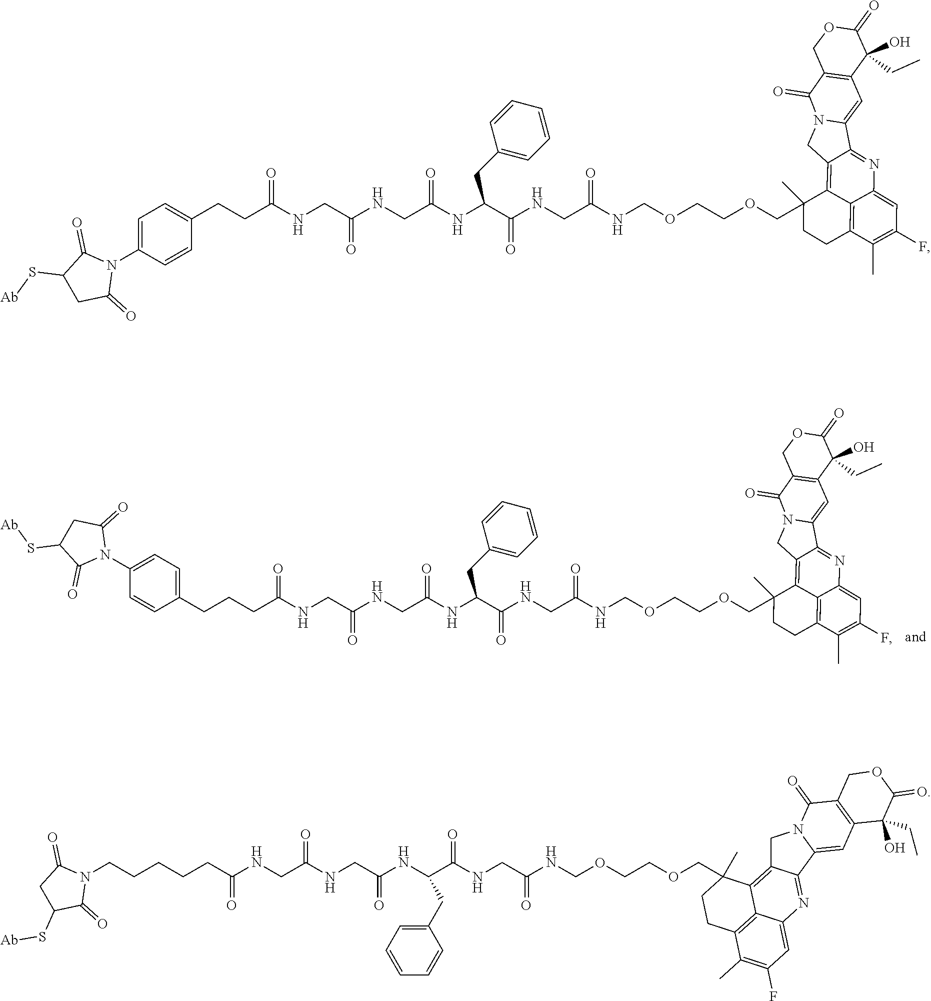

164-175. (canceled)


SEQUENCE LISTING (46 sequences)

SEQ ID NO: 1 - protein, 8 aa, Synthetic construct
GFTFSNAW

SEQ ID NO: 2 - protein, 10 aa, synthetic construct
IKSKTDGGTT

SEQ ID NO: 3 - protein, 9 aa, synthetic construct
TTGPDDLDY

SEQ ID NO: 4 - DNA, 354 bp, synthetic construct
  source 1..354: mol_type = other DNA
gaggtgcagctggtggagtccgggggaggcctggtcaagcctggggggtccctgagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctctatctgcaaatgaacagcctgaaaaccgaggacacagccgtgtattactgtaccacaggccctgacgatcttgactactggggccagggaaccccggtcaccgtctcctca

SEQ ID NO: 5 - protein, 118 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGPDDLDYWGQGTPVTVSS

SEQ ID NO: 6 - DNA, 1341 bp, synthetic construct
  source 1..1341: mol_type = other DNA
gaggtgcagctggtggagtccgggggaggcctggtcaagcctggggggtccctgagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctctatctgcaaatgaacagcctgaaaaccgaggacacagccgtgtattactgtaccacaggccctgacgatcttgactactggggccagggaaccccggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 7 - protein, 447 aa, synthetic construct
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGPDDLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 6 aa, synthetic construct
QSISSY

SEQ ID NO: 9 - skipped

SEQ ID NO: 10 - protein, 9 aa, synthetic construct
QQYDNLPIT

SEQ ID NO: 11 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagcggcagtggatctgggacagatttcactctcaccatcagcagcctgcaacctgaagattttgcaacttactactgtcaacagtatgataatctcccgatcaccttcggccaagggacacgactggagattaaa

SEQ ID NO: 12 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYDNLPITFGQGTRLEIK

SEQ ID NO: 13 - DNA, 642 bp, synthetic construct
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagcggcagtggatctgggacagatttcactctcaccatcagcagcctgcaacctgaagattttgcaacttactactgtcaacagtatgataatctcccgatcaccttcggccaagggacacgactggagattaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 14 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQYDNLPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 8 aa, synthetic construct
GGSFSGYY

SEQ ID NO: 16 - protein, 7 aa, synthetic construct
INHSGST

SEQ ID NO: 17 - protein, 9 aa, synthetic construct
AREGVYEDY

SEQ ID NO: 18 - DNA, 345 bp, synthetic construct
  source 1..345: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtatattactgtgcgagagagggtgtctacgaggactactggggccagggaaccctggtcaccgtctcctca

SEQ ID NO: 19 - protein, 115 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREGVYEDYWGQGTLVTVSS

SEQ ID NO: 20 - DNA, 1211 bp, synthetic construct
  source 1..1211: mol_type = other DNA
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtatattactgtgcgagagagggtgtctacgaggactactggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttct

SEQ ID NO: 21 - protein, 444 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAREGVYEDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 6 aa, synthetic construct
QSVSNY

SEQ ID NO: 23 - skipped

SEQ ID NO: 24 - protein, 9 aa, synthetic construct
QQRSNWPLT

SEQ ID NO: 25 - DNA, 321 bp, synthetic construct
  source 1..321: mol_type = other DNA
gaaattgtgttgacacagtctccagccaccctgtctttgtctccaggggaaagagccaccctctcctgcagggccagtcagagtgttagcaactacttagcctggtaccaacagaaacctggccaggctcccaggctcctcatctatgatgcctacaacagggccactggcatcccagccaggttcagtggcagtgggtctgggacagacttcactctcaccatcagcagcctagagcctgaagattttgcagtttattactgtcagcagcgtagcaactggcctctcaccttcggccaagggacacgactggagattaaa

SEQ ID NO: 26 - protein, 107 aa, synthetic construct
EIVLTQSPATLSLSPGERATLSCRASQSVSNYLAWYQQKPGQAPRLLIYDAYNRATGIPARFSGSGSGTDFTLTISSLEPEDFAVYYCQQRSNWPLTFGQGTRLEIK

SEQ ID NO: 27 - DNA, 642 bp, synthetic construct
  source 1..642: mol_type = other DNA
gaaattgtgttgacacagtctccagccaccctgtctttgtctccaggggaaagagccaccctctcctgcagggccagtcagagtgttagcaactacttagcctggtaccaacagaaacctggccaggctcccaggctcctcatctatgatgcctacaacagggccactggcatcccagccaggttcagtggcagtgggtctgggacagacttcactctcaccatcagcagcctagagcctgaagattttgcagtttattactgtcagcagcgtagcaactggcctctcaccttcggccaagggacacgactggagattaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 28 - protein, 214 aa, synthetic construct
EIVLTQSPATLSLSPGERATLSCRASQSVSNYLAWYQQKPGQAPRLLIYDAYNRATGIPARFSGSGSGTDFTLTISSLEPEDFAVYYCQQRSNWPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 8 aa, synthetic construct
GYTFTGYY

SEQ ID NO: 30 - protein, 8 aa, synthetic construct
INPNSGGT

SEQ ID NO: 31 - protein, 12 aa, synthetic construct
VRDQVQLERFDS

SEQ ID NO: 32 - DNA, 357 bp, synthetic construct
  source 1..357: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgtgagagatcaggtacaactggaacggttcgactcctggggccagggaaccctggtcaccgtctcctca

SEQ ID NO: 33 - protein, 119 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCVRDQVQLERFDSWGQGTLVTVSS

SEQ ID NO: 34 - DNA, 1344 bp, synthetic construct
  source 1..1344: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgtgagagatcaggtacaactggaacggttcgactcctggggccagggaaccctggtcaccgtctcctcagctagcactaaagggccttctgtatttcccttggccccgtccagcaaatcgacctcgggagggacagccgccctgggttgccttgtgaaagattatttccctgagccagttaccgtaagttggaacagtggggcgctgacaagtggtgtgcacacgtttcctgccgtcctgcaatcatcgggcttgtatagcctcagctctgtggtcactgtcccaagttcatcgctgggcactcagacgtatatttgcaatgtgaaccacaaaccttcaaatacaaaagtggataaacgcgtagaaccgaaatcgtgtgataaaactcacacatgcccgccatgcccggcacctgaactgcttggtggtcccagcgtgttcctgttcccgccgaagcctaaagatactctaatgatcagccgtacgccagaggtgacatgtgtcgtggttgacgtgtcccacgaagatcccgaagttaagttcaattggtatgttgatggtgtagaggtacacaatgctaagactaaacctcgcgaggagcagtacaattcgacctatcgtgtcgtgagcgttctgaccgtccttcaccaagattggcttaacggcaaagaatataagtgcaaggtaagcaataaagcacttccggccccaatcgagaaaaccatttccaaggccaaaggtcaaccaagagaaccccaggtgtatactcttccgccttctcgtgaggaaatgactaaaaatcaagtatcccttacgtgtctggttaaaggtttttatcctagcgatattgctgttgaatgggaatcgaacggtcagccggagaataattataaaacaacgccacccgtcctggatagcgacggctcattttttctgtatagcaaactgactgtagataaatcacggtggcagcagggcaatgtattcagttgctccgttatgcatgaagcgttacataatcactacacgcagaaatctcttagtctttcacccggt

SEQ ID NO: 35 - protein, 448 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCVRDQVQLERFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 6 aa, synthetic construct
QDISNY

SEQ ID NO: 37 - skipped

SEQ ID NO: 38 - protein, 9 aa, synthetic construct
QQYDNLPPT

SEQ ID NO: 39 - DNA, 321 bp, synthetic construct
  source 1..321: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccctcccactttcggccctgggaccaaggtggaaatcaaa

SEQ ID NO: 40 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTFGPGTKVEIK

SEQ ID NO: 41 - DNA, 642 bp, synthetic construct
  source 1..642: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctccctcccactttcggccctgggaccaaggtggaaatcaaacgtacggtagctgccccttcagtttttatctttccgccgtctgacgagcagttaaaatccgggaccgcttctgtagtttgcctgctgaataatttttatccgcgtgaggctaaagtacaatggaaagtcgacaatgctttgcagtcgggaaattcacaggaaagtgttacggagcaggattctaaagattccacatattcactcagctccacccttacactgagcaaagccgactatgaaaaacataaagtttacgcatgtgaggtgacgcaccaaggattatccagtccggtcacaaaatcgtttaaccgcggtgagtgt

SEQ ID NO: 42 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPPTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 4 aa, synthetic construct
GGFG

SEQ ID NO: 44 - protein, 4 aa, synthetic construct
EGGF

SEQ ID NO: 45 - protein, 4 aa, synthetic construct
SGGF

SEQ ID NO: 46 - protein, 4 aa, synthetic construct
KGGF